(19) United States
(12) Patent Application Publication
(10) Pub. No.: US 2003/0176368 A1
(43) Pub. Date: Sep. 18, 2003

(54) Synthesis of epothilones, intermediates thereto and analogues thereof

(76) Inventors: Samuel J. Danishefsky, Englewood, NJ (US); Kaustav Biswas, Thouand Oaks, NY (US); Mark Chappell, Noblesville, IN (US); Hong Lin, New York, NY (US); Jon T. Njardarson, New York, NY (US); Chul Bom Lee, Princeton, NJ (US); Alexy Rivkin, New York, NY (US); Ting-Chao Chou, Paramus, NJ (US)

Correspondence Address: Choate, Hall & Stewart, Exchange Place, 53 State Street, Boston, MA, 02109, US

(21) Appl. No.: 10/236,135

(22) Filed: Sep. 6, 2002

Related U.S. Application Data
(60) Provisional application No. 60/317,637, filed on Sep. 6, 2001.
(60) Provisional application No. 60/351,576, filed on Oct. 26, 2001.

(51) Int. Cl. C07H 17/08; C 07D 4 9/02; A61K 31/7048; A61K 31/427; A61K 31/38; A61K 31/365; C07D 417/02

(52) U.S. Cl. 514/28; 514/365; 514/183; 514/431; 514/450; 536/7.4; 548/203; 540/451; 549/9; 549/266

(57) ABSTRACT

The present invention provides compounds of formula (I):

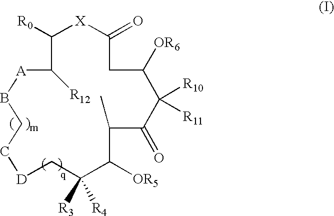

as described generally and in classes and subclasses herein. The present invention additionally provides pharmaceutical compositions comprising compounds of formula (I) and provides methods of treating cancer comprising administering a compound of formula (I).

### PRIORITY INFORMATION

The present application claims priority under 35 U.S.C. §119(e) to co-pending provisional applications Nos. 60/317,637, filed Sep. 6, 2001, entitled “Synthesis of Epothilones, Intermediates Thereto and Analogues Thereof”, and 60/351,576, filed Oct. 26, 2001, entitled “Synthesis of Epothilones, Intermediates Thereto and Analogues Thereof”, the entire contents of which are incorporated herein by reference

### GOVERNMENT SUPPORT

The invention was supported in part by Grant CA-28824 from the National Institutes of Health and by Postdoctoral Fellowships for Chulbom Lee (U.S. Army, Grant DAMD 17-98-1-8155), Mark D Chappell (NIH, Grant F32GM199721), Kaustav Biswas (U.S Army, Grant DAMD 17-98-1-8155), Hong Lin (Cancer Pharmacology Training Grant T32-CA62948-07), and Alexey Rivkin (Cancer Pharmacology Training Grant NIH-TEW-CA62948-07). The U.S. government may have certain rights in this invention.

### BACKGROUND OF THE INVENTION

Epothilones A and B (2a and 2b, Scheme 1) are naturally occurring cytotoxic macrolides that were isolated from a cellulose degrading mycobacterium, Sorangium cellulosum (Höfle et al. Angew. Chem., Int. Ed. Engl. 1996, 35, 1567 and J. Antibiot. 1996, 49, 560; incorporated herein by reference). Despite their vastly different structures, epothilones A and B share the same mechanism of action as paclitaxel (Taxol®) which involves growth inhibition of tumor cells by tubulin polymerization and stabilization of microtubule assemblies (Bollag et al. Cancer Res. 1995, 55, 2325; incorporated herein by reference). In spite of its unquestioned clinical value as the front-line chemotherapeutic agent, Taxol® is far from an ideal drug. Its marginal water solubility necessitates recourse to formulation vehicles such as cremophores that pose their own risks and management issues (Essayan et al. J. Allergy Clin. Immunol. 1996, 97, 42; incorporated herein by reference). Moreover, Taxol® is vulnerable to deactivation through multiple drug resistance (MDR) mechanism (Giannakakou et al. J. Biol. Chem. 1997, 272, 17118; incorporated herein by reference). By comparison, epothilones A and B have been shown to possess a greater therapeutic profile. In particular, it has been demonstrated that epothilones A and B retain remarkable potency against MDR tumor cells (Kowalski et. al Mol. Biol. Cell 1995, 6, 2137; incorporated herein by reference). Additionally, the increased water solubility in comparison to paclitaxel may be useful for the formulability of epothilones. While the naturally occurring compound, epothilone B (2b, EpoB, in Scheme 1), is the most potent member of this family, it unfortunately possesses, at least in xenograft mice, a worrisomely narrow therapeutic index (Su et al. Angew. Chem. Int. Ed. Engl. 1997, 36, 1093; Harris et al. J. Org. Chem. 1999, 64, 8434; incorporated herein by reference).

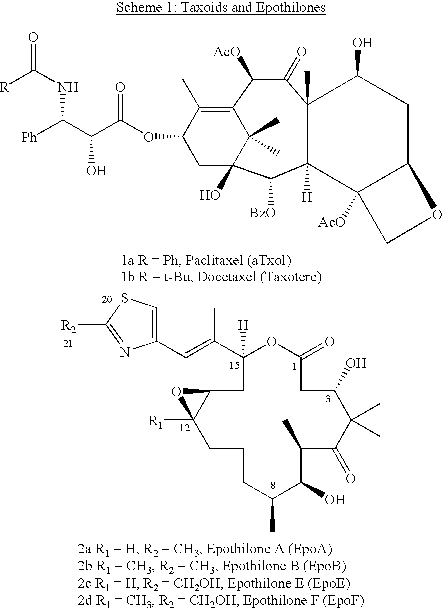

Given the limited therapeutic index of EpoB, another class of compounds, the 12,13-desoxy compounds, was investigated for their ability to provide an improved therapeutic profile (see, U.S. Pat. Nos. 6,242,469, 6,284,781, 6,300,355, 6,369,234, 6,204,388, 6,316,630; each of which is incorporated herein by reference). In vivo experiments conducted on various mouse models demonstrated that 12,13-desoxyepothilone B (3b, dEpoB in Scheme 2) possesses therapeutic potential against various sensitive and resistant human tumors in mice xenografts (Chou et al. Proc. Natl. Acad. Sci. U.S.A. 1998, 95, 9642 and 15798; incorporated herein by reference). Recently, the therapeutic superiority of these desoxyepothilones over other anticancer agents has been demonstrated by thorough comparative studies (Chou et al. Proc. Natl. Acad. Sci. U.S.A. 2001, 98, 8113; incorporated herein by reference). Due to its impressive in vivo profile, dEpoB has been advanced through toxicology evaluations in dogs, in expectation of human trials anticipating its deployment as an anticancer drug.

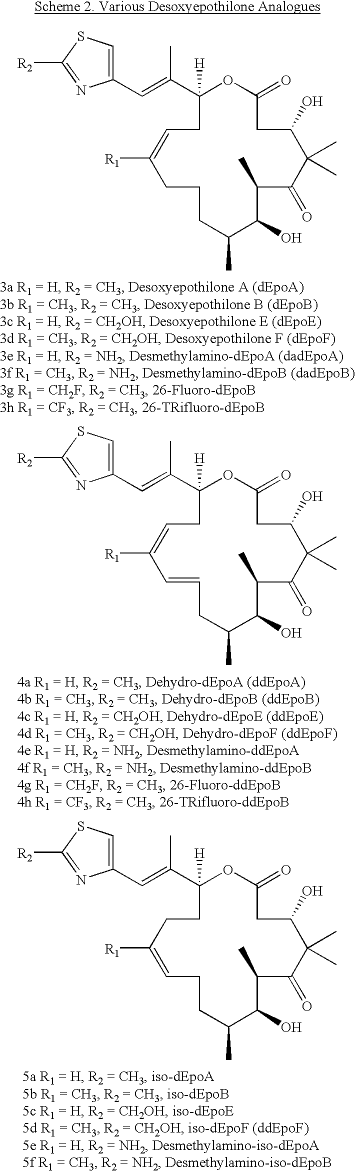

Despite the promising therapeutic utility of the 12,13-desoxyepothilones, it would be desirable to investigate additional analogues as well as additional synthetic methodologies for the synthesis of existing epothilones, desoxyepothilones and analogues thereof, as well as novel analogues thereof. In particular, given the interest in the therapeutic utility of this class of compounds, it would also be desirable to develop methodologies capable of providing signficant quantities of any epothilones or desoxyepothilones previously described, or those described herein, for clinical trials and for large-scale preparation.

### DESCRIPTION OF THE DRAWINGS

FIG. 1 depicts an exemplary synthesis of a thiazolyl-containing western fragment.

FIG. 2 depicts the synthesis of chiral aldehydes (8a), (8b) and (8c).

FIG. 3 depicts exemplary syntheses of an intermediate.

FIG. 4 depicts the conversion of exemplary alkyl intermediates to different macrocyclization precursors.

FIGS. 5A and 5B depict exemplary substrates for macrocyclization via the aldol route.

FIGS. 6A and 6B depict exemplary substrates for the macrocyclization via the acylation route.

FIG. 7 depicts various exemplary macrocyclization methods.

FIG. 8 depicts various exemplary macrocyclization methods.

FIG. 9 depicts the synthetic route for Epo-490 and dEpoB via acylation.

FIG. 10 depicts the synthetic route for Epo-490 via aldol condensation.

FIG. 11 depicts the synthetic route for 21-hydroxy Epo-490.

FIGS. 12A and 12B depict the synthetic route for 26-CF₃ Epothilone D.

FIG. 13 depicts the synthesis of analogues of Epo-490.

FIG. 14 depicts tumor size in nude mice bearing human mammary carcinoma MX-1 following Epo 490, or dEpoB treatment (32 days).

FIG. 15 depicts body weight in nude mice bearing human mammary carcinoma MX-1 following Epo 490, or dEpoB treatment (32 days).

FIG. 16 depicts depicts tumor size in nude mice bearing human mammary carcinoma MX-1 following Epo 490, or dEpoB treatment (50 days).

FIG. 17 depicts body weight in nude mice bearing human mammary carcinoma MX-1 following Epo 490, or dEpoB treatment (50 days).

FIG. 18 depicts an exemplary synthesis of Homo-Epo-490.

FIG. 19 depicts exemplary syntheses of fragments used in the synthesis of epothilones and desoxyepothilones.

FIG. 20 depicts an exemplary synthesis of dEpoB.

FIG. 21 illustrates the increased stability of Epo490 in human versus nude mice plasma. dEpoB in murine plasma is shown as a comparison. See Chou et al. Proc. Natl. Acad. Sci. USA 98:8113, 2001, incorporated herein by reference, for details.

FIG. 22 depicts an exemplary synthesis of 27-trifluoro-[17]EpoD-490.

FIG. 23 depicts an examplary synthesis of the lactam version of Epo490 using the ring closing metathesis route.

FIG. 24 depicts an exemplary synthesis of epothilones via a ring closing metathesis route.

FIG. 25 shows a comparison of the IC₅₀ of various epothilones in CCRF-CEM cell lines. Data for taxol, VP-16, and VBL are shown for comparison.

FIG. 26 is a table of IC₅₀ values for Epothilones against CCRF-CEM cell growth.

FIGS. 27A-D shows the relative cytotoxicity of epothilones against human leukemic cell in vitro. The numbers in parentheses (x) are IC₅₀ values in CCRF-CEM sensitive cell lines; the numbers in square brackets [x] are IC₅₀ values in CCRF-CEM/VBL resistant cells lines; and the numbers in curvy brackets {x} are IC₅₀ values in CCRF-CEM/Taxol resistant cell lines.

FIG. 28 depicts the therapeutic effect of 4-desmethyl EpoB in nude mice bearing human mammary carcinoma MX-1 xenograft.

FIG. 29 depicts the body weight changes of human mammary carcinoma (MX-1) tumor xenograft bearing nude mice following treatment with 4-desmethyl EpoB.

FIG. 30 depicts the therapeutic effect of oxazole-Epo490 in nude mice bearing human colon carcinoma HCT-116 xenograft.

FIG. 31 depicts the body weight changes of HCT-116 xenograft bearing nude mice following treatment with oxazole-Epo490.

FIG. 32 depicts the therapeutic effect of oxazole EpoD and oxazole EpoB in nude mice bearing human colon carcinoma HCT-116 xenograft.

FIG. 33 depicts the body weight change of human colon carcinoma HCT-116 tumor xenograft bearing nude mice following treatment with oxazole-EpoD and oxazole-EpoB.

FIG. 34 depicts the therapeutic effect of dEpoB and 14-OH-EpoD in nude mice bearing MX-1 xenograft.

FIG. 35 depicts the therapeutic effect of dEpoB nad 14-OH-EpoD in nude mice bearing MX-1 xenograft.

FIG. 36 depicts the therapeutic effect of 12-ethyl-dEpo (26-methyl-EpoD) and 14-methyl EpoD against MX-1 xenograft in nude mice with respect to tumor size.

FIG. 37 depicts the therapeutic effect of 12-ethyl-dEpo (26-methyl-EpoD) and 14-methyl EpoD against MX-1 xenograft in nude mice with respect to body weight.

FIG. 38 depicts an exemplary synthesis of 4-desmethyl analogues.

FIG. 39 depicts an exemplary synthesis of epothilones analogues with substituents at C-14 (FIG. 39A) and analogues with substitutents at both C-14 and C-11 (FIG. 39B).

FIG. 40 depicts an exemplary synthesis of epothilone analogues with a benzthiazole substituent at C-15.

### DESCRIPTION OF THE INVENTION

The present invention provides novel epothilones and novel synthetic methodologies enabling access to such epothilones having a broad range of biological and pharmacological activity. In certain embodiments, the inventive compounds are useful in the treatment of cancer.

1) General Description of Compounds of the Invention

The compounds of the invention include compounds of the general formula (I) as further defined below:

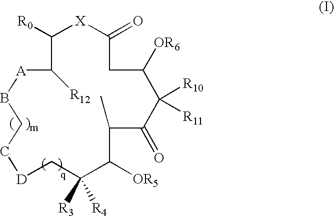

wherein R₀ is a substituted or unsubstituted aryl, heteroaryl, arylalkyl, arylalkenyl, arylalkynyl, heteroarylalkyl, heteroarylalkenyl, or heteroarylalkynyl moiety; in certain embodiments, R₀ is a arylalkyl, arylalkenyl, heteroarylalkyl, or heteroarylalkenyl moiety; in other embodiments, R₀ is a heteroarylalkenyl moiety; in certain embodiments, R₀ is a heteroarylalkyl moiety; in other embodiments, R₀ is a 5-7 membered aryl or heteroaryl moiety; in yet other embodiments, R₀ is an 8-12 membered bicyclic aryl or heteroaryl moiety; in still other embodiments, R₀ is a bicyclic moiety wherein a phenyl ring is fused to a heteroaryl or aryl moiety; in other embodiments, R₀ is a bicylic moiety wherein a phenyl ring is fused to a thiazole, oxazole, or imidazole moiety; in yet other embodiments, R₀ is a subsituted or unsubstituted phenyl moiety.

R₃ and R₄ are each independently hydrogen; or substituted or unsubstituted, linear or branched, cyclic or acyclic aliphatic, heteroaliphatic, aryl, heteroaryl, arylalkyl, arylalkenyl, arylalkynyl, heteroarylalkyl, heteroarylalkenyl, or heteroarylalkynyl moiety, optionally substituted by one or more of hydroxy, protected hydroxy, alkoxy, carboxy, carboxaldehyde, linear or branched alkyl or cyclic acetal, fluorine, amino, protected amino, amino substituted with one or two alkyl or aryl moieties, N-hydroximino, or N-alkoxyimino; in certain embodiments, R₃ and R₄ are each independently hydrogen, fluorine, or lower alkyl; in other other embodiments, R₃ and R₄ are each independently hydrogen or methyl; in still another emobidments, R₃ is methyl, and R₄ is hydrogen.

R₅ and R₆ are each independently hydrogen or a protecting group; in certain embodiments, R₅ and R₆ are both hydrogen;

R₁₀ and R₁₁ are each independently hydrogen; or substituted or unsubstituted, linear or branched, cyclic or acyclic aliphatic, heteroaliphatic, aryl, heteroaryl, arylalkyl, arylalkenyl, arylalkynyl, heteroarylalkyl, heteroarylalkenyl, or heteroarylalkynyl moiety, optionally substituted by one or more of hydroxy, protected hydroxy, alkoxy, carboxy, carboxaldehyde, linear or branched alkyl or cyclic acetal, fluorine, amino, protected amino, amino substituted with one or two alkyl or aryl moieties, N-hydroximino, or N-alkoxyimino; in certain embodiments, R₁₀ and R₁₁ are each independently hydrogen, fluorine, or lower alkyl; in other embodiments, R₁₀ and R₁₁ are each independently hydrogen or methyl; in still other embodiments, R₁₀ and R₁₁ are both methyl; in yet another emboidments, one of R₁₀ and R₁₁ is hydrogen and the other is methyl;

R₁₂ is hydrogen; halogen, hydroxy, alkoxy, amino, dialkylamino, alkylamino, fluoro, or substituted or unsubstituted, linear or branched, cyclic or acyclic aliphatic, heteroaliphatic, aryl, heteroaryl, arylalkyl, arylalkenyl, arylalkynyl, heteroarylalkyl, heteroarylalkenyl, or heteroarylalkynyl moiety, optionally substituted by one or more of hydroxy, protected hydroxy, alkoxy, carboxy, carboxaldehyde, linear or branched alkyl or cyclic acetal, fluorine, amino, protected amino, amino substituted with one or two alkyl or aryl moieties, N-hydroximino, or N-alkoxyimino; in certain embodiments, R₁₂ is hydrogen, halogen, hydroxy, amino, or substituted or unsubstituted, linear or branched, cyclic or acyclic aliphatic or heteroaliphatic; in other embodiments, R₁₂ is fluorine; in other embodiments, R₁₂ is methyl; in yet other embodiments, R₁₂ is hydroxy; in still other embodiments, R₁₂ is hydrogen.

X is O, S, C(R₇)₂, or NR₇, wherein each occurrence of R₇ is independently hydrogen or lower alkyl; in certain embodiments, X is O; in other embodiments, X is NH;

m is an integer from 0 to 3 and q is an integer from 1 to 3 with the limitation that the sum of m+q is an integer from 1 to 4; in certain embodiments, the sum of m+q is an integer from 2 to 4; in other embodiments, the sum of m+q is 1;

A—B represents CR_{A}═CR_{B}—; C(R_{A})₂—C(R_{B})₂—; or C(R_{A})₂—CR_{B}═;

C—D represents —CR_{C}═CR_{D—}; —C(R_{C})₂—C(R_{D})₂—; ═CR_{C}—C(R_{D})₂—; or —C≡C—;

when m is 0, B—C represents ═CR_{B}—CR_{C}═; —C(R_{B})₂—CR_{C}═; ═CR_{B}—C(R_{C})₂—; ═CR_{B}—C≡; or —C(R_{B})₂—C(R_{C})₂—;

wherein each occurrence of R_{A} is independently hydrogen; halogen; —OR_{A′}; —SR_{A′}; —N(R_{A′})₂; —C(O)OR_{A′}; —C(O)R_{A′}; —CONHR_{A′}; —O(C═O)R_{A′}; —O(C═O)OR_{A′}; —NR_{A′}(C═O)R_{A′}; N₃; N₂R_{A′}; cyclic acetal; or cyclic or acyclic, linear or branched aliphatic, heteroaliphatic, aryl, or heteroaryl, optionally substituted with one or more of hydrogen; halogen; —OR_{A′}; —SR_{A′}; —N(R_{A′})₂; —C(O)OR_{A′}; —C(O)R_{A′}; —CONHR_{A′}; —O(C═O)R_{A′}; —O(C═O)OR_{A′}; —NR_{A′}(C═O)R_{A′}; N₃; N₂R_{A′}; cyclic acetal; or cyclic or acyclic, linear or branched substituted or unsubstituted aliphatic, heteroaliphatic, aryl, or heteroaryl moiety; or is an epothilone, desoxyepothilone, or analogues thereof; or is a polymer; carbohydrate; photoaffinity label; or radiolabel;

R_{B} is, independently for each occurrence, hydrogen; halogen; —OR_{B′}; —SR_{B′}; —N(R_{B′})₂; —C(O)OR_{B′}; —C(O)R_{B′}; —CONHR_{B′}; —O(C═O)R_{B′}; —O(C═O)OR_{B′}; —NR_{B′}(C═O)R_{B′}; N₃; N₂R_{B′}; cyclic acetal; or cyclic or acyclic, linear or branched aliphatic, heteroaliphatic, aryl, or heteroaryl, optionally substituted with one or more of hydrogen; halogen; —OR_{B′}; —SR_{B′}; —N(R_{B′})₂; —C(O)OR_{B′}; —C(O)R_{B′}; —CONHR_{B′}; —O(C═O)R_{B′}; —O(C═O)OR_{B′}; —NR_{B′}(C═O)R_{B′}; N₃; N₂R_{B′}; cyclic acetal; or cyclic or acyclic, linear or branched substituted or unsubstituted aliphatic, heteroaliphatic, aryl, or heteroaryl moiety; or is an epothilone, desoxyepothilone, or analogues thereof; or is a polymer; carbohydrate; photoaffinity label; or radiolabel; in certain embodiments,:R_{B} is hydrogen,

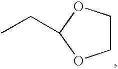

methyl, ethyl, propyl, butyl, pentyl, hexyl, cyclopropyl, cyclobutyl, cyclopentyl, or cyclohexyl, each unsubstituted or optionally substituted with one or more occurrences of halogen, —OH, —OR_{B′}, NH₂, or N(R_{B′})₂, or any combination thereof, wherein each occurrence of R_{B′} is independently hydrogen, alkyl, aryl, or a protecting group; in other embodiments, R_{B} is hydrogen, methyl, or ethyl; in still other embodiments, R_{B} is methyl; in yet other embodiments, R_{B} is —CF₃, —CH₂F, or —CHF₂;

R_{C} is, independently for each occurrence, hydrogen; halogen; —OR_{C′}; —SR_{C′}; —N(R_{C′})₂; —C(O)OR_{C′}; —C(O)R_{C′}; —CONHR_{C′}; —O(C═O)R_{C′}; —O(C═O)OR_{C′}; —NR_{C′}(C═O)R_{C′}; N₃; N₂R_{C′}; cyclic acetal; or cyclic or acyclic, linear or branched aliphatic, heteroaliphatic, aryl, or heteroaryl, optionally substituted with one or more of hydrogen; halogen; —OR_{C′}; —SR_{C′}; —N(R_{C′})₂; —C(O)OR_{C′}; —C(O)R_{C′}; —CONHR_{C′}; —O(C═O)R_{C′}; —O(C═O)OR_{C′}; —NR_{C′}(C═O)R_{C′}; N₃; N₂R_{C′}; cyclic acetal; or cyclic or acyclic, linear or branched substituted or unsubstituted aliphatic, heteroaliphatic, aryl, or heteroaryl moiety; or is an epothilone, desoxyepothilone, or analogues thereof; or is a polymer; carbohydrate; photoaffinity label; or radiolabel; in certain embodiments, R_{C} is halogen, alkyl, hydroxy, or amino; in other embodiments, R_{C} is fluorine; in yet other embodiments, R_{C} is hydroxy;

R_{D} is, independently for each occurrence, hydrogen; halogen; —OR_{D′}; —SR_{D′}; —N(R_{D′})₂; —C(O)OR_{D′}; —C(O)R_{D′}; —CONHR_{D′}; —O(C═O)R_{D′}; —O(C═O)OR_{D′}; —NR_{D′}(C═O)R_{D′}; N₃; N₂R_{D′}; cyclic acetal; or cyclic or acyclic, linear or branched aliphatic, heteroaliphatic, aryl, or heteroaryl, optionally substituted with one or more of hydrogen; halogen; —OR_{D′}; —SR_{D′}; —N(R_{D′})₂; —C(O)OR_{D′}; —C(O)R_{D′}; —CONHR_{D′}; —O(C═O)R_{D′}; —O(C═O)OR_{D′}; —NR_{D′}(C═O)R_{D′}; N₃; N₂R_{D′}; cyclic acetal; or cyclic or acyclic, linear or branched substituted or unsubstituted aliphatic, heteroaliphatic, aryl, or heteroaryl moiety; or is an epothilone, desoxyepothilone, or analogues thereof; or is a polymer; carbohydrate; photoaffinity label; or radiolabel; or

wherein any two of R_{A}, R_{B}, R_{C} or R_{D} taken together may form a cyclic moiety and may be linked through an oxygen, sulfur, carbon or nitrogen atom, or any two adjacent groups R_{A}, R_{B}, R_{C}, or R_{D}, taken together, may form a 3-6-membered substituted or unsubstituted aliphatic, heteroaliphatic, aryl or heteroaryl ring;

wherein each occurrence of R_{A′}, R_{B′}, R_{C′} and R_{D′} is independently hydrogen; a protecting group; a linear or branched, substituted or unsubstituted, cyclic or acyclic, aliphatic, heteroaliphatic, aryl, heteroaryl, arylalkyl, arylalkenyl, arylalkynyl, arylalkenyl, arylalkynyl, or heteroarylalkyl, heteroarylalkenyl, heteroarylalkynyl moiety; or is an epothilone, desoxyepothilone, or analogues thereof; or a polymer; carbohydrate; photoaffinity label; or radiolabel; and pharmaceutically acceptable derivatives thereof.

In certain embodiments, the compounds of formula (I′) have the stereochemistry as indicated in formula (I′):

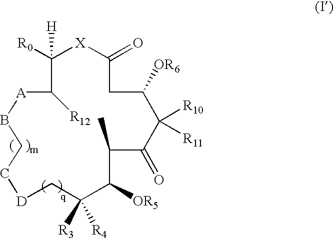

wherein R₀, R₃, R₄, R₅, R₆, R₁₀, R₁₁, R₁₂, A, B, C, D, X, m, and q are as previously defined.

In another embodiment, the compounds wherein R₀ is further defined are of the formula (I″):

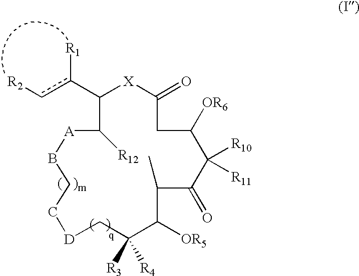

wherein R₁ is hydrogen, lower alkyl, or in conjunction with R₂ may form a cyclic, heterocyclic, aryl, or heteroaryl moitey; in certain embodiments, R₁ is methyl;

R₂ is a substituted or unsubstituted aryl, heteroaryl, arylalkyl, arylalkenyl, arylalkynyl, arylalkenyl, arylalkynyl, heteroarylalkyl, heteroarylalkenyl, or heteroarylalkynyl moiety, which may in conjunction with R₁ form a cyclic, heterocyclic, aryl, or heteroaryl moiety;

the dashed line represents a bond or the absence of a bond; and

R₃, R₄, R₅, R₆, R₁₀, R₁₁, R₁₂, A, B, C, D, X, m, and q are as previously defined. In certain embodiments, R₁ and R₂ in conjunction may form a 5-7-membered monocyclic moiety or a 8-12-membered bicyclic moiety. In other embodiments, R₁ and R₂ in conjunction form a 5-7 membered heterocyclic moiety or a 8-12-membered biheterocyclic moiety. In yet other embodiments, R₁ and R₂ in conjunction form a bicyclic moiety in which a benzylic ring is fused to an aryl or heteroaryl moiety.

In certain embodiments, compounds as described above and/or in subclasses herein inlcude those compounds wherein R₀ or R₁ and R₂ in conjuction may be:

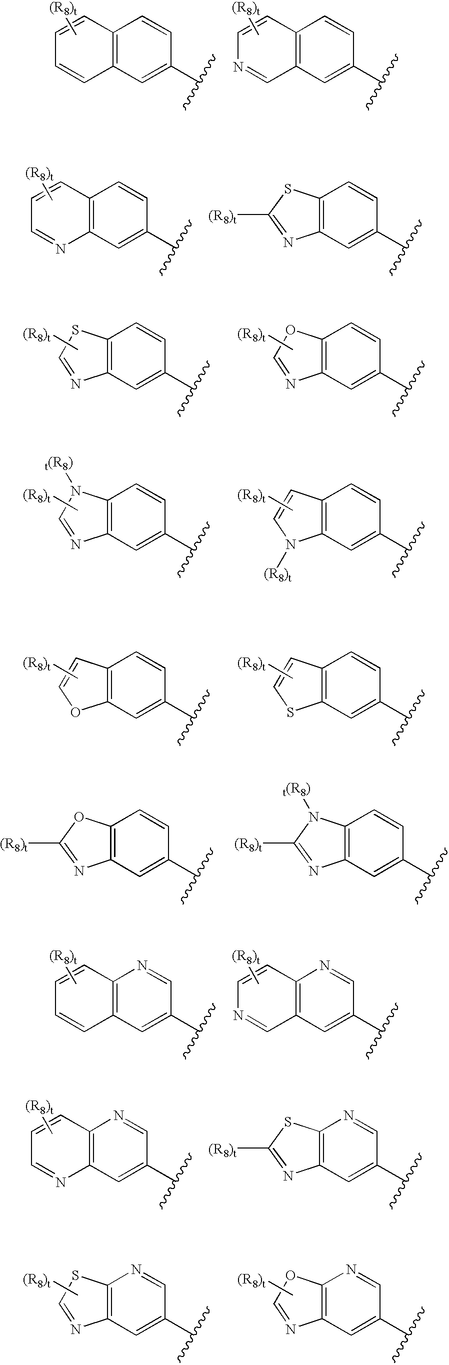

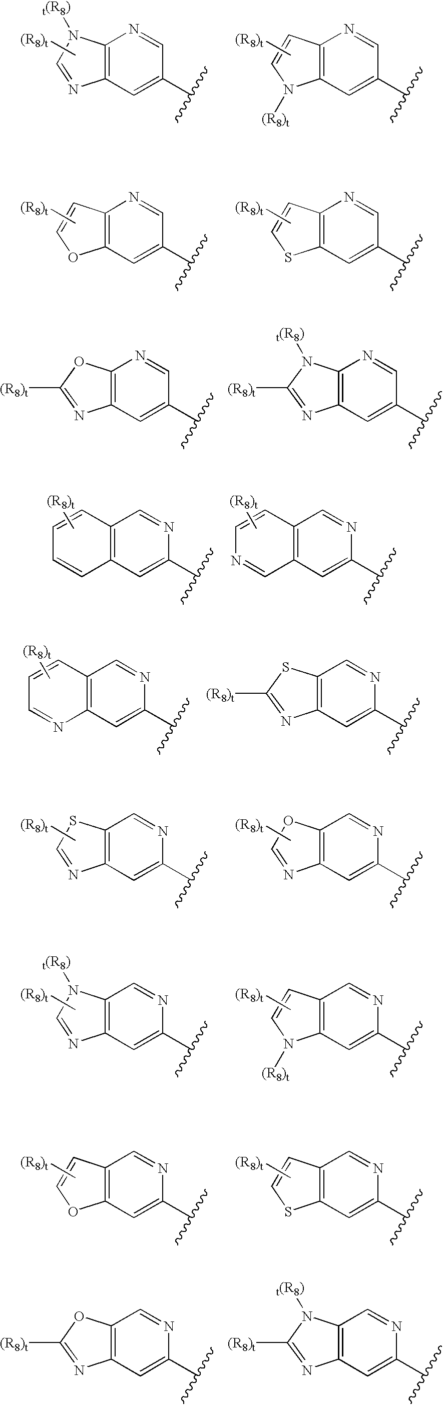

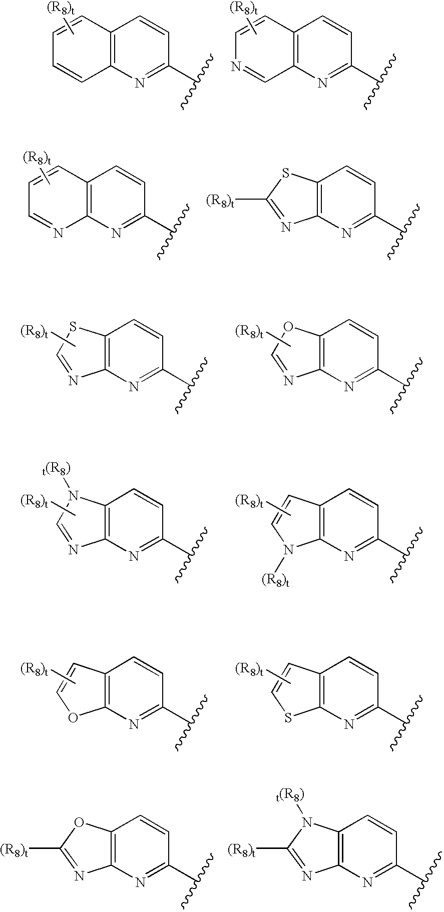

wherein each occurrence of R₈ is independently hydrogen, halogen, —OR₉, —SR₉, —N(R₉)₂, —(CV₂)ₙOR₉, —(CV₂)ₙN(R₉)₂, —(CV₂)ₙSR₉, —(C═O)R₉, —O(C═O)R₉, —(C═O)OR₉, —O(C═O)OR₉; —NH(C═O)R₉, —NH(C═O)OR₉, —(C═O)NHR₉, or a cyclic or acyclic, linear or branched aliphatic, heteroaliphatic, aryl, heteroaryl, arylalkyl, arylalkenyl, arylalkynyl, or heteroarylalkyl, heteroarylalkenyl, heteroarylalkynyl moiety optionally substituted with one or more occurrences of halogen, —OR₉, —SR₉, —N(R₉)₂, —(CV₂)ₙOR₉, —(CV₂)ₙN(R₉)₂, —(CV₂)ₙSR₉, —(C═O)R₉, —O(C═O)R₉, —(C═O)OR₉, —O(C═O)OR₉;—NH(C═O)R₉, —NH(C═O)OR₉, —(C═O)NHR₉, or a cyclic or acyclic, linear or branched, substituted or unsubstituted aliphatic, heteroaliphatic, aryl, heteroaryl, arylalkyl, arylalkenyl, arylalkynyl, or heteroarylalkyl, heteroarylalkenyl, heteroarylalkynyl moiety,

wherein each occurrence of R₉ is independently hydrogen; a protecting group; a cyclic or acyclic, linear or branched, substituted or unsubstituted aliphatic, heteroaliphatic, aryl, or heteroaryl moiety; or is an epothilone, desoxyepothilone or analogues thereof; a polymer; carbohydrate; photoaffinity label; or radiolabel;

wherein each occurrence of V is independently hydrogen, halogen, hydroxyl, thio, amino, alkylamino, or protected hydroxyl, thio or amino; each occurrence of t is independently 0, 1 or 2; and each occurrence of n is independently 0-10.

Alternatively, R₁ and R₂ are not joined in a ring structure so that compounds are of the formula (I′″):

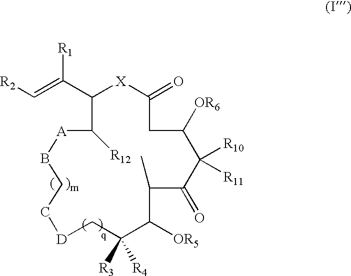

wherein R₁ is hydrogen or lower alkyl;

R₂ is a substituted or unsubstituted aryl, heteroaryl, arylalkyl, arylalkenyl, arylalkynyl, arylalkenyl, arylalkynyl, heteroarylalkyl, heteroarylalkenyl, or heteroarylalkynyl moiety; and

R₃, R₄, R₅, R₆, R₁₀, R₁₁, R₁₂, A, B, C, D, X, m, and q are as previously defined.

In certain embodiments, the compounds of the formula (I′″) have the stereochemistry as indicated in formula (I″″):

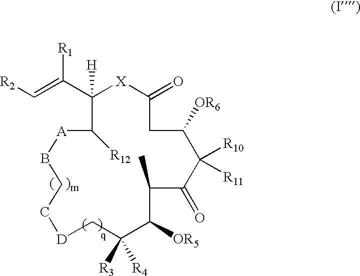

wherein R₁, R₂, R₃, R₄, R₅, R₆, R₁₀, R₁₁, R₁₂, A, B, C, D, X, m, and q are as previously defined.

In certain embodiments, compounds of formula (I)-(I″″) are provided wherein R₁, R₃-R₆, R₁₀, R₁₁, R₁₂, A-D, m, q, and X are as previously defined and R₀ or R₂ is an aryl, heteroaryl, arylalkyl, arylalkenyl, arylalkynyl, heteroarylalkyl, heteroarylalkenyl, or heteroarylalkynyl moiety optionally substituted with one or more occurrences of R₈, wherein each occurrence of R₈ is independently hydrogen, halogen, —OR₉, —SR₉, —N(R₉)₂, —(CV₂)ₙOR₉, —(CV₂)ₙN(R₉)₂, —(CV₂)ₙSR₉, —(C═O)R₉, —O(C═O)R₉, —(C═O)OR₉, —O(C═O)OR₉; —NH(C═O)R₉, —NH(C═O)OR₉, —(C═O)NHR₉, or a cyclic or acyclic, linear or branched aliphatic, heteroaliphatic, aryl, heteroaryl, arylalkyl, arylalkenyl, arylalkynyl, or heteroarylalkyl, heteroarylalkenyl, heteroarylalkynyl moiety optionally substituted with one or more occurrences of halogen, —OR₉, —SR₉, —N(R₉)₂, —(CV₂)ₙOR₉, —(CV₂)ₙN(R₉)₂, —(CV₂)ₙSR₉, —(C═O)R₉, —O(C═O)R₉, —(C═O)OR₉, —O(C═O)OR₉; —NH(C═O)R₉, —NH(C═O)OR₉, —(C═O)NHR₉, or a cyclic or acyclic, linear or branched, substituted or unsubstituted aliphatic, heteroaliphatic, aryl, heteroaryl, arylalkyl, arylalkenyl, arylalkynyl, or heteroarylalkyl, heteroarylalkenyl, heteroarylalkynyl moiety,

wherein each occurrence of R₉ is independently hydrogen; a protecting group; a cyclic or acyclic, linear or branched, substituted or unsubstituted aliphatic, heteroaliphatic, aryl, or heteroaryl moiety; or is an epothilone, desoxyepothilone or analogues thereof; a polymer; carbohydrate; photoaffinity label; or radiolabel;

wherein each occurrence of V is independently hydrogen, halogen, hydroxyl, thio, amino, alkylamino, or protected hydroxyl, thio or amino; and

each occurrence of n is 0-10.

In other embodiments, compounds of formula (I′) and (I″″) are provided wherein R₁, R₃-R₆, R₁₀, R₁₁, R₁₂, A-D, m, q, and X are as previously defined and R₂ is one of:

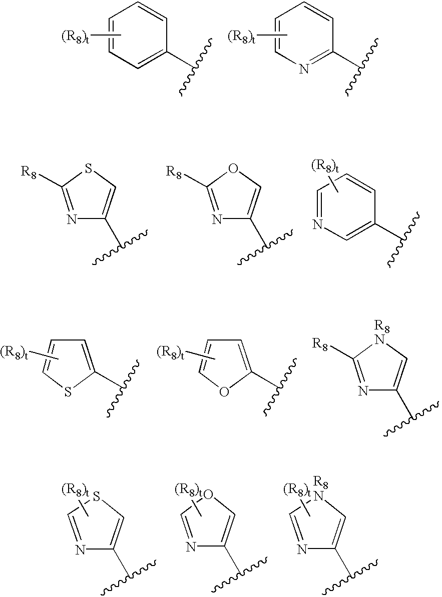

wherein each occurrence of R₈ is independently hydrogen, halogen, —OR₉, —SR₉, —N(R₉)₂, —(CV₂)ₙOR₉, —(CV₂)ₙN(R₉)₂, —(CV₂)ₙSR₉, —(C═O)R₉, —O(C═O)R₉, —(C═O)OR₉, —O(C═O)OR₉; —NH(C═O)R₉, —NH(C═O)OR₉, —(C═O)NHR₉, or a cyclic or acyclic, linear or branched aliphatic, heteroaliphatic, aryl, heteroaryl, arylalkyl, arylalkenyl, arylalkynyl, or heteroarylalkyl, heteroarylalkenyl, heteroarylalkynyl moiety optionally substituted with one or more occurrences of halogen, —OR₉, —SR₉, —N(R₉)₂, —(CV₂)ₙOR₉, —(CV₂)ₙN(R₉)₂, —(CV₂)ₙSR₉, —(C═O)R₉, —O(C═O)R₉, —(C═O)OR₉, —O(C═O)OR₉;—NH(C═O)R₉, —NH(C═O)OR₉, —(C═O)NHR₉, or a cyclic or acyclic, linear or branched, substituted or unsubstituted aliphatic, heteroaliphatic, aryl, heteroaryl, arylalkyl, arylalkenyl, arylalkynyl, heteroarylalkyl, heteroarylalkenyl, or heteroarylalkynyl moiety,

wherein each occurrence of R₉ is independently hydrogen; a protecting group; a cyclic or acyclic, linear or branched, substituted or unsubstituted aliphatic, heteroaliphatic, aryl, or heteroaryl moiety; or is an epothilone, desoxyepothilone or analogues thereof; a polymer; carbohydrate; photoaffinity label; or radiolabel;

wherein each occurrence of V is independently hydrogen, halogen, hydroxyl, thio, amino, alkylamino, or protected hydroxyl, thio or amino; each occurrence of t is independently 0, 1 or 2; and each occurrence of n is independently 0-10.

In yet other embodiments, compounds of formula (I′) and (I″″) are provided wherein R₁, R₃-R₆, R₁₀, R₁₁, R₁₂, A-D, m, q, and X are as previously defined and R₂ is one of:

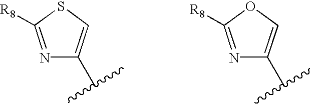

wherein each occurrence of R₈ is independently hydrogen, halogen, —OR₉, —SR₉, —N(R₉)₂, —(CV₂)ₙOR₉, —(CV₂)ₙN(R₉)₂, —(CV₂)ₙSR₉, —(C═O)R₉, —O(C═O)R₉, —(C═O)OR₉, —O(C═O)OR₉, —NH(C═O)R₉, —NH(C═O)OR₉, —(C═O)NHR₉, or a cyclic or acyclic, linear or branched aliphatic, heteroaliphatic, aryl, heteroaryl, arylalkyl, arylalkenyl, arylalkynyl, or heteroarylalkyl, heteroarylalkenyl, heteroarylalkynyl moiety optionally substituted with one or more occurrences of halogen, —OR₉, —SR₉, —N(R₉)₂, —(CV₂)ₙOR₉, —(CV₂)ₙN(R₉)₂, —(CV₂)ₙSR₉, —(C═O)R₉, —O(C═O)R₉, —(C═O)OR₉, —O(C═O)OR₉; —NH(C═O)R₉, —NH(C═O)OR₉, —(C═O)NHR₉, or a cyclic or acyclic, linear or branched, substituted or unsubstituted aliphatic, heteroaliphatic, aryl, heteroaryl, arylalkyl, arylalkenyl, arylalkynyl, or heteroarylalkyl, heteroarylalkenyl, heteroarylalkynyl moiety,

wherein each occurrence of R₉ is independently hydrogen; a protecting group; a cyclic or acyclic, linear or branched, substituted or unsubstituted aliphatic, heteroaliphatic, aryl, or heteroaryl moiety; or is an epothilone, desoxyepothilone or analogues thereof; a polymer; carbohydrate; photoaffinity label; or radiolabel;

wherein each occurrence of V is independently hydrogen, halogen, hydroxyl, thio, amino, alkylamino, or protected hydroxyl, thio or amino; each occurrence of t is independently 0, 1 or 2; and each occurrence of n is independently 0-10.

In still other embodiments, compounds of formula (I′) and (I″″) are provided wherein R₁, R₃-R₆, R₁₀, R₁₁, R₁₂, A-D, m, q, and X are as previously defined and R₂ is one of:

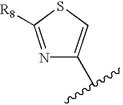

wherein each occurrence of R₈ is independently hydrogen, halogen, —OR₉, —SR₉, —N(R₉)₂, —(CV₂)ₙOR₉, —(CV₂)ₙN(R₉)₂ , —(CV₂)ₙSR₉, —(C═O)R₉, —O(C═O)R₉, —(C═O)OR₉; —O(C═O)OR₉, —NH(C═O)R₉, —NH(C═O)OR₉, —(C═O)NHR₉, or a cyclic or acyclic, linear or branched aliphatic, heteroaliphatic, aryl, heteroaryl, arylalkyl, arylalkenyl, arylalkynyl, or heteroarylalkyl, heteroarylalkenyl, heteroarylalkynyl moiety optionally substituted with one or more occurrences of halogen, —OR₉, —SR₉, —N(R₉)₂, —(CV₂)ₙOR₉, —(CV₂)ₙN(R₉)₂, —(CV₂)ₙSR₉, —(C═O)R₉, —O(C═O)R₉, —(C═O)OR₉, —O(C═O)OR₉; —NH(C═O)R₉, —NH(C═O)OR₉, —(C═O)NHR₉, or a cyclic or acyclic, linear or branched, substituted or unsubstituted aliphatic, heteroaliphatic, aryl, heteroaryl, arylalkyl, arylalkenyl, arylalkynyl, or heteroarylalkyl, heteroarylalkenyl, heteroarylalkynyl moiety,

wherein each occurrence of R₉ is independently hydrogen; a protecting group; a cyclic or acyclic, linear or branched, substituted or unsubstituted aliphatic, heteroaliphatic, aryl, or heteroaryl moiety; or is an epothilone, desoxyepothilone or analogues thereof; a polymer; carbohydrate; photoaffinity label; or radiolabel;

wherein each occurrence of V is independently hydrogen, halogen, hydroxyl, thio, amino, alkylamino, or protected hydroxyl, thio or amino; each occurrence of t is independently 0, 1 or 2; and each occurrence of n is independently 0-10.

In yet other embodiments, compounds of formula (IΔ) and (I″″) are provided wherein R₁, R₃-R₆, R₁₀, R₁₁, R₁₂, A-D, m, q, and X are as previously defined and R₂ is one of:

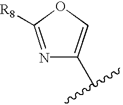

wherein each occurrence of R₈ is independently hydrogen, halogen, —OR₉, —SR₉, —N(R₉)₂, —(CV₂)ₙOR₉, —(CV₂)ₙN(R₉)₂, —(CV₂)ₙSR₉, —(C═O)R₉, —O(C═O)R₉, —(C═O)OR₉, —O(C═O)OR₉; —NH(C═O)R₉, —NH(C═O)OR₉, —(C═O)NHR₉, or a cyclic or acyclic, linear or branched aliphatic, heteroaliphatic, aryl, heteroaryl, arylalkyl, arylalkenyl, arylalkynyl, or heteroarylalkyl, heteroarylalkenyl, heteroarylalkynyl moiety optionally substituted with one or more occurrences of halogen, —OR₉, —SR₉, —N(R₉)₂, —(CV₂)ₙOR₉, —(CV₂)ₙN(R₉)₂, —(CV₂)ₙSR₉, —(C═O)R₉, —O(C═O)R₉, —(C═O)OR₉, —O(C═O)OR₉; —NH(C═O)R₉, —NH(C═O)OR₉, —(C═O)NHR₉, or a cyclic or acyclic, linear or branched, substituted or unsubstituted aliphatic, heteroaliphatic, aryl, heteroaryl, arylalkyl, arylalkenyl, arylalkynyl, or heteroarylalkyl, heteroarylalkenyl, heteroarylalkynyl moiety,

wherein each occurrence of R₉ is independently hydrogen; a protecting group; a cyclic or acyclic, linear or branched, substituted or unsubstituted aliphatic, heteroaliphatic, aryl, or heteroaryl moiety; or is an epothilone, desoxyepothilone or analogues thereof, a polymer; carbohydrate; photoaffinity label; or radiolabel;

wherein each occurrence of V is independently hydrogen, halogen, hydroxyl, thio, amino, alkylamino, or protected hydroxyl, thio or amino; each occurrence of t is independently 0, 1 or 2; and each occurrence of n is independently 0-10.

In another embodiment, the compounds of the invention include compounds of the general formula (II) as further defined below:

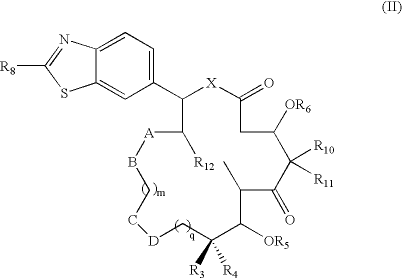

wherein R₃, R₄, R₅, R₆, R₈, R₁₀ R₁₁, R₁₂, X, A-D, m and q are as previously defined.

In certain embodiments, the compounds of formula (II) have the stereochemistry as indicated in formula (II′):

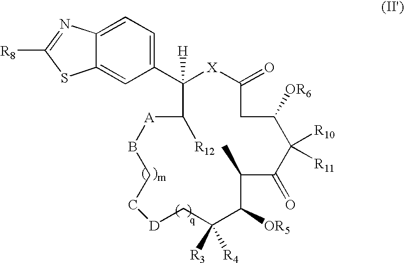

wherein R₃, R₄, R₅, R₆, R₈, R₁₀, R₁₁, R₁₂, X, A-D, m and q are as previously defined.

In certain other embodiments, compounds as described above and/or in subclasses herein are provided wherein R₈ is —CH₃, —CH₂OH, or —CH₂NH₂.

In certain other embodiments, compounds as described above and in subclasses herein are provided wherein when A—B is —C(R_{A})═C(R_{B})—, the double bond is in the Z configuration. In certain other embodiments, compounds as described above and in subclasses herein are provided wherein when C—D is —C(R_{C})═C(R_{D})—, the double bond is in the E configuration.

In certain embodiments, compounds as described above and in subclasses herein are provided wherein when A—B is a carbon-carbon double bond or an epoxide, and R_{B} is a hydrogen or methyl, then R_{A}, R_{C}, or R_{D} is a moiety other than H.

2) Featured Classes of Compounds

It will be appreciated that for compounds as generally described above, certain classes of compounds are of special interest. As one of skill in the art would appreciate, the provisions for each of the variables of the inventive compounds as described herein may be mixed and matched to yield various subclasses of compounds. These subclasses of compounds as would be appreciated by one of skill in this art can be prepared using any of the methods described herein or using methods described in the art. The following featured classes of compound are only exemplary and are not meant to be limiting as to the various subclasses of compounds described herein. For example, one class of compounds of special interest includes those compounds of the invention as described above and herein, wherein q is 1, m is 0, 1, 2, or 3, and A—B represents —CR_{A}═CR_{B} and C—D represents —CR_{C}═CR_{D}— and the compound has the structure:

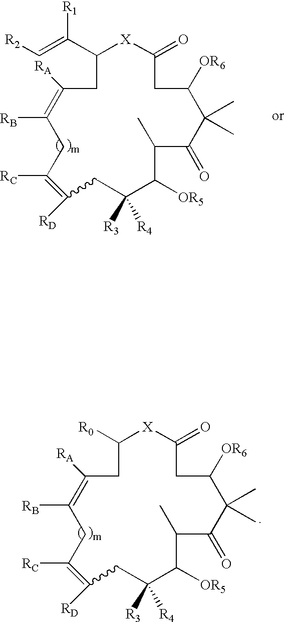

Another class of compounds of special interest includes those compounds of the invention as described above and herein, wherein m is 1, q is 1 and A—B represents —CR_{A}═CR_{B} and C—D represents —CR_{C}═CR_{D}— and the compound has the structure:

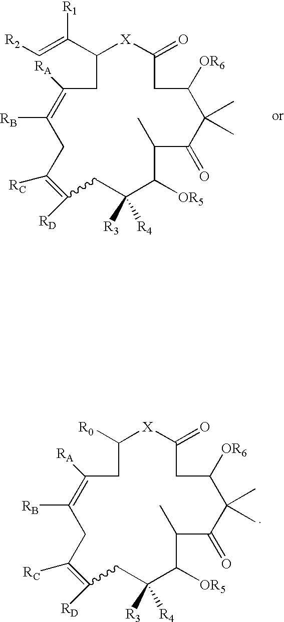

Still another class of compounds of special interest includes those compounds of the invention as described above and herein, wherein m is 2, q is 1 and A—B represents —CR_{A}═CR_{B} and C—D represents —CR_{C}═CR_{D}— and the compound has the structure:

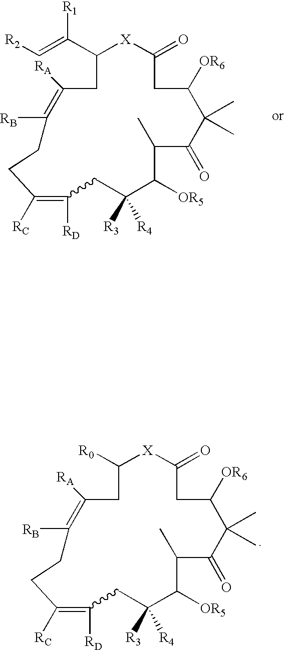

Yet another class of compounds of special interest includes those compounds of the invention as described above and herein, wherein m is 3, q is 1 and A—B represents —CR_{A}═CR_{B} and C—D represents —CR_{C}═CR_{D}— and the compound has the structure:

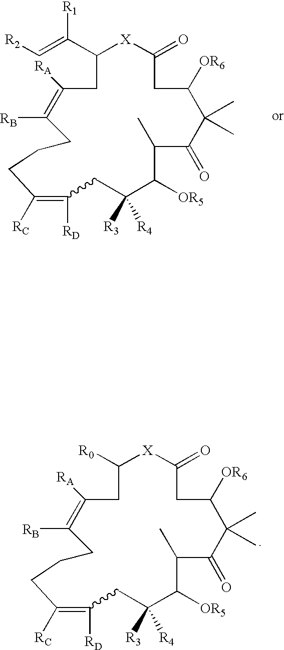

Yet another class of compounds of special interest includes those compounds of the invention as described above and herein, whereini m is 0, q is 1, A—B represents —CR_{A}═CR_{B}— and C—D represents —CR_{C}═CR_{D}— and the compound has the structure:

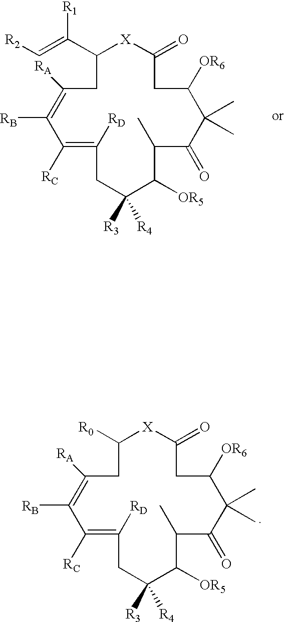

The following structures illustrate several exemplary types of compounds of these classes. Others will be readily apparent to the reader:

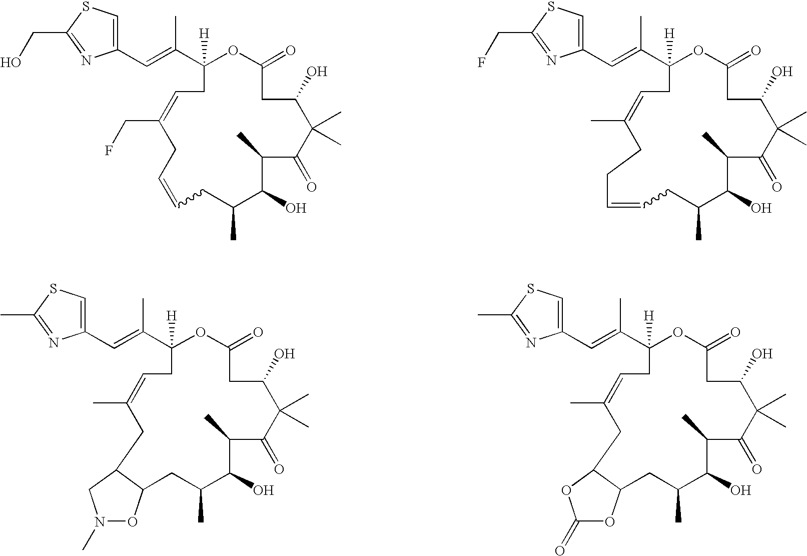

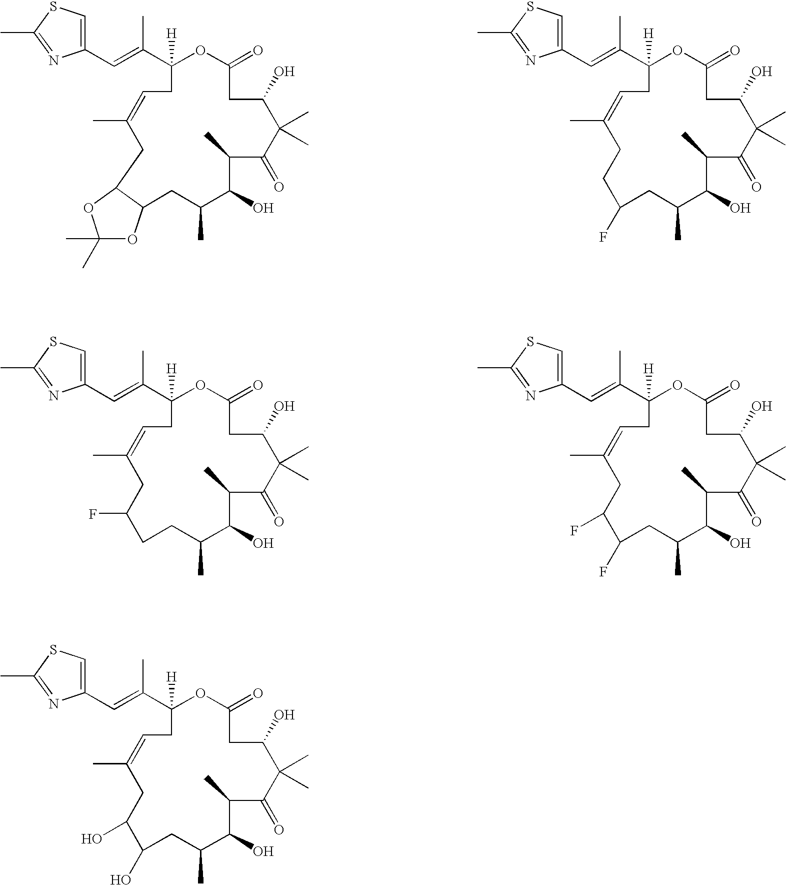

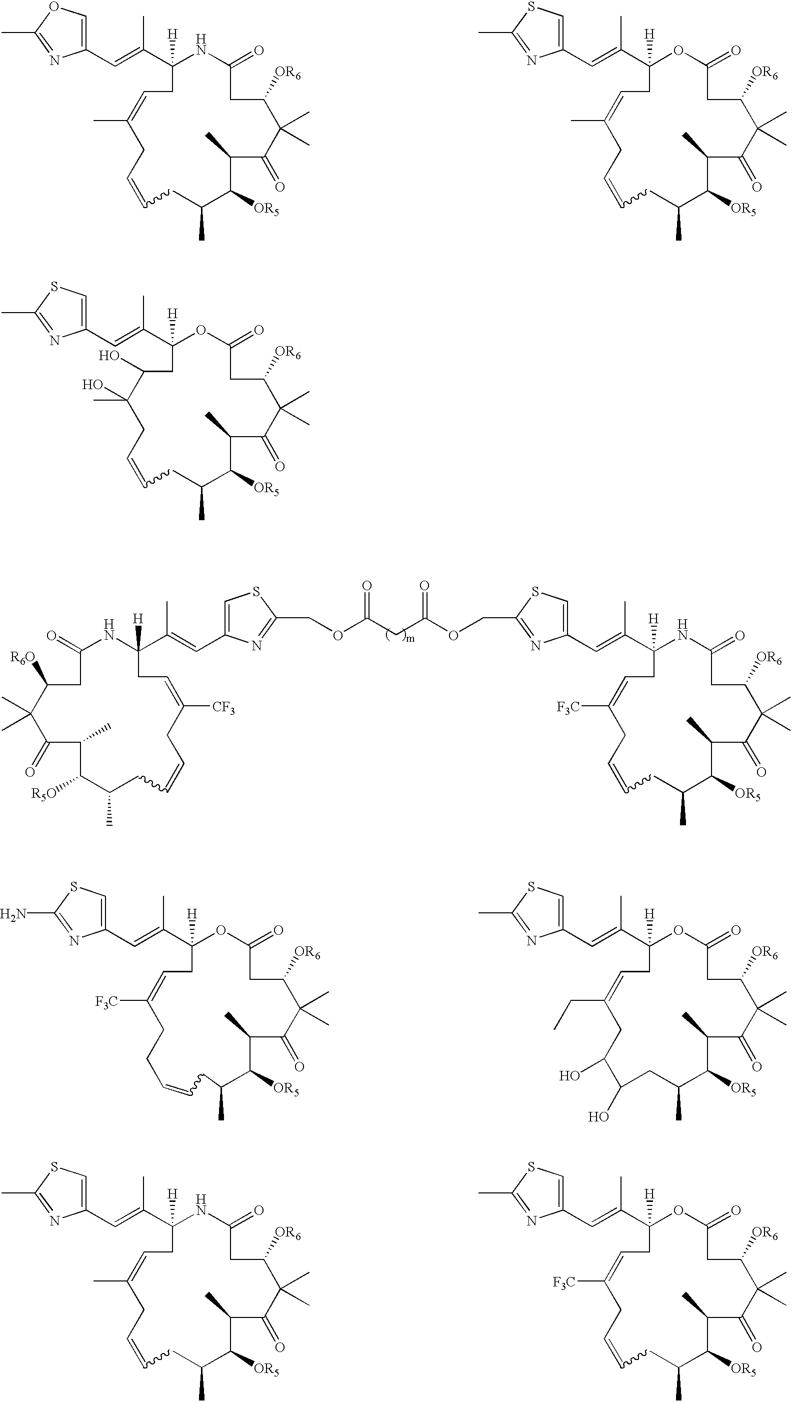

A number of important subclasses of each of the foregoing classes, and each of the other classes of compounds described herein (e.g. intermediates (F), (G), (H), (I) and (J), and subclasses thereof, as described in more detail in the Synthetic Methodology section herein) deserve separate mention; these subclasses include subclasses of the foregoing classes in which:

i) X is O or NH;

ii) X is O;

iii) R₃ is methyl and R₄ is hydrogen;

iv) R₅ and R₆ are both hydrogen;

v) one or both of R₅ and R₆ are an oxygen protecting group;

vi) one or both of R₅ and R₆ are hydrogen, t-butyldimethylsilyl, triethylsilyl, triisobutylsilyl, triisopropylsilyl, trimethylsilyl, triphenylsilyl, or 2,2,2-trichloroethoxycarbonyl;

vii) R₂ is an aryl, heteroaryl, arylalkyl, arylalkenyl, arylalkynyl, or heteroarylalkyl, heteroarylalkenyl, heteroarylalkynyl moiety optionally substituted with one or more occurrences of R₈, wherein each occurrence of R₈ is independently hydrogen, halogen, —OR₉, —SR₉, —N(R₉)₂, —(CV₂)ₙOR₉, —(CV₂)ₙN(R₉)₂, —(CV₂)ₙSR₉, —(C═O)R₉, —O(C═O)R₉, —(C═O)OR₉, —O(C═O)OR₉; —NH(C═O)R₉, —NH(C═O)OR₉, —(C═O)NHR₉, or a cyclic or acyclic, linear or branched aliphatic, heteroaliphatic, aryl, heteroaryl, arylalkyl, arylalkenyl, arylalkynyl, or heteroarylalkyl, heteroarylalkenyl, heteroarylalkynyl moiety optionally substituted with one or more occurrences of halogen, —OR₉, —SR₉, —N(R₉)₂, —(CV₂)ₙOR₉, —(CV₂)ₙN(R₉)₂, —(CV₂)ₙSR₉, —(C═O)R₉, —O(C═O)R₉, —(C═O)OR₉, —O(C═O)OR₉; —NH(C═O)R₉, —NH(C═O)OR₉, —(C═O)NHR₉, or a cyclic or acyclic, linear or branched, substituted or unsubstituted aliphatic, heteroaliphatic, aryl, heteroaryl, arylalkyl, arylalkenyl, arylalkynyl, or heteroarylalkyl, heteroarylalkenyl, heteroarylalkynyl moiety,

wherein each occurrence of R₉ is independently hydrogen; a protecting group; a cyclic or acyclic, linear or branched, substituted or unsubstituted aliphatic, heteroaliphatic, aryl, or heteroaryl moiety; or is an epothilone, desoxyepothilone or analogues thereof; a polymer; carbohydrate; photoaffinity label; or radiolabel;

wherein each occurrence of V is independently hydrogen, halogen, hydroxyl, thio, amino, alkylamino, or protected hydroxyl, thio or amino; and

each occurrence of n is 0-10;

viii) R₂ is one of:

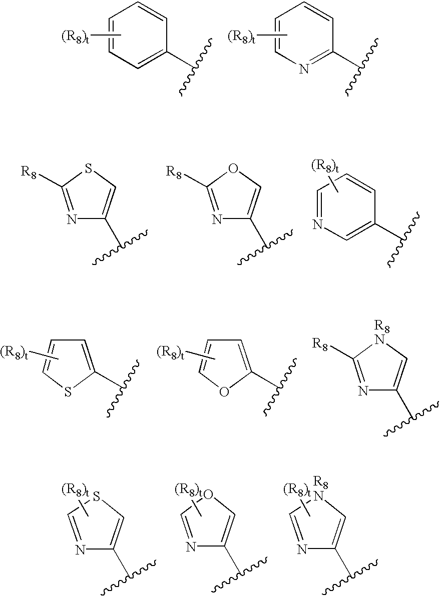

wherein each occurrence of R₈ is independently hydrogen, halogen, —OR₉, —SR₉, —N(R₉)₂, —(CV₂)ₙOR₉, —(CV₂)ₙN(R₉)₂, —(CV₂)ₙSR₉, —(C═O)R₉, —O(C═O)R₉, —(C═O)OR₉; —O(C═O)OR₉, —NH(C═O)R₉, —NH(C═O)OR₉, —(C═O)NHR₉, or a cyclic or acyclic, linear or branched aliphatic, heteroaliphatic, aryl, heteroaryl, arylalkyl, arylalkenyl, arylalkynyl, or heteroarylalkyl, heteroarylalkenyl, heteroarylalkynyl moiety optionally substituted with one or more occurrences of halogen, —OR₉, —SR₉, —N(R₉)₂, —(CV₂)ₙOR₉, —(CV₂)ₙN(R₉)₂, —(CV₂)ₙSR₉, —O(C═O)R₉, —(C═O)OR₉, —O(C═O)OR₉; —NH(C═O)R₉, —NH(C═O)OR₉, —(C═O)NHR₉, or a cyclic or acyclic, linear or branched, substituted or unsubstituted aliphatic, heteroaliphatic, aryl, heteroaryl, arylalkyl, arylalkenyl, arylalkynyl, or heteroarylalkyl, heteroarylalkenyl, heteroarylalkynyl moiety,

wherein each occurrence of R₉ is independently hydrogen; a protecting group; a cyclic or acyclic, linear or branched, substituted or unsubstituted aliphatic, heteroaliphatic, aryl, or heteroaryl moiety; or is an epothilone, desoxyepothilone or analogues thereof; a polymer; carbohydrate; photoaffinity label; or radiolabel;

wherein each occurrence of V is independently hydrogen, halogen, hydroxyl, thio, amino, alkylamino, or protected hydroxyl, thio or amino; each occurrence of t is independently 0, 1 or 2; and each occurrence of n is independently 0-10;

ix) R₂ is one of

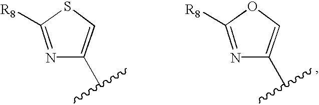

wherein each occurrence of R₈ is independently hydrogen, halogen, —OR₉, —SR₉, —N(R₉)₂, —(CV₂)ₙOR₉, —(CV₂)ₙN(R₉)₂, —(CV₂)ₙSR₉, —(C═O)R₉, —O(C═O)R₉, —(C═O)OR₉, —O(C═O)OR₉; —NH(C═O)R₉, —NH(C═O)OR₉, —(C═O)NHR₉, or a cyclic or acyclic, linear or branched aliphatic, heteroaliphatic, aryl, heteroaryl, arylalkyl, arylalkenyl, arylalkynyl, or heteroarylalkyl, heteroarylalkenyl, heteroarylalkynyl moiety optionally substituted with one or more occurrences of halogen, —OR₉, —SR₉, —N(R₉)₂, —(CV₂)ₙOR₉, —(CV₂)ₙN(R₉)₂, —(CV₂)ₙSR₉, —(C═O)R₉, —O(C═O)R₉, —(C═O)OR₉, —O(C═O)OR₉; —NH(C═O)R₉, —NH(C═O)OR₉, —(C═O)NHR₉, or a cyclic or acyclic, linear or branched, substituted or unsubstituted aliphatic, heteroaliphatic, aryl, heteroaryl, arylalkyl, arylalkenyl, arylalkynyl, or heteroarylalkyl, heteroarylalkenyl, heteroarylalkynyl moiety,

wherein each occurrence of R₉ is independently hydrogen; a protecting group; a cyclic or acyclic, linear or branched, substituted or unsubstituted aliphatic, heteroaliphatic, aryl, or heteroaryl moiety; or is an epothilone, desoxyepothilone or analogues thereof; a polymer; carbohydrate; photoaffinity label; or radiolabel;

wherein each occurrence of V is independently hydrogen, halogen, hydroxyl, thio, amino, alkylamino, or protected hydroxyl, thio or amino; and

each occurrence of n is 0-10;

x) R₂ is

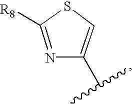

wherein each occurrence of R₈ is independently hydrogen, halogen, —OR₉, —SR₉, —N(R₉)₂, —(CV₂)ₙOR₉, —(CV₂)ₙN(R₉)₂, —(CV₂)ₙSR₉, —(C═O)R₉, —O(C═O)OR₉, —(C═O)OR₉, —O(C═O)OR₉; —NH(C═O)R₉, —NH(C═O)OR₉, —(C═O)NHR₉, or a cyclic or acyclic, linear or branched aliphatic, heteroaliphatic, aryl, heteroaryl, arylalkyl, arylalkenyl, arylalkynyl, or heteroarylalkyl, heteroarylalkenyl, heteroarylalkynyl moiety optionally substituted with one or more occurrences of halogen, —OR₉, —SR₉, —N(R₉)₂, —(CV₂)ₙOR₉, —(CV₂)ₙN(R₉)₂, —(CV₂)ₙSR₉, —(C═O)R₉, —O(C═O)R₉, —(C═O)OR₉, —O(C═O)OR₉; —NH(C═O)R₉, —NH(C═O)OR₉, —(C═O)NHR₉, or a cyclic or acyclic, linear or branched, substituted or unsubstituted aliphatic, heteroaliphatic, aryl, heteroaryl, arylalkyl, arylalkenyl, arylalkynyl, or heteroarylalkyl, heteroarylalkenyl, heteroarylalkynyl moiety,

wherein each occurrence of R₉ is independently hydrogen; a protecting group; a cyclic or acyclic, linear or branched, substituted or unsubstituted aliphatic, heteroaliphatic, aryl, or heteroaryl moiety; or is an epothilone, desoxyepothilone or analogues thereof, a polymer; carbohydrate; photoaffinity label; or radiolabel;

wherein each occurrence of V is independently hydrogen, halogen, hydroxyl, thio, amino, alkylamino, or protected hydroxyl, thio or amino; and

each occurrence of n is 0-10;

xi) R₂ is

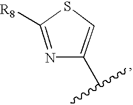

wherein each occurrence of R₈ is independently —OR₉, N(R₉)₂, —(CV₂)ₙOR₉, —(CV₂)ₙN(R₉)₂, —(C═O)R₉, or a substituted or unsubstituted lower alkyl or heteroalkyl moiety,

wherein each occurrence of R₉ is independently hydrogen; a protecting group; a cyclic or acyclic, linear or branched, substituted or unsubstituted aliphatic, heteroaliphatic, aryl, or heteroaryl moiety; or is an epothilone, desoxyepothilone or analogues thereof; a polymer; carbohydrate; photoaffinity label; or radiolabel;

wherein each occurrence of V is independently hydrogen, halogen, hydroxyl, thio, amino, alkylamino, or protected hydroxyl, thio or amino; and

each occurrence of n is 0-10;

xii) R₂ is

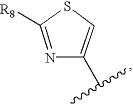

wherein each occurrence of R₈ is independently —OH, —NH₂, —CH₂OH, —CH₂NH₂, —(C═O)H, or methyl,

xiii) R_{B} is hydrogen, halogen, —(C═O)H, —(C═O)R_{B′}, —OH, —OR_{B′}, —SR_{B′}, —SH, NH₂, N(R_{B′})₂, —O(C═O)R_{B′}, cyclic acetal, or an alkyl, heteroalkyl, aryl, heteroaryl, arylalkyl or heteroarylalkyl, heteroarylalkenyl, heteroarylalkynyl moiety optionally substituted by one or more occurrences of halogen, —(C═O)H, —(C═O)R_{B′}, —OH, —OR_{B′}, —SR_{B′}, —SH, NH₂, N(R_{B′})₂, —O(C═O)R_{B′} or any combination thereof;

wherein each occurrence of R_{B′} is independently hydrogen, a protecting group, or a linear or branched, substituted or unsubstituted, cyclic or acyclic, aliphatic, heteroaliphatic, aryl, heteroaryl, arylalkyl, arylalkenyl, arylalkynyl, or heteroarylalkyl, heteroarylalkenyl, heteroarylalkynyl moiety;

xiv) R_{B} is hydrogen, halogen, —(C═O)H, —(C═O)R_{B′}, —OH, —OR_{B′}, —SR_{B′}, —SH, NH₂, N(R_{B′})₂, —O(C═O)R_{B′}, cyclic acetal, or an alkyl, heteroalkyl, aryl, heteroaryl, arylalkyl or heteroarylalkyl, heteroarylalkenyl, heteroarylalkynyl moiety optionally substituted by one or more occurrences of halogen, —(C═O)H, —(C═O)R_{B′}, —OH, —OR_{B′}, —SR_{B′}, —SH, NH₂, N(R_{B′})₂, —O(C═O)R_{B′} or any combination thereof;

wherein each occurrence of R_{B′} is independently hydrogen, a protecting group, or a linear or branched, substituted or unsubstituted, cyclic or acyclic, aliphatic, heteroaliphatic, aryl, heteroaryl, arylalkyl, arylalkenyl, arylalkynyl, or heteroarylalkyl, heteroarylalkenyl, heteroarylalkynyl moiety;

xv) R_{B} is hydrogen, a cyclic acetal, or is an alkyl, heteroalkyl, cycloalkyl, or cycloheteroalkyl moiety optionally substituted with one or more occurrences of halogen, —OH, —OR_{B′}, NH₂, or N(R_{B′})₂, or any combination thereof, wherein each occurrence of R_{B′} is independently hydrogen, alkyl, aryl or a protecting group;

xvi) R_{B} is hydrogen,

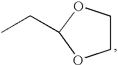

methyl, ethyl, propyl, butyl, pentyl, hexyl, cyclopropyl, cyclobutyl, cyclopentyl, or cyclohexyl, each unsubstituted or optionally substituted with one or more occurrences of halogen, —OH, —OR_{B′}, NH₂, or N(R_{B′})₂, or any combination thereof, wherein each occurrence of R_{B′} is independently hydrogen, alkyl, aryl, or a protecting group;

xvii) R_{B} is hydrogen, methyl, ethyl, propyl, butyl, pentyl, hexyl, cyclopropyl, cyclobutyl, cyclopentyl, or cyclohexyl;

xviii) R_{B} is methyl, ethyl, propyl, butyl, pentyl, hexyl, cyclopropyl, cyclobutyl, cyclopentyl, or cyclohexyl, each substituted with one or more occurrences of fluorine;

xix) R_{B} is methyl, ethyl, propyl, butyl, pentyl, hexyl, cyclopropyl, cyclobutyl, cyclopentyl, or cyclohexyl, each substituted with one or more occurrences of —OH or —OR_{B′}, wherein each occurrence of R_{B′} is independently hydrogen, alkyl, aryl or a protecting group;

xx) R_{B} is —CH₂OR_{B′}, —CH₂CH₂OR_{B′}, —CH₂CH₂CH₂OR_{B′}, —CH₂CH₂CH₂CH₂OR_{B′}, —CH₂CH₂CH₂CH₂CH₂OR_{B′}, or —CH₂CH₂CH₂CH₂CH₂CH₂OR_{B′}, wherein each occurrence of R_{B′} is hydrogen or a protecting group;

xxi) R_{B} is methyl, ethyl, propyl, butyl, pentyl, hexyl, cyclopropyl, cyclobutyl, cyclopentyl, or cyclohexyl, each substituted with one or more occurrences of —NH₂ or —N(R_{B′})₂, wherein each occurrence of R_{B′} is independently hydrogen, alkyl, aryl or a protecting group;

xxii) R_{B} is —(CH₂)_{q}CF₃, —(CH₂)_{q}CFH₂, or —(CH₂)_{q}CF₂H, wherein q is an integer from 0 to6;

xxiii) R_{B} is —(CH₂)_{q}CF₃, —(CH₂)_{q}CFH₂, or —(CH₂)_{q}CF₂H, wherein q is 0 and R_{B} is —CF₃, —CFH₂ or —CF₂H;

xxiv) one occurrence of R_{C} and one occurrence of R_{D} taken together are a substituted or unsubstituted 3-6-membered cyclic aliphatic or heteroaliphatic moiety, or are a 3-6-membered substituted or unsubstituted aryl or heteroaryl moiety;

xxv) one occurrence of R_{C} and one occurrence of R_{D} taken together are a substituted or unsubstituted 3-6-membered cyclic aliphatic or heteroaliphatic moiety, or are a 3-6-membered substituted or unsubstituted aryl or heteroaryl moiety;

xxvi) one occurrence of R_{B} and one occurrence of R_{C} taken together are a substituted or unsubstituted 3-6-membered cyclic aliphatic or heteroaliphatic moiety, or are a 3-6-membered substituted aryl or heteroaryl moiety;

xxvii) A—B—(CH₂)ₘ—C—D is

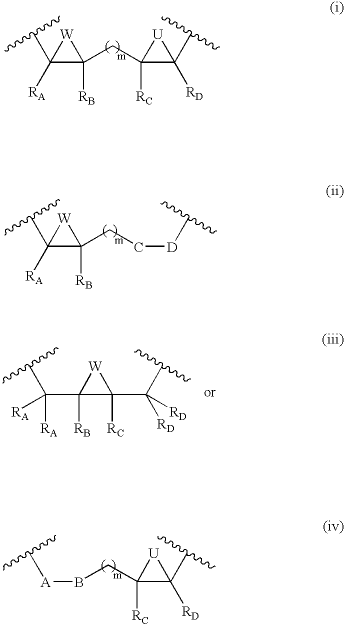

wherein W and U are each independently O, S, S═O, SO₂, NR_{W}, NR_{U}, C(R_{W})₂ or C(R_{U})₂, wherein each occurrence of R_{W} or R_{U} is independently hydrogen, substituted or unsubstituted, branched or unbranched, cyclic or acyclic, aliphatic, heteroaliphatic, aryl, or heteroaryl; halogen, hydroxyl; protected hydroxyl; thio; protected thio; or substituted or unsubstituted amino;

xxviii) m is 1 and q is 1;

xxix) m is 2 and q is 1;

xxx) m is 3 and q is 1;

xxxi) R₁ and R₂ taken together are a substituted or unsubstituted 5-7-membered cyclic moiety;

xxxii) R₁ and R₂ taken together are a substituted or unsubstituted 8-12-membered bicyclic moiety; and

xxxiii) R₀ is

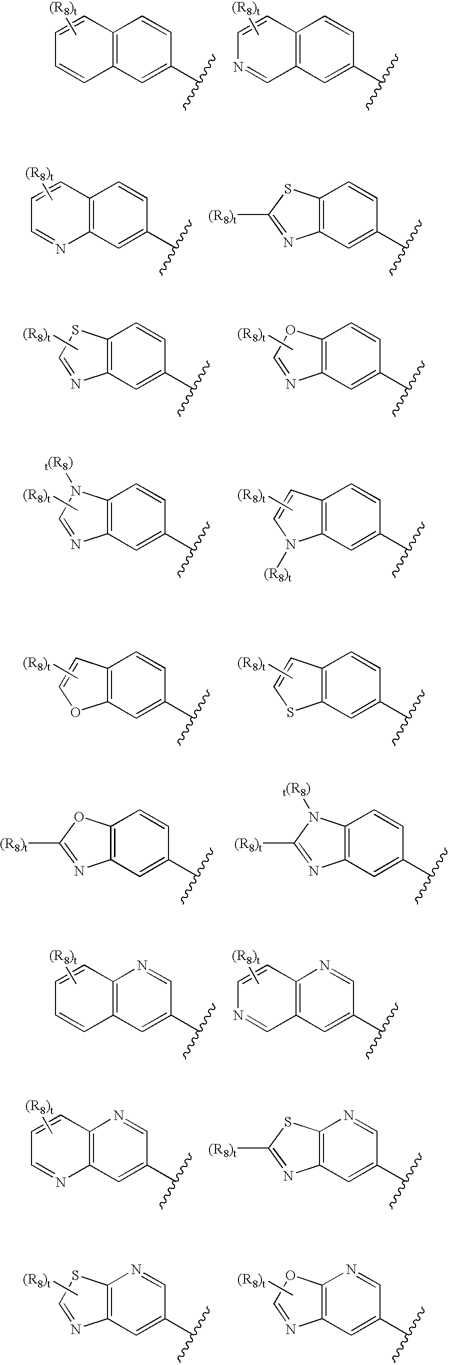

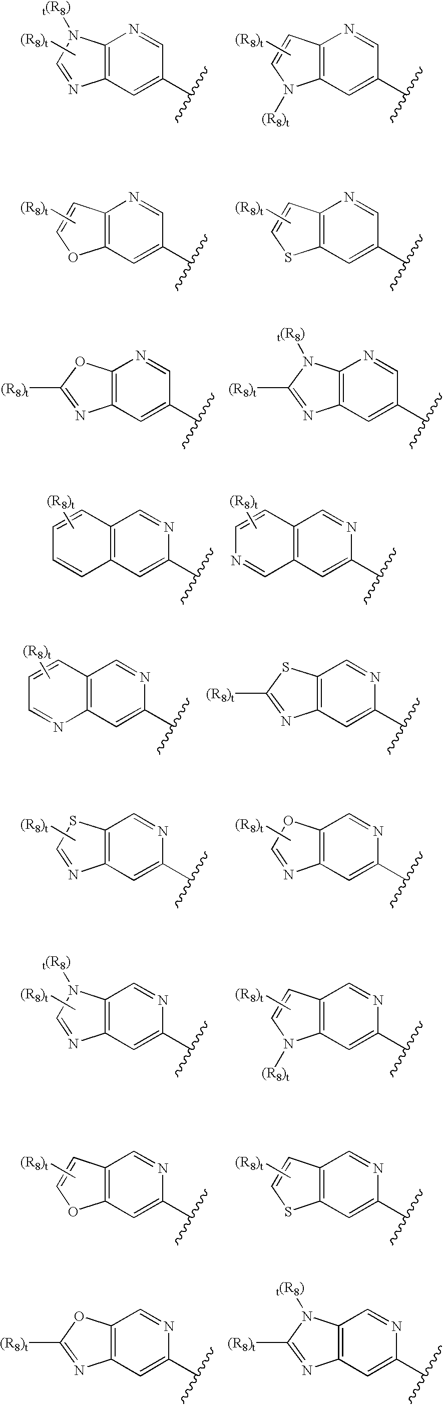

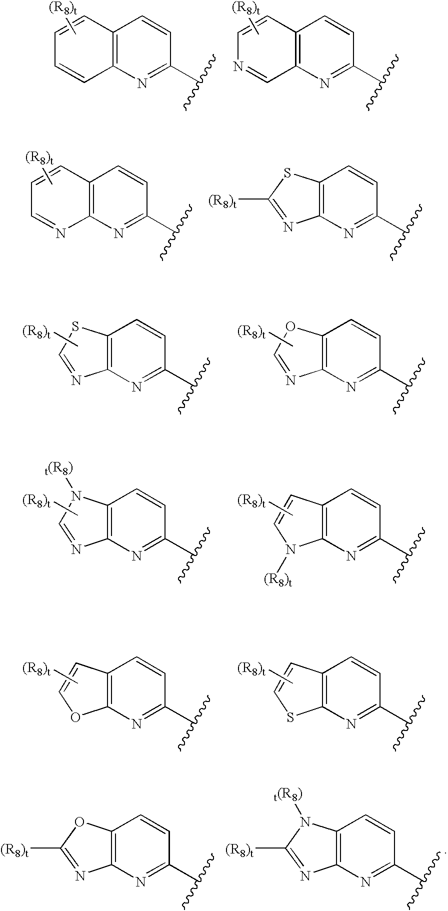

xxxiv) R₀ is

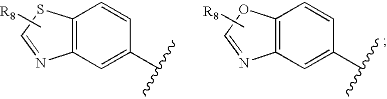

xxxv) R₀ is

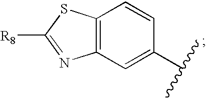

xxxvi) R₀ is

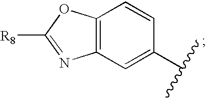

As the reader will appreciate, compounds of particular interest include, among others, those which share the attributes of one or more of the foregoing subclasses. Some of those subclasses are illustrated by the following sorts of compounds:

I) Compounds of the Formula:

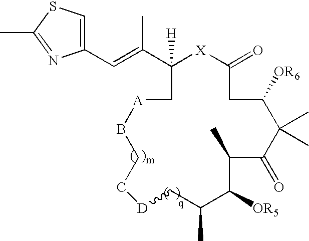

wherein the compound is defined as described generically and in classes and subclasses above.

In certain embodiments, X is O or NH; R₅ and R₆ are hydrogen, t-butyldimethylsilyl, triethylsilyl, triisobutylsilyl, triisopropylsilyl, trimethylsilyl, triphenylsilyl, or 2,2,2-trichloroethoxycarbonyl; and R_{B} is hydrogen, a cyclic acetal, or is an alkyl, heteroalkyl, cycloalkyl, or cycloheteroalkyl moiety that is unsubstituted or is optionally substituted with one or more occurrences of halogen, —OH, —OR_{B′}, NH₂, or N(R_{B′})₂, or any combination thereof, wherein each occurrence of R_{B′} is independently hydrogen, alkyl, aryl or a protecting group.

In certain other embodiments of the compounds as described directly above, R_{B} is hydrogen,

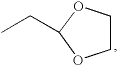

methyl, ethyl, propyl, butyl, pentyl, hexyl, cyclopropyl, cyclobutyl, cyclopentyl, or cyclohexyl, each unsubstituted or optionally substituted with one or more occurrences of halogen, —OH, —OR_{B′}, NH₂, or N(R_{B′})₂, or any combination thereof, wherein each occurrence of R_{B′} is independently hydrogen, alkyl, aryl, or a protecting group.

In certain embodiments m is 1 and q is 1. In certain other embodiments, m is 2 and q is 1. In still other embodiments, m is 3 and q is 1.

II) Compounds of the Formula:

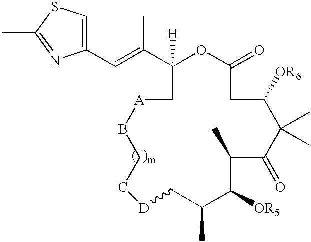

wherein the compound is defined as described generically and in classes and subclasses above.

In certain embodiments, R₅ and R₆ are hydrogen, t-butyldimethylsilyl, triethylsilyl, triisobutylsilyl, triisopropylsilyl, trimethylsilyl, triphenylsilyl, or 2,2,2-trichloroethoxycarbonyl; and R_{B} is hydrogen, a cyclic acetal, or is an alkyl, heteroalkyl, cycloalkyl, or cycloheteroalkyl moiety that is unsubstituted or is optionally substituted with one or more occurrences of halogen, —OH, —OR_{B′}, NH₂, or N(R_{B′})₂, or any combination thereof, wherein each occurrence of R_{B′} is independently hydrogen, alkyl, aryl or a protecting group.

In certain other embodiments of the compounds as described directly above R_{B} is hydrogen,

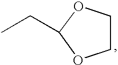

methyl, ethyl, propyl, butyl, pentyl, hexyl, cyclopropyl, cyclobutyl, cyclopentyl, or cyclohexyl, each unsubstituted or optionally substituted with one or more occurrences of halogen, —OH, —OR_{B′}, NH₂, or N(R_{B′})₂, or any combination thereof, wherein each occurrence of R_{B′} is independently hydrogen, alkyl, aryl, or a protecting group. In certain embodiments, m is 1, 2 or 3.

III) Compounds of the Formula:

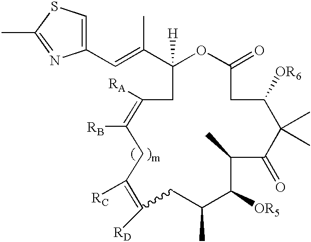

wherein the compound is defined as described generically above.

In certain embodiments, R₅ and R₆ are hydrogen, t-butyldimethylsilyl, triethylsilyl, triisobutylsilyl, triisopropylsilyl, trimethylsilyl, triphenylsilyl, or 2,2,2-trichloroethoxycarbonyl; R_{A} is hydrogen, lower alkyl, hydroxyl, or protected hydroxyl; R_{B} is hydrogen, a cyclic acetal, or is an alkyl, heteroalkyl, cycloalkyl, or cycloheteroalkyl moiety that is unsubstituted or is optionally substituted with one or more occurrences of halogen, —OH, —OR_{B′}, NH₂, or N(R_{B′})₂, or any combination thereof, wherein each occurrence of R_{B′} is independently hydrogen, alkyl, aryl or a protecting group; R_{C} is hydrogen, lower alkyl, hydroxyl, or protected hydroxyl; and R_{D} is hydrogen, lower alkyl, hydroxyl, or protected hydroxyl.

In certain other embodiments of the compounds as described directly above R_{B} is hydrogen,

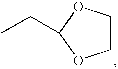

methyl, ethyl, propyl, butyl, pentyl, hexyl, cyclopropyl, cyclobutyl, cyclopentyl, or cyclohexyl, each unsubstituted or optionally substituted with one or more occurrences of halogen, —OH, —OR_{B′}, NH₂, or N(R_{B′})₂, or any combination thereof, wherein each occurrence of R_{B′} is independently hydrogen, alkyl, aryl, or a protecting group.

In still other embodiments of the compounds as described directly above, R_{A}, R_{C} and R_{D} are each hydrogen; and R_{B} is hydrogen,

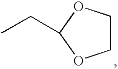

methyl, ethyl, propyl, butyl, pentyl, hexyl, cyclopropyl, cyclobutyl, cyclopentyl, or cyclohexyl, each unsubstituted or optionally substituted with one or more occurrences of halogen, —OH, —OR_{B′}, NH₂, or N(R_{B′})₂, or any combination thereof, wherein each occurrence of R_{B′} is independently hydrogen, alkyl, aryl, or a protecting group.

In still other embodiments of the compounds as described directly above, R_{A}, R_{C} and R_{D} are each hydrogen; and R_{B} is CF₃, CF₂H, or CH₂F.

In certain other embodiments, m is 1, 2 or 3.

In still other embodiments, m is 1, 2 or 3; R_{A}, R_{C} and R_{D} are each hydrogen; R_{B} is methyl; and R₅ and R₆ are each independently hydrogen, t-butyldimethylsilyl, triethylsilyl, triisobutylsilyl, triisopropylsilyl, trimethylsilyl, triphenylsilyl, or 2,2,2-trichloroethoxycarbonyl, and the compound has any one of the structures:

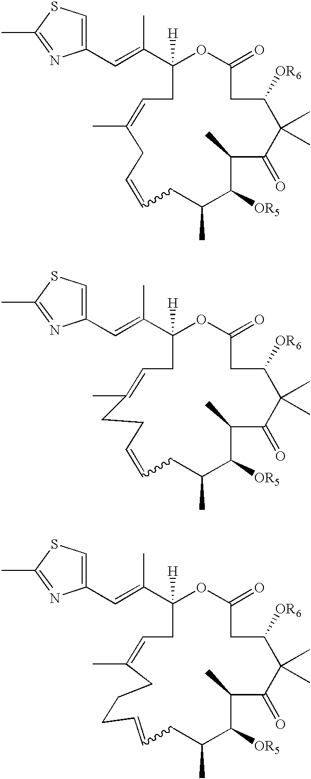

In still other compounds of special interest, R₅ and R₆ are each hydrogen. In yet other compounds of special interest, R₆ is triethylsilyl and R₅ is 2,2,2-trichloroethoxycarbonyl. In other compounds of special interest, R₅ is hydrogen and R₆ is triethylsilyl.

IV) Compounds of the Formula:

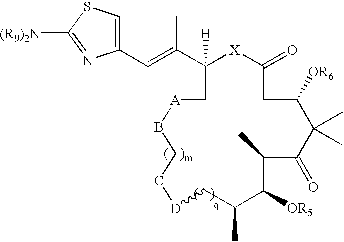

wherein the compound is defined as described generically and in classes and subclasses above.

In certain embodiments, X is O or NH; R₅ and R₆ are hydrogen, t-butyldimethylsilyl, triethylsilyl, triisobutylsilyl, triisopropylsilyl, trimethylsilyl, triphenylsilyl, or 2,2,2-trichloroethoxycarbonyl; R_{B} is hydrogen, a cyclic acetal, or is an alkyl, heteroalkyl, cycloalkyl, or cycloheteroalkyl moiety that is unsubstituted or is optionally substituted with one or more occurrences of halogen, —OH, —OR_{B′}, NH₂, or N(R_{B′})₂, or any combination thereof, wherein each occurrence of R_{B′} is independently hydrogen, alkyl, aryl or a protecting group; and R₉ is hydrogen, a protecting group or lower alkyl.

In certain other embodiments of the compounds as described directly above, R_{B} is hydrogen,

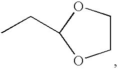

methyl, ethyl, propyl, butyl, pentyl, hexyl, cyclopropyl, cyclobutyl, cyclopentyl, or cyclohexyl, each unsubstituted or optionally substituted with one or more occurrences of halogen, —OH, —OR_{B′}, NH₂, or N(R_{B′})₂, or any combination thereof, wherein each occurrence of R_{B′} is independently hydrogen, alkyl, aryl, or a protecting group.

In certain embodiments m is 1 and q is 1. In certain other embodiments, m is 2 and q is 1. In still other embodiments, m is 3 and q is 1.

V) Compounds of the Formula:

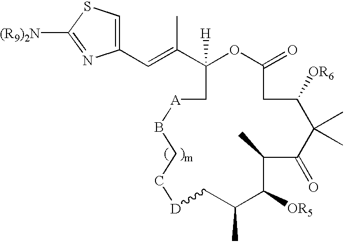

wherein the compound is defined as described generically and in classes and subclasses above.

In certain embodiments, R₅ and R₆ are hydrogen, t-butyldimethylsilyl, triethylsilyl, triisobutylsilyl, triisopropylsilyl, trimethylsilyl, triphenylsilyl, or 2,2,2-trichloroethoxycarbonyl; R_{B} is hydrogen, a cyclic acetal, or is an alkyl, heteroalkyl, cycloalkyl, or cycloheteroalkyl moiety that is unsubstituted or is optionally substituted with one or more occurrences of halogen, —OH, —OR_{B′}, NH₂, or N(R_{B′})₂, or any combination thereof, wherein each occurrence of R_{B′} is independently hydrogen, alkyl, aryl or a protecting group; and R₉ is hydrogen, a protecting group or lower alkyl.

In certain embodiments, m is 1, 2 or 3.

In certain other embodiments of the compounds as described directly above R_{B} is hydrogen,

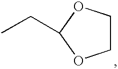

methyl, ethyl, propyl, butyl, pentyl, hexyl, cyclopropyl, cyclobutyl, cyclopentyl, or cyclohexyl, each unsubstituted or optionally substituted with one or more occurrences of halogen, —OH, —OR_{B′}, NH₂, or N(R_{B′})₂, or any combination thereof, wherein each occurrence of R_{B′} is independently hydrogen, alkyl, aryl, or a protecting group.

VI) Compounds of the Formula:

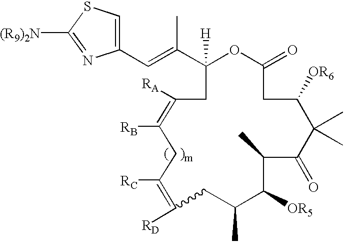

wherein the compound is defined as described generically above.

In certain embodiments, R₅ and R₆ are hydrogen, t-butyldimethylsilyl, triethylsilyl, triisobutylsilyl, triisopropylsilyl, trimethylsilyl, triphenylsilyl, or 2,2,2-trichloroethoxycarbonyl; R_{A} hydrogen, lower alkyl, hydroxyl, or protected hydroxyl; R_{B} is hydrogen, a cyclic acetal, or is an alkyl, heteroalkyl, cycloalkyl, or cycloheteroalkyl moiety that is unsubstituted or is optionally substituted with one or more occurrences of halogen, —OH, —OR_{B′}, NH₂, or N(R_{B′})₂, or any combination thereof, wherein each occurrence of R_{B′} is independently hydrogen, alkyl, aryl or a protecting group; and R₉ is hydrogen, a protecting group or lower alkyl; R_{C} is hydrogen, lower alkyl, hydroxyl, or protected hydroxyl; and R_{D} is hydrogen, lower alkyl, hydroxyl, or protected hydroxyl.

In certain other embodiments of the compounds as described directly above R_{B} is hydrogen,

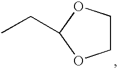

methyl, ethyl, propyl, butyl, pentyl, hexyl, cyclopropyl, cyclobutyl, cyclopentyl, or cyclohexyl, each unsubstituted or optionally substituted with one or more occurrences of halogen, —OH, —OR_{B′}, NH₂, or N(R_{B′})₂, or any combination thereof, wherein each occurrence of R_{B′} is independently hydrogen, alkyl, aryl, or a protecting group.

In still other embodiments of the compounds as described directly above, R_{A}, R_{C} and R_{D} are each hydrogen; and R_{B} is CF₃, CF₂H, or CH₂F.

In certain embodiments, m is 1, 2 or 3.

In still other embodiments of the compounds as described directly above, R_{A}, R_{C} and R_{D} are each hydrogen; R₉ is hydrogen or lower alkyl; and R_{B} is hydrogen,

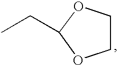

methyl, ethyl, propyl, butyl, pentyl, hexyl, cyclopropyl, cyclobutyl, cyclopentyl, or cyclohexyl, each unsubstituted or optionally substituted with one or more occurrences of halogen, —OH, —OR_{B′}, NH₂, or N(R_{B′})₂, or any combination thereof, wherein each occurrence of R_{B′} is independently hydrogen, alkyl, aryl, or a protecting group.

In still other embodiments, R_{A}, R_{C} and R_{D} are each hydrogen; R_{B} is methyl; and R₅ and R₆ are each independently hydrogen, t-butyldimethylsilyl, triethylsilyl, triisobutylsilyl, triisopropylsilyl, trimethylsilyl, triphenylsilyl, or 2,2,2-trichloroethoxycarbonyl; and each occurrence of R₉ is hydrogen, and the compound has the structure:

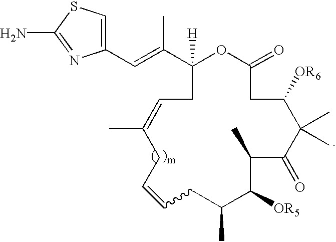

In certain embodiments, m is 1, 2 or 3. In still other compounds of special interest, R₅ and R₆ are each hydrogen. In yet other compounds of special interest, R₆ is triethylsilyl and R₅ is 2,2,2-trichloroethoxycarbonyl. In other compounds of special interest, R₅ is hydrogen and R₆ is triethylsilyl.

VII) Compounds of the Formula:

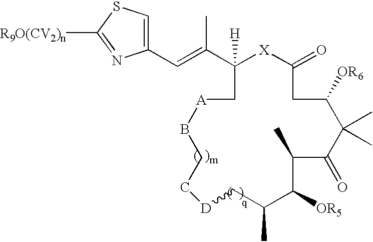

wherein the compound is defined as described generically and in classes and subclasses above.

In certain embodiments, X is O or NH; R₅ and R₆ are hydrogen, t-butyldimethylsilyl, triethylsilyl, triisobutylsilyl, triisopropylsilyl, trimethylsilyl, triphenylsilyl, or 2,2,2-trichloroethoxycarbonyl; R_{B} is hydrogen, a cyclic acetal, or is an alkyl, heteroalkyl, cycloalkyl, or cycloheteroalkyl moiety that is unsubstituted or is optionally substituted with one or more occurrences of halogen, —OH, —OR_{B′}, NH₂, or N(R_{B′})₂, or any combination thereof, wherein each occurrence of R_{B′} is independently hydrogen, alkyl, aryl or a protecting group; and R₉ is hydrogen, a protecting group, or lower alkyl, each occurrence of V is hydrogen and n is 0 or 1.

In certain other embodiments of the compounds as described directly above, R_{B} is hydrogen,

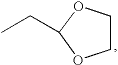

methyl, ethyl, propyl, butyl, pentyl, hexyl, cyclopropyl, cyclobutyl, cyclopentyl, or cyclohexyl, each unsubstituted or optionally substituted with one or more occurrences of halogen, —OH, —OR_{B′}, NH₂, or N(R_{B′})₂, or any combination thereof, wherein each occurrence of R_{B′} is independently hydrogen, alkyl, aryl, or a protecting group.

In certain embodiments m is 1 and q is 1. In certain other embodiments, m is 2 and q is 1. In still other embodiments, m is 3 and q is 1.

VIII) Compounds of the Formula:

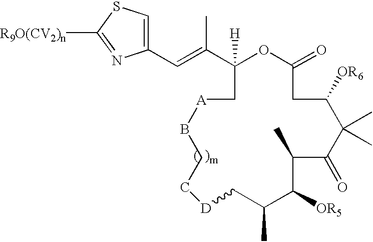

wherein the compound is defined as described generically and in classes and subclasses above.

In certain embodiments, R₅ and R₆ are hydrogen, t-butyldimethylsilyl, triethylsilyl, triisobutylsilyl, triisopropylsilyl, trimethylsilyl, triphenylsilyl, or 2,2,2-trichloroethoxycarbonyl; R_{B} is hydrogen, a cyclic acetal, or is an alkyl, heteroalkyl, cycloalkyl, or cycloheteroalkyl moiety that is unsubstituted or is optionally substituted with one or more occurrences of halogen, —OH, —OR_{B′}, NH₂, or N(R_{B′})_{2,} or any combination thereof, wherein each occurrence of R_{B′} is independently hydrogen, alkyl, aryl or a protecting group; and R₉ is hydrogen, a protecting group or lower alkyl, each occurrence of V is hydrogen and n is 0 or 1.

In certain other embodiments of the compounds as described directly above R_{B} is hydrogen,

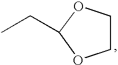

methyl, ethyl, propyl, butyl, pentyl, hexyl, cyclopropyl, cyclobutyl, cyclopentyl, or cyclohexyl, each unsubstituted or optionally substituted with one or more occurrences of halogen, —OH, —OR_{B′}, NH₂, or N(R_{B′})₂, or any combination thereof, wherein each occurrence of R_{B′} is independently hydrogen, alkyl, aryl, or a protecting group.

In certain embodiments, m is 1, 2 or 3.

IX) Compounds of the Formula:

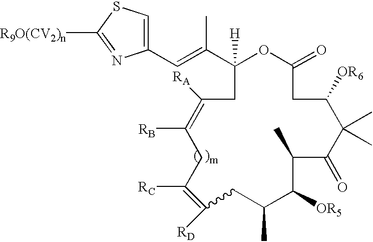

wherein the compound is defined as described generically above.

In certain embodiments, R₅ and R₆ are hydrogen, t-butyldimethylsilyl, triethylsilyl, triisobutylsilyl, triisopropylsilyl, trimethylsilyl, triphenylsilyl, or 2,2,2-trichloroethoxycarbonyl; R_{A} is hydrogen, lower alkyl, hydroxyl, or protected hydroxyl; R_{B} is hydrogen, a cyclic acetal, or is an alkyl, heteroalkyl, cycloalkyl, or cycloheteroalkyl moiety that is unsubstituted or is optionally substituted with one or more occurrences of halogen, —OH, —OR_{B′}, NH₂, or N(R_{B′})₂, or any combination thereof, wherein each occurrence of R_{B′} is independently hydrogen, alkyl, aryl or a protecting group; R_{C} is hydrogen, lower alkyl, hydroxyl, or protected hydroxyl;. and R_{D} is hydrogen, lower alkyl, hydroxyl, or protected hydroxyl; and R₉ is hydrogen, a protecting group, or lower alkyl, each occurrence of V is hydrogen and n is 0 or 1.

In certain other embodiments of the compounds as described directly above R_{B} is hydrogen,

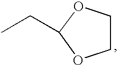

methyl, ethyl, propyl, butyl, pentyl, hexyl, cyclopropyl, cyclobutyl, cyclopentyl, or cyclohexyl, each unsubstituted or optionally substituted with one or more occurrences of halogen, —OH, —OR_{B′}, NH₂, or N(R_{B′})₂, or any combination thereof, wherein each occurrence of R_{B′} is independently hydrogen, alkyl, aryl, or a protecting group.

In still other embodiments of the compounds as described directly above, R_{A}, R_{C} and R_{D} are each hydrogen; and R_{B} is CF₃, CF₂H, or CH₂F.

In still other embodiments of the compounds as described directly above, R_{A}, R_{C} and R_{D} are each hydrogen; R₉ is hydrogen or lower alkyl; and R_{B} is hydrogen,

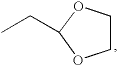

methyl, ethyl, propyl, butyl, pentyl, hexyl, cyclopropyl, cyclobutyl, cyclopentyl, or cyclohexyl, each unsubstituted or optionally substituted with one or more occurrences of halogen, —OH, —OR_{B′}, NH₂, or N(R_{B′})₂, or any combination thereof, wherein each occurrence of R_{B′} is independently hydrogen, alkyl, aryl, or a protecting group.

In certain embodiments, m is 1, 2 or 3.

In still other embodiments, R_{A}, R_{C} and R_{D} are each hydrogen; R_{B} is methyl; and R₅ and R₆ are each independently hydrogen, t-butyldimethylsilyl, triethylsilyl, triisobutylsilyl, triisopropylsilyl, trimethylsilyl, triphenylsilyl, or 2,2,2-trichloroethoxycarbonyl; and R₉ is hydrogen, n is 1 and each occurrence of V is hydrogen, and the compound has the structure:

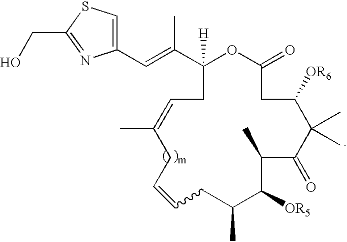

In still other compounds of special interest, m is 1, 2 or 3 and R₅ and R₆ are each hydrogen. In yet other compounds of special interest, R₆ is triethylsilyl and R₅ is 2,2,2-trichloroethoxycarbonyl. In other compounds of special interest, R₅ is hydrogen and R₆ is triethylsilyl.

X) Compounds of the Formula:

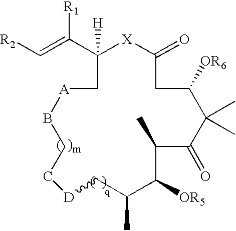

as defined generally and in classes and subclasses herein, wherein at least one occurrence of R_{B} is CF₃, CF₂H, or CH₂F.

In certain embodiments for the compounds described directly above, X is O. In other embodiments for the compounds described directly above, R_{A}, R_{C} and R_{D} are hydrogen. In certain other embodiments, X is O or NH; R₅ and R₆ are hydrogen, t-butyldimethylsilyl, triethylsilyl, triisobutylsilyl, triisopropylsilyl, trimethylsilyl, triphenylsilyl, or 2,2,2-trichloroethoxycarbonyl .

XI) Compounds of the Formula:

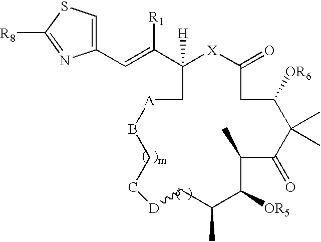

wherein at least one occurrence of R_{B} is CF₃, CF₂H, or CH₂F.

In certain embodiments for the compounds described directly above, X is O. In other embodiments for the compounds described directly above, R_{A}, R_{C} and R_{D} are hydrogen. In certain other embodiments, X is O or NH; R₅ and R₆ are hydrogen, t-butyldimethylsilyl, triethylsilyl, triisobutylsilyl, triisopropylsilyl, trimethylsilyl, triphenylsilyl, or 2,2,2-trichloroethoxycarbonyl.

In certain embodiments m is 1 and q is 1. In certain other embodiments, m is 2 and q is 1. In still other embodiments, m is 3 and q is 1.

XII) Compounds of the Formula:

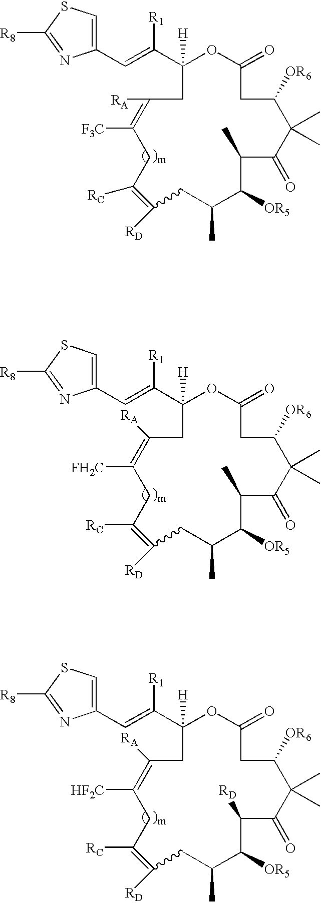

as defined generally and in classes and subclasses herein.

In certain embodiments, m is 1, 2 or 3.

In certain embodiments for the compounds described directly above, R_{A}, R_{C} and R_{D} are hydrogen and R₅ and R₆ are hydrogen, t-butyldimethylsilyl, triethylsilyl, triisobutylsilyl, triisopropylsilyl, trimethylsilyl, triphenylsilyl, or 2,2,2-trichloroethoxycarbonyl.

XIII) 10,11-dehydro Analogues:

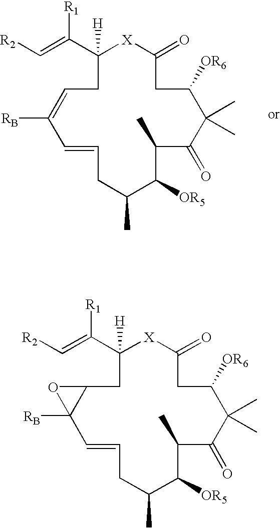

as defined generally and in classes and subclasses herein.

In certain embodiments, R_{B} is hydrogen,

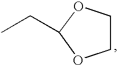

methyl, ethyl, propyl, butyl, pentyl, hexyl, cyclopropyl, cyclobutyl, cyclopentyl, or cyclohexyl, each unsubstituted or optionally substituted with one or more occurrences of halogen, —OH, —OR_{B′}, NH₂, or N(R_{B′})₂, or any combination thereof, wherein each occurrence of R_{B′} is independently hydrogen, alkyl, aryl, or a protecting group.

In certain embodiments, R_{B} is hydrogen, methyl, or ethyl. In certain other embodiments, R_{B} is methyl.

In other embodiments, R_{B} is —CH₂F, —CHF₂, or —CF₃. In certain embodiments, R_{B} is —CF₃.

In other embodiments, R₂ is thiazole or substituted thiazole. In some embodiments, R₂ is oxazole or substituted oxazole.

In some embodiments, when R₂ is thiazole or oxazole and R₁ is methyl, R_{B} is —CH₂F, —CHF₂, or —CF₃.

In some embodiments, R₂ is one of:

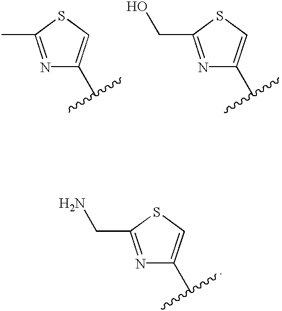

In other embodiments, R₂ is one of:

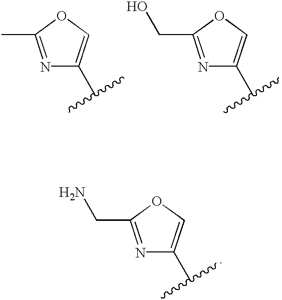

In certain embodiments, R₁ is methyl.

In some embodiments, R₁ is methyl; and R₂ is one of:

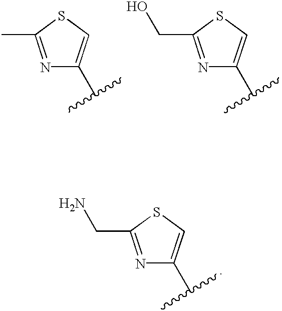

In other embodiments, R₁ is methyl; and R₂ is one of:

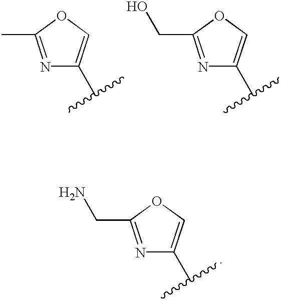

XIV) Substitutions at C-11:

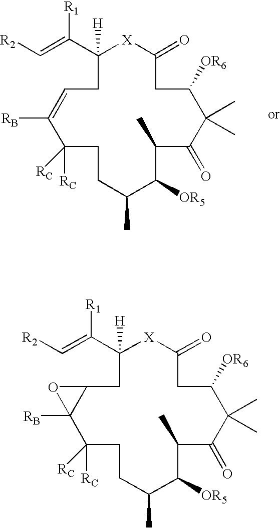

as defined generally and in classes and subclasses herein.

In certain embodiments, R_{B} is hydrogen,

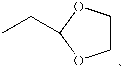

methyl, ethyl, propyl, butyl, pentyl, hexyl, cyclopropyl, cyclobutyl, cyclopentyl, or cyclohexyl, each unsubstituted or optionally substituted with one or more occurrences of halogen, —OH, —OR_{B′}, NH₂, or N(R_{B′})₂, or any combination thereof, wherein each occurrence of R_{B′} is independently hydrogen, alkyl, aryl, or, a protecting group.

In certain embodiments, R_{B} is hydrogen, methyl, or ethyl.

In certain embodiments, R_{B} is hydrogen or methyl. In other embodiments, R_{B} is methyl.

In certain embodiments, one or both of R₃ and R₄ are fluorine, hydroxy, alkoxy, alkylamino, dialkyl amino, or amino.

In certain embodiments, R_{C} and R_{C} are taken together to be C═O.

In other embodiments, one or both R_{C} and R_{C} are fluorine.

In still other embodiments, one or R_{C} and R_{C} is hydrogen, and the other is fluorine.

In other embodiments, R_{B} is —CH₂F, —CHF₂, or —CF₃. In certain embodiments, R_{B} is —CF₃.

In other embodiments, R₂ is thiazole or substituted thiazole. In some embodiments, R₂ is oxazole or substituted oxazole.

In some embodiments, when R₂ is thiazole or oxazole and R₁ is methyl, either one or both of R₃ and R₄ are not hydrogen. In some embodiments, when R₂ is thiazole or oxazole and R₁ is methyl, either one or both of R₃ and R₄ are fluorine. In some embodiments, when R₂ is thiazole or oxazole and R₁ is methyl, either one or both of R₃ and R₄ are hydroxy, amino, alkoxy, alkylamino, or dialkylamino.

In some embodiments, R₂ is one of:

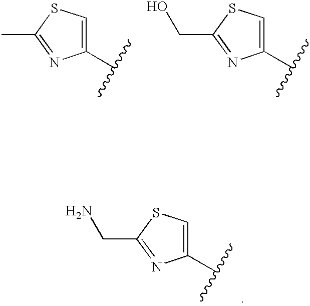

In other embodiments, R₂ is one of:

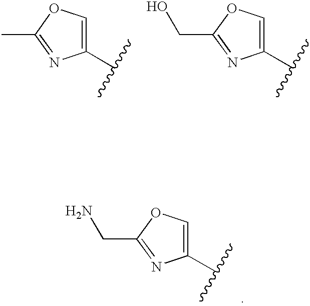

In certain embodiments, R₁ is methyl.

In some embodiments, R₁ is methyl; and R₂ is one of:

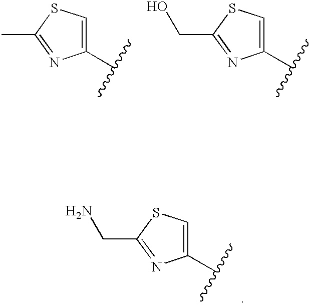

In other embodiments, R₁ is methyl; and R₂ is one of:

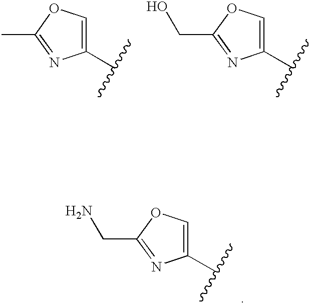

XV) Fluorine Substitution at C-26:

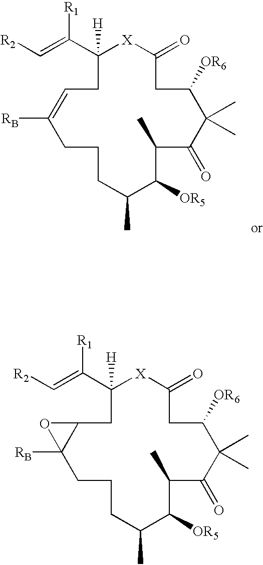

as defined generally and in classes and subclasses herein.

In certain embodiments, R_{B} is —CH₂F, CHF₂, or —CF₃.

In other embodiments, R_{B} is —CF₃.

In other emodiments, R₂ is thiazole or oxazole and R₁ is methyl, R_{B} is —CH₂F, CHF₂, or —CF₃.

In some embodiments, R₂ is one of:

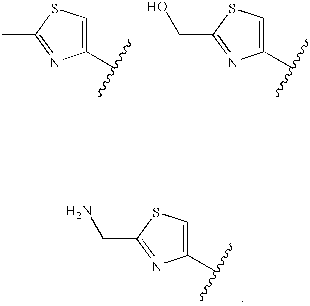

In other embodiments, R₂ is one of:

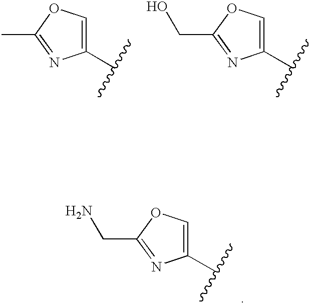

In certain embodiments, R₁ is methyl.

In some embodiments, R₁ is methyl; and R₂ is one of:

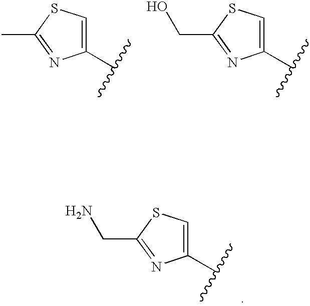

In other embodiments, R₁ is methyl; and R₂ is one of:

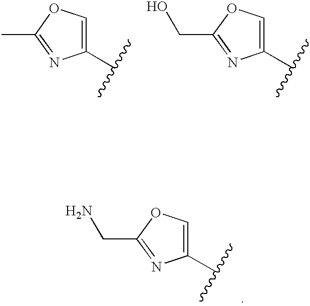

XVI) 4-desmethyl Analogues:

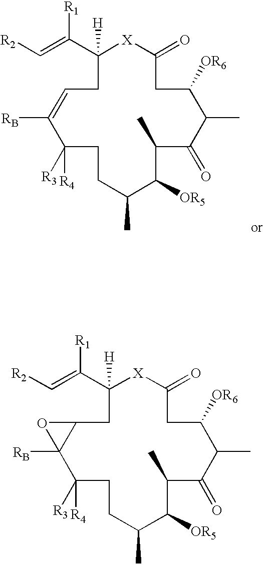

as defined generally and in classes and subclasses herein.

In some embodiments, C-4 is in the S-configuration.

In other embodiments, C-4 is in the R-configuration.

In yet other embodiments, R_{B} is hydrogen,

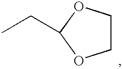

methyl, ethyl, propyl, butyl, pentyl, hexyl, cyclopropyl, cyclobutyl, cyclopentyl, or cyclohexyl, each unsubstituted or optionally substituted with one or more occurrences of halogen, —OH, —OR_{B′}, NH₂, or N(R_{B′})₂, or any combination thereof, wherein each occurrence of R_{B′} is independently hydrogen, alkyl, aryl, or a protecting group.

In certain embodiments, R_{B} is hydrogen, methyl, or ethyl. In other embodiments, R_{B} is hydrogen or methyl. In certain embodiments, R_{B} is methyl.

In other embodiments, R_{B} is —CH₂F, —CHF₂, or —CF₃. In certain embodiments, R_{B} is —CF₃.

In certain embodiments, one or both of R₃ and R₄ are fluorine, hydroxy, alkoxy, alkylamino, dialkyl amino, or amino.

In certain embodiments, R₃ and R₄ are taken together to be C═O.

In other embodiments, one or both R₃ and R₄ are fluorine.

In still other embodiments, one or R₃ and R₄ is hydrogen, and the other is fluorine.

In yet other embodiments, R₃ and R₄ are both hydrogen.

In other embodiments, R₂ is thiazole or substituted thiazole. In some embodiments, R₂ is oxazole or substituted oxazole.

In some embodiments, when R₂ is thiazole or oxazole and R₁ is methyl, either one or both of R₃ and R₄ are not hydrogen. In some embodiments, when R₂ is thiazole or oxazole and R₁ is methyl, either one or both of R₃ and R₄ are fluorine. In some embodiments, when R₂ is thiazole or oxazole and R₁ is methyl, either one or both of R₃ and R₄ are hydroxy, amino, alkoxy, alkylamino, or dialkylamino.

In some embodiments, R₂ is one of:

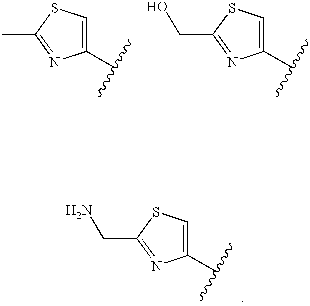

In other embodiments, R₂ is one of:

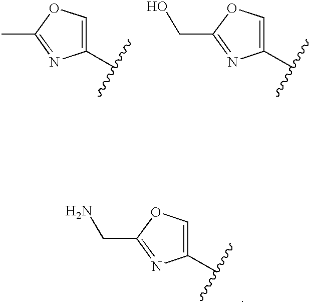

In certain embodiments, R₁ is methyl.

In some embodiments, R₁ is methyl; and R₂ is one of:

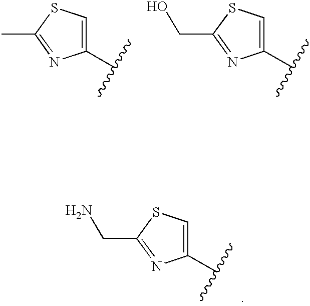

In other embodiments, R₁ is methyl; and R₂ is one of:

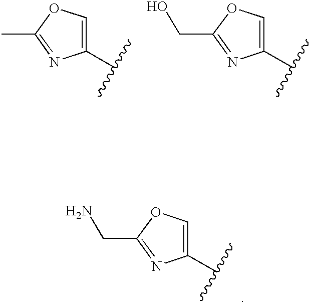

XVII) Ring-Expanded Analogues:

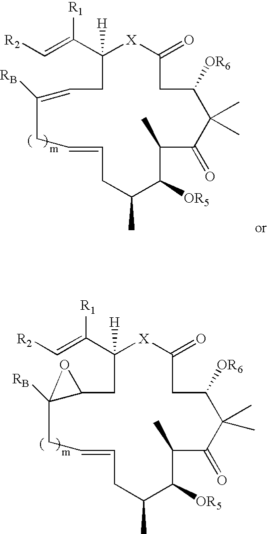

as defined generally and in classes and subclasses herein.

In certain embodiments, m is 0, 1, 2, or 3.

In other embodiments, m is 0. In yet other embodiments, m is 1.

In yet other embodiments, R_{B} is hydrogen,

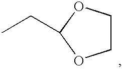

methyl, ethyl, propyl, butyl, pentyl, hexyl, cyclopropyl, cyclobutyl, cyclopentyl, or cyclohexyl, each unsubstituted or optionally substituted with one or more occurrences of halogen, —OH, —OR_{B′}, NH₂, or N(R_{B′})₂, or any combination thereof, wherein each occurrence of R_{B′} is independently hydrogen, alkyl, aryl, or a protecting group.

In certain embodiments, R_{B} is hydrogen, methyl, or ethyl.

In certain embodiments, R_{B} is hydrogen, or methyl.

In certain embodiments, R_{B} is —CH₂F, —CHF₂, or —CF₃. In certain embodiments, R_{B} is —CF₃.

In other embodiments, R₂ is thiazole or substituted thiazole. In some embodiments, R₂ is oxazole or substituted oxazole.

In certain embodiments, R₁ is methyl.

In certain embodiments, when R₁ is methyl, R₂ is substituted or unsubstituted thiazole or oxazole, and m is 0, R_{B} is not hydrogen or methyl.

In certain embodiments, when R₁ is methyl, R₂ is substituted or unsubstituted thiazole or oxazole, and m is 0, R_{B} is —CH₂F, —CHF₂, or —CF₃.

In some embodiments, R₂ is one of:

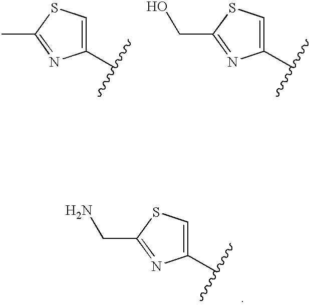

In other embodiments, R₂ is one of:

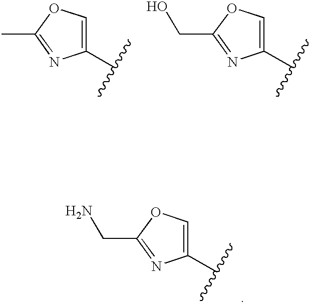

In some embodiments, R₁ is methyl; and R₂ is one of:

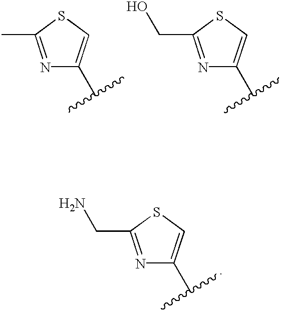

In other embodiments, R₁ is methyl; and R₂ is one of:

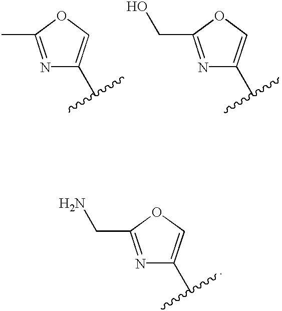

XVIII) Substitutions at C-14:

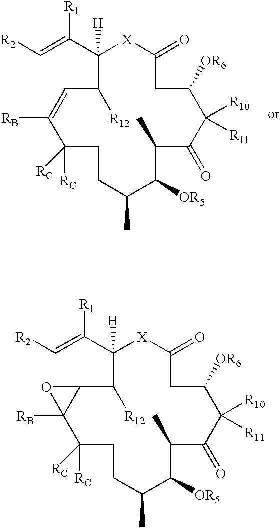

as defined generally and in classes and subclasses herein.

In certain embodiments, R₁₂ is halogen, alkyl, hydroxy, alkoxy, amino, alkylamino, or dialkylamino.

In certain embodiments, R₁₂ is fluorine. In other emobidments, R₁₂ is methyl, ethyl, propyl, or butyl. In certain other embodiments, R₁₂ is not hydroxy or methyl.

In certain embodiments, when R₁₂ is hydroxy or methyl, R₁₀ or R₁₁ is not methyl. In other embodiments, when R₁₂ is hydroxy or methyl, at least one of R₁₀ and R₁₁ is hydrogen.

In certain embodiments, when R₁₂ is hydroxy or methyl, at least one R_{C} is not hydrogen. In other embodiments, when R₁₂ is hydroxy or methyl, at least one R_{C} is fluorine.

In certain embodiments, when R₁₂ is hydroxy or methyl, R_{B} is —CH₂F, —CHF₂, or —CF₃.

In some embodiments, R₂ is one of:

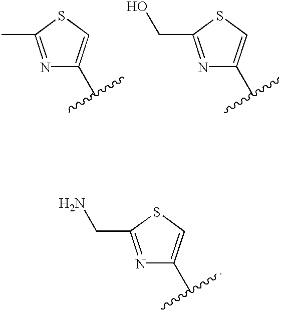

In other embodiments, R₂ is one of:

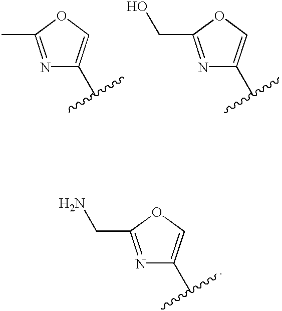

In certain embodiments, R_{B} is hydrogen,

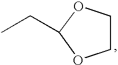

methyl, ethyl, propyl, butyl, pentyl, hexyl, cyclopropyl, cyclobutyl, cyclopentyl, or cyclohexyl, each unsubstituted or optionally substituted with one or more occurrences of halogen, —OH, —OR_{B′}, NH₂, or N(R_{B′})₂, or any combination thereof, wherein each occurrence of R_{B′} is independently hydrogen, alkyl, aryl, or a protecting group.

In certain embodiments, R_{B} is hydrogen, methyl, or ethyl.

In certain embodiments, R_{B} is hydrogen, or methyl. In certain embodiments, R_{B} is methyl.

In certain embodiments, R_{B} is —CH₂F, —CHF₂, or —CF₃. In certain embodiments, R_{B} is —CF₃.

In other embodiments, R₂ is thiazole or substituted thiazole. In some embodiments, R₂ is oxazole or substituted oxazole.

In certain embodiments, R₁ is methyl.

In some embodiments, R₁ is methyl; and R₂ is one of:

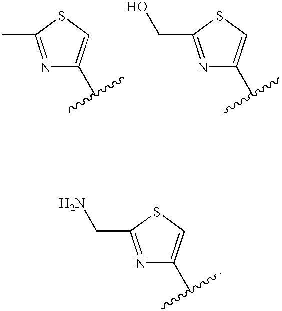

In other embodiments, R₁ is methyl; and R₂ is one of:

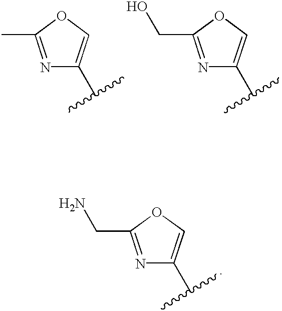

It will be appreciated that some of the foregoing classes and subclasses of compounds can exist in various isomeric forms. The invention encompasses the compounds as individual isomers substantially free of other isomers and alternatively, as mixtures of various isomers, e.g., racemic mixtures of stereoisomers. Additionally, the invention encompasses both (Z) and (E) double bond isomers unless otherwise specifically designated. Thus, compounds of the invention generally depicted in structure described herein encompass those structures in which double bonds are (Z) or (E). The invention also encompasses tautomers of specific compounds as described above. In addition to the above-mentioned compounds per se, this invention also encompasses pharmaceutically acceptable derivatives of these compounds and compositions comprising one or more compounds of the invention and one or more pharmaceutically acceptable excipients or additives.

Compounds of this invention which are of particular interest include those which:

exhibit cytotoxic or growth inhibitory effect on cancer cell lines maintained in vitro or in animal studies using a scientifically acceptable cancer cell xenograft model;

exhibit the ability to polymerize tubulin and stabilize microtubule assemblies;

exhibit enhanced water solubility over epothilones A, B, C or D, or paclitaxel, or additionally or alternatively exhibit sufficient solubility to be formulated in an aqueous medium; and

exhibit a therapeutic profile (e.g., optimum safety and curative effect) that is superior to that of epothilone B or paclitaxel.

This invention also provides a pharmaceutical preparation comprising at least one of the compounds as described above and herein, or a pharmaceutically acceptable derivative thereof, which compounds are capable of inhibiting the growth of or killing cancer cells, and, in certain embodiments of special interest are capable of inhibiting the growth of or killing multidrug resistant cancer cells. In certain embodiments, the pharmaceutical preparation also comprises as solubilizing or emulsifying agent such as Cremophor (polyoxyl 35 castor oil) or Solutol (polyethylene glycol 660 12-hydroxystrearate).

The invention further provides a method for inhibiting tumor growth and/or tumor metastasis. In certain embodiments of special interest, the invention provides a method of treating cancers by inhibiting tumor growth and/or tumor metastasis for tumors multidrug resistant cancer cells. The method involves the administration of a therapeutically effective amount of the compound or a pharmaceutically acceptable derivative thereof to a subject (including, but not limited to a human or animal) in need of it. In certain embodiments, specifically for treating cancers comprising multidrug resistant cancer cells, the therapeutically effective amount is an amount sufficient to kill or inhibit the growth of multidrug resistant cancer cell lines. In certain embodiments, the inventive compounds are useful for the treatment of solid tumors.

3) Compounds and Definitions

As discussed above, this invention provides novel compounds with a range of biological properties. Compounds of this invention have biological activities relevant for the treatment of diseases or other disorders such as proliferative diseases, including, but not limited to cancer.

Compounds of this invention include those specifically set forth above and described herein, and are illustrated in part by the various classes, subgenera and species disclosed elsewhere herein. In general, when referring to one exemplary compound, Epo-490, it will be appreciated that this compound is identical to that of ddEpoB, and that the two terms are used interchangeably herein. Additionally, when referring to another exemplary compound Homo-Epo-490, it will be appreciated that this compound is identical to that of homo-ddEpoB, and that the two terms are used interchageably herein.

It will be appreciated by one of ordinary skill in the art that asymmetric centers may exist in the compounds of the present invention. Thus, inventive compounds and pharmaceutical compositions thereof may be in the form of an individual enantiomer, diastereomer or geometric isomer, or may be in the form of a mixture of stereoisomers. In certain embodiments, the compounds of the invention are enantiopure compounds. In certain other embodiments, a mixtures of stereoisomers or diastereomers are provided. Additionally, the invention encompasses both (Z) and (E) double bond isomers (or cis and trans isomers) unless otherwise specifically designated. Thus, compounds of the invention generally depicted in structures described herein encompass those structures in which double bonds are (Z) or (E).

Additionally, the present invention provides pharmaceutically acceptable derivatives of the inventive compounds, and methods of treating a subject using these compounds, pharmaceutical compositions thereof, or either of these in combination with one or more additional therapeutic agents. The phrase, “pharmaceutically acceptable derivative”, as used herein, denotes any pharmaceutically acceptable salt, ester, or salt of such ester, of such compound, or any other adduct or derivative which, upon administration to a, patient, is capable of providing (directly or indirectly) a compound as otherwise described herein, or a metabolite or residue thereof. Pharmaceutically acceptable derivatives thus include among others pro-drugs. A pro-drug is a derivative of a compound, usually with significantly reduced pharmacological activity, which contains an additional moiety that is susceptible to removal in vivo yielding the parent molecule as the pharmacologically active species. An example of a pro-drug is an ester that is cleaved in vivo to yield a compound of interest. Pro-drugs of a variety of compounds, and materials and methods for derivatizing the parent compounds to create the pro-drugs, are known and may be adapted to the present invention. Certain exemplary pharmaceutical compositions and pharmaceutically acceptable derivatives will be discussed in more detail herein below.

Certain compounds of the present invention, and definitions of specific functional groups are also described in more detail below. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in “Organic Chemistry”, Thomas Sorrell, University Science Books, Sausalito: 1999, the entire contents of which are incorporated herein by reference. Furthermore, it will be appreciated by one of ordinary skill in the art that the synthetic methods, as described herein, utilize a variety of protecting groups. By the term “protecting group”, has used herein, it is meant that a particular functional moiety, e.g., O, S, or N, is temporarily blocked so that a reaction can be carried out selectively at another reactive site in a multifunctional compound. In preferred embodiments, a protecting group reacts selectively in good yield to give a protected substrate that is stable to the projected reactions; the protecting group must be selectively removed in good yield by readily available, preferably nontoxic reagents that do not attack the other funcational groups; the protecting group forms an easily separable derivative (more preferably without the generation of new stereogenic centers); and the protecting group has a minimum of additional functionality to avoid further sites of reaction. As detailed herein, oxygen, sulfur, nitrogen and carbon protecting groups may be utilized. Exemplary protecting groups are detailed herein, however, it will be appreciated that the present invention is not intended to be limited to these protecting groups; rather, a variety of additional equivalent protecting groups can be readily identified using the above criteria and utilized in the method of the present invention. Additionally, a variety of protecting groups are described in “Protective Groups in Organic Synthesis” Third Ed. Greene, T. W. and Wuts, P. G., Eds., John Wiley & Sons, New York: 1999, the entire contents of which are hereby incorporated by reference.

It will be appreciated that the compounds, as described herein, may be substituted with any number of substituents or functional moieties. In general, the term “substituted” whether preceded by the term “optionally” or not, and substituents contained in formulas of this invention, refer to the replacement of hydrogen radicals in a given structure with the radical of a specified substituent. When more than one position in any given structure may be substituted with more than one substituent selected from a specified group, the substituent may be either the same or different at every position. As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. For purposes of this invention, heteroatoms such as nitrogen may have hydrogen substituents and/or any permissible substituents of organic compounds described herein which satisfy the valencies of the heteroatoms. Furthermore, this invention is not intended to be limited in any manner by the permissible substituents of organic compounds. Combinations of substituents and variables envisioned by this invention are preferably those that result in the formation of stable compounds useful in the treatment, for example of proliferative disorders, including, but not limited to cancer. The term “stable”, as used herein, preferably refers to compounds which possess stability sufficient to allow manufacture and which maintain the integrity of the compound for a sufficient period of time to be detected and preferably for a sufficient period of time to be useful for the purposes detailed herein.

The term “aliphatic”, as used herein, includes both saturated and unsaturated, straight chain (i.e., unbranched), branched, cyclic, or polycyclic aliphatic hydrocarbons, which are optionally substituted with one or more functional groups. As will be appreciated by one of ordinary skill in the art, “aliphatic” is intended herein to include, but is not limited to, alkyl, alkenyl, alkynyl, cycloalkyl, cycloalkenyl, and cycloalkynyl moieties. Thus, as used herein, the term “alkyl” includes straight, branched and cyclic alkyl groups. An analogous convention applies to other generic terms such as “alkenyl”, “alkynyl” and the like. Furthermore, as used herein, the terms “alkyl”, “alkenyl”, “alkynyl” and the like encompass both substituted and unsubstituted groups. In certain embodiments, as used herein, “lower alkyl” is used to indicate those alkyl groups (cyclic, acyclic, substituted, unsubstituted, branched or unbranched) having 1-6 carbon atoms.

In certain embodiments, the alkyl, alkenyl and alkynyl groups employed in the invention contain 1-20 aliphatic carbon atoms. In certain other embodiments, the alkyl, alkenyl, and akynyl groups employed in the invention contain 1-10 aliphatic carbon atoms. In yet other embodiments, the alkyl, alkenyl, and alkynyl groups employed in the invention contain 1-8 aliphatic carbon atoms. In still other embodiments, the alkyl, alkenyl, and alkynyl groups employed in the invention contain 1-6 aliphatic carbon atoms. In yet other embodiments, the alkyl, alkenyl, and alkynyl groups employed in the invention contain 1-4 carbon atoms. Illustrative aliphatic groups thus include, but are not limited to, for example, methyl, ethyl, n-propyl, isopropyl, cyclopropyl, —CH₂-cyclopropyl, allyl, n-butyl, sec-butyl, isobutyl, tert-butyl, cyclobutyl, —CH₂-cyclobutyl, n-pentyl, sec-pentyl, isopentyl, tert-pentyl, cyclopentyl, —CH₂-cyclopentyl, n-hexyl, sec-hexyl, cyclohexyl, —CH₂-cyclohexyl moieties and the like, which again, may bear one or more substituents. Alkenyl groups include, but are not limited to, for example, ethenyl, propenyl, butenyl, 1-methyl-2-buten-1-yl, and the like. Representative alkynyl groups include, but are not limited to, ethynyl, 2-propynyl (propargyl), 1-propynyl and the like.

The term “alkoxy”, or “thioalkyl” as used herein refers to an alkyl group, as previously defined, attached to the parent molecular moiety through an oxygen atom or through a sulfur atom. In certain embodiments, the alkyl group contains 1-20 alipahtic carbon atoms. In certain other embodiments, the alkyl group contains 1-10 aliphatic carbon atoms. In yet other embodiments, the alkyl, alkenyl, and alkynyl groups employed in the invention contain 1-8 aliphatic carbon atoms. In still other embodiments, the alkyl group contains 1-6 aliphatic carbon atoms. In yet other embodiments, the alkyl group contains 1-4 aliphatic carbon atoms. Examples of alkoxy, include but are not limited to, methoxy, ethoxy, propoxy, isopropoxy, n-butoxy, tert-butoxy, neopentoxy and n-hexoxy. Examples of thioalkyl include, but are not limited to, methylthio, ethylthio, propylthio, isopropylthio, n-butylthio, and the like.

The term “alkylamino” refers to a group having the structure —NHR′ wherein R′ is alkyl, as defined herein. In certain embodiments, the alkyl group contains 1-20 aliphatic carbon atoms. In certain other embodiments, the alkyl group contains 1-10 aliphatic carbon atoms. In yet other embodiments, the alkyl, alkenyl, and alkynyl groups employed in the invention contain 1-8 aliphatic carbon atoms. In still other embodiments, the alkyl group contains 1-6 aliphatic carbon atoms. In yet other embodiments, the alkyl group contains 1-4 aliphatic carbon atoms. Examples of alkylamino include, but are not limited to, methylamino, ethylamino, iso-propylamino and the like.

Some examples of substituents of the above-described aliphatic (and other) moieties of compounds of the invention include, but are not limited to aliphatic; heteroaliphatic; aryl; heteroaryl; arylalkyl; heteroarylalkyl, heteroarylalkenyl, heteroarylalkynyl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂Ch₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ wherein each occurrence of Rₓ independently includes, but is not limited to, aliphatic, heteroaliphatic, aryl, heteroaryl, arylalkyl, arylalkenyl, arylalkynyl, or heteroarylalkyl, heteroarylalkenyl, heteroarylalkynyl, heteroarylalkenyl, heteroarylalkynyl, arylalkenyl, arylalkynyl, wherein any of the aliphatic, heteroaliphatic, arylalkyl, arylalkenyl, arylalkynyl, or heteroarylalkyl, heteroarylalkenyl, heteroarylalkynyl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substituents are illustrated by the specific embodiments shown in the Examples that are described herein.

In general, the terms “aryl” and “heteroaryl”, as used herein, refer to stable mono- or polycyclic, heterocyclic, polycyclic, and polyheterocyclic unsaturated moieties having preferably 3-14 carbon atoms, each of which may be substituted or unsubstituted. Substituents include, but are not limited to, any of the previously mentioned substitutents, i.e., the substituents recited for aliphatic moieties, or for other moieties as disclosed herein, resulting in the formation of a stable compound. In certain embodiments of the present invention, “aryl” refers to a mono- or bicyclic carbocyclic ring system having one or two aromatic rings including, but not limited to, phenyl, naphthyl, tetrahydronaphthyl, indanyl, indenyl and the like. In certain embodiments of the present invention, the term “heteroaryl”, as used herein, refers to a cyclic aromatic radical having from five to ten ring atoms of which one ring atom is selected from S, O and N; zero, one or two ring atoms are additional heteroatoms independently selected from S, O and N; and the remaining ring atoms are carbon, the radical being joined to the rest of the molecule via any of the ring atoms, such as, for example, pyridyl, pyrazinyl, pyrimidinyl, pyrrolyl, pyrazolyl, imidazolyl, thiazolyl, oxazolyl, isooxazolyl, thiadiazolyl,oxadiazolyl, thiophenyl, furanyl, quinolinyl, isoquinolinyl, and the like.

It will be appreciated that aryl and heteroaryl groups (including bicyclic aryl groups) can be unsubstituted or substituted, wherein substitution includes replacement of one, two or three of the hydrogen atoms thereon independently with any one or more of the following moieties including, but not limited to: aliphatic; heteroaliphatic; aryl; heteroaryl; arylalkyl; heteroarylalkyl, heteroarylalkenyl, heteroarylalkynyl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ wherein each occurrence of Rₓ independently includes, but is not limited to, aliphatic, heteroaliphatic, aryl, heteroaryl, arylalkyl, arylalkenyl, arylalkynyl, or heteroarylalkyl, heteroarylalkenyl, heteroarylalkynyl, heteroarylalkenyl, heteroarylalkynyl, arylalkenyl, arylalkynyl, wherein any of the aliphatic, heteroaliphatic, arylalkyl, arylalkenyl, arylalkynyl, or heteroarylalkyl, heteroarylalkenyl, heteroarylalkynyl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substitutents are illustrated by the specific embodiments shown in the Examples that are described herein.

The term “cycloalkyl”, as used herein, refers specifically to groups having three to seven, preferably three to ten carbon atoms. Suitable cycloalkyls include, but are not limited to cyclopropyl, cyclobutyl, cyclopentyl, cyclohexyl, cycloheptyl and the like, which, as in the case of other aliphatic, heteroaliphatic or hetercyclic moieties, may optionally be substituted with substituents including, but not limited to aliphatic; heteroaliphatic; aryl; heteroaryl; arylalkyl; heteroarylalkyl, heteroarylalkenyl, heteroarylalkynyl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ wherein each occurrence of Rₓ independently includes, but is not limited to, aliphatic, heteroaliphatic, aryl, heteroaryl, arylalkyl, arylalkenyl, arylalkynyl, or heteroarylalkyl, heteroarylalkenyl, heteroarylalkynyl, heteroarylalkenyl, heteroarylalkynyl, arylalkenyl, arylalkynyl, wherein any of the aliphatic, heteroaliphatic, arylalkyl, arylalkenyl, arylalkynyl, or heteroarylalkyl, heteroarylalkenyl, heteroarylalkynyl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substitutents are illustrated by the specific embodiments shown in the Examples that are described herein.

The term “heteroaliphatic”, as used herein, refers to aliphatic moieties that contain one or more oxygen, sulfur, nitrogen, phosphorus or silicon atoms, e.g., in place of carbon atoms. Heteroaliphatic moieties may be branched, unbranched, cyclic or acyclic and include saturated and unsaturated heterocycles such as morpholino, pyrrolidinyl, etc. In certain embodiments, heteroaliphatic moieties are substituted by independent replacement of one or more of the hydrogen atoms thereon with one or more moieties including, but not limited to aliphatic; heteroaliphatic; aryl; heteroaryl; arylalkyl; heteroarylalkyl, heteroarylalkenyl, heteroarylalkynyl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ wherein each occurrence of Rₓ independently includes, but is not limited to, aliphatic, heteroaliphatic, aryl, heteroaryl, arylalkyl, arylalkenyl, arylalkynyl, or heteroarylalkyl, heteroarylalkenyl, heteroarylalkynyl, heteroarylalkenyl, heteroarylalkynyl, arylalkenyl, arylalkynyl, wherein any of the aliphatic, heteroaliphatic, arylalkyl, arylalkenyl, arylalkynyl, or heteroarylalkyl, heteroarylalkenyl, heteroarylalkynyl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substitutents are illustrated by the specific embodiments shown in the Examples that are described herein.

The terms “halo” and “halogen” as used herein refer to an atom selected from fluorine, chlorine, bromine and iodine.

The term “haloalkyl” denotes an alkyl group, as defined above, having one, two, or three halogen atoms attached thereto and is exemplified by such groups as chloromethyl, bromoethyl, trifluoromethyl, and the like.

The term “heterocycloalkyl” or “heterocycle”, as used herein, refers to a non-aromatic 5-, 6- or 7-membered ring or a polycyclic group, including, but not limited to a bi- or tri-cyclic group comprising fused six-membered rings having between one and three heteroatoms independently selected from oxygen, sulfur and nitrogen, wherein (i) each 5-membered ring has 0 to 1 double bonds and each 6-membered ring has 0 to 2 double bonds, (ii) the nitrogen and sulfur heteroatoms may be optionally be oxidized, (iii) the nitrogen heteroatom may optionally be quaternized, and (iv) any of the above heterocyclic rings may be fused to a benzene ring. Representative heterocycles include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl. In certain embodiments, a “substituted heterocycloalkyl or heterocycle” group is utilized and as used herein, refers to a heterocycloalkyl or heterocycle group, as defined above, substituted by the independent replacement of one, two or three of the hydrogen atoms thereon with but are not limited to aliphatic; heteroaliphatic; aryl; heteroaryl; arylalkyl; heteroarylalkyl, heteroarylalkenyl, heteroarylalkynyl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ wherein each occurrence of Rₓ independently includes, but is not limited to, aliphatic, heteroaliphatic, aryl, heteroaryl, arylalkyl, arylalkenyl, arylalkynyl, heteroarylalkyl, heteroarylalkenyl, or heteroarylalkynyl, heteroarylalkenyl, heteroarylalkynyl, arylalkenyl, arylalkynyl, wherein any of the aliphatic, heteroaliphatic, arylalkyl, arylalkenyl, arylalkynyl, or heteroarylalkyl, heteroarylalkenyl, heteroarylalkynyl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substitutents are illustrated by the specific embodiments shown in the Examples which are described herein.

“Labeled”: As used herein, the term “labeled” is intended to mean that a compound has at least one element, isotope or chemical compound attached to enable the detection of the compound. In general, labels fall into three classes: a) isotopic labels, which may be radioactive or heavy isotopes, including, but not limited to, ²H, ³H, ³²P, ³⁵S, ⁶⁷Ga, ^{99m}Tc(Tc-99m), ¹¹¹In, ¹²³I, ¹²⁵I, ¹⁶⁹Yb and ¹⁸⁶Re; b) immune labels, which may be antibodies or antigens; and c) colored or fluorescent dyes. It will be appreciated that the labels may be incorporated into the compound at any position that does not interfere with the biological activity or characteristic of the compound that is being detected. In certain embodiments of the invention, photoaffinity labeling is utilized for the direct elucidation of intermolecular interactions in biological systems (e.g. to probe the epothilone binding site in a tubulin dimer). A variety of known photophores can be employed, most relying on photoconversion of diazo compounds, azides, or diazirines to nitrenes or carbenes (See, Bayley, H., Photogenerated Reagents in Biochemistry and Molecular Biology (1983), Elsevier, Amsterdam.), the entire contents of which are hereby incorporated by reference. In certain embodiments of the invention, the photoaffinity labels employed are o-, m- and p-azidobenzoyls, substituted with one or more halogen moieties, including, but not limited to 4-azido-2,3,5,6-tetrafluorobenzoic acid.

“Polymer”: The term “polymer”, as used herein, refers to a composition comprising chains that may be open, closed, linear, branched or cross-linked of repeating units (monomers) that may be the same or different. It will be appreciated that in certain embodiments the term polymer refers to biopolymers, which, as used herein, is intended to refer to polymeric materials found in nature or based upon those materials found in nature, including, but not limited to nucleic acids, peptides, and mimetics thereof. In certain other embodiments, the term polymer refers to synthetic polymers, such as biodegradable polymers or other polymeric materials. It will be appreciated that polymeric solid supports are also encompassed by the polymers of the present invention. Inventive compounds can be attached to polymeric supports and thus certain synthetic modifications can be conducted on the solid phase. As used herein, the term “solid support” is meant to include, but is not limited to, pellets, disks, capillaries, hollow fibers, needles, pins, solid fibers, cellulose beads, pore-glass beads, silica gels, polystyrene beads optionally cross-linked with divinylbenzene, grafted co-poly beads, poly-acrylamide beads, latex beads, dimethylacrylamide beads optionally crosslinked with N-N′-bis-acryloylethylenediamine, and glass particles coated with a hydrophobic polymer. One of ordinary skill in the art will realize that the choice of particular solid support will be limited by the compatability of the support with the reaction chemistry being utilized. An exemplary solid support is a Tentagel amino resin, a composite of 1) a polystyrene bead crosslinked with divinylbenzene and 2) PEG (polyethylene glycol). Tentagel is a particularly useful solid support because it provides a versatile support for use in on-bead or off-bead assays, and it also undergoes excellent swelling in solvents ranging from toluene to water.

4) Synthetic Methodology:

As described above, the synthesis of certain epothilones, desoxyepothilones and analogues thereof have been previously described (see, U.S. Pat. Nos. 6,242,469, 6,284,781, 6,300,355, and 6,204,388; U.S. patent applications Ser. Nos. 09/797,027 and 09/796,959; and PCT Publication Nos. WO 99/01124, WO99/43653 and WO01/64650, the entire contents of which are hereby incorporated by reference). In recognition of the need for improved or additional synthetic methodologies to efficiently generate epothilones, desoxyepothilones and analogues thereof in large quantities, the present invention provides an efficient and modular route for the synthesis of epothilones, desoxyepothilones and analogues thereof. Although the synthesis of certain exemplary compounds is described in the Exemplification herein, it will be appreciated that this methodology is generally applicable to the generation of analogues and conjugates as discussed above for each of the classes and subclasses described herein, and as described in more detail below.

In general, the methods of the present invention represent a modular approach to the synthesis of desoxyepothilones whereby compounds having the structure (I) or a subset of compounds of structure (I) having the structure (II) depicted below can be synthesized from two or more of the intermediates (A), (B), (C), (D) and (E), in any order.

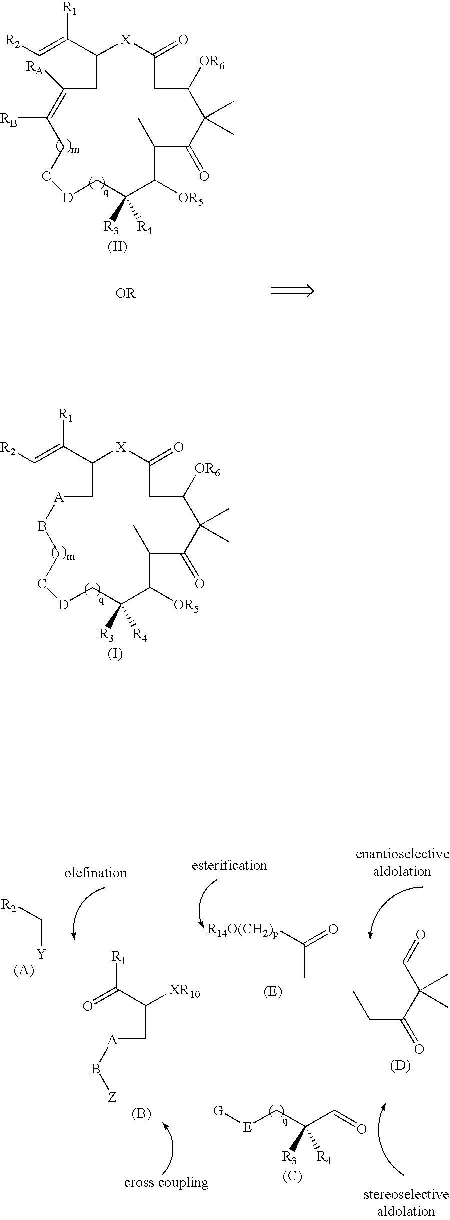

In general, the methods of the invention comprise reacting two or more of components (A), (B), (C), (D), or (E) to generate an intermediate resulting from the coupling of said two or more components, which intermediate can then be reacted with one or more reagents, or alternatively or additionally, can be further reacted with one or more of components (A), (B), (C), (D), or (E), or any coupled combination thereof, to generate compounds of formula (I) or (II).

In certain other embodiments two of (A), (B), (C), (D), or (E) are reacted to generate an intermediate resulting from the coupling of any two of (A), (B), (C), (D), or (E), which intermediate is then reacted with one or more additional reagents, or alternatively or additionally is reacted with one or more of (A), (B), (C), (D), or (E) or any coupled combination thereof to generate compounds of formula (I) or (II).

In certain other embodiments three of (A), (B), (C), (D), or (E) are reacted to generate an intermediate resulting from the coupling of any three of (A), (B), (C), (D), or (E), which intermediate is then reacted with one or more additional reagents, or alternatively or additionally is reacted with one or more of (A), (B), (C), (D), or (E) or any coupled combination thereof to generate compounds of formula (I) or (II).

In still other embodiments four of (A), (B), (C), (D), or (E) are reacted to generate an intermediate resulting from the coupling of any four of (A), (B), (C), (D), or (E), which intermediate is then reacted with one or more additional reagents, or alternatively or additionally is reacted with one or more of (A), (B), (C), (D), or (E) or any coupled combination thereof to generate compounds of formula (I) or (II).

In yet other embodiments each of (A), (B), (C), (D), or (E) is reacted to generate an intermediate resulting from the coupling of each of (A), (B), (C), (D), or (E), which intermediate is then reacted with one or more additional reagents to generate compounds of formula (I) or (II).

In certain embodiments of special interest, each of the components (A), (B), (C), (D), and (E) or four of the components (B), (C), (D), and (E) can be reacted in any order under suitable conditions to generate a cyclization precursor having any one of the structures (F), (G), (H), (I), or (J), which cyclization precursors can be reacted under a variety of conditions with a macrocyclization reagent, as depicted generally below, to generate a compound having the structure (I):

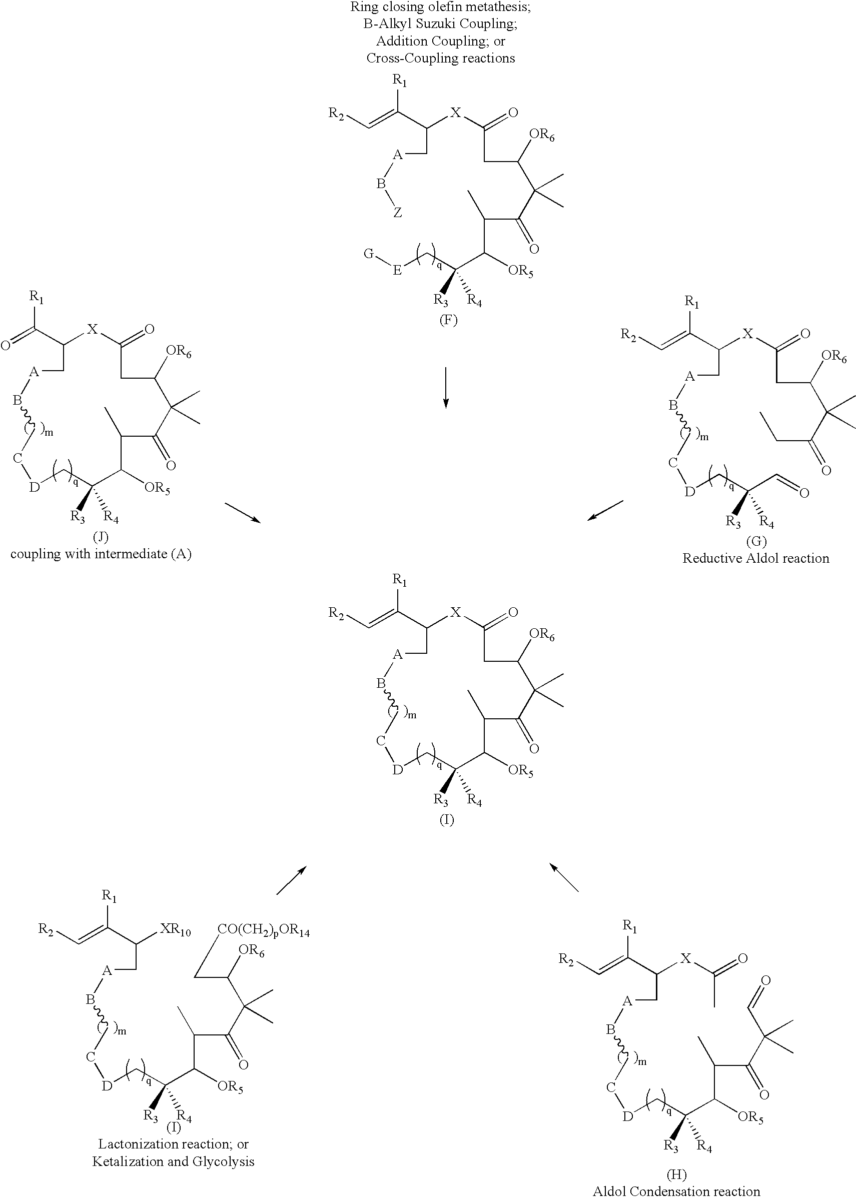

In certain other embodiments of special interest, A—B represents CR_{A}═CR_{B}, and thus component (B) has the structure (B2):

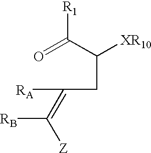

and each of the components (A), (B2), (C), (D), and (E), or four of the components (B2), (C), (D), and (E) can be reacted in any order under suitable conditions to generate a cyclization precursor having any one of the structures (F2), (G2), (H2), (I2), or (J2), which cyclization precursors can be reacted under a variety of conditions with a macrocyclization reagent, as depicted generally below, to generate a compound having the structure (II):

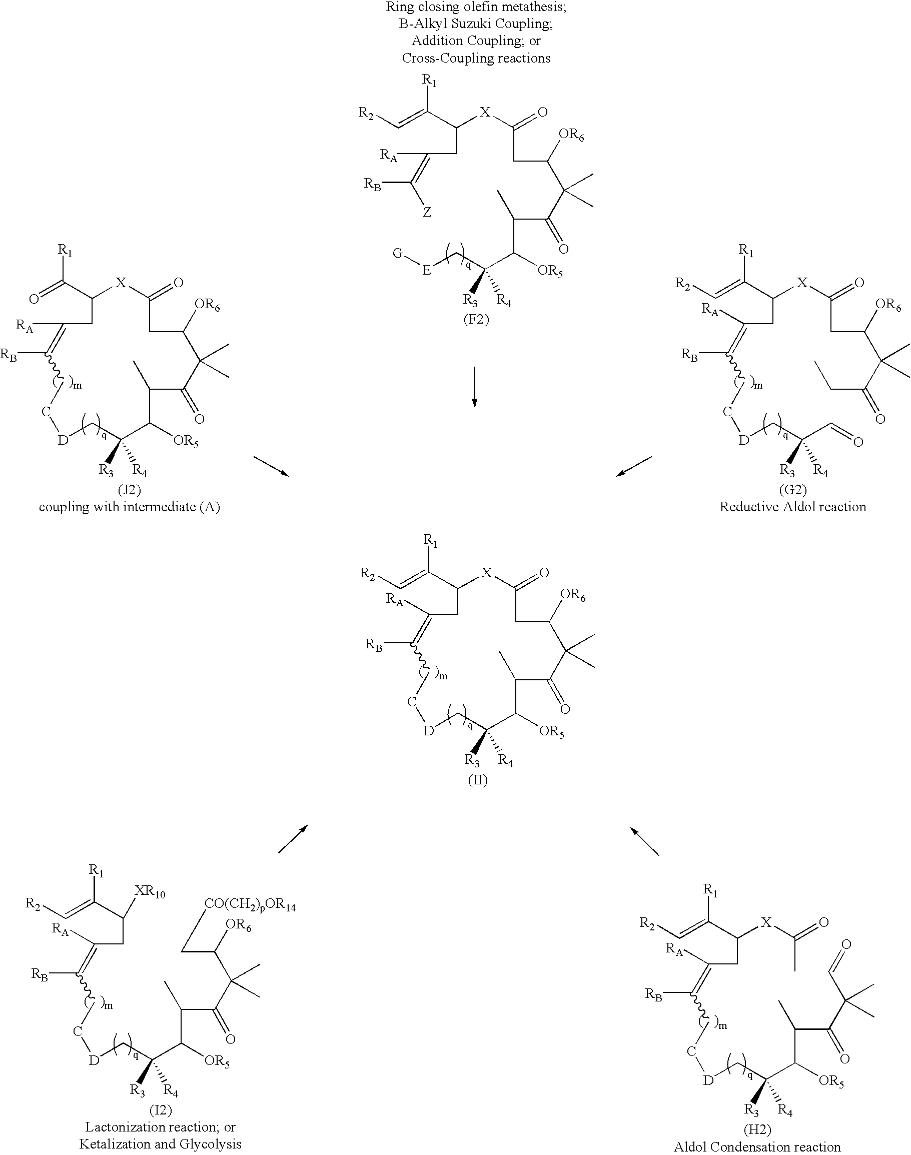

To approach the compounds as described above and in various classes and subclasses herein, a strategy has been developed which features a convergent and modular nature with control of relevant selectivities at each step as depicted above. The conciseness of the syntheses of the key intermediates, as described in more detail herein, readily allow for large scale preparation and easy structural variation in each synthetic segment. In particular, the present investigation has led to significant improvement in the preparation of the polypropionate domain (C+D+E) (which serves as a widely applicable intermediate for accessing various analogues) as well as the synthesis of individual segments. It should be noted that this modular approach, as depicted generically above, allows for all key bond-forming processes, except for the olefination, to be utilized for macrocyclization.

It will additionally be appreciated that the compounds as described above and herein, may be further reacted with one or more reagents to effect diversification of the compound or alternatively or additionally, may be reacted with one or more reagents to effect deprotection of any protected functional groups present in the molecule to generate a variety of compounds having structures (I) and (I′), and classes and subclasses thereof, as described in more detail above and herein. It will be appreciated that, in addition to the novel compounds represented by (I) and (II) and classes and subclasses thereof as described herein, the novel synthetic methodology described herein is also applicable to the synthesis of any epothilone, desoxyepothilone or analogue thereof. Signficantly, the present methodology allows for the rapid modification of a variety of diversifiable segments (e.g., X, R₂, R_{A}, R_{B}, R_{C}, R_{D}, etc.) and allows for the rapid modification of ring size (e.g., expansion to 17-, 18- and 19-membered rings) and thus easily affords a variety of epothilone, desoxyepothilones, and analogues thereof in large quantities.

In one embodiment of the general method described above, a method for the synthesis of a compound having the structure (I) is provided which compound is described generally herein and in classes and subclasses herein:

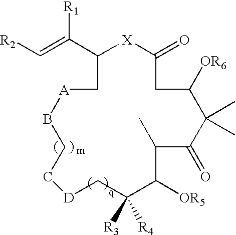

which method comprises:

(1) reacting each of the intermediates (A), (B), (C), (D), and (E) or reacting the intermediates (B), (C), (D), and (E):

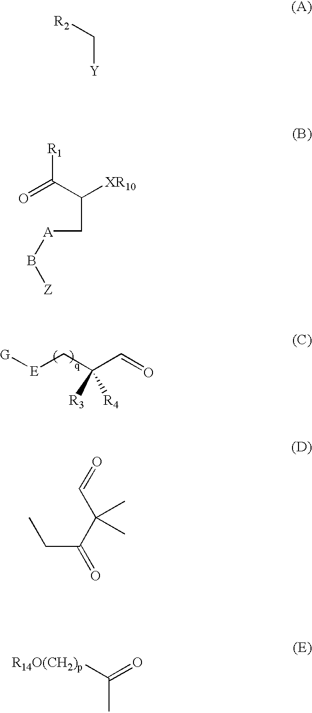

wherein A—B, R₁, R₂, R₃, R₄ and R_{B} are as defined generally herein and in classes and subclasses described herein, and wherein XR₁₀ is NR₇R₁₀, OR₁₀, SR₁₀ or C(R₇)₂R₁₀, wherein R₁₀ is hydrogen, a protecting group, or —(C═O)CH₃; Y is halogen, or a phosphorus ylide; Z is halogen or —(CH₂)ₘ—CR₁₆═C(R₁₇)₂, wherein R₁₆ is hydrogen or a heteroatom substituent (e.g., O, N, S or halogen) and each occurrence of R₁₇ is independently hydrogen or a heteroatom substituent (e.g., O, N, S or halogen); R₁₄ is hydrogen or a protecting group; G-E together represent HC≡C, or CR₁₅R_{C}═CR_{D}, wherein R_{C} and R_{D} are as defined herein, R₁₅ is hydrogen, disubstituted borane, trisubstituted tin, or trisubstituted silane; m is 0-3; q is 1-3; and p is 0-2,

in any order and under suitable conditions to generate an intermediate having any one of the structures (F), (G), (H), (I) or (J):

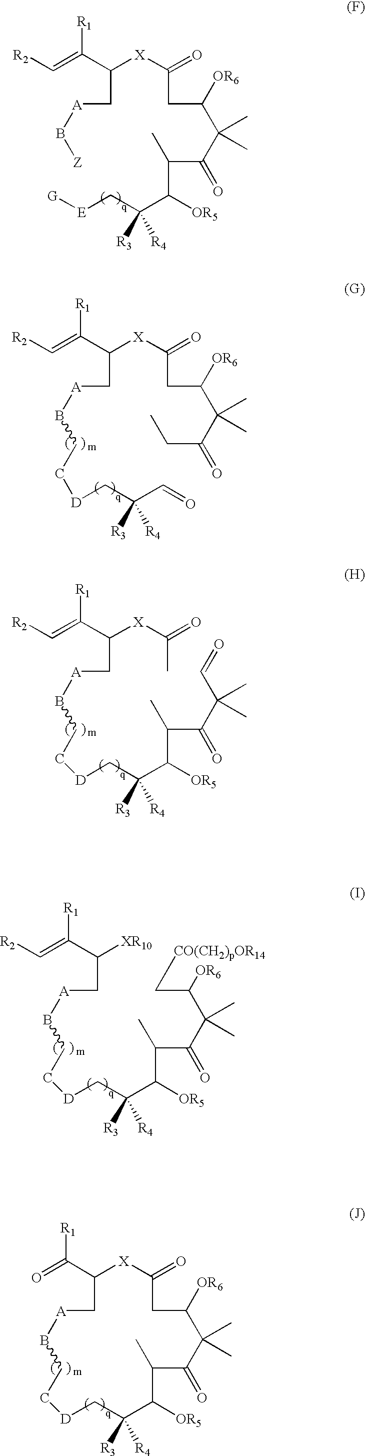

(2) reacting any one of the intermediates (F), (G), (H), or (I), in the presence of a macrocyclization reagent, or reacting the intermediate (J) with (A) under suitable conditions, and optionally further reacting with one or more additional reagents to generate the compound (I).

In certain embodiments of the method as described above, the sum of m and q is 1, 2, 3, 4 or 5.

In certain other embodiments of the method as described above, the sum of m and q is 2, 3 or 4.

In still other embodiments, q is 1 and m is 0, 1, 2, or 3. In yet other embodiments, q is 1 and m is 1, 2 or 3.

In one exemplary embodiment, a method for the synthesis of a compound having the structure (II) is provided:

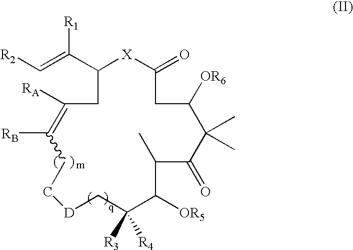

(1) reacting each of the intermediates (A), (B2), (C), (D), and (E) or reacting the intermediates (B2), (C), (D), and (E):

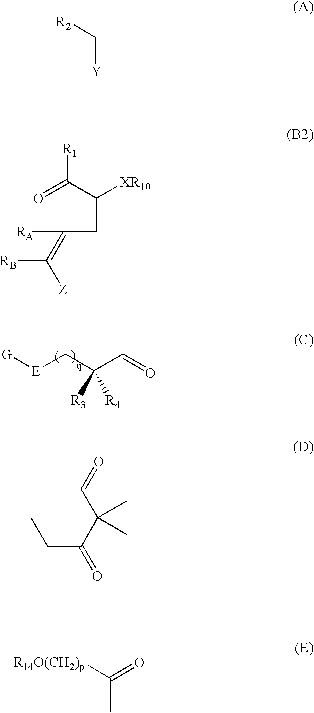

wherein R₁, R₂, R₃, R₄ R_{A} and R_{B} are as defined above, and wherein XR₁₀ is NR₇R₁₀, OR₁₀, SR₁₀ or C(R₇)₂R₁₀, wherein R₁₀ is hydrogen, a protecting group, or —(C═O)CH₃; Y is halogen, or a phosphorus ylide; Z is halogen or —(CH₂)ₘ—CR₁₆═C(R₁₇)₂, wherein R₁₆ is hydrogen or a heteroatom substituent (e.g., O, N, S or halogen) and each occurrence of R₁₇ is independently hydrogen or a heteroatom substituent (e.g., O, N, S or halogen); R₁₄ is hydrogen or a protecting group; G-E together represent HC≡C, or CR₁₅R_{C}═CR_{D}, wherein R_{C} and R_{D} are as defined herein, R₁₅ is hydrogen, disubstituted borane, trisubstituted tin, or trisubstituted silane; m is 0-3; q is 1-3; and p is 0-2,

in any order and under suitable conditions to generate an intermediate having any one of the structures (F2), (G2), (H2), (I2) or (J2):

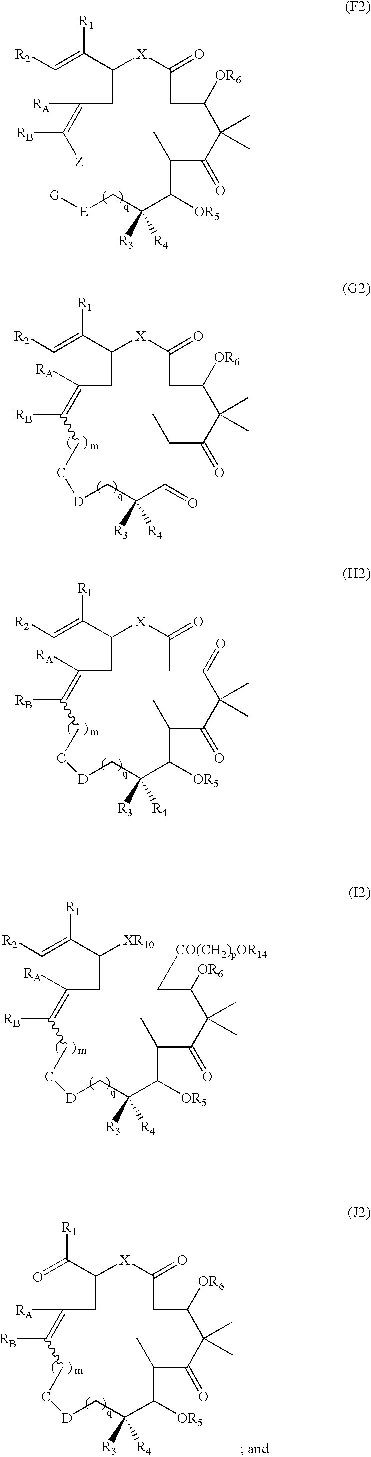

(2) reacting any one of the intermediates (F2), (G2), (H2), or (I2), in the presence of a macrocyclization reagent, or reacting the intermediate (J2) with (A2) under suitable conditions, and optionally further reacting with one or more additional reagents to generate the compound (II).

In certain embodiments of the method as described above, the sum of m and q is 1, 2, 3, 4, or 5.

In certain other embodiments of the method as described above, the sum of m and q is 2, 3 or 4.

In still other embodiments, q is 1 and m is 0, 1, 2, or 3. In yet other embodiments, q is 1 and m is 1, 2 or 3.

In certain embodiments, the method further comprises reacting the compound (II) with one or more additional reagents to generate a compound having the structure (I) as depicted and defined above and herein and in classes and subclasses described herein.

In certain embodiments for each of the methods generally described above, it may be desirable to generate compounds (I) or (II), wherein in compounds (I) and (II), R₂ is a substituted thiazolyl moiety and thus the compounds have the structures:

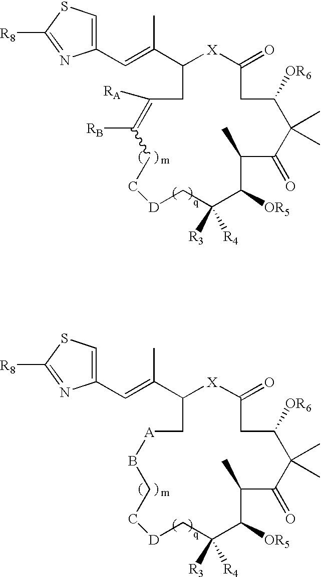

Either of these compounds can be generated from intermediates and methods as described generically above, wherein the intermediate (A) may have the structure (A3):

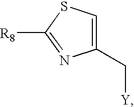

and thus for the intermediates (F), (G), (H), (I) and (J), R₁ is methyl and R₂ is a substituted thiazolyl moiety and may have the structures depicted directly below (F3), (G3), (H3), (I3) and (J3), and as described in the various classes and subclasses herein:

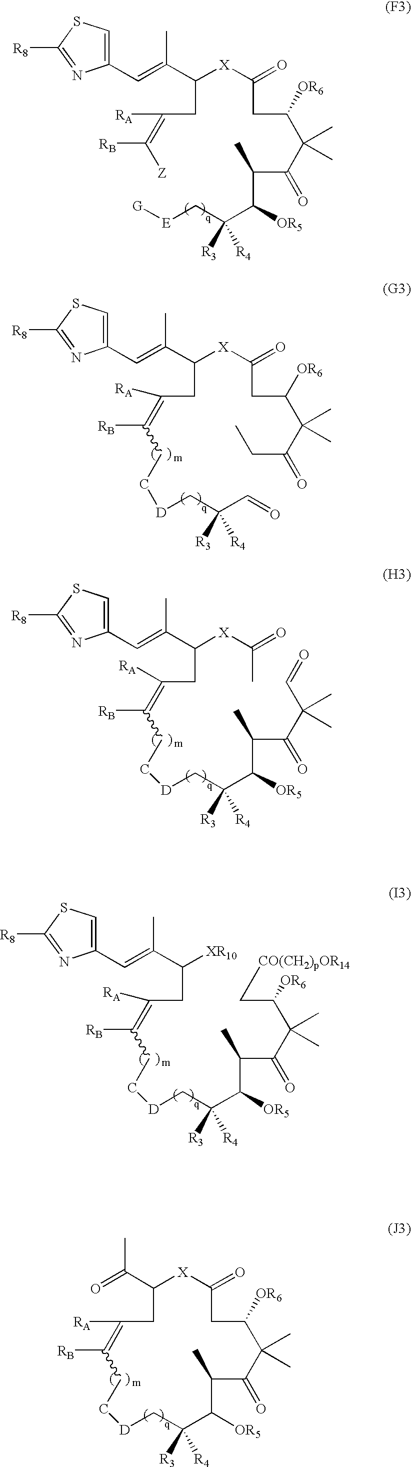

It will be appreciated that in certain embodiments of the compounds and intermediates described directly above, X is O. Additionally, the methodology described directly above and more generically herein may be utilized for any of the compounds, classes, subclasses and species thereof as described above and herein.

For example, the inventive methodology can be utilized, in one exemplary embodiment, to combine fragments (A) and (B) (or (B2))to generate an intermediate (K) or (K2):

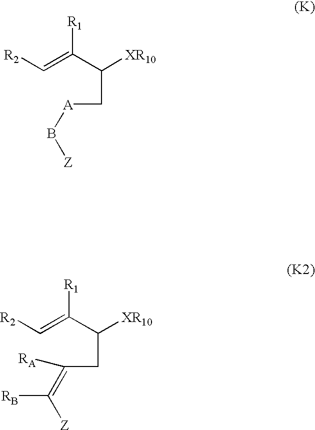

and (C), (D) and (E) are reacted as described generally above to generate intermediate (L):

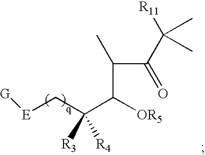

These two fragments can then be coupled via an aldolization or via esterification to generate the intermediate (F) or (F2)

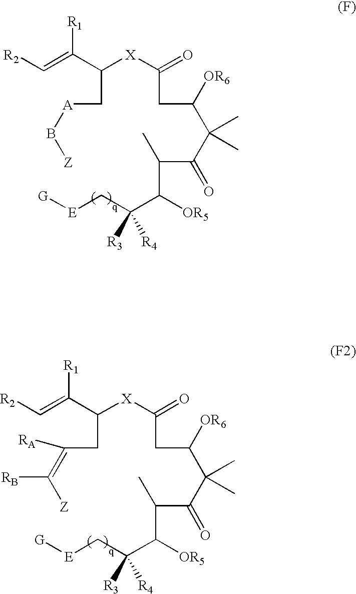

In certain other embodiments, R₂ is a substituted thiazolyl moiety and fragments (A) and (B2) are reacted to generate the intermediate (M):

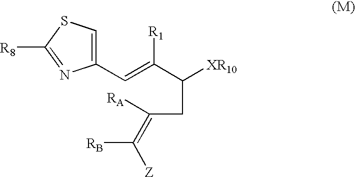

Fragments (C), (D) and (E) are then reacted to generate the intermediate (L):

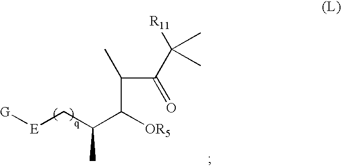

These two intermediates (M) and (L) can be coupled via aldolization or esterification as described generally above to generate the intermediate (N):

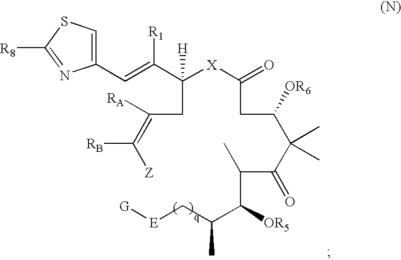

In certain embodiments for the methodology described generally above and herein, R₈ is methyl, amino or CH₂OH.

a) Preparation of Macrocyclization Precursors:

As described generally above, the intermediates (A), (B), (C), (D) and (E) (or subsets thereof) may be reacted in any order to generate the intermediates (F), (G), (H), (I) and (J) as described above. When referring to specific intermediates and fragments in this section, it will be understood that the fragments include those generally described as well as subsets thereof (e.g., (B2)).

In general, intermediates (B) and (E) may be reacted at any stage using an esterification reaction (or analog thereof) (even after (B) and (E) may have been reacted with other fragments), as described previously. Additionally, fragments (D) and (E) may be reacted at any stage using a enantioselective aldolation (even after (D) and (E) may have been reacted with other fragments).

In general, intermediates (A) and (B) may be reacted at any stage (for example, even after (B) has been reacted with (C) or (E), or any other combinations) under suitable olefination conditions to effect coupling of the two fragments. In certain exemplary embodiments, the fragments can be joined via a Wittig-type olefination, or any variation thereof, which involves the reaction of the phosphorus ylide and a ketone to yield an olefin (and phosphine oxide).

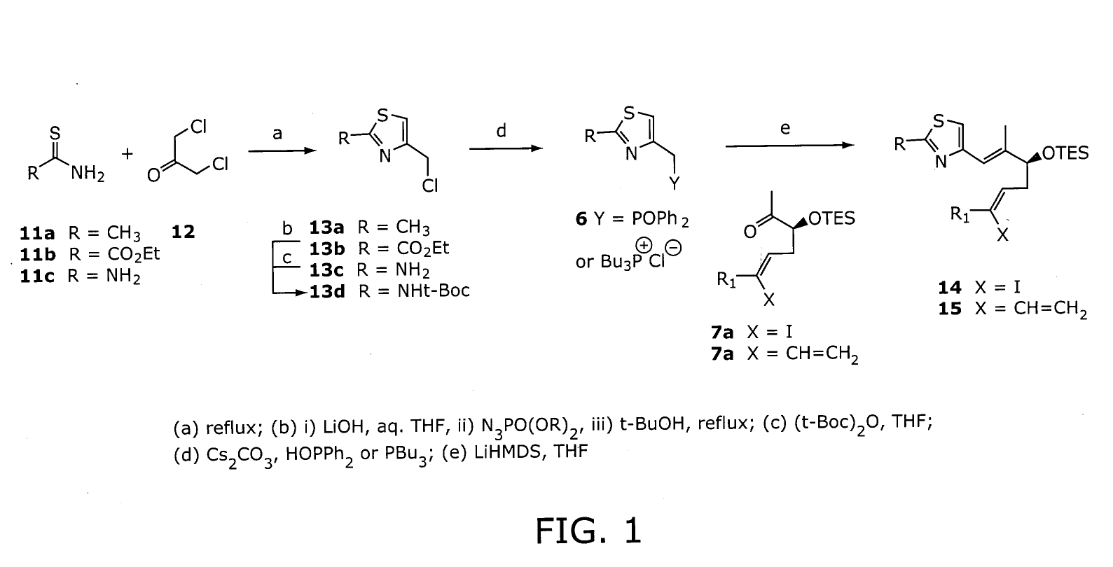

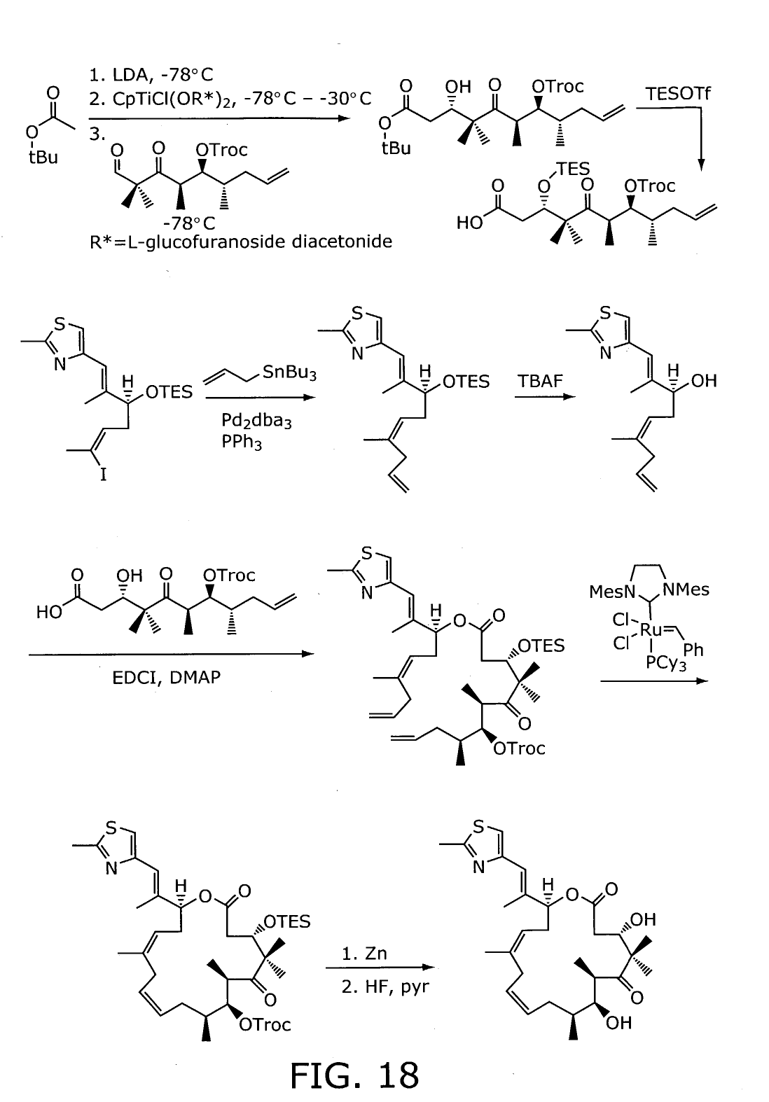

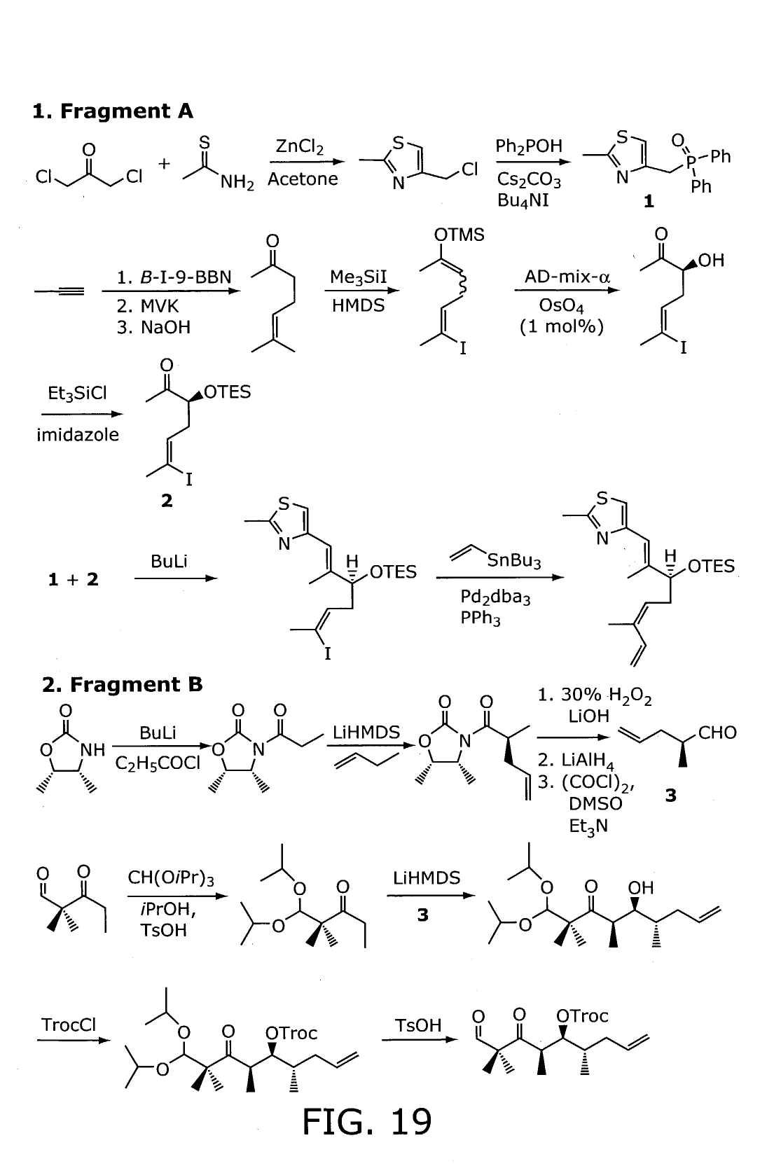

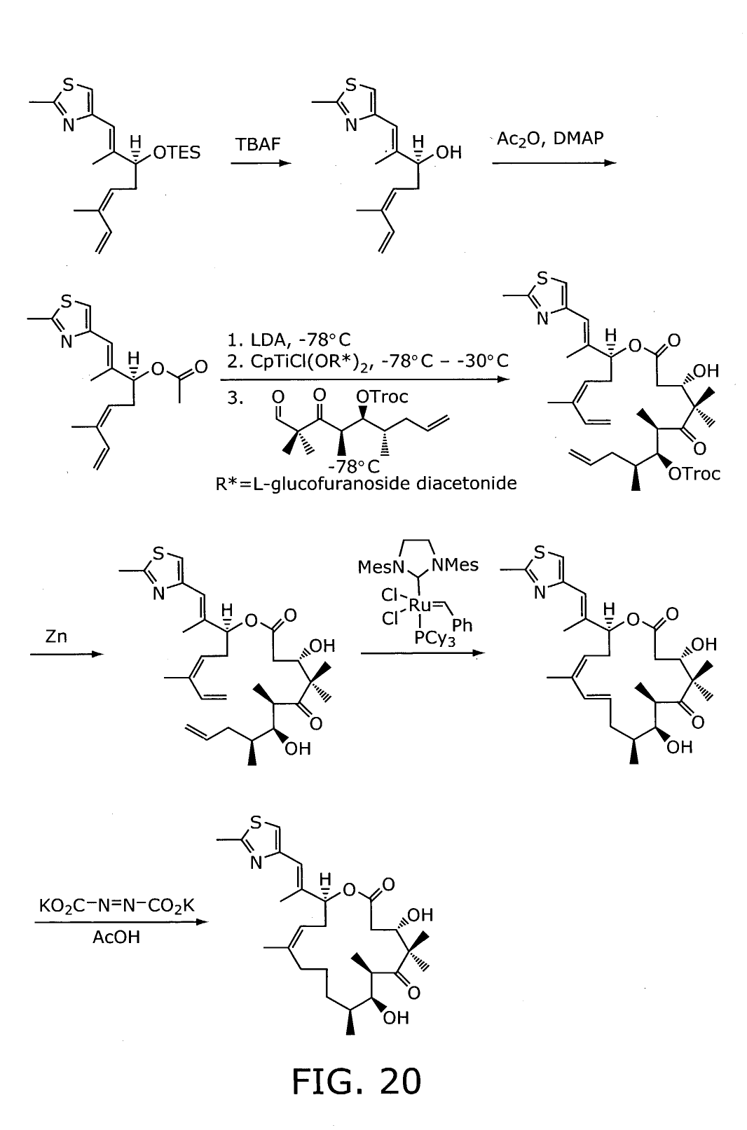

In one exemplary embodiment, as depicted in FIG. 1, the western fragment (14 or 15) involves the Wittig type olefination to connect segments 6 and 7 (FIG. 1) with control of olefin geometry. This sequence proved efficient in multigram scale for the practical synthesis of dEpoB (R=CH₃) and dEpoF (R=CO₂Et and CH₂OTroc). For the synthesis of the 20-desmethyl-20-amino derivatives, 2-aminothiazole 13c is prepared from the condensation of thiourea (11c) and 1,3-dichloroacetone (12). Alternatively, thiazole 13b or 14b (R=CO₂Et) can be converted to the corresponding 2-aminothizole derivatives by Curtius type rearrangement via an acyl azide (R=CON₃). In order to prepare a substrate for macrocyclization using the ring closing olefin metathesis, either iodide 7a or 14 can be easily vinylated to 7b or 15 by a palladium catalyzed cross coupling reaction. For example, Stille coupling of tributylvinylstannane with 14a (R=CH₃, C-15 protecting group=TBS) afforded 15a in 67% yield. Additionally, as shown in FIG. 18, ring expanded analogues (e.g., 17-, 18- and 19-membered macrocycles) can be prepared by modifying the left (western) fragment. For example, to make a 17-membered macrocycle, allyltributylstannane can be utilized in the Stille coupling reaction, as shown in FIG. 18. It will be appreciated that other stannanes can be utilized to generate intermediates for other macrocycles (e.g., 18- and 19-membered rings), and that other reactions to generate suitable intermediates (for 17-, 18-, 19- or 20-membered rings) can be utilized (e.g., generation of a Grignard reagent suitable for a desired ring size, and utilizing a Pd coupling reaction to generate a desired left wing moiety). Furthermore, the descriptions exemplified for 16-membered macrocycles herein can also be applied to the synthesis of other rings, including, but not limited to 17-, 18- and 19-membered macrocycles.

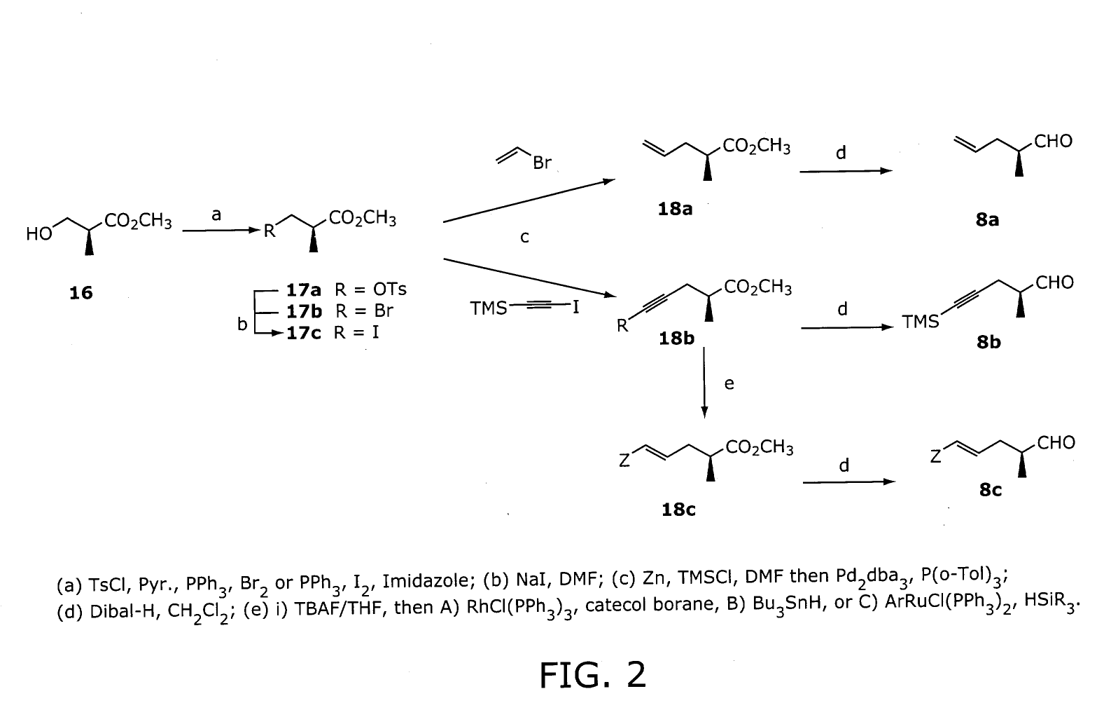

In general, intermediates (C) and (D) can be joined by a stereoselective aldol reaction (even after (C) and (D) have been joined to other fragments as depicted above). In one exemplary embodiment, as depicted in FIG. 2, a new synthesis developed in the present study uses the commercial 16 as the source of chirality as the precursor to fragment (C), and the Jackson type coupling reaction to introduce the alkene or alkyne functions. After activation of 16 to iodide 17c (in one or two steps), the cross coupling reaction of an organozinc reagent derived from 17c with vinyl bromide and acetylenic iodide generates 18a and 18b, respectively. Reduction of the methyl ester with Dibal-H gives the desired aldehyde 8a. It is also noteworthy that alkyne 18b becomes a precursor of a variety of functionalized alkenes (Z=BR₂, SnR₃, SiR₃, etc., wherein R can be halogen, alkyl, or aryl, as described herein) that can be utilized for the cross coupling with 7a such as Suzuki, Stille, Hiyama reactions.

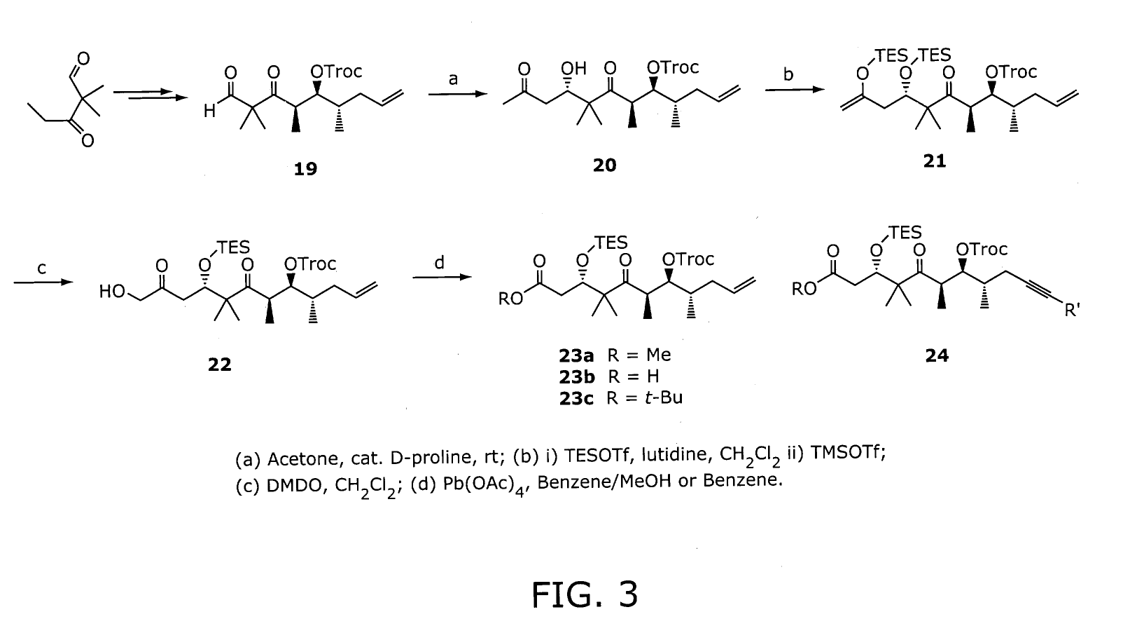

In one exemplary embodiment, as depicted in FIG. 3, the union of aldehyde 8a and ketoaldehyde 9 was achieved by the stereoselective aldol reaction the diisopropylacetal of 9 with 8a. After protection with a Troc group and hydrolysis of the diisopropyl acetal group, ketoaldehyde 19 was obtained, thus setting the stage for the second aldol reaction. Previously, an addition of a chiral titano acetate with aldehyde 19 afforded the t-butyl ester 23c (Wu et al. Angew. Chem. Int. Ed. Engl. 2000, 39, 4505). The proline catalyzed asymmetric aldol reaction of 19 with acetone (List et al. J. Am. Chem. Soc. 2000, 122, 2395) smoothly proceeded to afford the desired C3(S)-20 as a single isomer in high yield. Treatment of the aldol adduct 20 sequentially with TESOTf and TMSOTf induced protection of the C3 alcohol and regioselective formation of silyl enol ether 21. A chemoselective Rubottom type oxidation of 21 with 2,2-dimethyldioxirane (DMDO) generated hydroxyketone 22 which underwent a one carbon oxidative cleavage reaction by the agency of lead tetraacetate to give rise to methyl ester 23a. Thus, the Eastern wing fragments such as 23 and 24 are readily prepared with high efficiency by the sequential aldol reactions.

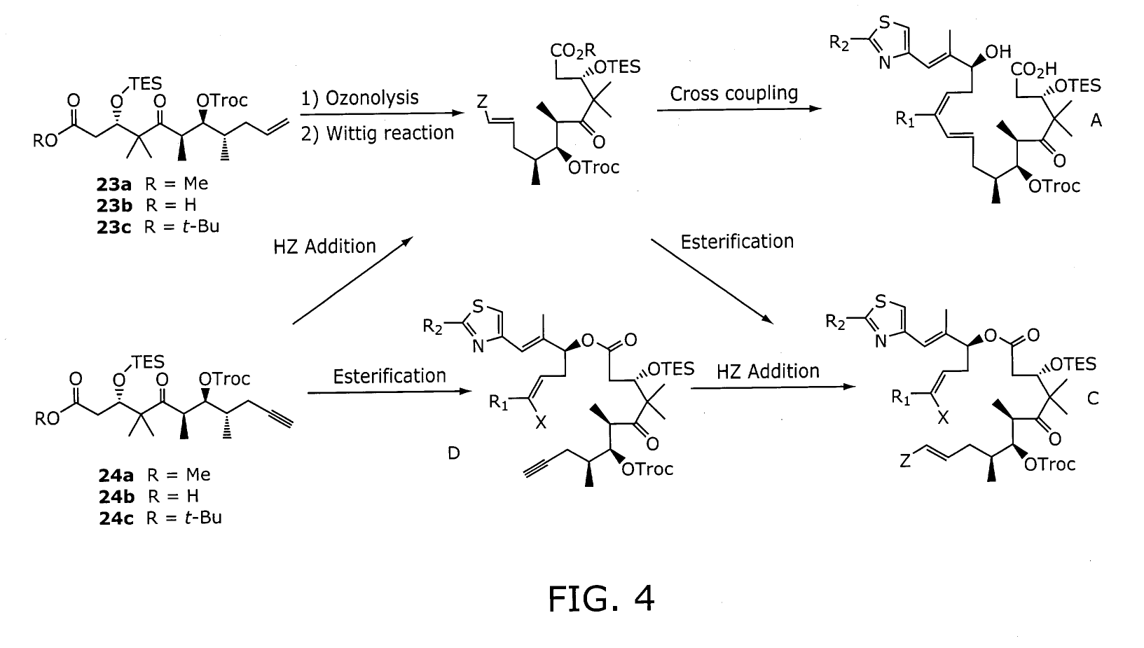

It will also be appreciated that the eastern fragment for some of the different macrocyclization strategies depicted in FIG. 4 can also be obtained from olefin 23 by ozonolysis and olefination of the resulting aldehyde with a suitable Wittig reagent. As shown in FIG. 4, this intermediate can be advanced to the desired ddEpo analog by cross-coupling or esterification manifolds (A or C). Similarly, alkynes 24 can also be advanced to the diene analogs by esterification and conversion to vinylic compounds C (Z=Sn, B, Si, etc.).

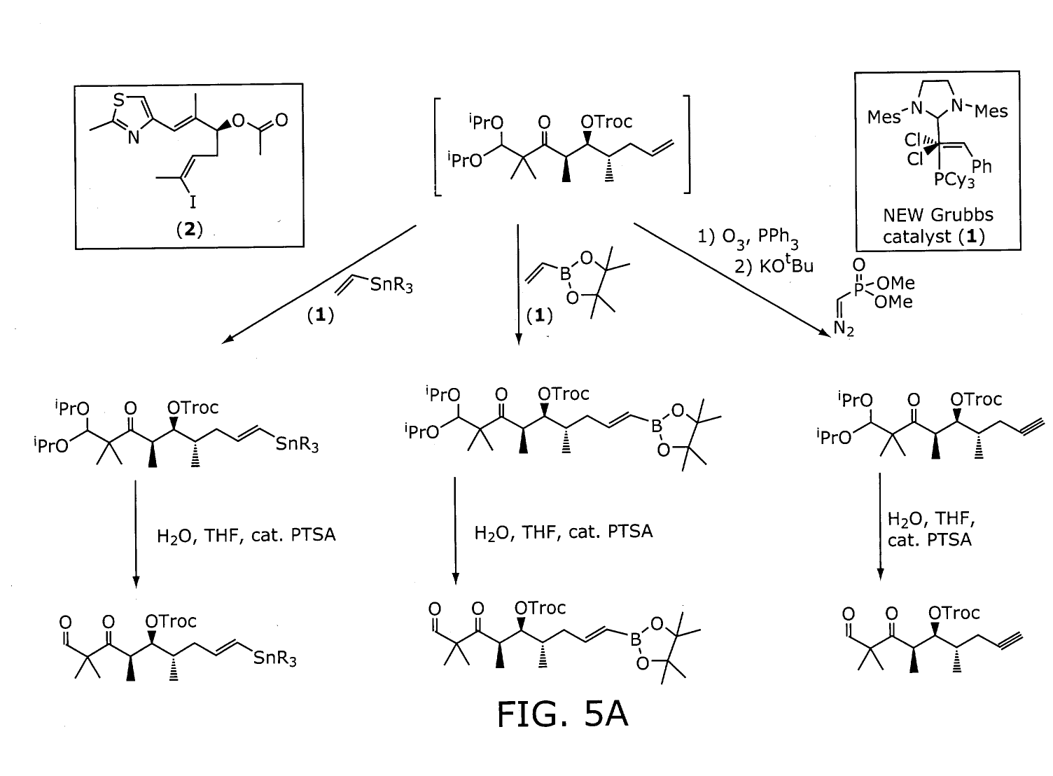

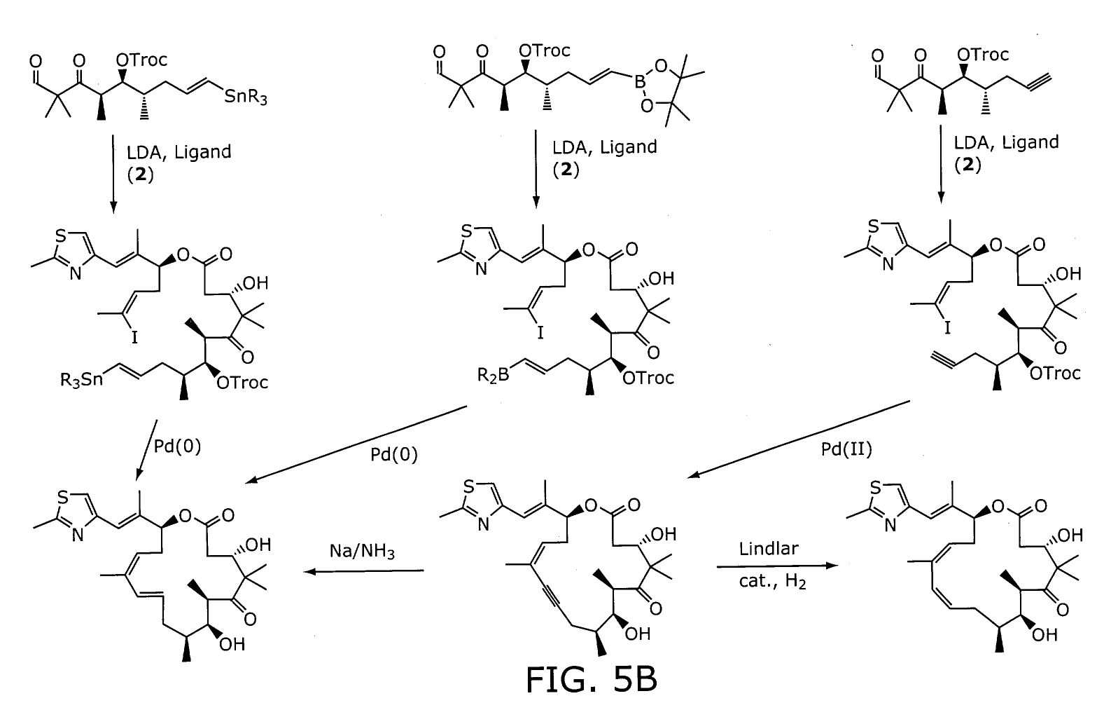

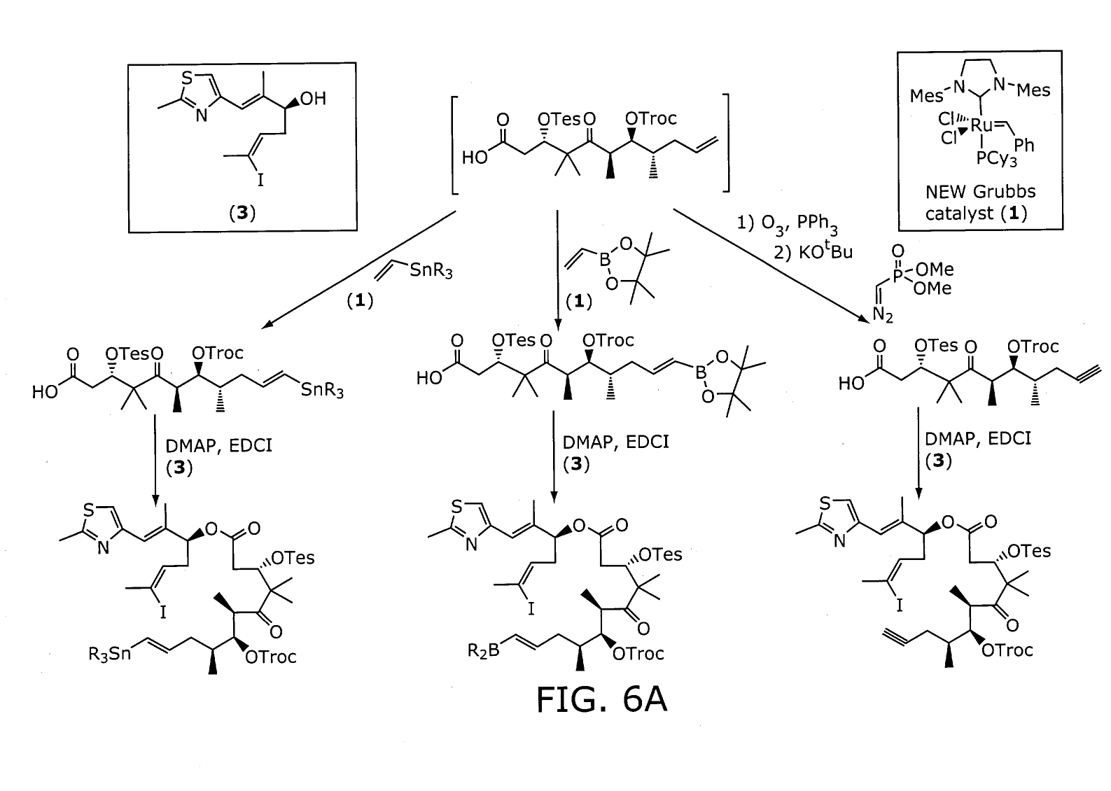

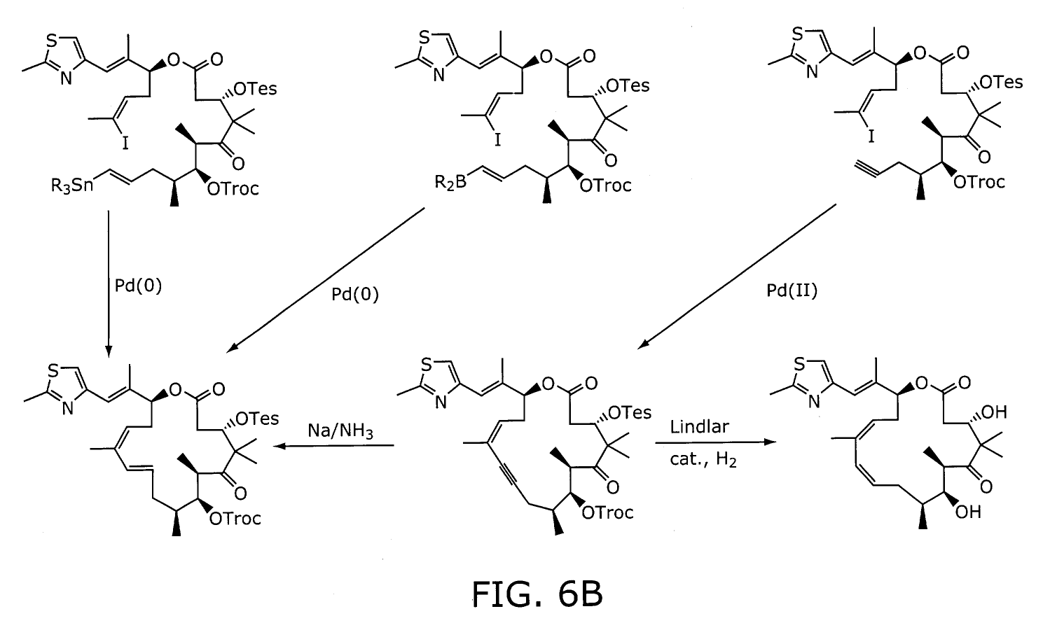

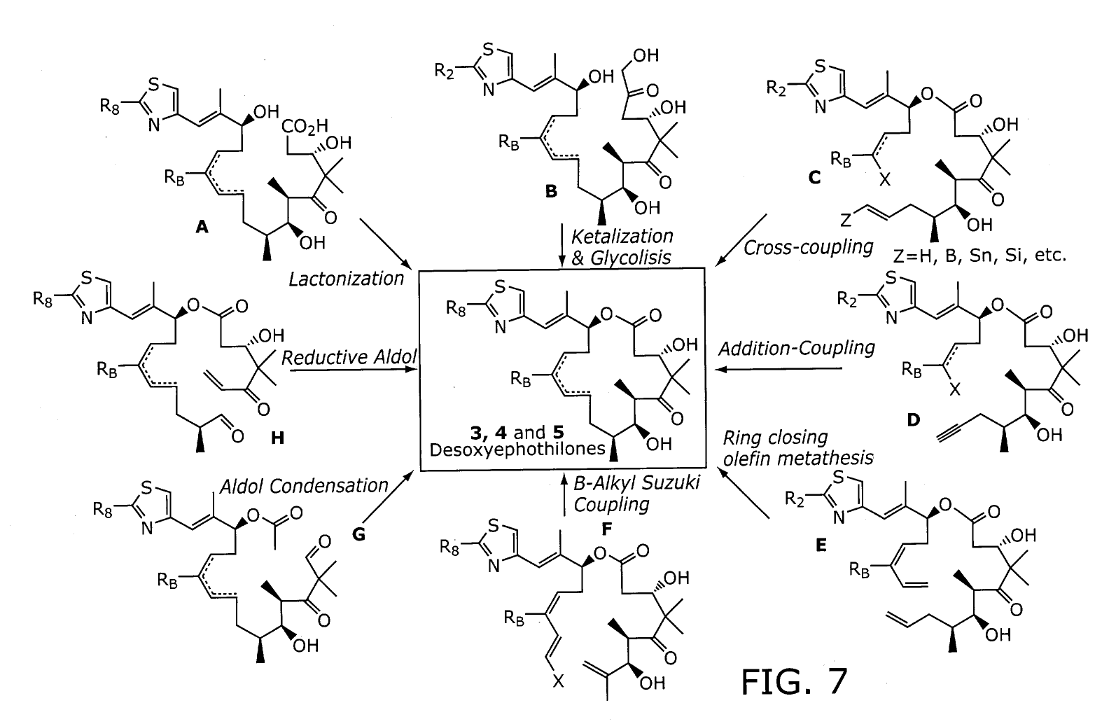

It will be appreciated that additional guidance for the preparation of various fragments can be found in the Exemplification herein and in the Figures (see, for Example, FIGS. 5A, 5B, 6A, and 6B).

b) Macrocyclization Reactions:

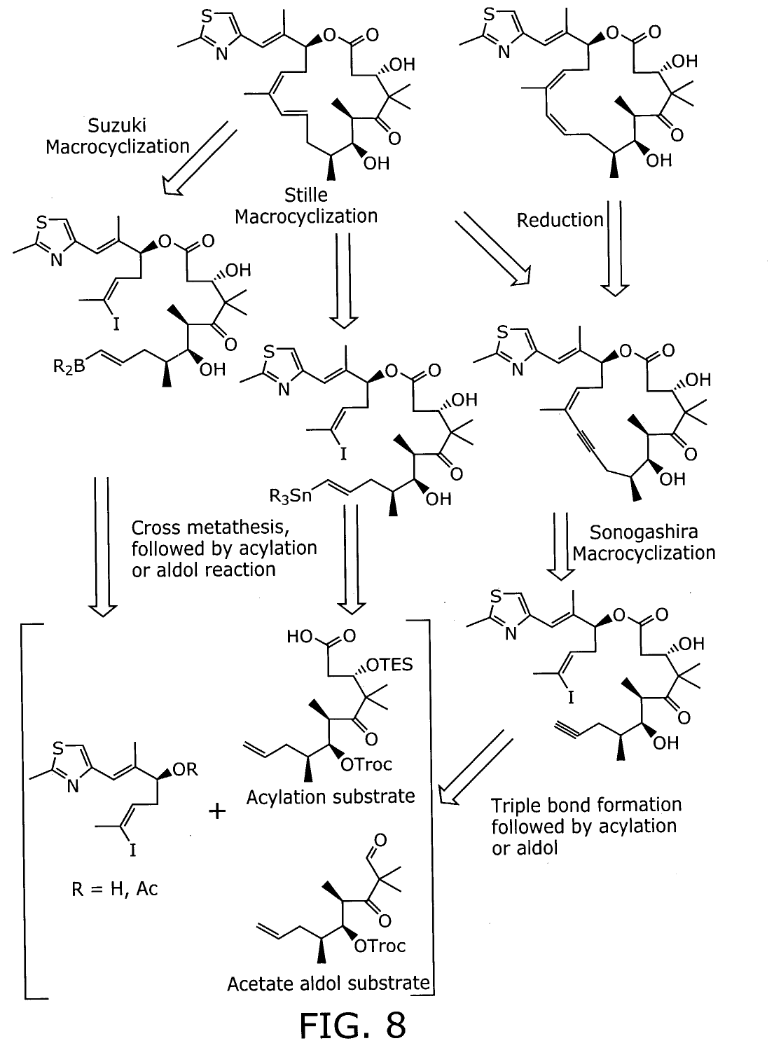

As described generally above, each of the fragments can synthesized, diversified, if desired, and ultimately be combined to generate a cyclization precursor, which can then be cyclized using a variety of synthetic methods. A number of strategies for macrocylization are depicted generally herein and in FIGS. 7 and 8. It will be appreciated that although FIGS. 7 and 8 depict strategies for the synthesis of 16-membered rings, this methodology can also be applied to the synthesis of larger ring structures, e.g., 17- 18- and 19-membered macrocycles, as described generally herein and in FIG. 18. In addition to the classical and yet most fruitful macrolactonization approach using a hydroxy acid of general type A, the new aldol reaction (D+E) fashioning the polypropionate domain provides a hydroxy ketone B for macro-ketalization. The glycolysis of the in situ formed macro-hemiketal then furnishes the macrolactone. While the B-alkyl Suzuki reaction has been conducted prior to the macrocyclization in previous Epo and dEpo syntheses, these types of cross-coupling reactions can be employed as a ring forming process when performed subsequent to the esterification. In particular, various metal catalyzed reactions (e.g., Heck, Suzuki, Stille, Hiyama, etc.) using corresponding substrates of type C (X=halide, Z=H, BR₂, SnR₃, SiR₃, wherein R is halogen, alkyl or aryl, for example) may be used for the macrocyclization en route to ddepos. As depicted in FIG. 7, it is also possible to fashion acetylenic substrate D to obtain a structure of type C and to subject C in situ to macrocyclization via metal-catalyzed “addition/cross coupling” procedures. Additionally, the ddEpo skeleton can be formulated in a direct manner using ring closing olefin metathesis E. A substrate directed stereoselective hydroboration of an allylic system as F (Still et al J. Am. Chem. Soc. 1983, 105, 2487) followed by a Suzuki coupling of the resultant B-alkylborane represents a novel macrocyclization method. The two aldol units (C1-C3 and C5-C7) present in the epothilones present themselves as the strategic bond for macrocyclization. The C2-C3 connectivity has been successfully achieved using substrate of type G in our first generation synthesis. While this strategy can also be applied to the synthesis of new analogues, a novel Mukaiyama aldol reaction may produce the desired macrocycle. The requisite enolate equivalent can be generated by the conjugated reduction of enone H under the catalysis of group 9 and 10 metals (Co, Rh, Ir, Pd, Pt) in the presence of the sensitive aldehyde (Morken et al J. Am. Chem. Soc. 1999, 121, 12202; 2000, 122, 4528; Krische et al J. Am. Chem. Soc. 2001, 123, 5112). It should be noted that the modularity of the approach described generally herein readily allows for the change of sequences, thus providing high synthetic flexibility.

c) Diversification:

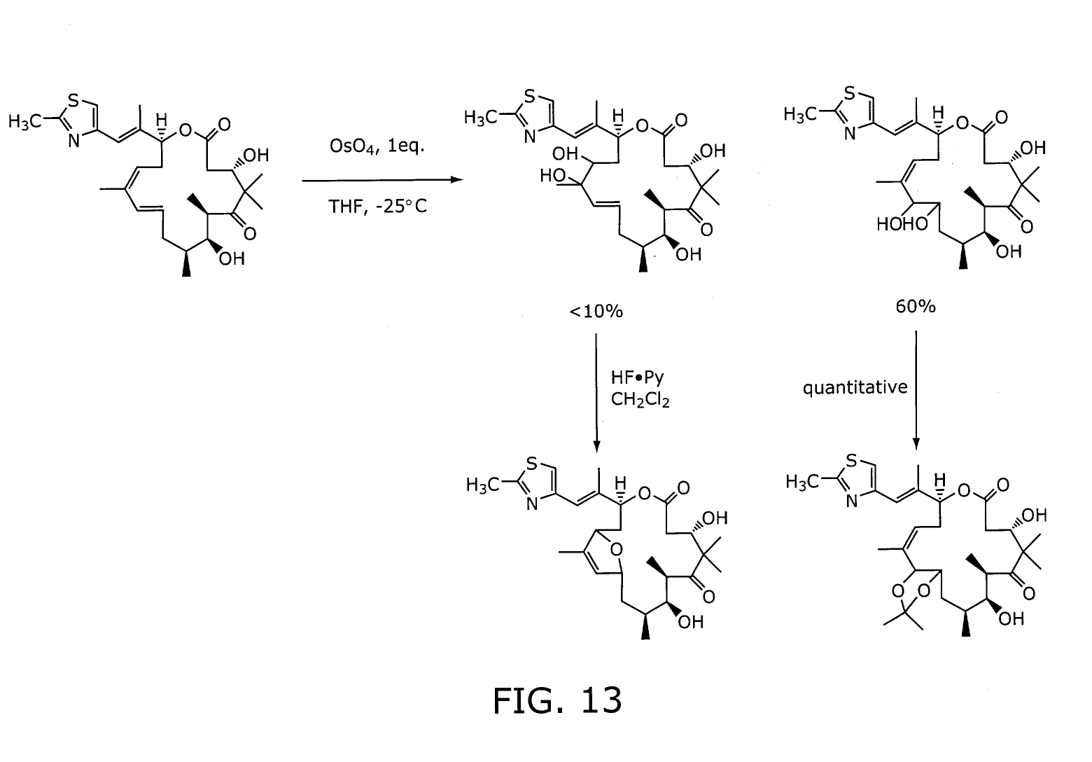

As mentioned above, it will also be appreciated that each of the components used in the synthesis of analogues can be diversified either before synthesis or alternatively after the construction of the macrocycle. As used herein, the term “diversifying” or “diversify” means reacting an inventive compound (I) or (II), or any of the precursor fragments (e.g., (A), (B), (C), etc.) as defined herein (or any classes or subclasses thereof) at one or more reactive sites to modify a functional moiety or to add a functional moiety (e.g., nucleophilic addition of a substrate). Described generally herein are a variety of schemes to assist the reader in the synthesis of a variety of analogues, either by diversification of the intermediate components or by diversification of the macrocyclic structures as described herein, and classes and subclasses thereof. It will also be appreciated that although many of the schemes herein depict 16-membered macrocycles, the reactions described herein may also be applied to other ring structures (for example to 17-, 18- and 19-membered ring structures). For example, FIG. 13 depicts the diversification of Epo-490 using OsO₄ to generate the tetraol (See also Exemplification). Further reaction with 2,2-dimethoxypropane additionally generates the acetonide (see Exemplification and FIG. 13). It will be appreciated that a variety of diversification reactions can be employed to generate novel analogues. As but a few examples, epoxidation and aziridation can be conducted to generate epoxide, and aziridine analogues of compounds described herein. Additionally, addition across either double bond will generate additional diversity (at either R_{A}, R_{B}, R_{C} or R_{D} positions). In addition to diversification after macrocyclization, it will be understood that diversification can occur prior to macrocyclization (e.g., epoxidation, aziridation, reduction at a C₁₂₋₁₃ double bond could occur prior to Suzuki macrocyclization, Stille macrocyclization, etc., to describe just one example). For additional guidance available in the art, the practitioner is directed to “Advanced Organic Chemistry”, March, J. John Wiley & Sons, 1992, the entire contents of which are hereby incorporated by reference.

It will also be appreciated that diversification is also intended to encompass the preparation of water soluble, multiply presented epothilone analogues and compounds attached to polymers and other supports and carbohydrates. In but one example, if a 21-amino analogue is prepared as detailed herein, the hydroxyl moiety can be reacted with aspartic anhydride. Ring opening by the 21-amino group and liberation of the a-amino group by mild deprotection (Troc, Zn/AcOH) generates a zwitter ion which provides enhanced water solubility. Additionally, C21 functional groups (e.g., OH, amino, etc. as described for certain of the inventive compounds herein) lend themselves as a staging point for introduction of various amino acids or peptids containing hydrophilic side chains to increase the water solubility. Using a 21-amino or hydroxy compound a N-protected oligopeptide can be attached to the epothilone domain through the C-terminal coupling. Furthermore, 21-functionalized epothilones can be readily conjugated with glucose or lactose to generate water soluble analogues. Additionally, the preparation of epothilone dimers is carried out by linking two halves of epothilones with a covalent linker (e.g., diacid, diamines, diols having varied lengths) via a coupling reaction. Additional functionalization reactions include those in which the compounds as described above and herein are multiply presented on dendrimers or polymers or are linked to a biodegradable polymer. As described herein the term “epothilones, desoxyepothilones and analogues thereof” is intended to encompass epothilones and desoxyepothilones previously reported as well as inventive epothilones and desoxyepothilones as described in more detail herein. Thus, it will be appreciated that an inventive epothilone or desoxyepothilone as described herein may be linked to another inventive compound or may be linked to a previously reported compound (or other known therapeutic agent). Each of the general methodologies described above for the diversification of compounds having 16-membered rings can also be applied to larger ring structures, including, but not limited to, 17-, 18- and 19-membered macrocycles.

5) Uses, Formulation and Administration

Pharmaceutical Compositions

As discussed above, the present invention provides novel compounds having antitumor and antiproliferative activity, and thus the inventive compounds are useful for the treatment of cancer. Accordingly, in another aspect of the present invention, pharmaceutical compositions are provided, wherein these compositions comprise any one of the compounds as described herein, and optionally comprise a pharmaceutically acceptable carrier. In certain embodiments, these compositions optionally further comprise one or more additional therapeutic agents. In certain other embodiments, the additional therapeutic agent is an anticancer agent, as discussed in more detail herein.

It will also be appreciated that certain of the compounds of present invention can exist in free form for treatment, or where appropriate, as a pharmaceutically acceptable derivative thereof. According to the present invention, a pharmaceutically acceptable derivative includes, but is not limited to, pharmaceutically acceptable salts, esters, salts of such esters, or any other adduct or derivative which upon administration to a patient in need is capable of providing, directly or indirectly, a compound as otherwise described herein, or a metabolite or residue thereof, e.g., a prodrug.

As used herein, the term “pharmaceutically acceptable salt” refers to those salts which are, within the scope of sound medical judgement, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describe pharmaceutically acceptable salts in detail in J. Pharmaceutical Sciences, 66: 1-19 (1977), incorporated herein by reference. The salts can be prepared in situ during the final isolation and purification of the compounds of the invention, or separately by reacting the free base function with a suitable organic acid. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Additionally, as used herein, the term “pharmaceutically acceptable ester” refers to esters which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include formates, acetates, propionates, butyrates, acrylates and ethylsuccinates.

Furthermore, the term “pharmaceutically acceptable prodrugs” as used herein refers to those prodrugs of the compounds of the present invention which are, within the scope of sound medical judgment, suitable for use in contact with the tissues of humans and lower animals with undue toxicity, irritation, allergic response, and the like, commensurate with a reasonable benefit/risk ratio, and effective for their intended use, as well as the zwitterionic forms, where possible, of the compounds of the invention. The term “prodrug” refers to compounds that are rapidly transformed in vivo to yield the parent compound of the above formula, for example by hydrolysis in blood. A thorough discussion is provided in T. Higuchi and V. Stella, Pro-drugs as Novel Delivery Systems, Vol. 14 of the A.C.S. Symposium Series, and in Edward B. Roche, ed. Bioreversible Carriers in Drug Design, American Pharmaceutical Association and Pergamon Press, 1987, both of which are incorporated herein by reference.

As described above, the pharmaceutical compositions of the present invention additionally comprise a pharmaceutically acceptable carrier, which, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Fifteenth Edition, E. W. Martin (Mack Publishing Co., Easton, Pa., 1975) discloses various carriers used in formulating pharmaceutical compositions and known techniques for the preparation thereof. Except insofar as any conventional carrier medium is incompatible with the anti-cancer compounds of the invention, such as by producing any undesirable biological effect or otherwise interacting in a deleterious manner with any other component(s) of the pharmaceutical composition, its use is contemplated to be within the scope of this invention. Some examples of materials which can serve as pharmaceutically acceptable carriers include, but are not limited to, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; Cremophor; Solutol; excipients such as cocoa butter and suppository waxes; oils such as peanut oil, cottonseed oil; safflower oil; sesame oil; olive oil; corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and; magnesium stearate, as well as coloring agents, releasing agents, coating agents, sweetening, flavoring and perfuming agents, preservatives and antioxidants can also be present in the composition, according to the judgment of the formulator.

Uses of Compounds and Pharmaceutical Compositions.

In yet another aspect, according to the methods of treatment of the present invention, tumor cells are killed, or their growth is inhibited by contacting said tumor cells with an inventive compound or composition, as described herein. Thus, in still another aspect of the invention, a method for the treatment of cancer is provided comprising administering a therapeutically effective amount of an inventive compound, or a pharmaceutical composition comprising an inventive compound to a subject in need thereof, in such amounts and for such time as is necessary to achieve the desired result. In certain embodiments of the present invention a “therapeutically effective amount” of the inventive compound or pharmaceutical composition is that amount effective for killing or inhibiting the growth of tumor cells. The compounds and compositions, according to the method of the present invention, may be administered using any amount and any route of administration effective for killing or inhibiting the growth of tumor cells. Thus, the expression “amount effective to kill or inhibit the growth of tumor cells”, as used herein, refers to a sufficient amount of agent to kill or inhibit the growth of tumor cells. The exact amount required will vary from subject to subject, depending on the species, age, and general condition of the subject, the severity of the infection, the particular anticancer agent, its mode of administration, and the like. The anticancer compounds of the invention are preferably formulated in dosage unit form for ease of administration and uniformity of dosage. The expression “dosage unit form” as used herein refers to a physically discrete unit of anticancer agent appropriate for the patient to be treated. It will be understood, however, that the total daily usage of the compounds and compositions of the present invention will be decided by the attending physician within the scope of sound medical judgment. The specific therapeutically effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

Furthermore, after formulation with an appropriate pharmaceutically acceptable carrier in a desired dosage, the pharmaceutical compositions of this invention can be administered to humans and other animals orally, rectally, parenterally, intracisternally, intravaginally, intraperitoneally, topically (as by powders, ointments, or drops), bucally, as an oral or nasal spray, or the like, depending on the severity of the infection being treated. In certain embodiments of the invention, the inventive compounds as described herein are formulated by conjugating with water soluble chelators, or water soluble polymers such as polyethylene glycol as poly (1-glutamic acid), or poly (1-aspartic acid), as described in U.S. Pat. No. 5,977,163, the entire contents of which are hereby incorporated by reference. In certain embodiments, the compounds of the invention may be administered orally or parenterally at dosage levels of about 0.01 mg/kg to about 50 mg/kg, preferably from about 0.1 mg/kg to about 40 mg/kg, and more preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar—agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

As discussed above, the compounds of the present invention are useful as anticancer agents, and thus may be useful in the treatment of cancer, by effecting tumor cell death or inhibiting the growth of tumor cells. In general, the inventive anticancer agents are useful in the treatment of cancers and other proliferative disorders, including, but not limited to breast cancer, cervical cancer, colon and rectal cancer, leukemia, lung cancer, melanoma, multiple myeloma, non-Hodgkin's lymphoma, ovarian cancer, pancreatic cancer, prostate cancer, and gastric cancer, to name a few. In certain embodiments, the inventive anticancer agents are active against leukemia cells and melanoma cells, and thus are useful for the treatment of leukemias (e.g., myeloid, lymphocytic, myelocytic and lymphoblastic leukemias) and malignant melanomas. In still other embodiments, the inventive anticancer agents are active against solid tumors and also kill and/or inhibit the growth of multidrug resistant cells (MDR cells).

It will also be appreciated that the compounds and pharmaceutical compositions of the present invention can be employed in combination therapies, that is, the compounds and pharmaceutical compositions can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an inventive compound may be administered concurrently with another anticancer agent), or they may achieve different effects (e.g., control of any adverse effects).

For example, other therapies or anticancer agents that may be used in combination with the inventive anticancer agents of the present invention include surgery, radiotherapy (in but a few examples, γ-radiation, neutron beam radiotherapy, electron beam radiotherapy, proton therapy, brachytherapy, and systemic radioactive isotopes, to name a few), endocrine therapy, biologic response modifiers (interferons, interleukins, and tumor necrosis factor (TNF) to name a few), hyperthermia and cryotherapy, agents to attenuate any adverse effects (e.g., antiemetics), and other approved chemotherapeutic drugs, including, but not limited to, alkylating drugs (mechlorethamine, chlorambucil, Cyclophosphamide, Melphalan, Ifosfamide), antimetabolites (Methotrexate), purine antagonists and pyrimidine antagonists (6-Mercaptopurine, 5-Fluorouracil, Cytarabile, Gemcitabine), spindle poisons (Vinblastine, Vincristine, Vinorelbine, Paclitaxel), podophyllotoxins (Etoposide, Irinotecan, Topotecan), antibiotics (Doxorubicin, Bleomycin, Mitomycin), nitrosoureas (Carmustine, Lomustine), inorganic ions (Cisplatin, Carboplatin), enzymes (Asparaginase), and hormones (Tamoxifen, Leuprolide, Flutamide, and Megestrol), to name a few. For a more comprehensive discussion of updated cancer therapies see, http://www.nci.nih.gov/, a list of the FDA approved oncology drugs at http://www.fda.gov/cder/cancer/druglistframe.htm, and The Merck Manual, Seventeenth Ed. 1999, the entire contents of which are hereby incorporated by reference.

In still another aspect, the present invention also provides a pharmaceutical pack or kit comprising one or more containers filled with one or more of the ingredients of the pharmaceutical compositions of the invention, and in certain embodiments, includes an additional approved therapeutic agent for use as a combination therapy. Optionally associated with such container(s) can be a notice in the form prescribed by a governmental agency regulating the manufacture, use or sale of pharmaceutical products, which notice reflects approval by the agency of manufacture, use or sale for human administration.

### EQUIVALENTS

The representative examples which follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. It should further be appreciated that the contents of those cited references are incorporated herein by reference to help illustrate the state of the art. The following examples contain important additional information, exemplification and guidance which can be adapted to the practice of this invention in its various embodiments and equivalents thereof.

### EXEMPLIFICATION

### Example 1

Synthesis of Epo-490 Analogues:

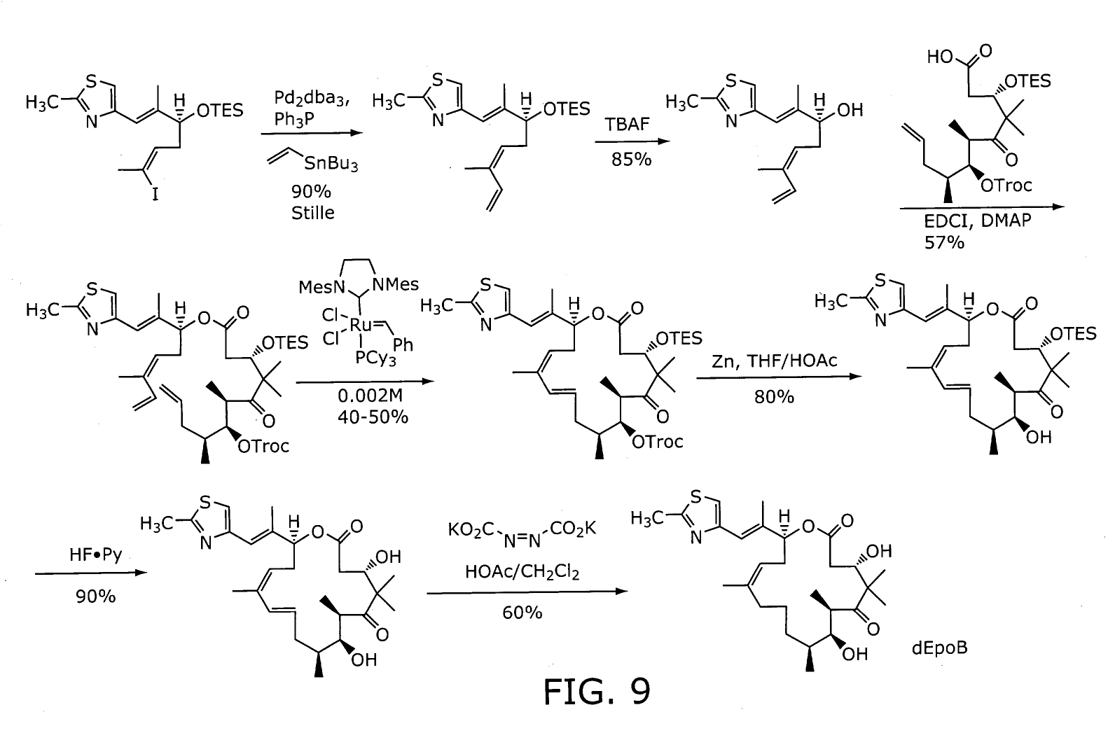

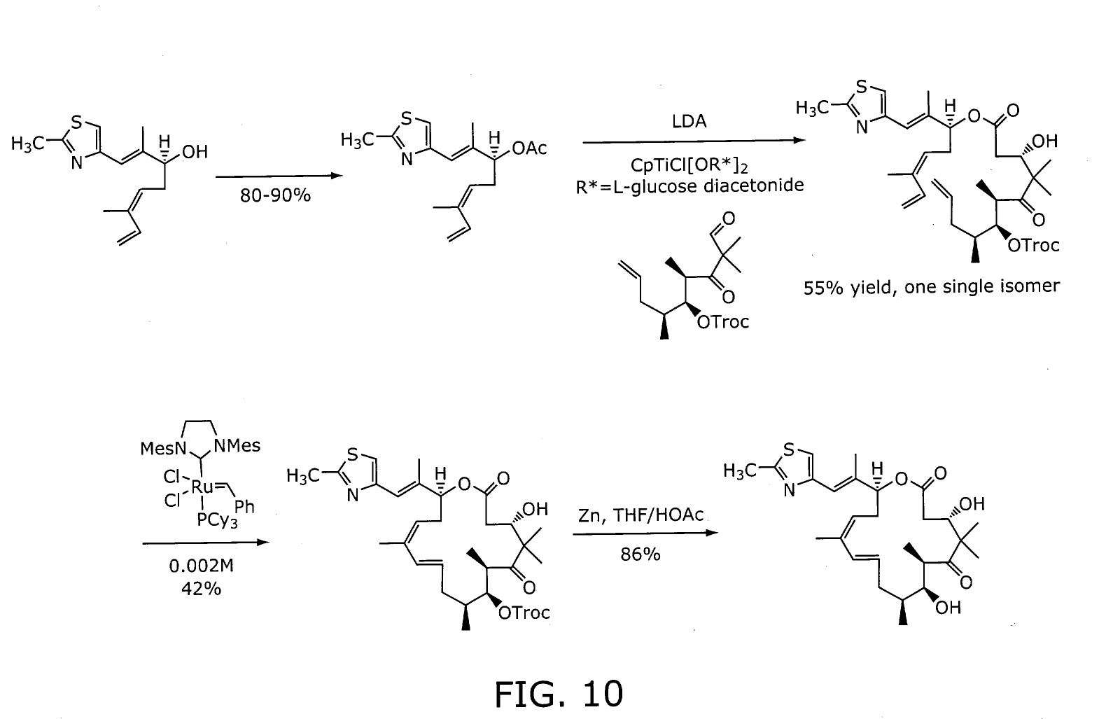

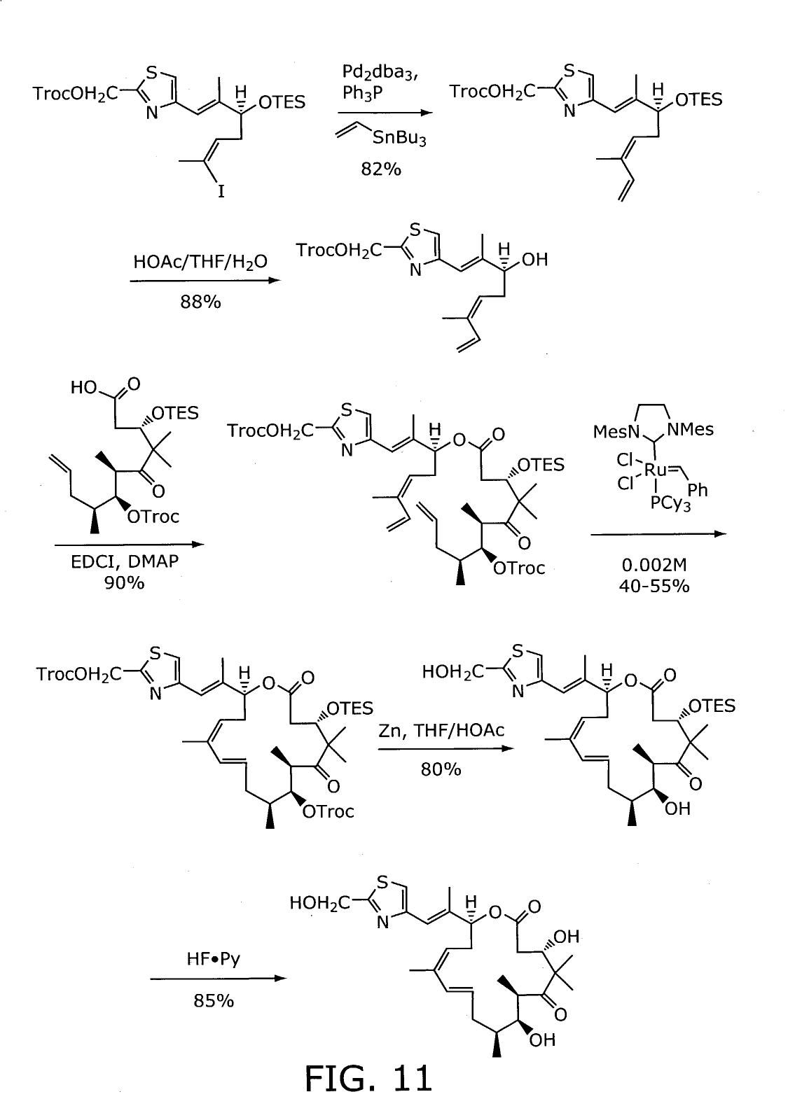

Described herein are a number of exemplary compounds, the structures and syntheses of which are also depicted in FIGS. 9, 10, and 13.

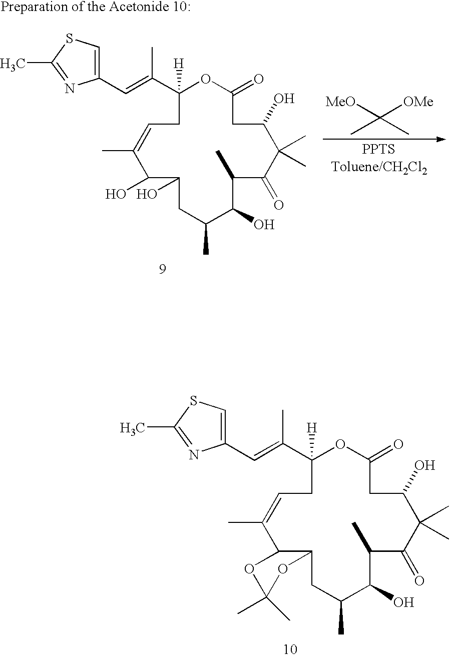

Acetonide 10: To a stirred mixture of 9 (3.0 mg, mmol) in toluene (0.1 mL) and CH₂Cl₂ (0.1 mL) were added a few crystals of PPTS and 2,2-dimethoxypropane (0.2 mL). The reaction mixture was stirred at room temperature for 3 h, before being concentrated in vacuo and purified using silica gel chromatography employing 50% EtOAc/hexane as the eluent, which afforded 3.2 mg (99% yield) of acetonide 10.

Acetonide 10: [α]_{D+}39° (c 0.16, CHCl₃); ¹H NMR (400 MHz, CDCl₃) 6.90 (s, 1), 6.55 (s, 1), 5.42 (dd, 1, J=11.5, 2.7), 5.14 (d, 1, J=10.2), 4.39 (d, 1, J=8.9) , 4.31 (dd, 1, J=11.1, 2.7), 3.86 (ddd, 1, J=9.7, 8.9, 1.3), 3.61 (m, 1), 3.54 (br s, 1, OH), 3.17 (br s, 1, OH), 3.04 (qd, 1, J=6.6, 3.1), 2.74 (ddd, 1, J=15.5, 11.5, 10.2), 2.62 (s, 3), 2.40 (dd, 1, J=14.6, 11.1), 2.27 (m, 1), 2.15 (dd, 1, J=14.6, 2.7), 2.00 (d, 3, J=0.9 ), 1.90 (m, 1), 1.69 (s, 3), 1.50 (ddd, 1, J=14.6, 4.4, 1.3), 1.38 (s, 3), 1.36 (s, 3), 1.37-1.33 (m, 1), 1.28 (s, 3), 1.19 (d, 3, J=6.6), 1.07 (d, 3, J=7.1), 0.97 (s, 3); ¹³C NMR (100 MHz, CDCl₃) 220.0, 170.2, 165.2, 151.6, 139.2, 133.1, 128.7, 119.3, 115.8, 108.7, 78.7, 78.5, 77.7, 75.6, 72.0, 53.8, 42.7, 39.7, 37.1, 34.6, 32.9, 27.6, 26.9, 23.0, 19.0, 18.3, 17.5, 16.1, 14.7; IR (neat) 3463, 2981, 2924, 1733, 1694, 1248, 1043, 737.

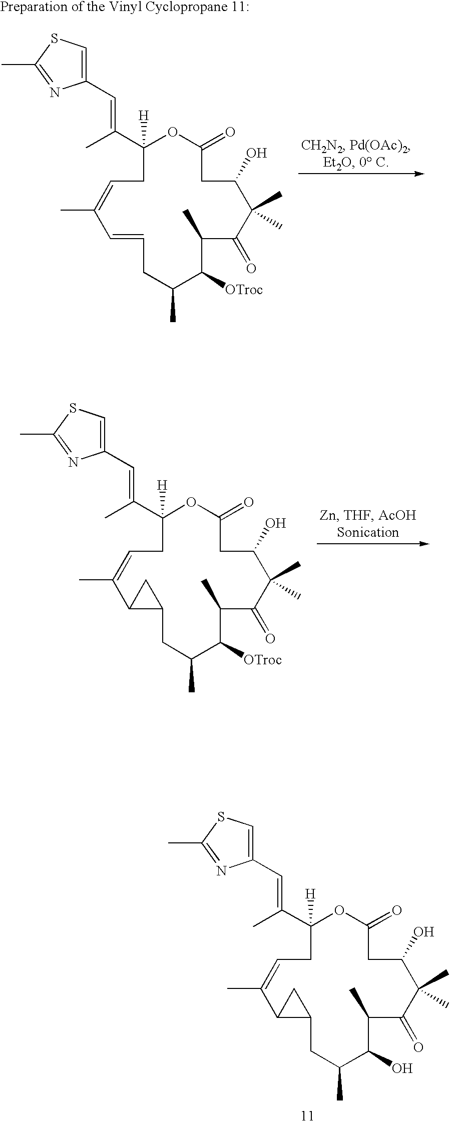

Treamtent of the Troc-protected Epo490 in diethyl ether with diazomethane in the presence of Pd(OAc)₃ at 0° C. resulted in a 30% yield of the protected vinyl cyclopropane. Deprotectection with Zn⁰ in THF with acetic acid and sonication afforded the vinyl cyclopropane 11 (Denmark et al. J. Org. Chem. 62:3375, 1997; incorporated herein by reference).

### Example 2

Synthesis of 21-hydroxy Epo-490:

As depicted in FIG. 11, 21-hydroxy Epo-490 is synthesized by coupling the thiazolyl fragment via esterification with the protected eastern fragment using EDCI and DMAP to generate the diene macrocyclization precursor. Subjecting the precursor to olefin metathesis conditions using a ruthenium catalyst reported by Grubbs and as depicted affords the protected macrocycle. Subsequent deprotection yields 21-hydroxy Epo-490.

### Example 3

Synthesis of 26-trifluoro-Epothilone D:

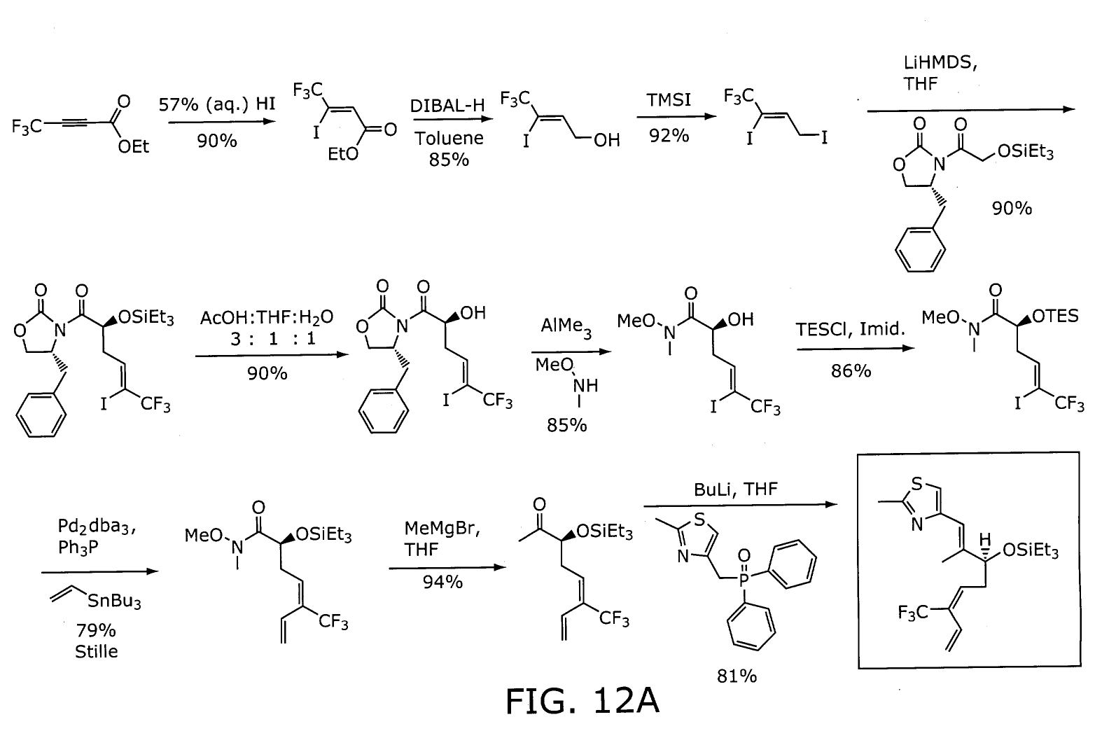

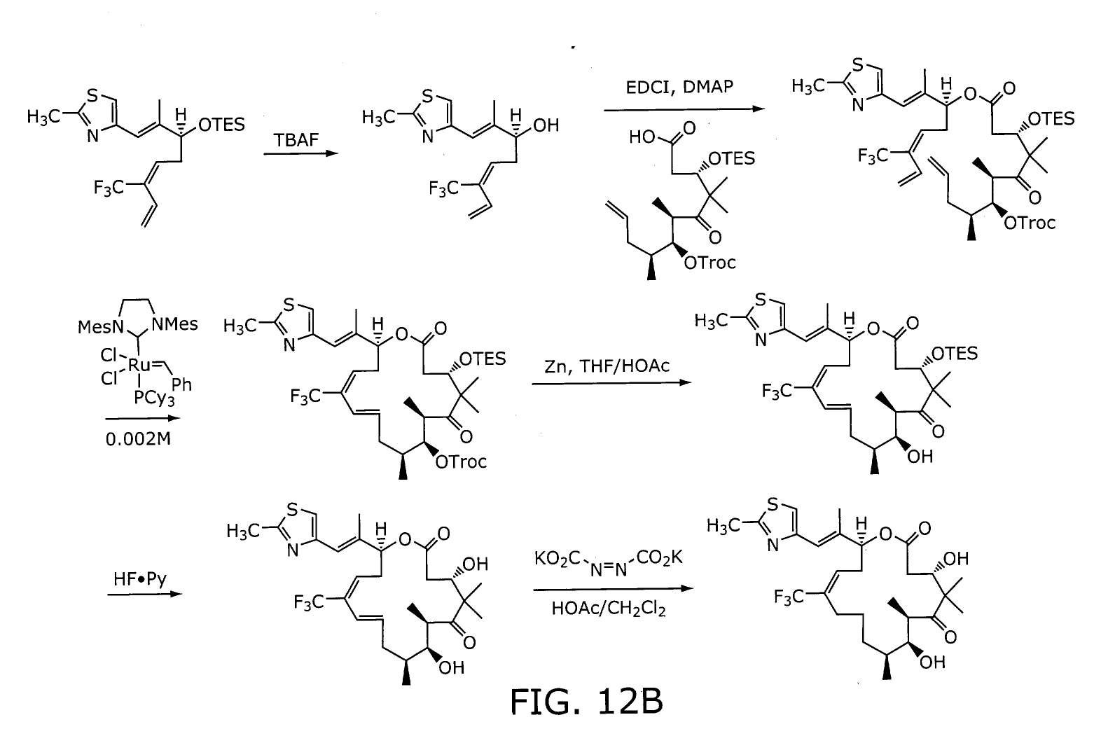

As depicted in FIG. 12, 26-trifluoro-epothilone D is synthesized by coupling the 26-trifluoro-thiazolyl fragment using esterification conditions to generate the diene cyclization precursor. Subsequent olefin metathesis using the ruthenium catalyst reported by Grubbs as described above, affords the protected macrocycle. Subsequent deprotection yields 26-trifluoro-Epo-490 and subsequent selective reduction yields 26-trifluoro-epothilone D.

### Example 4

Synthesis of [17]- and [18]Dehydrodesoxyepothiolones B:

Introduction: A convergent ring-closing metathesis strategy was employed for the syntheses of 10,11-dehydro-13,14-[17]desoxyepothilone B ([17]ddEpoB) and 10,11-dehydro-14,15-[18]desoxyepothilone B ([18]ddEpoB), which are 17- and 18 membered ring homologs of 10,11-dehydro-12,13-desoxyepothilone B ([16]ddEpoB or epothilone 490).

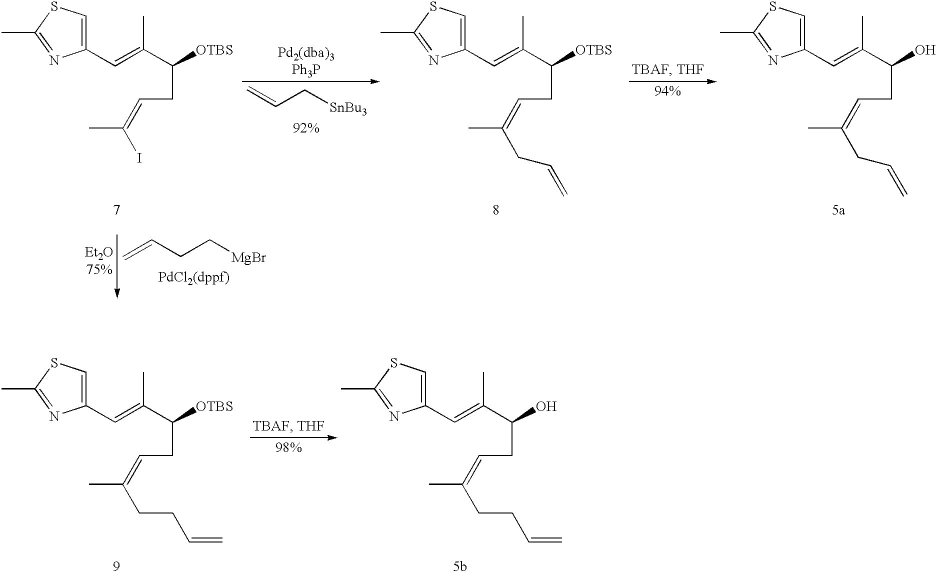

Compound 8: To a stirred solution of vinyl iodide 7 (250 mg, 0.539 mmol) in DMF (5 mL) were added allyltributyltin (0.536 g, 1.62 mmol, 3.0 equiv) and triphenylphosphine (56.5 mg, 0.216 mmol, 0.4 equiv), followed by Pd₂(dba)₃ (98.6 mg, 0.108 mmol, 0.2 equiv). The reaction mixture was stirred at room temperature for 12 h, diluted with Et₂O (10 mL) and water (10 mL). The aqueous layer was separated and extracted with Et₂O (2×10 mL). The combined organic extracts were dried over MgSO₄ and concentrated in vacuo. Purification using silica gel chromatography employing 5% EtOAc/pentane as the eluent afforded 1,4-diene 8 (187 mg, 92.1% yield) as a clear oil: [α]_{D} +16.8 (c 1.0, CHCl₃); IR (neat) 2995, 2927, 2855, 1635, 1506, 1471, 1255, 1182, 1074, 947, 836, 775 cm⁻¹; ¹H NMR (400 MHz, CDCl₃) δ6.92 (s, 1H), 6.46 (s,1H), 5.77-5.69 (m, 1H), 5.23 (t, J=7.2 Hz, 1H), 5.05 (dd, 1H, J=17.1, 1.6 Hz), 4.99 (dd, 1H, J=10.0, 1.5 Hz), 4.10 (t, 1H, J=6.6 Hz), 2.84-2.72 (m, 2H), 2.73 (s, 3H), 2.32-2.21 (m, 2H), 2.00 (s, 3H), 1.67 (s, 3H), 0.89 (s, 9H), 0.05 (s, 3H), 0.01 (s, 3H); ¹³C NMR (400 MHz, CDCl₃) δ164.5, 153.4, 142.7, 136.3, 134.6, 122.7, 118.9, 115.3, 115.1, 79.1, 36.8, 35.5, 26.0, 23.7, 19.4, 18.4, 14.1, −4.45, −4.73; HRMS (FAB) calcd. For C₂₁H₃₅NOSSi (M+H⁺) 378.2287, found 378.2286.

Compound 5a: To a stirred solution 1,4-Diene 8 (150 mg, 0.397 mmol) in THF (4 mL) at 0° C. was added tetrabutyl ammonium fluoride (1.0M in THF, 1.00 mL) and allowed to warm to room temperature. The reaction mixture was stirred at room temperature for 3 h, at which point it was diluted with water (5 mL) and Et₂O (10 mL). The aqueous layer was separated and extracted with Et₂O (2×10 mL) and EtOAc (10 mL). The combined organic layers were dried over Na₂SO₄ and concentrated in vacuo. Purification using silica gel chromatography employing 20% EtOAc/pentane as the eluent afforded alcohol 5a (97 mg, 94% yield) as a clear oil: [α]_{D} −20.3 (c 1.4, CHCl₃); IR (neat) 3384, 2970, 2912, 1635, 1506, 1436, 1374, 1185, 1028, 910, 729 cm⁻¹; ¹H NMR (400 MHz, CDCl₃) 6.95 (s, 1H), 6.56 (s, 1H), 5.81-5.71 (m, 1H), 5.26 (t, 1H, J=7.2 Hz), 5.07 (d, 1H, J=22 Hz), 5.02 (d, 1H, J=14 Hz), 4.16 (t, 1H, J 5.7 Hz), 3.10 (s, 1H, OH), 2.85 (d, 2H, J=6.1 Hz), 2.76 (s, 3H), 2.36 (t, 2H, J=6.8 Hz), 2.04 (s, 3H); 1.66 (s, 3H); ¹³C NMR (500 MHz, CDCl₃) δ164.6, 152.8, 142.1, 136.0, 135.8, 121.6, 118.9, 115.4, 115.3, 77.2, 36.6, 34.1, 23.6, 19.1, 14.3; HRMS (FAB) calcd. For C₁₅H₂₁NOS (M+H⁺) 264.1422, found 264.1422.

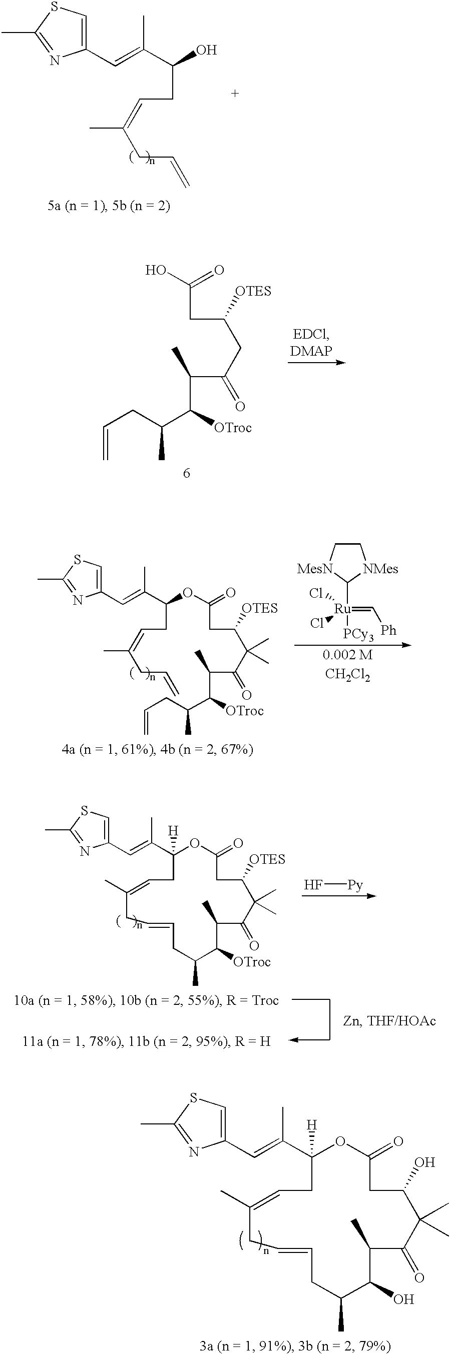

Compound 4a: Acid 6 was dried through azeotropic distillation. Freshly dried acid 6 (60 mg, 0.26 mmol, 1 equiv) in CH₂Cl₂ (5 mL) at 0° C. were added DMAP (73 mg, 0.37 mmol, 1.4 equiv) and EDCI (73 mg, 0.37 mmol, 1.4 equiv). After 15 minutes of stirring at 0° C., a solution of alcohol 5a (97 mg, 0.37 mmol, 1.4 equiv) dissolved in CH₂Cl₂ (2 mL) was added dropwise. The cooling bath was then removed and the reaction mixture stirred for 6 h. The crude reaction mixture is diluted with DCM (10 mL) and loaded onto silica and purified using silica gel chromatography employing 8% EtOAC/pentane as the eluent yielding ester 4a (133 mg, 61% yield) as a clear oil: [α]_{D} −19.1 (c 0.56, CDCl₃); IR (neat) 2958, 2876, 1756, 1700, 1456, 1382, 1250, 1180, 1093, 1065, 993, 926, 815, 735 cm⁻¹; ¹H NMR (400 MHz, CDCl₃) δ6.94 (s, 1H), 6.49 (s, 1H), 5.80-5.65 (m, 2H), 5.21 (t, 1H, J=6.8 Hz), 5.15 (t, 1H, J=6.4 Hz), 5.05-4.98 (m, 3H), 4.82 (d, 1H, J=12 Hz), 4.73 (dd, 1H, J=18.1, 3.6 Hz), 4.66 (d, 1H, J=12 Hz), 4.21 (dd, 1H, J=7.2, 3.1 Hz), 3.50-3.47 (m, 1H), 2.78 (d, 2H, J=6 Hz), 2.69 (s, 3H), 2.57 (dd, 1H, J=17.1, 3.1 Hz), 2.49-2.35 (m, 3H), 2.22-2.11 (m, 2H), 2.08 (s, 3H), 1.90-1.82 (m, 2H), 1.66 (s, 3H), 1.35 (s, 3H), 1.27-1.19 (m, 2H), 1.05 (d, 3H, J=6.7 Hz), 1.04 (s, 3H), 1.01-0.95 (m, 10H), 0.63 (q, 6H, J=7.6 Hz); ¹³C NMR (400 MHz, CDCl₃) δ215.6, 171.7, 164.9, 154.2, 137.6, 136.4, 136.1, 136.0, 121.4, 120.7, 117.6, 117.5, 116.7, 95.1, 82.3, 80.5, 75.6, 75.4, 53.8, 42.6, 40.1, 36.9, 34.8, 31.9, 30.1, 23.8, 22.6, 21.3, 19.8, 16.5, 14.9, 10.9, 7.4, 5.4; HRMS (FAB) calcd. For C₃₉H₆₀Cl₃NO₇SSi (M+H⁺) 820.3008, found 820.3007.

Compound 11a: Diene 4a (53 mg, 0.064 mmol) was dissolved in dry DCM (33 mL) and heated in the presence of tricyclohexylphosphine[1,3-bis(2,4,6-trimethylphenyl)-4,5-dihydroimidazol-2-ylidene][benzylidene]ruthenium (IV) dichloride (11 mg, 0.013 mmol) at reflux for 2.5 hours. The reaction mixture was cooled to room temperature and stripped onto silica and purified using silica gel chromatography employing 4-10% EtOAc/pentane gradient as the eluent to furnish a slightly impure triene 10a (30 mg, 58% yield) as an orange oil. This reaction was repeated three times on the same scale. A solution of triene 10a (80 mg, 0.1 mmol) in 1:1 THF/HOAc (4 mL) was then prepared and treated with Zn° (15 mg, nanosize). The reaction mixture was sonicated for 15 min at rt. More Zn° (15 mg, nanosize) was added, followed by sonication for a further 15 min at rt. The suspension was filtered through celite, followed by washing of the celite cake with EtOAc (25 mL). The combined filtrate was washed with saturated NaHCO₃ (10 mL), brine (10 mL), and dried over MgSO₄. Removal of the solvent in vacuo followed by purification of the residue on silica gel chromatography using 12% EtOAc/hexane as the eluent yielded alcohol 11a (41 mg, 78%): [α] −19.8° (c 1.0, CHCl₃); IR (neat) 3494, 2957, 1736, 1683, 1454, 1377, 1328, 1254, 1185, 1109 cm⁻¹; ¹H NMR (400 MHz, CDCl₃) δ6.95 (s, 1H), 6.57 (s, 1H), 5.59-5.53 (m, 2H), 5.42 (dt, J=14.8, 7.4 Hz, 1H), 5.10 (d, J=7.4 Hz, 1H), 4.28 (d, J=10.0 Hz, 1H), 3.91 (bs, 1H), 3.57 (d, J=9.6 Hz, 1H), 3.05 (q, J=6.7 Hz, 1H), 2.90 (dd, J=15.6, 5.8 Hz, 1H), 2.70-2.61 (m, 5H), 2.45 (dd, J=14.2, 10.1 Hz, 1H), 2.42-2.35 (m, 1H), 2.23 (d, J=13.6 Hz, 2H), 2.14 (s, 3H), 1.97-1.91 (m, 1H), 1.84-1.80 (m, 1H), 1.23 (s, 3H), 1.09 (d, J=6.9 Hz, 3H), 1.02 (s, 3H), 0.89 (d, J=6.9 Hz, 3H), 0.84 (t, J=7.9 Hz, 9H), 0.53 (q, J=7.9 Hz, 6H); ¹³C NMR (100 MHz, CDCl₃) δ221.5, 170.2, 164.5, 152.5, 137.1, 136.6, 128.8, 124.8, 121.7, 121.1, 116.6, 79.6, 77.2, 73.4, 71.5, 55.3, 41.2, 40.4, 35.4, 33.9, 32.5, 25.5, 24.1, 19.2, 17.7, 15.1, 14.8, 10.8, 6.9, 5.5; HRMS (FAB) calcd. for C₃₄H₅₆NO₅SSi (M+H⁺) 618.3648, found 618.3651.

Compound 3a: HF.Py (0.5 mL) was added to a solution of 11a (40 mg, 0.064 mmol) in THF (1.5 mL) in a plastic vial at 0° C. The resulting solution was stirred at room temperature for 90 min, and then carefully poured into saturated NaHCO₃ solution (5 mL), which was extracted with EtOAc (3×5 mL). The combined organic layers were washed with brine (5 mL), dried over MgSO₄ and concentrated in vacuo. The residue was purified using silica gel chromatography employing 30% EtOAc/hexane as the eluent, which furnished 3a (29 mg, 91% yield): [α] −120.2° (c 0.75, CHCl₃); IR (neat) 3482, 2966, 1733, 1683, 1456, 1251, 1065 cm⁻¹; ¹H NMR (400 MHz, CDCl₃) δ6.95 (s, 1H), 6.54 (s, 1H), 5.51 (dt, J=15.3, 4.8 Hz, 1H), 5.44-5.36 (m, 2H), 5.11 (d, J=7.8 Hz, 2H), 4.21 (d, J=10.4 Hz, 1H), 3.61-3.57 (m, 2H), 3.21 (q, J=6.7 Hz, 1H), 2.92 (dd, J=15.8, 4.0 Hz, 1H), 2.71 (s, 3H), 2.62-2.59 (m, 1H), 2.53-2.49 (m, 1H), 2.45-2.35 (m, 4H), 2.28 (dd, J=13.9, 1.6 Hz, 1H), 2.08 (s, 3H), 1.96-1.89 (m, 1H), 1.82-1.79 (m, 1H), 1.69 (s, 3H), 1.36 (s, 3H), 1.09 (d, J=6.9 Hz, 3H), 1.06 (s, 3H), 0.85 (d, J=6.9 Hz, 3H); ¹³C NMR (100 MHz, CDCl₃) δ222.1, 170.8, 165.2, 151.6, 139.3, 136.4, 128.2, 125.1, 121.7, 118.6, 115.6, 78.6, 72.1, 71.8, 53.7, 40.7, 39.5, 35.0, 34.4, 34.2, 32.4, 23.8, 21.5, 18.9, 17.6, 15.6, 15.5, 10.9; HRMS (FAB) calcd. for C₂₈H₄₁NNaO₅S (M+Na⁺) 526.2603, found 526.2619.

Compound 9: To a stirred solution of vinyl iodide 7 (250 mg, 0.54 mmol) in Et₂O (5 mL) at room temperature were added PdCl₂(dppf) (100 mg, 0.122 mmol, 0.227 equiv), followed by a solution of butenyl magnesium bromide (1.62 mmol, 3.0 equiv) in Et₂O (3 mL). The reaction mixture was stirred at room temperature for 12 h, diluted with Et₂O (10 mL) and water (10 mL). The aqueous layer was separated and extracted with Et₂O (2×10 mL). The combined organic extracts were dried over MgSO₄ and concentrated in vacuo. Purification using silica gel chromatography employing 5% EtOAc/pentane as the eluent afforded 1,5-diene 9 (158 mg, 75% yield) as a clear oil: [α]_{D} +38.2 (c 1.4, CDCl₃); IR (neat) 3072, 2931, 2861, 1637, 1508, 1472, 1249, 1179, 1073, 938, 832, 773 cm⁻¹; ¹H NMR (400 MHz, CDCl₃) δ6.91 (s, 1H), 6.45 (s, 1H), 5.80 (m, 1H), 5.16 (t, 1H, J=6.8 Hz), 4.99 (dd, 1H, J=17.5, 1.5 Hz), 4.95 (dd, 1H, J=10.1, 1.9 Hz), 4.08 (t, 1H, J=6.5 Hz), 2.70 (s, 3H), 2.30-2.20 (m, 2H), 2.13-2.20 (m, 4H), 1.83 (s, 3H), 1.67 (s, 3H), 0.88 (s, 9H), 0.04 (s, 3H), 0.00 (s, 3H); ¹³C NMR (400 MHz, CDCl₃) δ164.3, 153.2, 142.5, 138.6, 136.0, 122.0, 118.6, 115.0, 114.4, 79.0, 35.3, 32.2, 31.5, 25.8, 23.4, 19.2, 18.2, 13.9, −4.7, −4.9; HRMS (FAB) calcd. For C₂₂H₃₈NOSSi (M+H⁺) 392.2443, found 392.2442.

Alcohol 5b: To a stirred solution of 1,5-diene 9 (460 mg, 1.18 mmol), in THF (12 mL) at 0° C. was added tetrabutyl ammonium fluoride (1.0M in THF, 2.94 mL, 2.94 mmol). The reaction mixture was stirred at room temperature for 2 hours, at which point saturated ammonium chloride (10 mL) was added and the mixture was diluted with EtOAc (20 mL) and extracted 3× with EtOAc (20 mL), before being dried over Na₂SO₄ and concentrated in vacuo. Purificatio using silica gel chromatography employing 25% EtOAc/hexanes as the eluent afforded alcohol 5b (320 mg, 98% yield) as a clear oil: [α]_{D} +1.3 (c 1.4, CDCl₃); IR (neat) 3331, 2966, 2908, 2849, 1637, 1502, 1443, 1373, 1185, 1038, 908, 726 cm⁻¹; ¹H NMR (400 MHz, CDCl₃) δ6.94 (s, 1H), 6.56 (s, 1H), 5.82-5.76 (m, 1H), 5.19 (dt, 1H, J=7.2, 1.1 Hz), 5.02 (dd, 1H, J=17.0, 1.6 Hz), 4.95 (dd, 1H, J=10.0, 1.8 Hz), 4.15 (t, 1H, J=6.5 Hz), 2.70 (s, 3H), 2.35 (t, 2H, J=6.9 Hz), 2.17 (m, 4H), 2.05 (S, 3H), 1.99 (s, 1H, OH), 1.72 (s, 3H); ¹³C NMR (400 MHz, CDCl₃) δ164.5, 152.9, 141.7, 138.4, 138.3, 120.8, 118.9, 115.4, 114.7, 77.2, 34.1, 32.1, 31.4, 23.5, 19.2, 14.3; HRMS (FAB) calcd. For C₁₆H₂₄NOS (M+H⁺) 278.1579, found 278.1579.

Compound 4b: The 3-O-TES-6-O-Troc protected acid 6 was dried through azeotropic distillation from benzene. Freshly dried acid 6 (385 mg, 0.67 mmol) is dissolved in DCM (5 mL) and cooled to 0 C., at which point solid DMAP (115 mg, 0.94 mmol) and solid EDCI (180 mg, 0.94 mmol) are added. After stirring the reaction mixture at 0° C. for 15 min alcohol 5b (300 mg, 1.08 mmol) is added dropwise. The cooling bath is removed and stirring continued for another 2 hours. The crude reaction mixture is diluted with DCM (20 mL) and stripped onto silica and purified using silica gel chromatography employing 10% EtOAC/Hexanes as the eluent yielding ester 4b (375 mg, 67% yield) as a clear oil: [α]_{D} +1.5 (c 1.4, CDCl₃); IR (neat) 2955, 1731, 1702, 1455, 1378, 1243, 1179, 1096, 991, 926, 814, 732 cm⁻¹; ¹H NMR (400 MHz, CDCl₃) δ6.93 (s, 1H), 6.48 (s, 1H), 5.83-5.64 (m, 2H), 5.19 (t, 1H, J=6.9 Hz), 5.08 (t, 1H, J=6.5 Hz), 5.03-4.99 (m, 3H), 4.82 (d, 1H, J=12 Hz), 4.72 (dd, 1H, J=18.0, 3.5 Hz), 4.66 (d, 1H, J=12.0 Hz), 4.21 (dd, 1H, J=-7.1, 3.0 Hz), 3.51-3.45 (m, 1H), 2.69 (s, 3H), 2.58 (dd, 1H, J=17.3, 3.0 Hz), 2.49-2.34 (m, 3H), 2.26-2.15 (m, 2H), 2.11 (s, 3H), 2.05 (s, 3H), 1.90-1.82 (m, 2H), 1.67 (s, 3H), 1.35 (s, 3H), 1.27-1.19 (m, 2H), 1.06 (d, 3H, J=6.8 Hz), 1.00 (s, 3H), 0.97-0.85 (m, 11H), 0.63 (q, 6H, J=7.8 Hz); ¹³C NMR (400 MHz, CDCl₃) δ215.2, 171.3, 164.5, 154.0, 152.6, 138.3, 137.6, 137.2, 135.7, 121.1, 119.8, 117.,1, 116.3, 114.6, 94.7, 81.7, 79.7, 77.2, 75.2, 53.4, 42.2, 39.7, 36.6, 34.4, 32.1, 31.5, 23.4, 22.3, 20.9, 19.2, 15.6, 14.5, 10.6, 7.0, 5.0; HRMS (FAB) calcd. For C₄₀H₆₂Cl₃NNaO₇SSi (M+Na⁺) 856.2979, found 856.2984

Compound 10b: Diene 4b (85 mg, 0.102 mmol) was dissolved in dry DCM (51 mL) and heated in the presence of tricyclohexylphosphine[1,3-bis(2,4,6-trimethylphenyl)-4,5-dihydroimidazol-2-ylidene][benzylidene]ruthenium (IV) dichloride (13 mg, 0.015 mmol) at reflux for 2.5 hours. The reaction mixture was cooled to room temperature and stripped onto silica and purified using silica gel chromatography employing 4-10% EtOAc/Hexanes gradient as the eluent to furnish triene 10b (45 mg, 55% yield) as a clear oil: [α]_{D} +20.1 (c 1.4, CDCl₃); IR (neat) 2943, 2872, 1749, 1719, 1461, 1378, 1249, 1179, 1085, 961, 932, 814, 732 cm⁻¹; ¹H NMR (400 MHz, CDCl₃) δ7.01 (s, 1H), 6.48 (s, 1H), 5.54-5.38 (m, 2H), 5.11-5.06 (m, 2H), 4.95 (d, 1H, J=12.0 Hz), 4.9 (dd, 1H, J=10.9, 3.8 Hz), 4.63-4.62 (m, 1H), 4.60 (d, 1H, J 12.0 Hz), 3.37 (q, 1H, J=6.7 Hz), 3.10-3.04 (m, 1H), 2.87-2.80 (m, 1H), 2.72 (s, 3H), 2.50-2.27 (m, 4H), 2.19-2.05 (m, 2H), 2.12 (s, 3H), 1.94-1.79 (m, 3H), 1.67 (s, 3H), 1.20 (s, 3H), 1.14 (d, 3H, J=6.9 Hz); 1.11 (d, 3H, J=6.9 Hz), 0.97 (t, 9H, J=7.8 Hz), 0.95 (s, 3H), 0.62 (q, 6H, J=7.8 Hz);¹³C NMR (400 MHz, CDCl₃) δ215.1, 172.8, 164.6, 153.9, 152.5, 141.2, 136.0, 132.6, 126.6, 122.5, 118.4, 116.4, 95.0, 83.2, 77.9, 71.4, 68.2, 56.1, 40.9, 38.9, 34.8, 33.4, 33.1, 29.6, 23.1, 22.4, 19.2, 16.0, 15.4, 15.0, 11.3, 7.0, 5.1; HRMS (FAB) calcd. For C₃₈H₅₈Cl₃NNaO₇SSi (M+Na⁺) 828.2666, found 828.2668.

Compound 11b: To a solution of triene 10b (125 mg, 0.154 mmol) in a mixture of THF (1.5 mL) and acetic acid (1.5 mL) was added nanosize zinc (101 mg, 1.54 mmol). The reaction mixture was sonicated at room temperature for 20 minutes at which point it was diluted with EtOAc(20 mL) and filtered through a Celite Plug. The filter solution was washed with saturated bicarbonate (20 mL) and the aqueous phase was back-extracted twice with EtOAc (20 mL). The combined organic layers were dried over Na₂SO₄ and evaporated in vacuo to furnish alcohol, 11b (93 mg, 95% yield) as a clear oil: [α]_{D} +36.0 (c 1.4, CDCl₃); IR (neat) 3519, 2955, 2872, 1719, 1684, 1455, 1373, 1290, 1179, 1085, 1014, 973 cm⁻¹; ¹H NMR (400 MHz, CDCl₃) δ6.99 (s, 1H), 6.44 (s, 1H), 5.59-5.49 (m, 2H), 5.05 (t, 1H, J=7.6 Hz), 4.88 (dd, 1H, J=10.2, 4.2 Hz), 4.44 (t, 1H, J=4.2 Hz), 3.78 (d, 1H, J=10.0 Hz), 3.60 (s, 1H), 3.21 (q, 1H, J=6.9 Hz), 2.99-2.93 (m, 1H), 2.72 (s, 3H), 2.71-2.64 (m, 2H), 2.33 (s, 3H), 2.11 (s, 3H), 1.95-1.79 (m, 4H), 1.68 (s, 3H), 1.60 (s, 1H), 1.27 (s, 3H), 1.08 (d, 3H, J=6.9 Hz), 0.98-0.94 (m, 6H), 0.96 (t, 9H, J=7.8 Hz), 0.60 (q, 6H, J=7.8 Hz); ¹³C NMR (400 MHz, CDCl₃) δ222.5, 172.0, 164.7, 152.4, 141.1, 135.9, 132.0, 126.9, 122.5, 118.3, 116.6, 83.3, 71.7, 70.6, 55.6, 40.5, 40.1, 34.7, 34.0, 33.3, 33.1, 30.0, 23.1, 22.3, 19.3, 16.1, 15.1, 14.5, 10.1, 6.9, 5.0; HRMS (FAB) calcd. For C₃₅H₅₇NNaO₅SSi (M+Na⁺) 654.3624, found 654.3625.

Compound 3b: To a stirred solution of 11b,(93 mg, 0.147 mmol) in THF (10 mL) at 0 C. was added pyridine (1 mL) and HF-pyridine (1 mL) dropwise. When the addition was complete the cooling bath was removed and stirring continued at room temperature for 12 hours. The reaction mixture was diluted with EtOAc (50 mL) and washed with cold saturated bicarbonate (2×10 mL). The aqueous layer was back extracted with EtOAc (220 mL) and the combined organic layers were dried over Na₂SO₄ and evaporated in vacuo. The crude mixture was purified using silica gel chromatography employing 20-40% EtOAc/Hexanes gradient as the eluent to afford diol 3b (60 mg, 79% yield) as a clear oil: [α]_{D} −17.1 (c 1.4, CDCl₃); IR (neat) 3483, 2966, 2919, 1725, 1684, 1502, 1449, 1373, 1290, 1249, 1173, 973, 726cm⁻¹; ¹H NMR (400 MHz, CDCl₃) δ6.98 (s, 1H), 6.52 (s, 1H), 5.58-5.48 (m, 2H), 5.26 (t, 1H, J=6.2 Hz), 5.08 (t, 1H, J=7.1 Hz), 4.10-4.08 (m, 1H), 3.77 (d, 1H, J=8.4 Hz), 3.30 (s, 1H), 3.19-3.14 (m, 1H), 2.72 (s, 3H), 2.60-2.40 (m, 4H), 2.34-2.27 (m, 1H), 2.21-2.05 (m, 4H), 2.10 (s, 3H), 1.98-1.92 (m, 1H), 1.79-1.75 (m, 1H), 1.68 (s, 3H), 1.34 (s, 3H), 1.09 (s, 3H), 1.08 (d, 3H, J=7.4 Hz), 0.90 (d, 3H, J=7.0 Hz); ¹³C NMR (400 MHz, CDCl₃) δ221.8, 171.1, 164.8, 152.3, 139.6, 133.1, 126.4, 120.5, 119.0, 116.2, 79.8, 74.0, 71.5, 52.4, 42.2, 38.4, 34.6, 34.0, 32.7, 31.5, 31.1, 23.4, 21.8, 20.2, 19.2, 15.6, 10.6; HRMS (FAB) calcd. For C₂₉H₄₃NNaO₅S (M+Na⁺) 540.2760, found 540.2758.

Results and Discussion: The synthesis of the 17- and 18-membered ring homologs commenced with the conversion of the previously reported vinyl iodide 7 to the corresponding 1,4-diene 5a and 1,5-diene 5b (Chappell et al. Org. Lett. 2:1633, 2000; incorporated herein by reference). Reaction of the vinyl iodide 7, with allyltributyltin under Stille conditions, afforded the desired 1,4-diene 8 in 92% yield. Correspondingly, reaction of vinyl iodide 7 with butenylmagnesium bromide under the Tamao-Kumada-Corriu palladium(0) mediated coupling conditions provided the desired 1,5-diene 9 in 75% yield (Tamao et al. J. Am. Chem. Soc. 94:4374, 1972; Corriu et al. Chem. Comm. 144, 1972; each of which is incorporated herein by reference). It is likely that this reaction could be extended towards the synthesis of alternative unconjugated dienes, which could allow for the synthesis of even larger ring analogs. Finally, treatment of 1,4-diene 8 and 1,5-diene 9 with tetra-n-butylammonium fluoride accomplished deprotection of the secondary alcohol in high yield.

Esterification of the resultant allylic alcohols 5a and 5b with C1-C10 acid fragment 6 provided the corresponding RCM cyclization precursors in 61% (4a) and 67% (4b) yields, respectively. The ring-closing metathesis reaction of 1,4-diene 4a was then carried out using the second generation Grubbs catalyst (Reviews: Grubbs et al. Acc. Chem. Res. 28:446, 1995; Trnka et al. Acc. Chem. Res. 34:18, 2001; Alkene Metathesis in Organic Chemistry Ed.: Fürstner, A.; Springer, Berlin, 1998; Fürster Angew. Chem. Int. Ed. Engl. 39:3012, 2000; Schrock Top. Organomet. Chem. 1:1, 1998; each of which is incorporated herein by reference) in methylene chloride, which provided, as in our earlier study (Biswas et al. J. Am. Chem. Soc. 2002, in press; incorporated herein by reference), exclusively the trans isomer 10a in a yield of 58% . Using the same RCM reaction conditions with the 1,5-diene 4b provided exclusively the trans isomer 10b in 55% yield, along with recovered starting material. Finally, reductive cleavage of the 2,2,2-trichloroethoxycarbonyl protecting group with zinc and acetic acid followed by deprotection of triethylsilyl ether with HF-pyridine led to the [17]- and [18]ddEpoB (3a and 3b).

The fully synthetic [17]- and [18]ddEpoB have been evaluated against a variety of cell types to determine their antitumor potential. As shown in the table below, [17]ddEpoB (3a) exhibited high cytotoxic activity against a variety of sensitive and resistant tumor cell lines. Direct comparison of [17]ddEpoB (3a) with the previously reported [16]ddEpoB (2e) indicates that the new compound possesses comparable potency.

In vitro Cytotoxicities (IC₅₀) with tumor cell lines

| | IC₅₀ (μM) | | | |
|---|---|---|---|---|
| | [17]ddEpoB | [18]ddEpoB | [16]ddEpoB | dEpoB |
| Tumor Cell Lines | (3a) | (3b) | (2e) | (2b) |
| CCRF-CEM | 0.040 | 0.322 | 0.025 | 0.011 |
| CCRF-CEM/ | 0.126 | 0.870 | 0.091 | 0.015 |
| VBL₁₀₀ | | | | |
| CCRF-CEM/VM₁ | 0.055 | ND | 0.035 | 0.016 |
| CCRF-CEM/Taxol | 0.053 | 0.508 | 0.032 | 0.007 |

XTT assay following 72 h inhibition. CCRF-CEM is a human T-cell acute lymphoblastic leukemia cell line. The CCRF-CEM/_{VBL100}, CCRF-CEM/_{VM1}, and CCRF-CEM/_{Taxol} cell lines all overexpress P-glycoprotein and display a multidrug resistance phenotype to MDR-associated oncolytics. (Chou et al. Proc. Natl. Acad. Sci. USA 98:8113, 2001; incorporated herein by reference).

The in vitro tumor growth inhibition experiments demonstrated the new [17]ddEpoB (3a) analog possesses high in vitro antitumor activity, which is comparable to that of [16]ddEpoB (2e).

### Example 5

Synthesis of Epothilone 490

Previously disclosed and highly accessible building blocks were used to pursue a new approach to the epothilone synthesis problem. These building blocks are vinyl iodide 3, and aldehyde 4 (Scheme 1) (Lee et al. J. Am. Chem. Soc. 132:5249, 2001; incorporated herein by reference).

A convergent solution was used to accomplish the C1-C2 interpolation and the creation of the diene functionality. In the event, Stille coupling of 3 with vinyl n-tributyltin afforded 5. Cleavage of the silyl protecting group afforded 6. The final step in building the O-alkyl segment involved simple acetylation of the secondary alcohol (7, 98% from 3).

Deprotonation of the methyl group of the C15 acetate, as shown, was followed by conversion of the lithium ester enolate to the chiral titanium enolate as described by Duthaler (Duthaler et al. Angew. Chem. Int. Ed. Engl. 28:495, 1989; incorporated herein by reference). Treatment of this ensemble with aldehyde 4 accomplished union of the two major units leading to an 85% yield of the C3 S diastereomer (see product 8). Deprotection of the Troc group produced 9. RCM was accomplished by recourse to the second generation Grubbs ruthenium catalyst, 10, (Scholl et al. Tetrahedron Lett. 40:2247, 1999; incorporated herein by reference) leading to fully synthetic epothilone 490 (2) identical to an authentic sample. The formation of the E-10,11-double bond was highly stereoselective and helped to confirm the stereochemistry of epothilone 490 to be as shown.

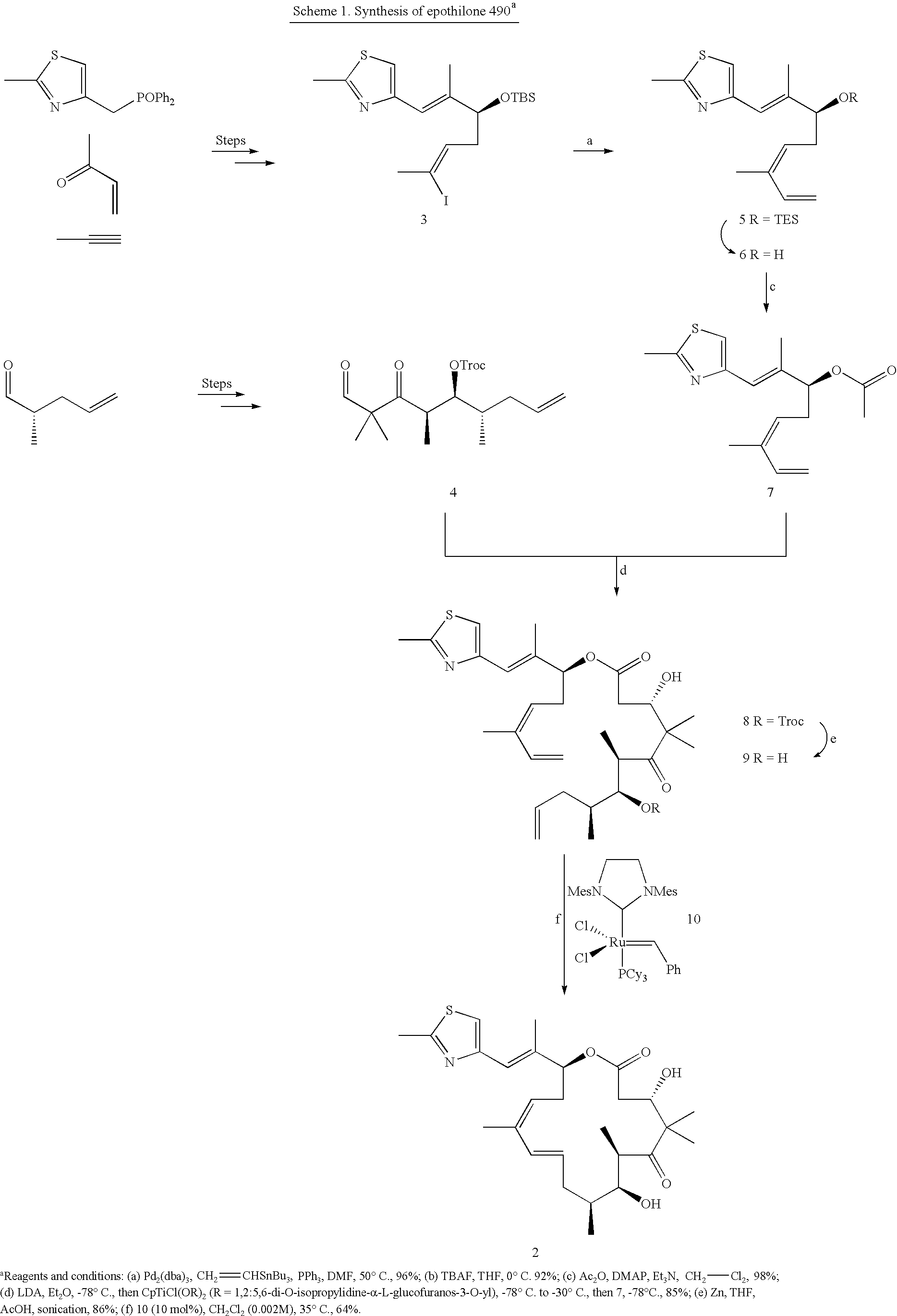

Next this highly concise new route was used to reach dEpoB. This goal was accomplished by positionally specific diimide reduction (Pasto et al. Org. React. 40:91, 1991; incorporated herein by reference) of fully synthetic 2 (86% yield, Scheme 2).

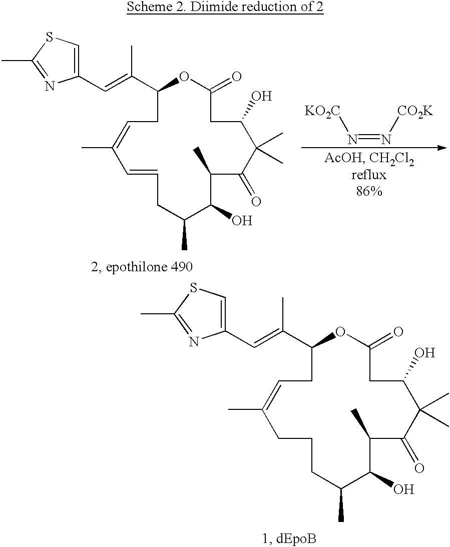

The synthesis and evaluation of some novel epothilones available via 2 is also reported. Encouraged by the selective reduction en route to 1, dienes in this series were subjected to dihydroxylation, epoxidation, and cyclopropanation conditions (Scheme 3). Treatment of 2 with catalytic osmium tetroxide in the presence of NMO resulted in the formation of a 10:1 mixture, where the major product is 12, as proven crystallographically (The minor product arises from the dihydroxylation of the 12,13-olefin.). Exposure of 2 to the action of DMDO, with the intent of generating an epoxide, gave rise to tetrahydrofuran-containing macrocycle 13 upon silica gel purification. Compound 13 arises from epoxidation of the 12,13-olefin and S_{N}2′ type participation of the C-7 hydroxyl group. Th stereochemistry of 13 was assigned based on the analysis of 2D COSY and NOESY spectra, assuming that all the existing stereochemistry remained untouched under the mild reaction conditions. Finally, treatment of 11, obtained by RCM of 8 (see Scheme 1), with diazomethane in the presence of Pd(OAc)₂, (Denmark et al. J. Org. Chem. 62:3375, 1997; incorporated herein by reference) followed by deprotection, afforded 15.

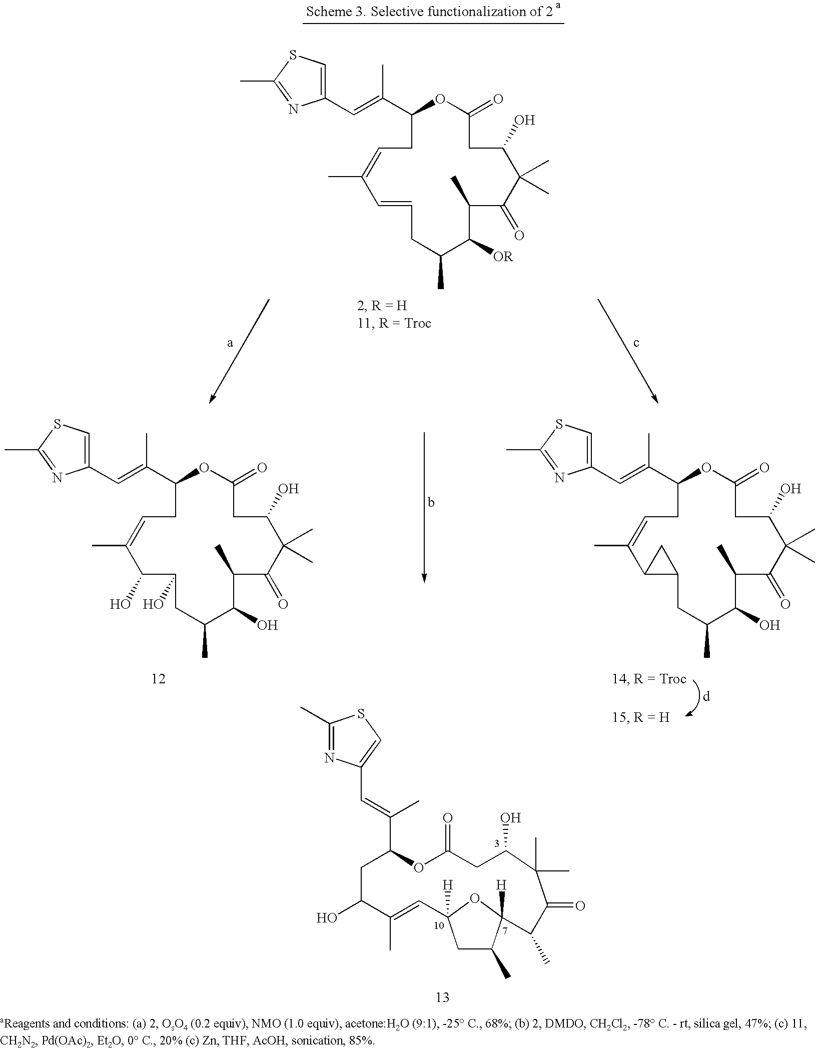

The new analogues obtained from epothilone 490 exhibited a range of in vitro cytotoxities, with 15 showing promising levels of inhibitory efficacy (Table 1, below).

TABLE 1
In vitro Cytotoxicities (IC₅₀) with tumor cell lines^{a}.

| | CCRF- | | | | |
|---|---|---|---|---|---|
| | CEM(C) | C/VBL₁₀₀ | C/VM₁ | C/Taxol | % Tubulin |
| Compound | (μM) | (μM) | (μM) | (μM) | Binding |
| 1 (dEpoB) | 0.011 | 0.015 | 0.016 | 0.007 | 100 |
| 2 (Epo490) | 0.025 | 0.091 | 0.035 | 0.032 | 89 |
| 21-OH-Epo490 | 0.030 | 0.202 | 0.061 | 0.051 | 77 |
| 12 (dihydroxy) | 1.001 | 99.0 | 2.35 | 16.76 | 31 |
| 13 (THF | 0.761 | 8.76 | n.d.^{b} | 4.24 | inactive |
| macrocycle) | | | | | |
| 15 (cyclopropyl) | 0.077 | 0.141 | n.d.^{b} | n.d.^{b} | 84 |
| [17]Epo490 | | | | | 94 |
| [18]Epo490 | | | | | 51 |

^{a}XIT assay following 72 h inhibition. CCRF-CEM is a human T-cell acute lymphoblastic leukemia cell line. The CCRF-CEM/VBL₁₀₀ cell liine is resistant to vinblastine, CCRF-CEM/VM1 to teniposide and CCRF-CEM/Taxol to taxol (Ref. 5).
^{b}not determined.

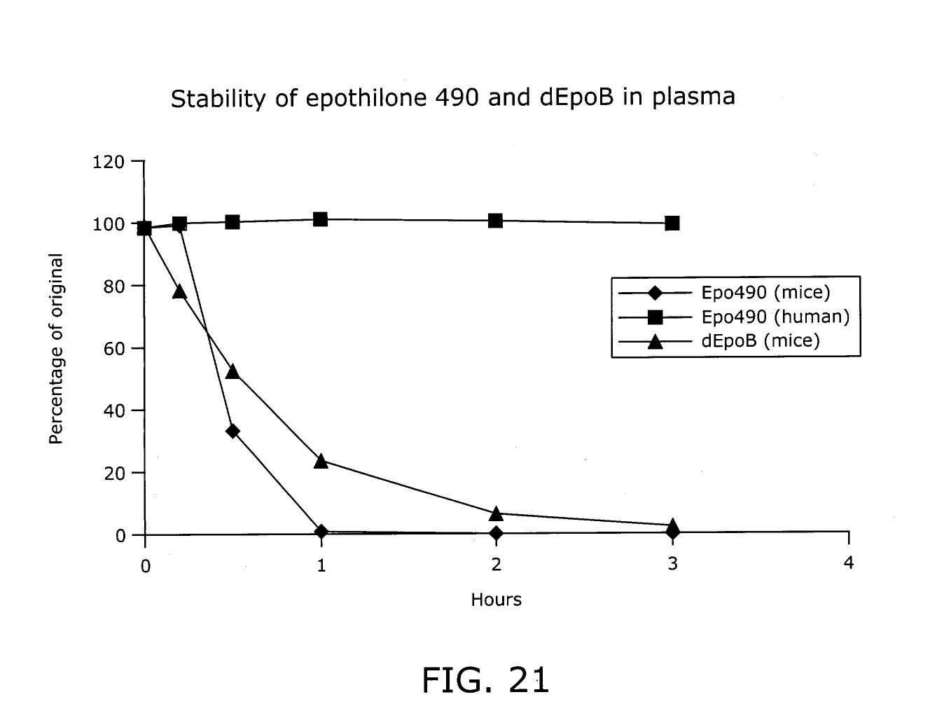

Epothilone 490 exhibited impressive cell growth inhibition across a range of drug-resistant tumors. Surprisingly, epothilone 490 did not demonstrate a statistically significant inhibitory effect on the growth of the implanted tumors, as compared to control mice (See Example 13). This result was surprising in view of the favorable results of the in vitro studies. However, the apparently disappointing murine in vivo results should be viewed in the context of reports that dEpoB itself evidenced a degree of bioinstability in murine plasma; yet had much longer plasma half-lives in higher organisms, including humans (Chou et al. J. Proc. Natl. Acad. Sci. USA 98:8113, 2001; incorporated herein by reference). The observed discrepancy in efficacy between mice and other mammals, including humans, has been ascribed to higher esterase levels in rodents. Indeed, on exposure of 1 and 2 to murine plasma, a faster degradation of epothilone 490 as compared to dEpoB was observed (FIG. 21), with the murine stability of 2 being measurably less than 1. However, no measurable degradation of 2 was observed after more than 3 hours of exposure in human plasma.

In view of such data, those having skill in the pharmacological arts will therefore understand that the observed discrepancy between the excellent in vitro activity of epothilone 490 and its degree of activity in the murine assay is likely to be merely an artifact of murine biochemistry.

### Example 6

Synthesis of 27-Trifluoro-10, 11-dehydro-13,14-[17]desoxyepothilone B

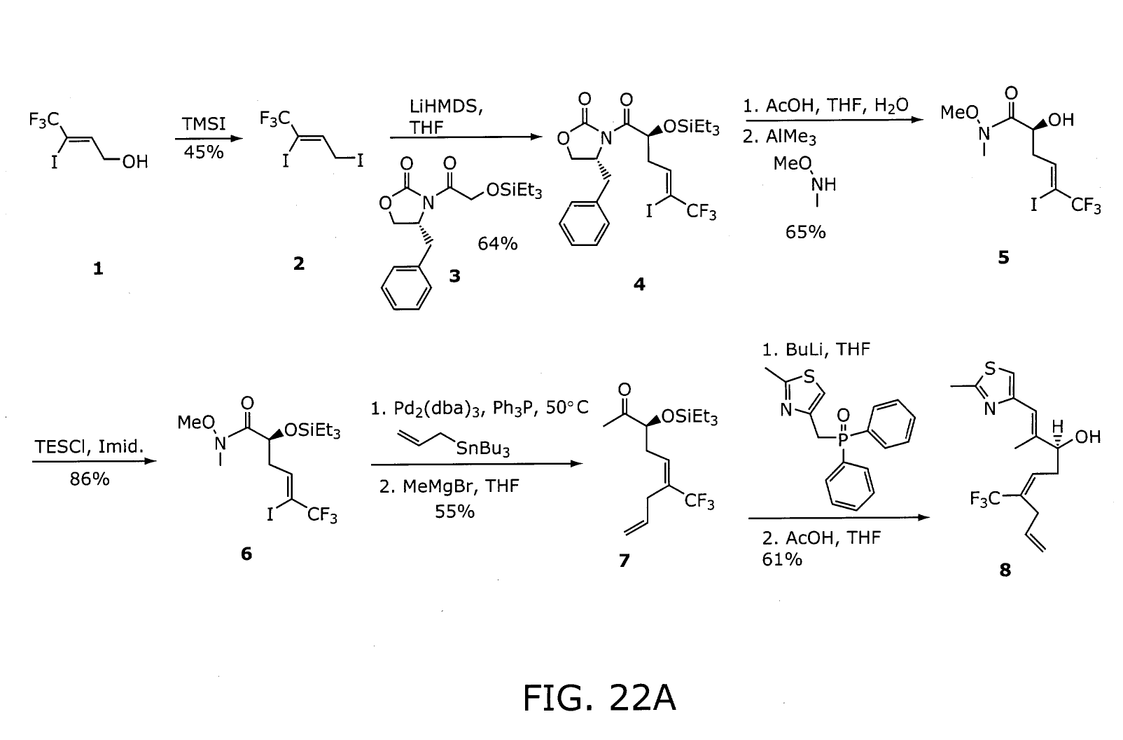

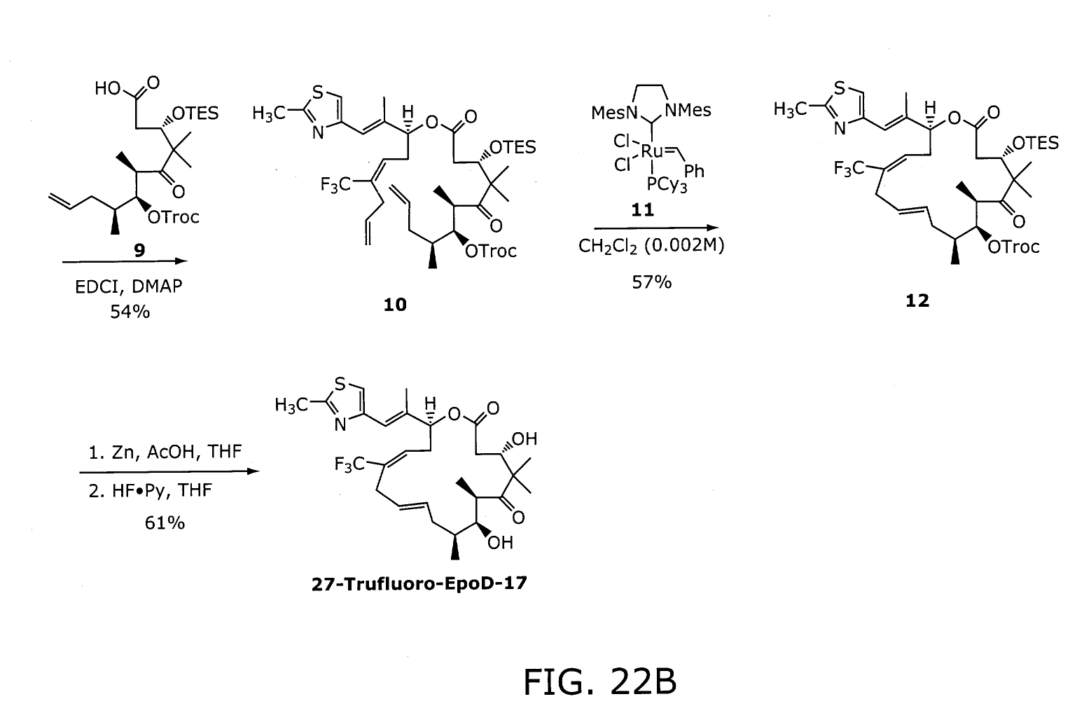

The synthesis of 27-trifluoro-10,11-dehydro-13,14-[17]desoxyepothilone B is shown in FIG. 22.

Accordingly, we undertook a synthesis of 10,11-dehydro-13,14-desoxy-27-trifluoro-[17]epothilone B (27-F₃-[17]ddEpoB, 19, Equation 1) via the ring-closing methathesis of 18, which contained the 1,4-diene required to accommodate the new goal.

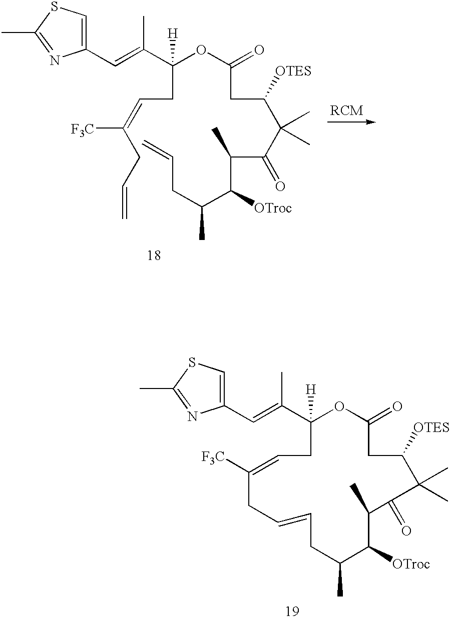

The synthesis of 27-F₃-[17]ddEpoB 19 was commenced by the preparation of the alkyl sector 21. Reaction of Weinreb amide 10 with allyltributyltin under Stille conditions followed by methyl Grignard addition gave the desired ketone 20. Condensation of ketone 20 with phosphine oxide 12 followed by deprotection of the triethylsilyl ether gave the fragment 21 in good yield.

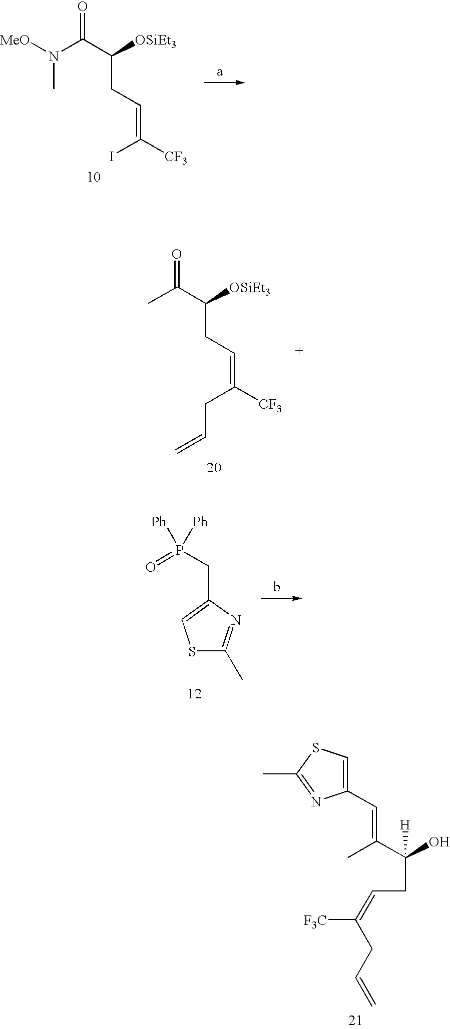

(a) i) Allyltributyltin, AIBN, Benzene, 80° C., 3 h, 74%; ii) MeMgBr, 0° C., 93%; (b) i) 7, n-BuLi THF, −78° C., 30 min.; 14, −78° C. to rt, 85%; iii) HOAc:THF:H₂O (3:1:1), 98%; (c) TMSI, CH₂Cl₂, 0° C., 92% .

Esterification of the resulting left fragment 21 with C1-C10 acid fragment 14 provided the RCM precursor 18 in 75% yield. The ring-closing methathesis reaction of 18 was then carried out using the second generation Grubbs catalyst in methylene chloride, providing exclusively the trans isomer 19 in 57% yield along with recovered starting material. Finally, reductive cleavage of the trichloro ethoxy carbonyl protecting group with zinc and acetic acid, followed by deprotection of the TES ether with HF-pyridine, provided 27-F₃-[17]ddEpoB (23).

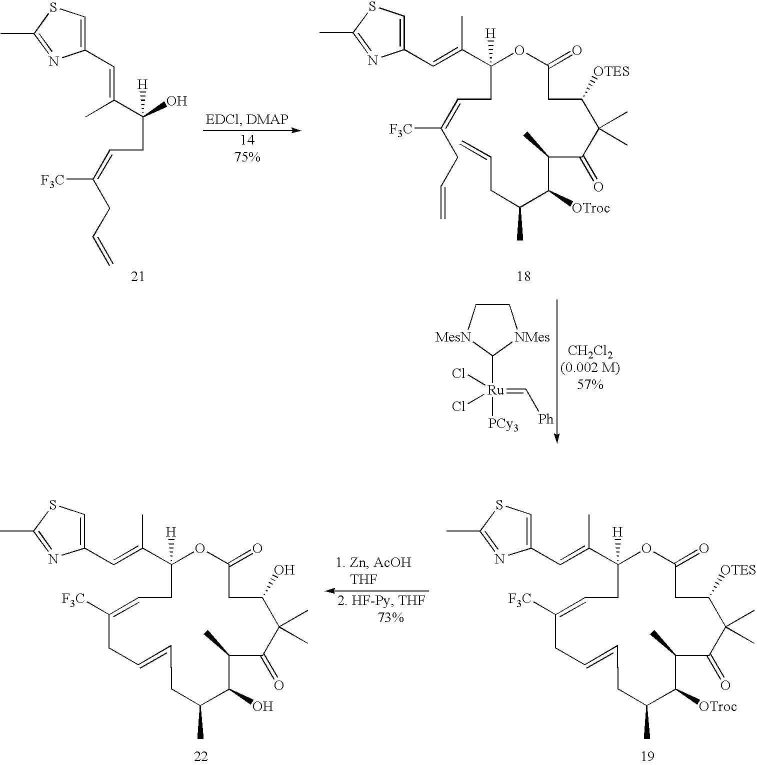

The fully synthetic 27-F₃-[17]ddEpoB (22) was evaluated for its cytotoxic activity. As shown in the table below, direct comparison of the previously reported [17]ddEpoB with 27-F₃-[17]ddEpoB 22 indicated that the new perfluorinated compound possessed a comparably high cytotoxic potency and is more stable in mouse blood plasma than is the parent [17]ddEpoB.

In vitro Cytotoxicities (IC₅₀) with tumor cell lines^{a}

| | | CCRF-CEM/ |
|---|---|---|
| | CCRF-CEM | VBL |
| Compound | (IC₅₀ (μM)) | (IC₅₀ (μM)) |
| 27-Tn-F- | 0.068 | 0.191 |
| [17]ddEpoB (22) | | |
| [17]ddEpoB | 0.040 | 0.126 |
| [16]ddEpoB | 0.020 | 0.068 |

^{a}XTT assay following 72 h inhibition, CCRF-CEM is a human T-cell acute lymphoblastic leukemia cell line. The CCRF-CEM/_{VβL00}¹ CCRF-CEM/_{VM1} and CCRF-CEM/_{Toxo]}cell lines all overexpress P-glycoprotein and display a multidrug resistance phenotype to MDR associated oncolytics (Chou et. al. Proc. Natl. Acad. Sci. USA 98:8113, 2001; incorporated herein by reference).

In summary, we have synthesized 27-F₃-[17]ddEpoB (22) and shown that the trifluoro substitution at the C-27 position maintains the cytotoxicity of the parent [17]ddEpoB and is more stable in murine plasma than the parent compound, ddEpoB.

### Experimentals

### Preparation of 1,1,1-Trifluoro-2,4-diiodo-but-2-ene

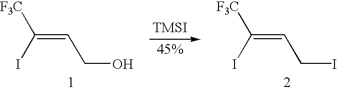

1,1,1-Trifluoro-2,4-diiodo-but-2-ene (2):

To a solution of allylic alcohol 1 (4.0 g, 0.016 mole) in 20 mL of CH₂Cl₂ under argon at 0° C. was added dropwise TMSI (11.3 mL, 0.0790 mole). The reaction mixture was allowed to warm to room temperature and stirred for 16 h. The reaction was cooled to 0° C. and slowly quenched with sat. aq. NaHCO₃ solution. The suspension was diluted with Et₂O (100 mL), washed with aq. NaHCO₃ (100 mL), sat. aq. Na₂S₂O₃ (100 mL), filtered, dried with NaSO₄, and concentrated. Chromatography on silica gel (pentane) provided allyl iodide 2 (2.58 g, 45% ) as a light yellow oil: ¹H NMR (400 MHz, CDCl₃) δ6.86 (t, J=8.4 Hz, 1H), 3.97 (d, J=8.4 Hz, 2H).

### Preparation of (R)-4-Benzyl-3-[(R)-5-iodo-2-triethylsilanoxy-4-hexenoyl]oxazolidin2-one

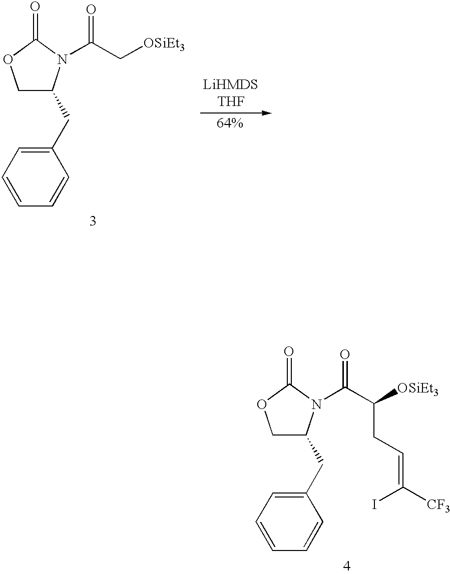

(R)-4-Benzyl-3-[(R)-5-iodo-2-triethylsilanoxy-4-hexenoyl]oxazolidin-2-one (4):

To a solution of imide 3 (3.36 g, 9.63 mmol) in THF (35 mL) was added dropwise a solution of LHMDS (1.0 M in THF, 10 mL) at −78° C. over 30 min. Then, a solution of 1,1,1-Trifluoro-2,4-diiodo-but-2-ene (2, 2.58 g, 7.13 mmol) in THF (10 mL) was added to the cooled enolate solution, and the resulting mixture was slowly warmed to rt over 12 h. The solution was quenched with sat. aq. NaHCO₃ (50 mL) and extracted with EtOAc (3×50 mL). The combined organic extract were washed with brine (100 mL), dried (NaSO₄), filtered, and concentrated. Chromatography on silica gel (10% EtOAc in hexanes) provided imide 4 (2.67 g, 64% ) as a light yellow oil: ¹H NMR (400 MHz, CDCl₃) δ7.34 (t, J=7.6 Hz, 1H), 7.28 (d, J=7.2 Hz, 1H), 7.21 (d, J=7.2 Hz, 2H), 6.74 (t, J=7.0 Hz, 1H), 5.44 (t, J=4.9 Hz, 1H), 4.70-4.64 (m, 1H), 4.18-4.11 (m, 2H), 3.21 (dd, J=13.2, 2.8 Hz, 1H), 2.77-2.67 (m, 2H), 2.68-2.63 (m, 1H), 0.89 (t, J=7.9 Hz, 9H), 0.58 (q, J=7.7 Hz, 6H).

### Preparation of N-Methoxy-N-methyl (S)-2-hydroxy-5-iodo-hex-4-enamide (5)

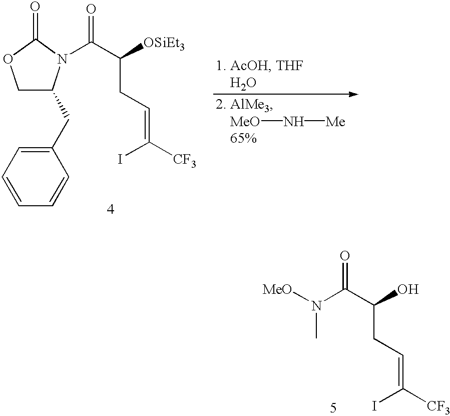

N-Methoxy-N-methyl (S)-2-hydroxy-5-iodo-hex-4-enamide (4): Alkylated imide 4 (4.00 g, 6.86 mmol) was dissolved in HOAc-THF-H₂O (3:1:1, 45 mL) and stirred at rt for 4 h. After the solvent was removed, the oily residue was dissolved in EtOAc (100 mL) and washed with 10% NaHCO₃ (2×50 mL), and brine (50 mL). The organic layer was dried (NaSO₄), filtered, and concentrated to give the corresponding hydroxy of imide 4, which was used for the subsequent reaction without further purification.

To a solution of N,O-dimethylhydroxylamine hydrochloride (2.7 g, 27.7 mmol) in THF (35 mL) was added dropwise a solution of AlMe₃ (2.0 M in toluene, 13.8 mL, 27.7 mmol) at 0° C. After the addition was complete, and the solution was allowed to warm to rt and stirred for 2 h. This solution was then cannulated into a solution of the crude alkylated glycolimide (prepared above) in THF (15 mL) at 0° C. After the addition the mixture was stirred at rt for 6 h. The reaction was quenched by the addition of a 1 N tartaric acid solution (30 mL), and the stirring was continued for 1 h. The organic layer was removed, and the aqueous layer was extracted with EtOAc (3×100 mL). The combined organic layers were dried (NaSO₄), filtered, and concentrated. Chromatography on silica gel (20% acetone in hexanes) provided N, O-dimethylamide 5 (1.51 g, 65% for two steps) as a clear oil: H NMR (400 MHz, CDCl₃) δ6.71 (t, J=6.6 Hz, 1H), 4.57-4.51 (m, 1H), 3.63 (s, 3H), 3.13 (s, 3H), 2.69-2.61 (m, 1H), 2.47 (dd, J=14.5, 6.8 Hz, 1H).

### Preparation of N-Methoxy-N-methyl (S)-2-triethylsilanoxy-5-iodo-hex-4-enamide (6)

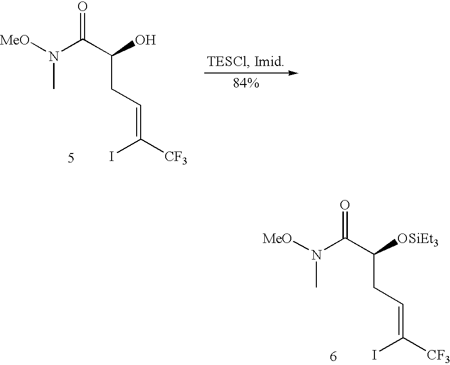

N-Methoxy-N-methyl (S)-2-triethylsilanoxy-5-iodo-hex-4-enamide (6). To a solution of N, O-dimethylamide 5 (5.00 g, 14.2 mmol) in DMF (70 mL) were added imidazole (3.86 g, 56.6 mmol) and TESCI (4.27 g, 28.3 mmol). After stirring at rt for 5 h, the reaction mixture was poured into H₂O (150 mL) and extracted with EtOAc (4×100 mL). The combined organic layers were washed with H₂O (2×100 mL) and dried (NaSO₄), filtered, and concentrated. Chromatography on silica gel (30% EtOAc in hexanes) provided TES-protected imide 6 (5.56 g, 84% ) as a light yellow oil: ¹H NMR (400 MHz, CDCl₃) δ6.81 (t, J=6.4 Hz, 1H), 4.70 (br t, 1H), 3.75 (s, 3H), 3.23 (br s, 3H), 2.67-2.63 (m, 2H), 0.94 (t, J=7.9 Hz, 9H), 0.61 (q, J=7.8 Hz, 6H).

### Preparation of 1,4-Diene-Ketone 7

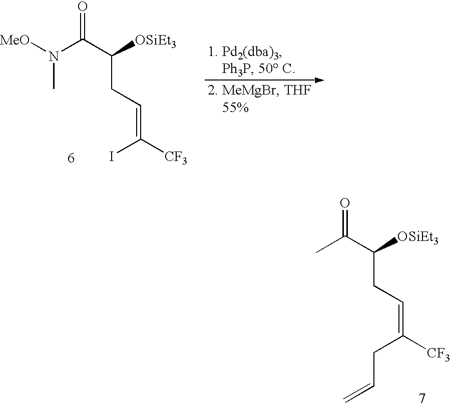

1,4-Diene-Ketone 7: To a stirred solution of vinyl iodide 6 (600 mg, 1.28 mmol) in DMF (60 mL) were added allyltributyltin (1.28 g, 3.85 mmol, 3.0 equiv) and triphenylphosphine (1.35 g, 5.14 mmol, 4 equiv), followed by Pd₂(dba)₃ (1.17 g, 1.28 mmol, 1.0 equiv). The reaction mixture was stirred at room temperature for 12 h, diluted with EtOAc (60 mL) and water (100 mL). The aqueous layer was separated and extracted with EtOAc (2×100 mL). The combined organic extracts were dried over NaSO₄ and concentrated in vacuo. Purification using silica gel chromatography employing 30% EtOAc in hexanes as the eluent afforded the desired corresponding impure 1,4-diene product as a yellow oil which was then dissolved in THF (50 mL) and cooled to 0° C. Methyl magnesium bromide (3.0 M in ether, 10 mmol). The solution was stirred at 0° C. for 1 h and then quenched with sat. aq. NH₄Cl (50 mL). The organic layer was removed and the aqueous layer was extracted with (3×50 mL). The combined organic layers were dried (NaSO₄), filtered, and concentrated. Chromatography on silica gel (10% EtOAc in hexanes) provided TES ether 7 (0.236 g, 55% for two steps) as a yellow oil: ¹H NMR (400 MHz, CHCl₃) δ6.21 (t, J=6.7 Hz, 1H), 5.70-5.61 (m, 1H), 5.09-5.02 (m, 2H), 4.01 (t, 1H), 2.90-2.82 (m, 2H), 2.51-2.41 (m, 2H), 2.11 (s, 3H), 0.95 (t, J=7.9 Hz, 9H), 0.61 (q, J=8.0 Hz, 6H).

### Preparation of 1,4-Diene-Thizaole 7

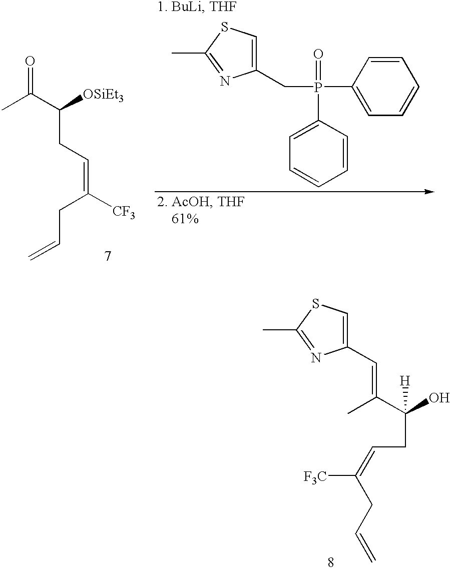

1,4-Diene-Thizaole 8: To a solution of Horner reagent (1.3 g, 4.16 mmol) in THF (10 mL) was added dropwise a solution of n-BuLi (1.6 M in Hexane, 2.6 mL) at −78° C. and allowed to stir at this temperature for 1 h. Then, a solution of ketone 7 (280 mg, 0.83 mmol) in THF (1 mL) was added and the solution allowed to warm to room temperature gradually over 4 h. The reaction mixture was quenched with sat. aq. NaHCO₃ (10 mL) and extracted with ether (3×10 mL). The combined organic layers were dried (NaSO₄), and concentrated. The resultant mother liquor was dissolved in a 3:1:1 solution of AcOH:THF:H₂O (5 mL) and stirred for 90 min at room temperature, at which point it was diluted with toluene (5 mL) and concentrated in vacuo. The residue was dissolved in EtOAc (10 mL) and washed with a saturated solution of saturated NaHCO₃ (2×10 mL). The aqueous layer was further extracted with EtOAc (10 mL). The combined organic layers were washed with brine (10 mL), dried over NaSO₄ and concentrated in vacuo. Purification using silica gel chromatography employing 5% EtOAc/hexane as the eluent afforded alcohol 8 (162 mg, 61% yield) as a clear oil: ¹H NMR (400 MHz, CDCl₃) δ6.97 (s, 1H), 6.56 (s, 1H), 6.30 (t, 1H), 5.71-5.61 (m, 1H), 5.17 (d, J=17.1 Hz, 1H), 4.99 (d, J=10.1 Hz, 1H), 4.25 (t, J=6.1 Hz, 1H), 2.97 (d, 2H, J=5.9 Hz), 2.70 (s, 3H), 2.46-2.43 (m, 2H), 2.05 (s, 3H), 1.61 (s, 1H, OH).

### Preparation of Ester 10

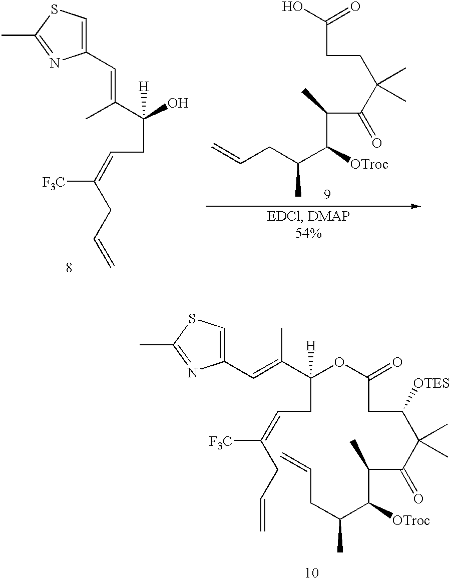

Ester10: To a stirred solution of alcohol 8 (100 mg, 0.32 mmol, 1.8 equiv) in CH₂Cl₂ (10 mL) at 0° C. were added EDCI (53 mg, 0.28 mmol, 1.6 equiv) and DMAP (34 mg, 0.28 mmol, 1.6 equiv). After 15 min, a solution of acid 9 (100 mg, 0.17 mmol, 1 equiv) dissolved in CH₂Cl₂ (5 mL) was added dropwise to the reaction mixture, which was warmed to room temperature and stirred for 6 h. At this point, the reaction was quenched by addition of water (5 mL). The aqueous layer was separated and extracted with Et₂O (2×10). The combined organic extracts were dried with NaSO₄ and concentrated in vacuo. Purification using silica gel chromatography employing 8% EtOAc/pentane as the eluent afforded ester 10 (120 mg, 54% yield) as a clear oil: ¹H NMR (400 MHz, CDCl₃) δ6.93 (s, 11H), 6.49 (s, 1H), 6.23 (t, J=7.2Hz, 1H), 5.74-5.63 (m, 1H), 5.30 (t, J=7.2 Hz, 1H), 5.27-4.96 (m, 6H), 4.81 (d, J=12.0 Hz, 1H), 4.76 (dd, J=7.7, 3.2 Hz, 1H), 4.71 (d, J=12.1 Hz, 1H), 4.21 (dd, J=6.8, 2.5 Hz, 1H), 3.50-3.39 (m, 1H), 2.91-2.72 (m, 3H), 2.54 (s, 3H), 2.26-2.16 (m, 2H), 2.06 (s, 3H), 1.94-1.81 (m, 2H), 1.36 (s, 3H), 1.00 (d, J=6.7 Hz, 3H), 1.01 (s, 3H), 0.97-0.93 (m, 12H), 0.63 (q, J=8.0 Hz, 6H).

### Preparation of Macrolide 12

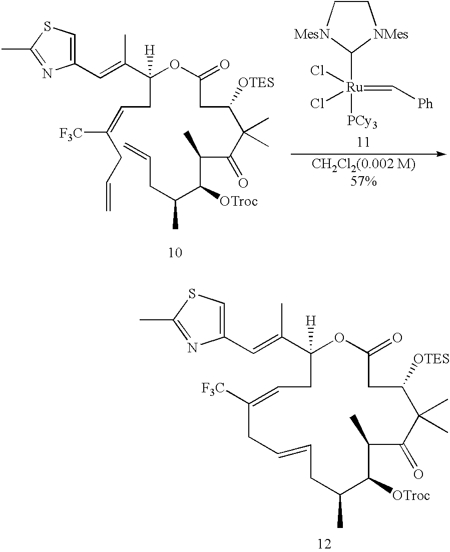

Macrolide 12: A solution of the ester 10 (50 mg, 0.0573 mmol) and tricyclohexylphosphine[1,3-bis(2,4,6-trimethylphenyl)-4,5-dihydroimidazole-2-ylidene-[benzyli dine]ruthenium(IV) dichloride (Grubb's catalyst) (11) (9.73 mg, 0.011 mmol, 0.2 eq.) in 29 mL of CH₂Cl₂ was stirred at 35° C. for 3 h. The solution was cooled to room temperature and passed through a plug of silica gel using 5% Et₂O/pentane which yielded 12 (28.1 mg, 57% ).

Macrolide 12: ¹H NMR (500 MHz, CDCl₃) 6.99 (s, 1H), 6.62 (s, 1H), 6.27 (t, 1H, J=15.1), 6.26 (t, 1H, J=15.1), 5.42-5.31 (m, 1H), 5.22 (dd, 1H, J=8.0), 4.81 (d, 1H, J=12 Hz), 4.79 (d, 1H, J=12 Hz), 4.16 (d, 1H, J=10 Hz), 3.21 (t, 1H, J=7.1 Hz), 3.10 (d, 1H, J=7.1 Hz), 2.78-2.74 (m, 2H), 2.72 (s, 3H), 2.46-2.42 (m, 2H), 2.44-2.37 (m, 3H), 2.03-1.98 (m, 2H), 1.71 (s, 3H), 1.05 (s, 3H), 1.05 (d, 6H, J=6.6, 6-Me & 8-Me), 0.92 (s, 3H), 0.81 (t, 9H, J=7.9 ), 0.51 (q, 6H, J=7.9).

### Preparation of 27-Trifluoro-EpoD-17

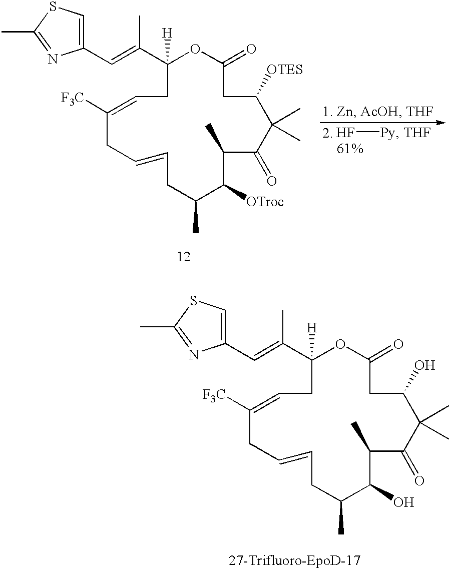

27-Trifluoro-EpoD-17: To a stirred solution of macrolide 12 (25 mg) in 1:1 THF/HOAc (1.2 mL) was added a spatula tip of nanosize Zn°. The reaction mixture was sonicated for 30 min and then filtered through celite, washing the celite cake with EtOAc. The combined filtrate was washed with saturated NaHCO₃, brine, and dried over Na₂SO₄. Removal of the solvent followed by purification of the residue on silica gel chromatography using 25% EtOAc/hexane as the eluent yielded the corresponding impure Troc-cleaved product. HF.Py (0.05 mL) was added to a solution of Troc-cleaved product in THF (0.2 mL) at 0° C. The resulting solution was allowed to warm to room temperature and allowed to stir for 3 h. TMSOMe (0.05 mL) was added dropwise to the reaction mixture which was then concentrated in vacuo. The residue was purified using silica gel chromatography employing 40% EtOAc/hexane as the eluent, which furnished 27-Trifluoro-EpoD-17 (10.1 mg, 61% yield for two steps).

AR-EpoD-17: ¹H NMR (500 MHz, CDCl₃) 7.00 (s, 1H), 6.54 (s, 1H), 6.15 (dt, 1H, J=10.1 Hz), 5.74 (d, 1H, J=8.1 Hz), 5.51-5.42 (m, 2H), 4.17 (d, 1H, J=8.9 Hz), 3.66 (d, 1H, J=6.5 Hz), 3.63 (bs, 1H), 3.20 (q, 1H, J=7.2), 3.00 (dd, 1H, J=10.1, 5.2 Hz), 2.80 (s, 3H), 2.66-2.47 (m, 3H), 2.46-2.21 (m, 4H), 1.94-1.89 (m, 1H), 1.61 (s, 3H), 1.25 (s, 3H), 1.06 (d, 3H, J=8.9 Hz), 0.94 (s, 3H), 0.80 (d, 3H, J=8.9 Hz).

### Example 7

Synthesis of 26-methyl-EpoD by RCM Route

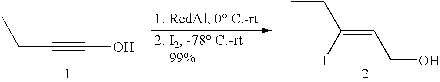

Synthesis of 2. RedAl (7.9 mL of a 65 wt. % solution in toluene, 35.66 mmol, 1.5 equiv) was added to 25 mL diethyl ether at 0° C., followed by a solution of 2-pentyn-1-ol (1, 2.0 g, 23.77 mmol). The reaction was allowed to warm to rt after 2 h, and stirred for 15 h. The white suspension was cooled to −78° C. and treated with a solution of iodine (9.1 g, 35.6 mmol, 1.5 equiv) in diethyl ether (10 mL) and THF (8 mL). The reaction was warmed to rt after 15 min, and stirred for 3 h. Aqueous Rochelle's salt solution (20 mL) was added, followed by stirring for 1 h. The suspension was diluted with diethyl ether (100 mL) and washed with saturated Na₂S₂O₃ solution (50 mL), brine (50 mL), dried (MgSO₄) and concentrated to afford 5.0 g (99% ) allyl alcohol 2 as a colorless oil: ¹H NMR (400 MHz, CDCl₃) δ5.84 (t, J=5.7 Hz, 1H), 4.20 (d, J=5.7Hz, 2H), 2.55 (q, J=7.2 Hz, 2H), 2.37 (s, 1H), 1.11 (t, J=7.2 Hz, 3H).

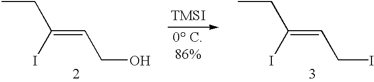

3. Allyl alcohol 2 (5.0 g, 23.58 mmol) was dissolved in methylene chloride (50 mL) and treated with trimethylsilyl iodide (5.0 g, 24.75 mmol, 1.05 equiv) after cooling to 0° C. After stirring for 30 min, the reaction mixture was diluted with methylene chloride (50 mL) and washed with saturated sodium bicarbonate solution (50 mL). The aqueous layer was extracted with methylene chloride (3×50 mL). The combined organic layers were washed with saturated Na₂S₂O₃ solution (50 mL), brine (50 mL), filtered through neutral alumina (eluting with 200 mL diethyl ether), dried (MgSO₄) and concentrated to afford 6.5 g (86% ) of diiodide 3 was a dark red liquid, which was stored over copper metal at −20° C. before use: ¹H NMR (400 MHz, CDCl₃) δ5.82 (t, J=8.1 Hz, 1H), 3.94 (d, J=8.1 Hz, 2H), 2.58 (q, J=7.3 Hz, 2H), 1.09 (t, J=7.3 Hz, 3H).

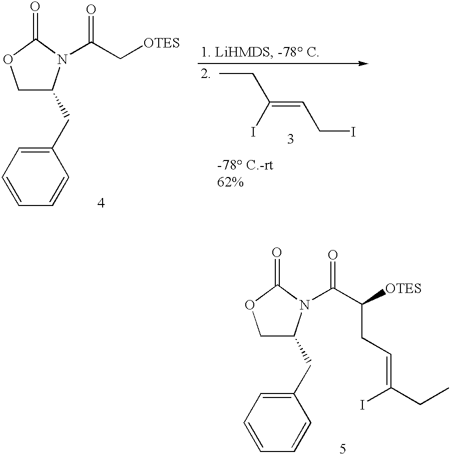

Synthesis of 5. LiHMDS (10.5 mL of a 1.0 M solution in THF, 10.49 mmol, 1.1 equiv) was added to a solution of 4 (3.33 g, 9.53 mmol) in THF (60 mL) at −78° C. After 30 min, a solution of allylic iodide 3 (3.7 g, 11.43 mmol, 1.2 equiv) in THF (5 mL) was slowly added to the reaction mixture. The reaction was allowed to warm to rt over 12 h, followed by dilution with ethyl acetate (100 mL). The solution was washed with saturated sodium bicarbonate solution (50 mL). The aqueous layer was extracted with ethyl acetate (2×50 mL). The combined organic layers were washed with saturated Na₂S₂O₃ solution (50 mL), brine (50 mL), dried (MgSO₄) and purified by silica gel chromatography (8-15% EtOAc/hexane) to give 3.1 g (62% ) of alkylated product 5 as an yellow oil: ¹H NMR (400 MHz, CDCl₃) [symbol] [symbol] 5.72 (t, J=6.4 Hz, 1H), 5.48 (t, J=5.1 Hz, 1H), 4.76-4.69 (m, 1H), 4.28-4.19 (m, 2H), 3.31 (dd, J=13.1, 3.3 Hz, 1H), 2.78-2.71 (m, 2H), 2.68-2.59 (m, 1H), 2.51 (q, J=7.2 Hz, 2H), 1.05 (t, J=7.2 Hz, 3H), 0.97 (t, J=7.8 Hz, 9H), 0.65 (q, J=7.8 Hz, 6H).

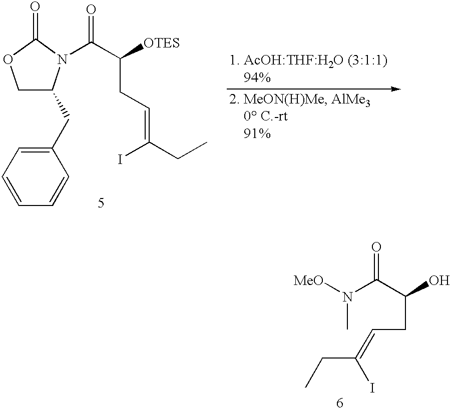

Synthesis of 6. Silyl ether 5 (1.05 g, 1.93 mmol) was dissolved in a 3:1:1 solution of AcOH:THF:water (15 mL) and stirred for 15 h. The reaction mixture was diluted with saturated sodium bicarbonate solution (20 mL) and extracted with ethyl acetate (3×25 mL). The combined organic layers were dried (MgSO₄) and purified by silica gel chromatography (40% EtOAc/hexane) to afford the desilylated product (779 mg, 94% ) as an yellow oil. A suspension of MeON(H)Me.HCl (1.56 g, 16.08 mmol, 5.0 equiv) in THF (30 mL) was cooled to 0° C. and treated with trimethylaluminum (8.0 mL of a 2.0 M solution in toluene, 16.08 mmol, 5.0 equiv). The reaction mixture was warmed to rt after 5 min, stirred for 30 min and cannulated into a solution of the desilylated product (1.38 g, 3.21 mmol) in THF (10 mL) at 0° C. The reaction mixture was warmed to rt after 10 min, stirred for 6 h, and treated with aqueous Rochelle's salt solution (25 mL) after cooling to 0° C. The suspension was stirred at rt for 30 min, and extracted with ethyl acetate (3×50 mL). The combined organic layers were dried (MgSO₄) and purified by silica gel chromatography (10% acetone/toluene) to afford Weinreb's amide 6 (911 mg, 91% ) as an yellow oil: ¹H NMR (400 MHz, CDCl₃) [symbol] 5.69 (t, J=6.6 Hz, 1H), 4.49 (m, 1H), 3.77 (s, 3H), 3.28 (s, 3H), 2.68-2.62 (m, 1H), 2.53 (q, J=7.2 Hz, 2H), 2.44-2.37 (m, 1H), 1.09 (t, J=7.2 Hz, 3H).

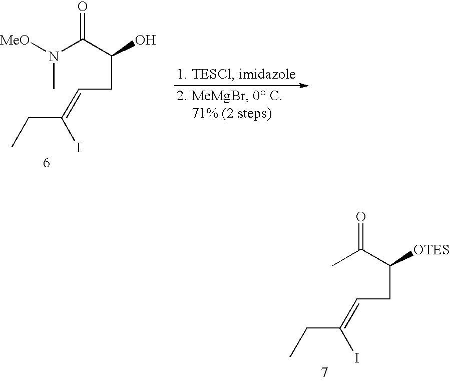

Synthesis of 7. Alcohol 6 (900 mg, 2.87 mmol) in DMF (25 mL) was treated with imidazole (1.17 g, 17.22 mmol, 6.0 equiv) and trimethylsilyl chloride (1.5 mL, 8.62 mmol, 3.0 equiv). The reaction was stirred at rt for 18 h, diluted with ethyl acetate (50 mL), washed with brine (50 mL), water (50 mL), brine (50 mL), dried (MgSO₄) and purified by silica gel chromatography (15% EtOAc/hexane) to afford silylated Weinreb's amide as product. It was dissolved in THF (15 mL) and cooled to 0° C., followed by the addition of MeMgBr (2.0 mL of a 3.0 M solution in diethyl ether, 6.0 mmol, 2.5 equiv). The reaction was maintained at 0° C. for 35 min, followed by quenching with dropwise addition of saturated sodium bicarbonate solution (5 mL), warmed to rt and stirred for 30 min with 10 mL saturated ammonium chloride solution to dissolve the salts. The suspension was extracted with ethyl acetate (3×30 mL), dried (MgSO₄) and purified by silica gel chromatography (4% EtOAc/hexane) to afford 776 mg (71% , 2 steps) of ketone 7 as a colorless oil: ¹H NMR (400 MHz, CDCl₃) [symbol] 5.52 (t, J=6.6 Hz, 1H), 4.10 (t, J=6.3 Hz, 1H), 2.54-2.41 (m, 4H), 2.19 (m, 3H), 1.09 (t, J=7.3 Hz, 3H), 0.96 (t, J=7.9 Hz, 9H), 0.62 (q, J=7.9 Hz, 6H).

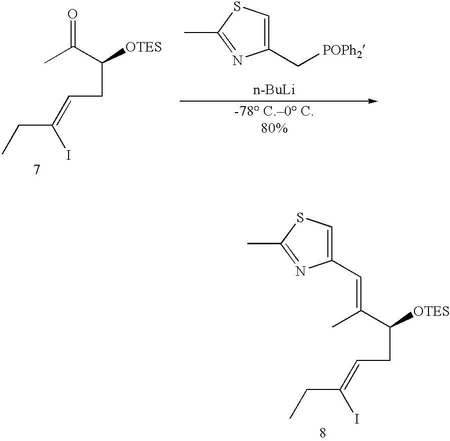

Synthesis of 8. A solution of thiazole phosphine oxide (950 mg, 3.02 mmol, 1.5 equiv) in THF (14 mL) was cooled to −78° C. and treated with n-butyllithium (1.2 mL of a 2.5 M solution in hexane, 3.02 mmol, 1.5 equiv). After stirring for 1 h at −78° C., a solution of ketone 7 (770 mg, 2.01 mmol) in THF (6 mL) at −78° C. was cannulated into the reaction mixture. The reaction was warmed to 0° C. after 1 h, and maintained at that temperature for 4 h. It was quenched with saturated sodium bicarbonate solution (20 mL) and extracted with ethyl acetate (3×30 mL), dried (MgSO₄) and purified by silica gel chromatography (4% EtOAc/hexane) to afford 762 mg (80% ) of 8 as an yellow oil: ¹H NMR (400 MHz, CDCl₃) [symbol] 6.94 (s, 1H), 6.50 (s, 1H), 5.54 (t, J=6.4 Hz, 1H), 4.23 (t, J=6.4 Hz, 1H), 2.72 (s, 3H), 2.49 (q, J=7.3 Hz, 2H), 2.40 (q, J=6.5 Hz, 2H), 2.03 (m, 3H), 1.03 (t, J=7.3 Hz, 3H), 0.95 (t, J=7.9 Hz, 9H), 0.61 (q, J=7.9 Hz, 6H).

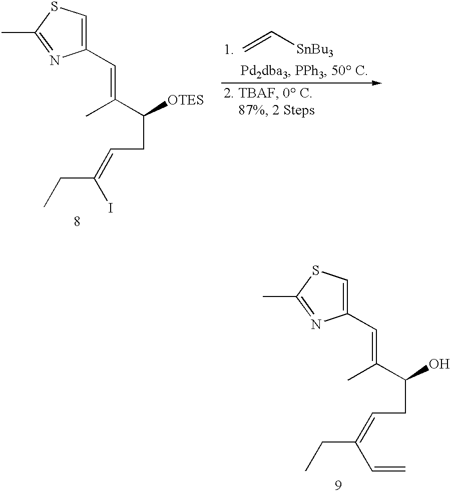

Synthesis of 9. Vinyl iodide 8 (470 mg, 0.98 mmol), tributylvinyltin (0.865 mL, 2.95 mmol, 3.0 equiv) and triphenylphosphine (103 mg, 0.392 mmol, 0.4 equiv) were mixed in degassed DMF (10 mL) and treated with Pd₂dba₃ catalyst (180 mg, 0.196 mmol, 0.2 equiv). The reaction mixture was heated at 50° C. for 3 h, cooled to rt, diluted with ethyl acetate (25 mL) and washed with brine (3×25 mL). The combined aqueous layers were extracted with ethyl acetate (25 mL). The combined organic layers were dried (MgSO₄) and purified by silica gel chromatography (4% EtOAc/hexane) to afford the diene product as an yellow oil, which was dissolved in THF (10 mL) and treated with TBAF (1.4 mL of a 1.0 M solution in THF, 1.35 mmol, 1.5 equiv) after cooling to 0° C. After 30 min, the reaction mixture was diluted with diethyl ether (25 mL) and washed with saturated sodium bicarbonate solution (25 mL). The aqueous layer was extracted with diethyl ether (2×25 mL). The combined organic layers were dried (MgSO₄) and purified by silica gel chromatography (4-12-28% EtOAc/hexane+1% Et₃N) to afford diene 9 (223 mg, 87% , 2 steps) as an yellow oil: ¹H NMR (400 MHz, CDCl₃) [symbol] 6.95 (s, 1H), 6.70 (dd, J=17.5, 11.0 Hz, 1H), 6.57 (s, 1H), 5.43 (t, J=7.8 Hz, 1H), 5.29 (d, J=17.5 Hz, 1H), 5.14 (d, J=11.0 Hz, 1H), 4.22 (t, J=6.6 Hz, 1H), 2.72 (s, 3H), 2.59-2.54 (m, 2H), 2.23 (q, J=7.3 Hz, 2H), 2.07 (s, 3H), 1.06 (t, J=7.3 Hz, 3H).

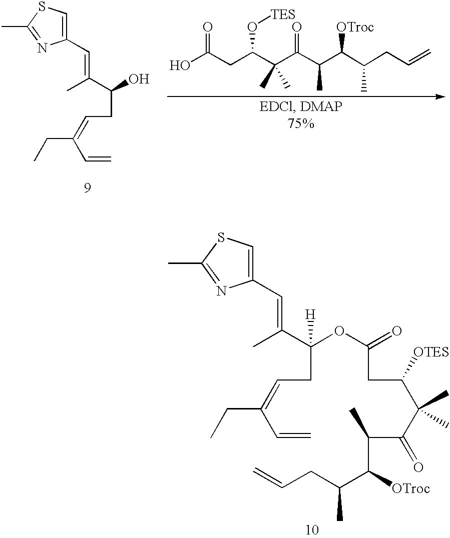

Synthesis of 10. To a stirred solution of alcohol 9 (221 mg, 0.84 mmol, 1 equiv) in methylene chloride (4 mL) at 0° C. were added EDCI (256 mg, 1.34 mmol, 1.6 equiv) and DMAP (163 mg, 1.34 mmol, 1.6 equiv). A solution of acid (482 mg, 0.84 mmol, 1 equiv) in methylene chloride (4 mL) was added to the reaction mixture in a dropwise fashion, which was warmed to rt. The reaction was concentrated after 2 h, and purified using silica gel chromatography (8% EtOAc/hexane) to afford ester 10 (487 mg, 71% yield) as a clear oil: ¹H NMR (400 MHz, CDCl₃) δ6.94 (s, 1H), 6.74 (dd, J=17.4, 11.1 Hz, 1H), 6.50 (s, 1H), 5.75-5.65 (m, 1H), 5.32-5.24 (m, 3H), 5.12 (d, J=11.1 Hz, 1H), 5.06-4.97 (m, 2H), 4.82 (d, J=12.0 Hz, 1H), 4.72 (dd, J=7.2, 2.9 Hz, 1H), 4.66 (d, J=12.0 Hz, 1H), 4.21 (dd, J=6.8, 3.2 Hz, 1H), 3.50-3.46 (m, 1H), 2.70 (s, 3H), 2.69-2.47 (m, 3H), 2.30-2.16 (m, 4H), 2.08 (s, 3H), 1.95-1.80 (m, 2H), 1.36 (s, 3H), 1.06 (d, J=6.7 Hz, 3H), 1.02 (t, J=7.3 Hz, 3H), 1.01 (s, 3H), 0.97-0.93 (m, 12H), 0.63 (q, J=7.9 Hz, 6H).

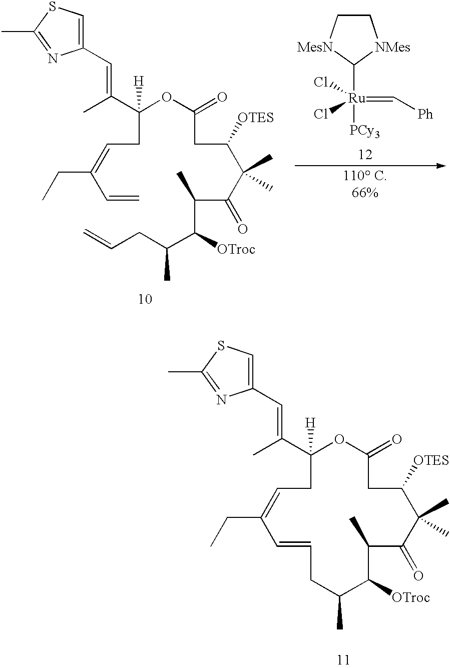

Synthesis of 11. A solution of compound 10 (130 mg, 0.158 mmol) in toluene (350 mL) was heated to reflux and treated with Grubbs catalyst 12 (27 mg, 0.031 mmol, 0.2 equiv). The reaction was heated at 110° C. for 25 min, cooled to rt by immersing in an ice-water bath, evaporated under reduced pressure and purified by silica gel chromatography (8% EtOAc/hexane) to afford metathesis product 11 (82 mg, 66% ): ¹H NMR (400 MHz, CDCl₃) [symbol] 6.97 (s, 1H), 6.63 (d, J=15.9 Hz, 1H), 6.57 (s, 1H), 5.72-5.64 (m, 1H), 5.40 (t, J=8.4 Hz, 1H), 5.20 (d, J=8.7 Hz, 1H), 5.13 (d, J=10.1 Hz, 1H), 4.81 (q, J=10.0 Hz, 2H), 4.07 (d, J=8.5 Hz, 1H), 3.33-3.26 (m, 1H), 2.88-2.77 (m, 2H), 2.72 (s, 3H), 2.54 (dd, J=16.5, 9.3 Hz, 1H), 2.42-2.36 (m, 1H), 2.23-2.09 (m, 5H), 2.05 (s, 3H), 2.05-2.00 (m, 2H), 1.89-1.85 (m, 1H), 1.19 (s, 3H), 1.12-1.08 (m, 9H), 1.01 (t, J=7.3 Hz, 3H), 0.87 (t, J=8.0 Hz, 9H), 0.54 (q, J=8.0 Hz, 6H); LRMS (ESI) calc. For C₃₇H₅₆Cl₃NO₇SSi 791.2, found 792.2 (M+H)⁺, 814.2 (M+Na)⁺.

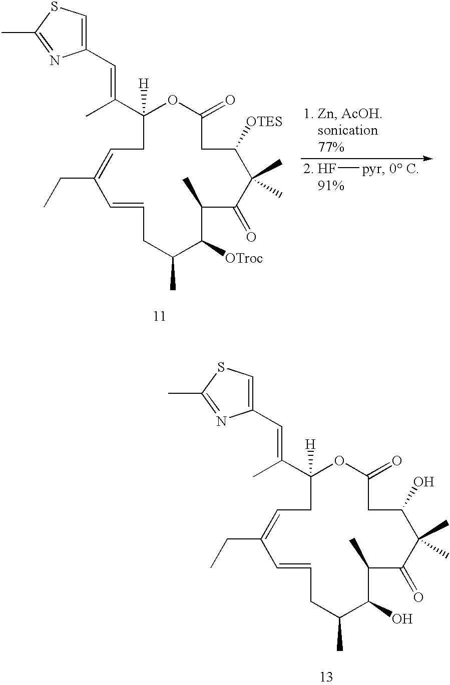

Synthesis of 13. Epothilone derivative 11 (51 mg, 0.064 mmol) was dissolved in a 1:1 solution of THF:AcOH (3 mL) and treated with Zn (nanosize activated, 10 mg). The reaction mixture was sonicated at rt for 15 min., More Zn was added (5 mg) followed by further sonication for 15 min. The suspension was filtered through a plug of celite, which was washed with ethyl acetate (50 mL), the filtrate concentrated to a volume of 10 mL, washed with saturated sodium bicarbonate solution (2×10 mL), brine (10 mL), dried (MgSO₄) and purified by silica gel chromatography (16% EtOAc/hexane) to afford the C-3 TES ether—C-7 alcohol (30 mg, 77% ) as a white solid. The C-3 TES ether-C-7 alcohol (70 mg, 0.11 mmol) was dissolved in THF (2 mL) in a plastic vial and cooled to 0° C. HFpyridine solution (0.5 mL) was added, and the reaction was stirred at 0° C. for 150 min. The reaction was quenched with saturated sodium bicarbonate solution (10 mL) and extracted with ethyl acetate (15 mL). The organic layer was washed with saturated sodium bicarbonate solution (10 mL), brine (10 mL), dried (MgSO₄) and purified by silica gel chromatography (20-32% EtOAc/hexane) to afford epothilone 13 (50 mg, 91% ) as a white solid: ¹H NMR (400 MHz, CDCl₃) [symbol] 6.97 (s, 1H), 6.58 (s, 1H), 6.37 (d, J=16.1 Hz, 1H), 5.83-5.75 (m, 1H), 5.31-5.24 (m, 2H), 4.25 (d, J=7.6 Hz, 1H), 3.75 (t, J=6.6 Hz, 1H), 3.69 (d, J=6.9 Hz, 1H), 3.29-3.25 (m, 1H), 3.22 (s, 1H), 2.85-2.81 (m, 1H), 2.72 (s, 3H), 2.60-2.56 (m, 1H), 2.45-2.39 (m, 1H), 2.35-2.31 (m, 2H), 2.18-2.14 (m, 2H), 2.01 (s, 3H), 2.01-1.99 (m, 2H), 1.87-1.85 (m, 1H), 1.28 (s, 3H), 1.12 (d, J=6.8 Hz, 3H), 1.07 (d, J=6.9 Hz, 3H), 1.04 (s, 3H), 0.99 (t, J=7.3 Hz, 3H); LRMS (ESI) calc. For C₂₈H₄₁NO₅S 503.2 found 504.1 (M+H)⁺, 526.0 (M+Na)⁺.

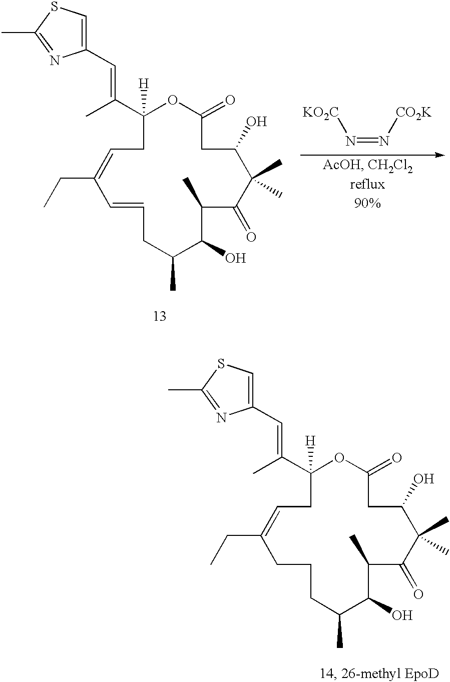

Synthesis of 14, 26-methyl Epo D. The diene 13 (30 mg, 0.05 mmol) was dissolved in methylene chloride (2 mL) and treated with AcOH (0.6 mL) and heated to reflux. The diazocaboxylate (14 mg, 0.5 mmol, 10.0 equiv) was added. After 2 h, more AcOH (2 mL) was added using a syringe pump, over a period of 4 h. The reaction was followed by HPLC. After refluxing for a total of 12 h, more diazo compound (7 mg, 0.25 mmol, 5.0 equiv) and AcOH (2 mL over 4 h) were added. After a total of 24 h, 1 mL AcOH was further added. The reaction was cooled to rt after 4 h, filtered, diluted with ethyl acetate (15 mL), washed with brine (10 mL), dried (MgSO₄) and purified by silica gel chromatography (10-15-35% EtOAc/hexane) to afford 26-methyl epothilone D 14 (27 mg, 90% ) as a white, which was identical to the previously described compound (Harris, Danishefsky J. Org. Chem. 1999, 64, 8434 and references therein).

### Example 8

Synthesis of C-11 Epothilone Analogues

The epothilone, 11-hydroxy-dEpoB, was synthesized from a macro-Nozaki precursor. The synthesis of the macro-Nozaki precursor was synthesized using the scheme below:

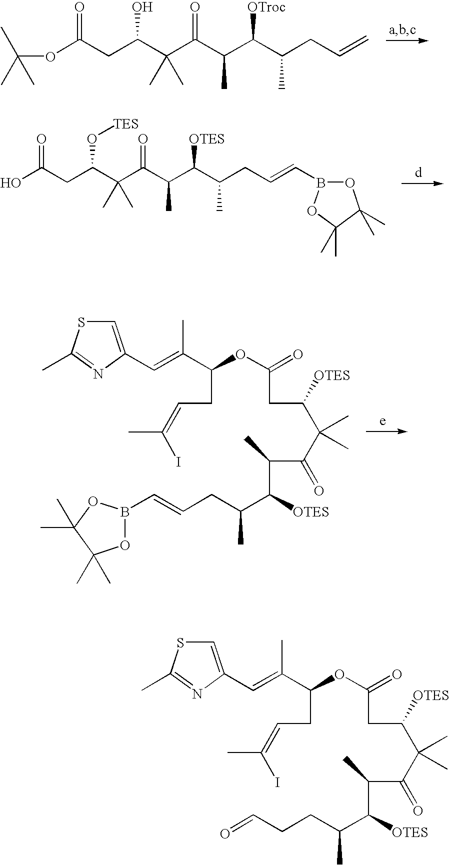

Conditions: a) nanosize zinc, AcOH/THF, sonication, rt (96% yield), b) TESOTf, 2,6-Lutidine, CH₂Cl₂, rt (65% yield), c) vinyl-pinacolboronate, Bis(tricyclohexylphosphine) benzylidine ruthenium (IV) dichloride (Grubbs catalyst), CH₂Cl₂, reflux (95% yield), d) EDCI, DMAP, Left-Epo Alochol, CH₂Cl₂, rt (67% yield), e) Me₃NO, THF, Reflux (90% yield)

As shown in the scheme below, the macrocycle was closed using a stereoselective macro-Nozaki reaction to yield 11-hydroxy-dEpoB. The other stereoisomer was obtained by oxidizing the C-11 hydroxyl group to the corresponding ketone using Dess-Martin reagent and reducing the resulting enone stereoselectively.

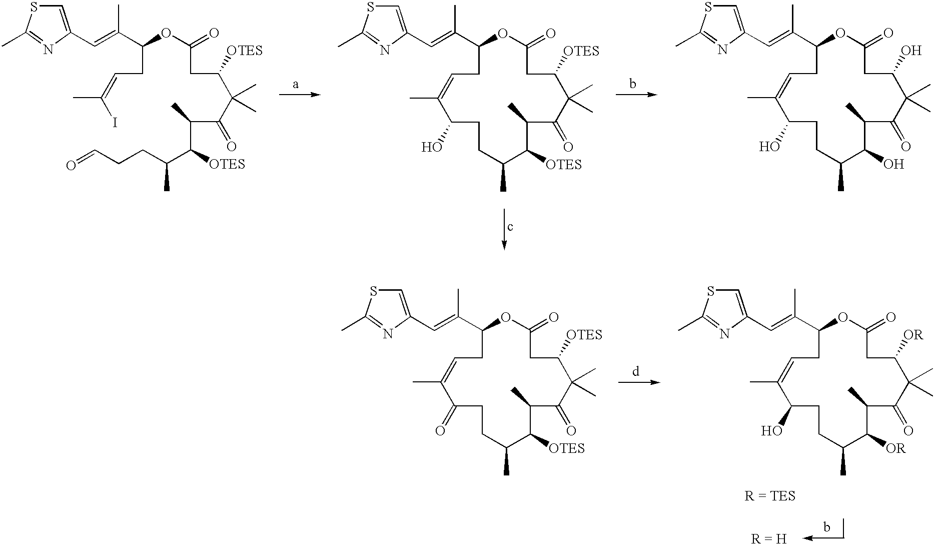

Conditions: a) CrCl₂, NiCl₂, 3:1 (DMF/THF), rt (40% yield), b) HF-pyridine, THF, rt (90% yield), c) Dess-Martin Periodinane, CH₂Cl₂, rt (95% yield), d) NaBH₄, CeCl₃, MeOH, −78° C. (70% yield)

The 11-hydroxy analogs were further modified to yield fluorinated epothilones. 11-fluoro, 13-fluoro, and 11,11-difluoro were obtained using the scheme below:

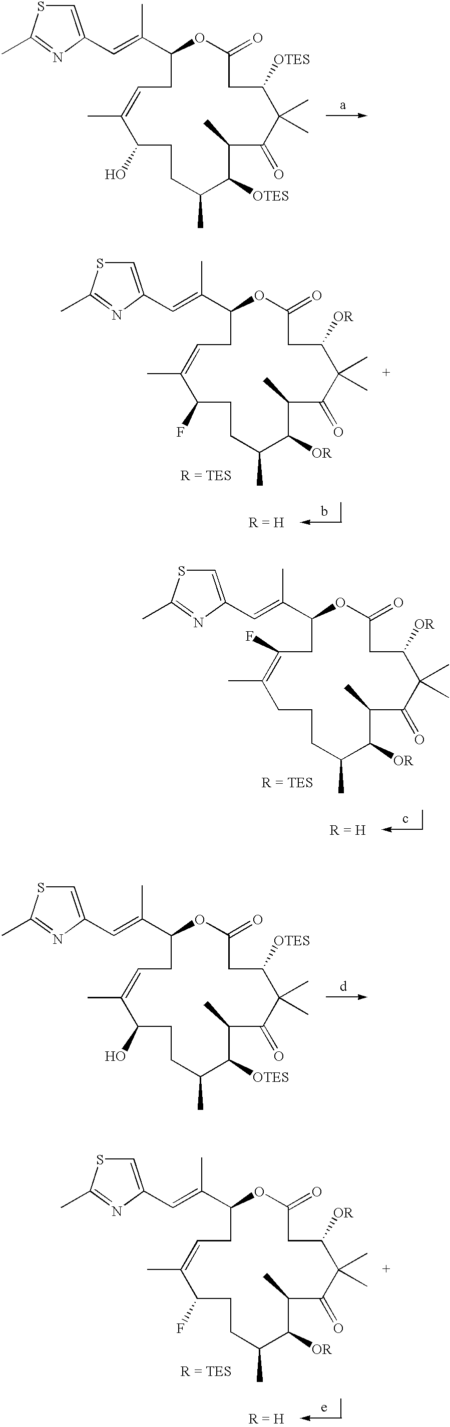

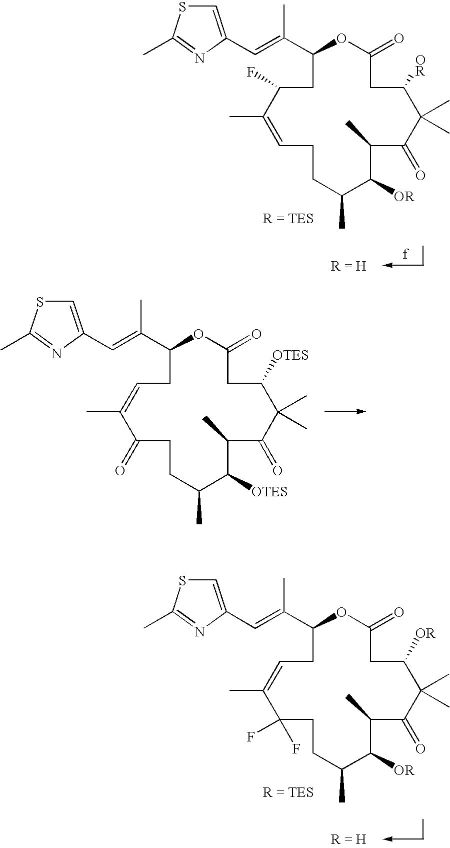

Conditions: a) DAST, CH₂Cl₂, −78° C., 3:1 S_{N}2/S_{N}2′ (11-Fluoro/13-Fluoro) (70% yield), b) HF-pyridine, THF, rt (9,0% yield), c) HF-pyridine, THF, rt, d) DAST, CH₂Cl₂, −78° C., 43:1 S_{N}2/S_{N}2′ (11-Fluoro/13-Fluoro) (65% yield), e) HF-pyridine, THF, rt (90% yield), f) HF-pyridine, THF, rt.

The 11-amino dEpoB can be obtained via reductive amination of the enone:

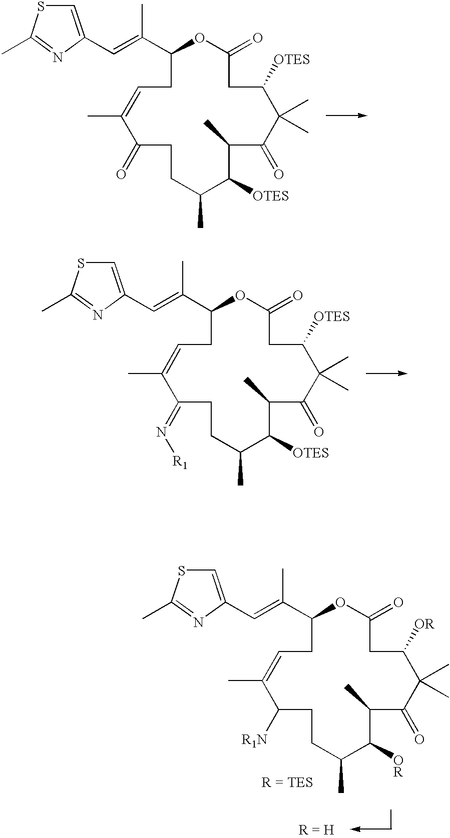

The 11-hydroxylalkyl and 11-alkyl Epo 490 can be obtained via addition to the enone.

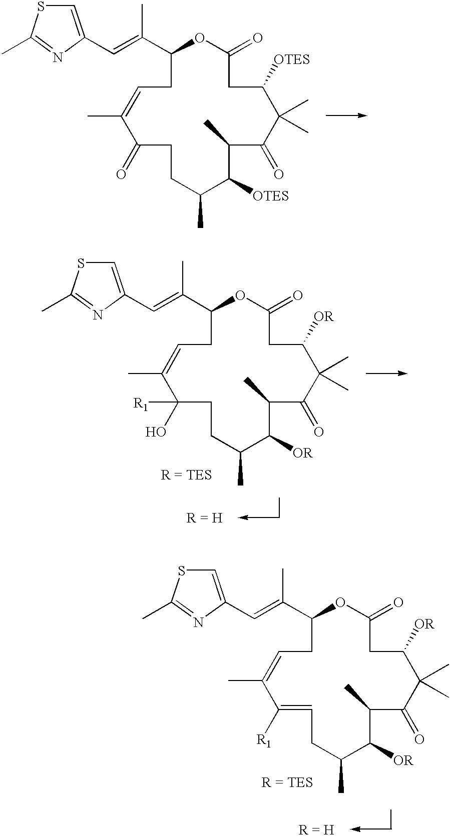

The cyclopropyl and epoxide analogs can be obtained by cyclopropanation or epoxidation of the allylic alcohol.

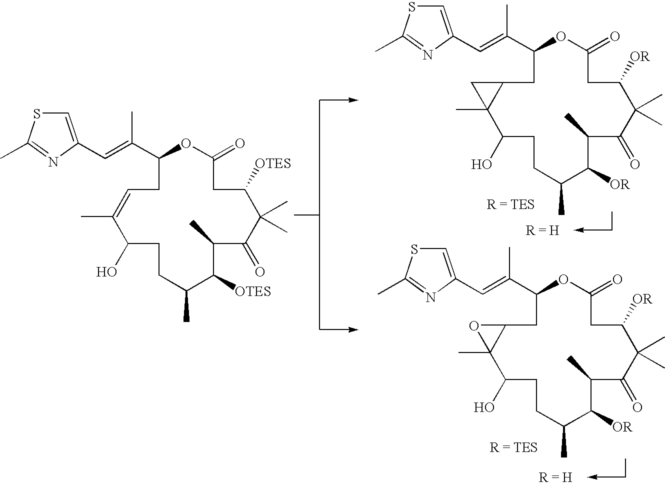

### Example 9

Synthesis of Oxazole-Containing Epothilone Analogues

The synthesis of 19-oxa epothilone 490 was accomplished following a synthetic route analogous to the one developed for the preparation of epothilone 490 as described herein. The scheme below details the reaction steps leading to 19-oxa epothilone 490 starting from methyl ketone 1 and carboxylic acid 2, which have been reported in the literature. 19-oxa epothilone D and 19-oxa epothilone B can then be prepared from 19-oxa epothilone 490 using known synthetic methods as shown.

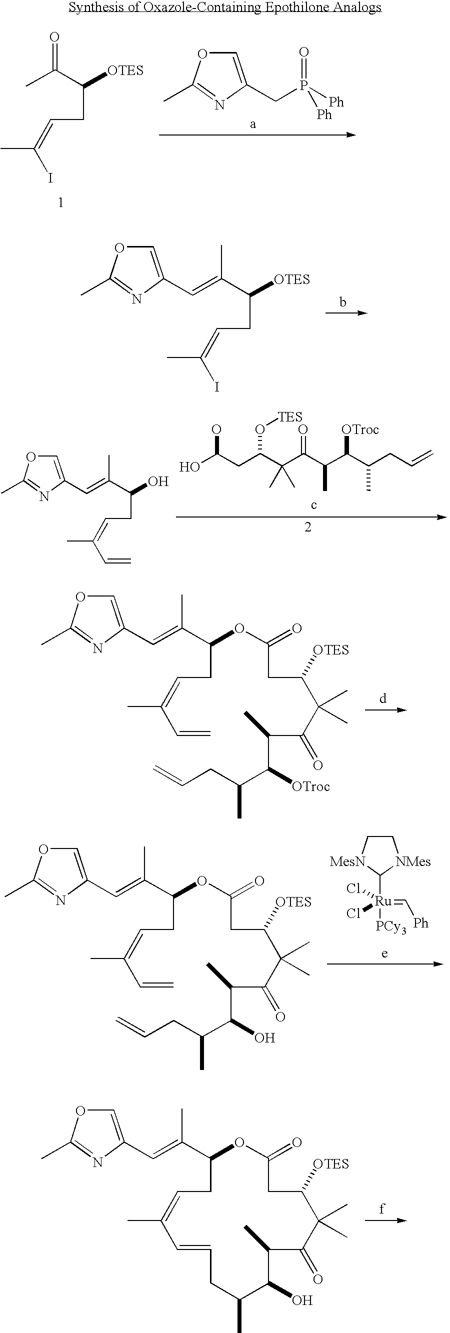

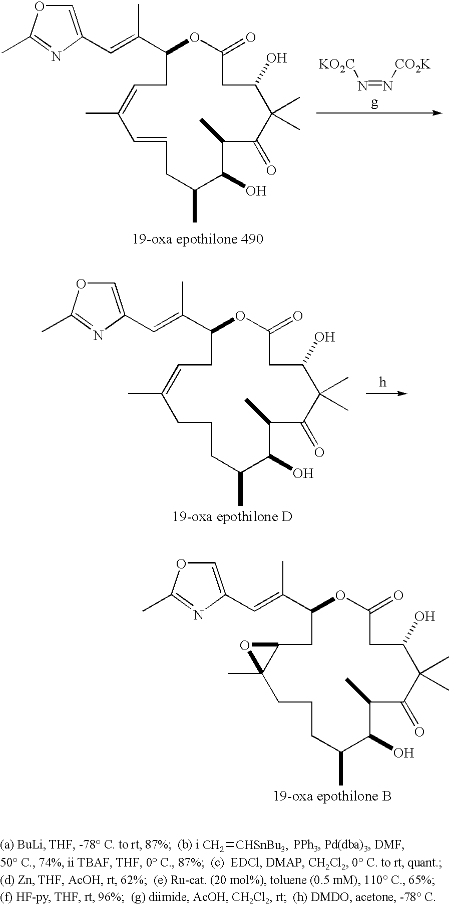

The in vitro cytoxicity of 19-oxaepothilone 490 was determined using several CCRF-CEM cell lines. The IC₅₀s for 19-oxaepothilone 490 and epothilone 490 are shown in the table below:

Cell Growth Inhibition (IC₅₀ in μM)

| Compound | CCRF-CEM | CCRF-CEM/VBL | CCRF-CEM/taxol |
|---|---|---|---|
| 19-oxa | 0.015 | 0.060 | 0.04 |
| epothilone 490 | | | |
| epothilone 490 | 0.009 | 0.023 | 0.013 |

### Example 10

Synthesis of C-15 Aza Analogue of Epo490

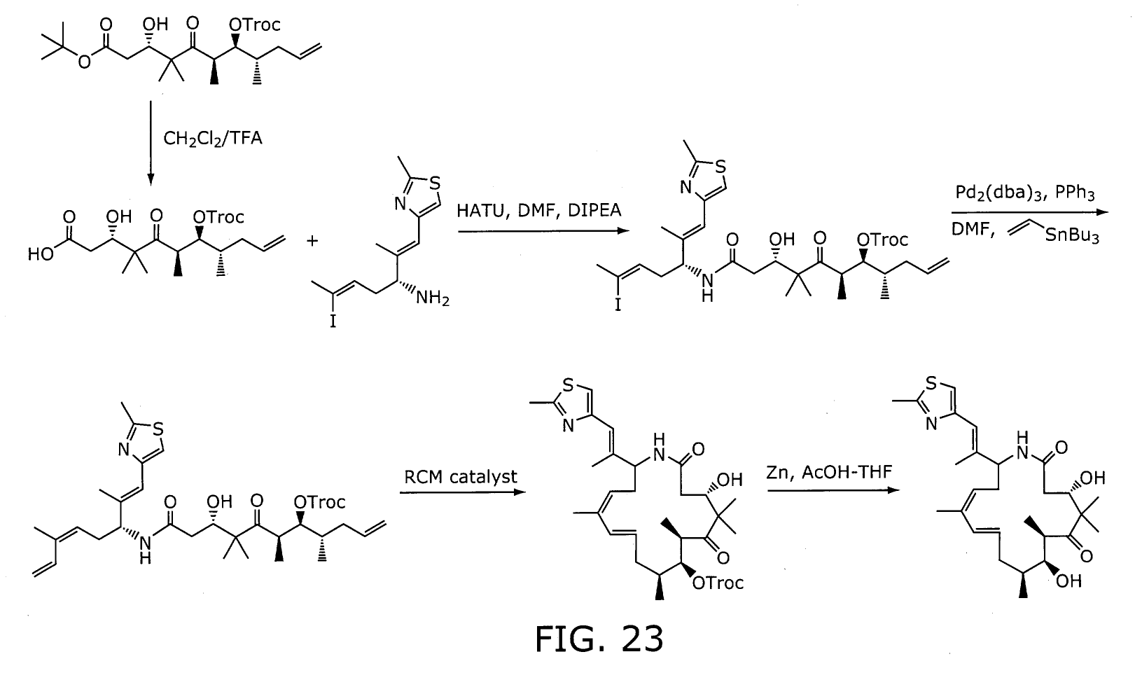

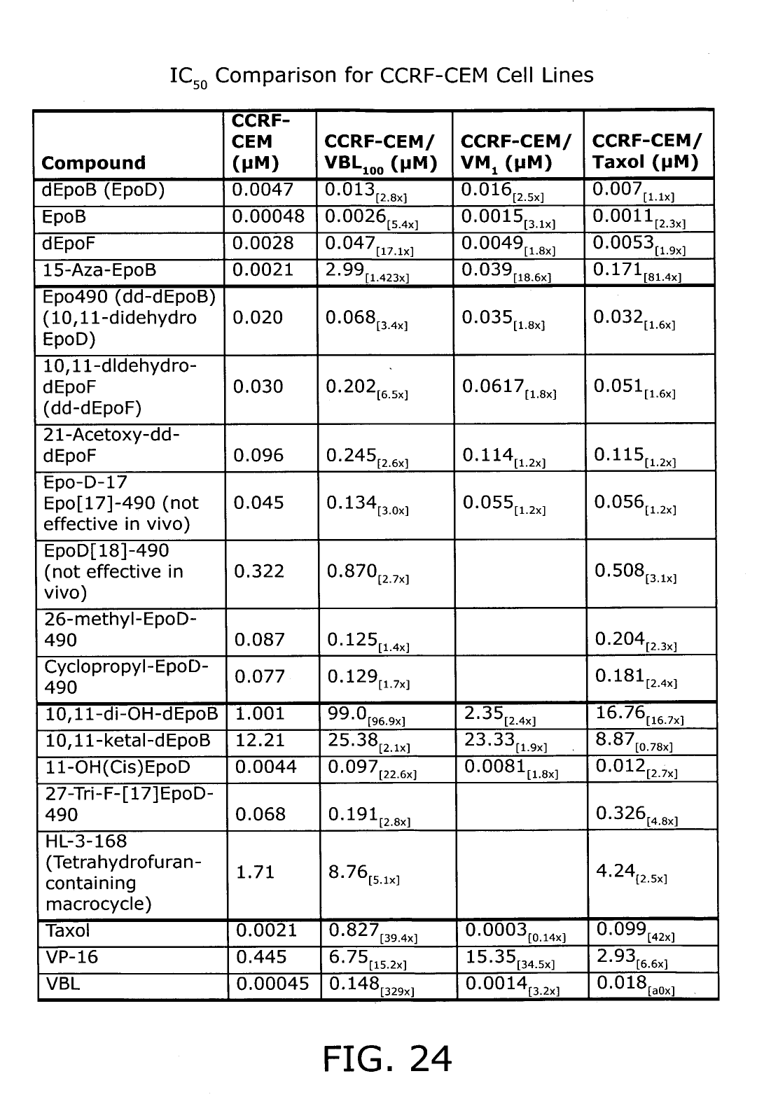

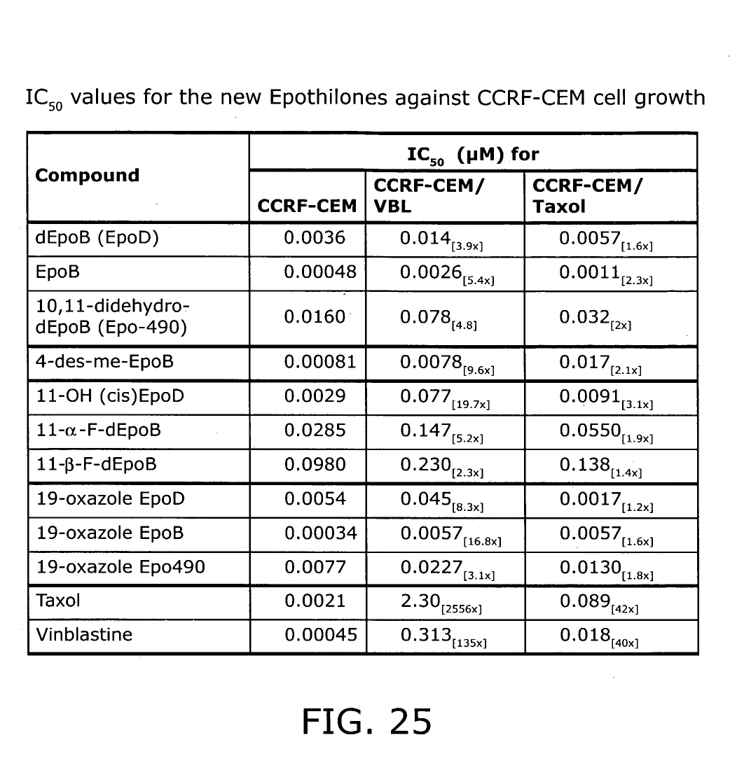

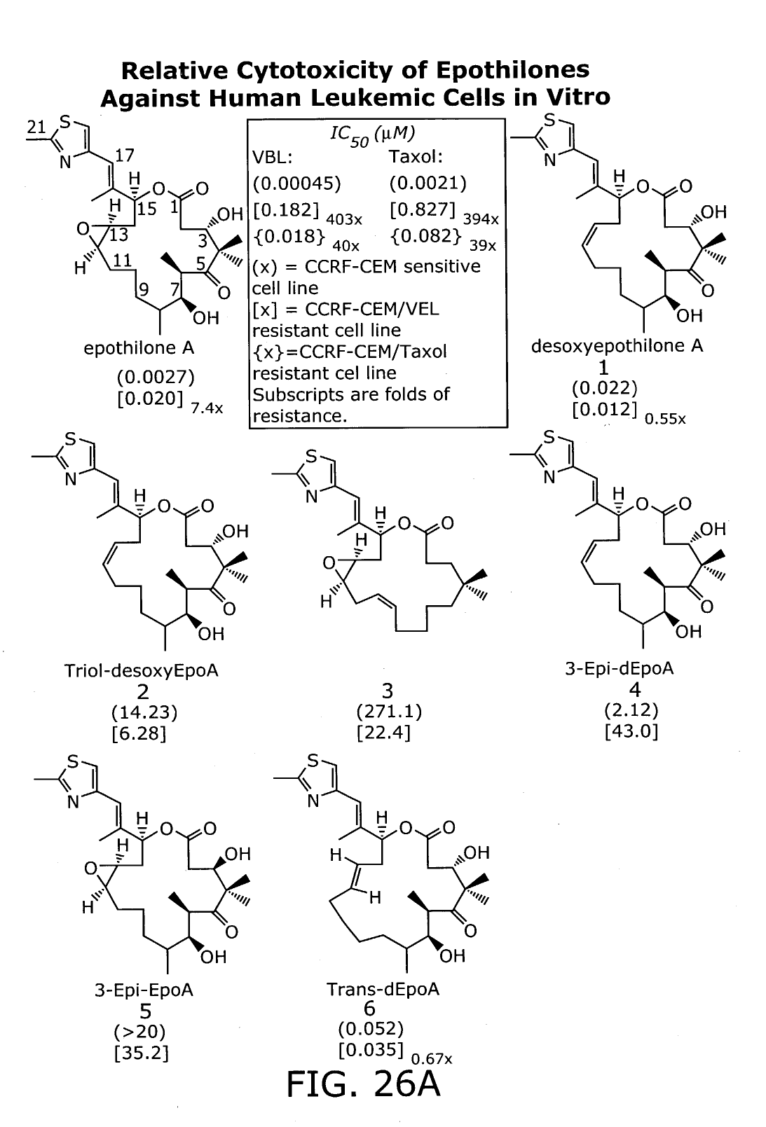

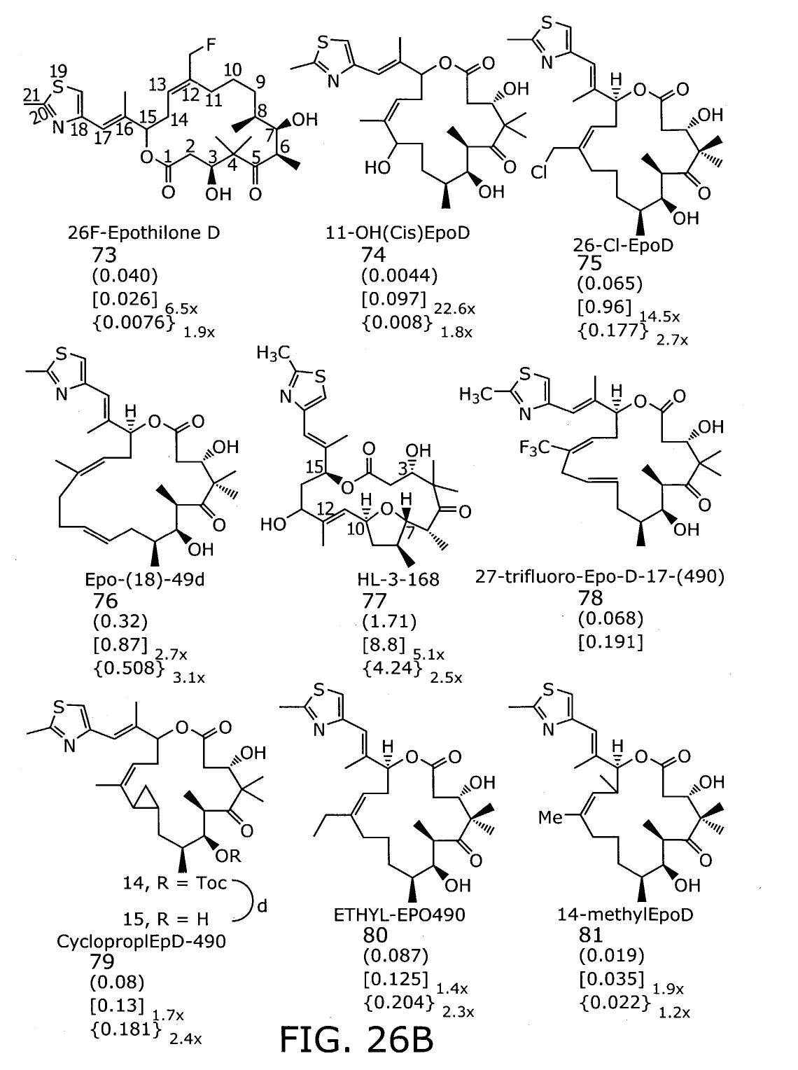

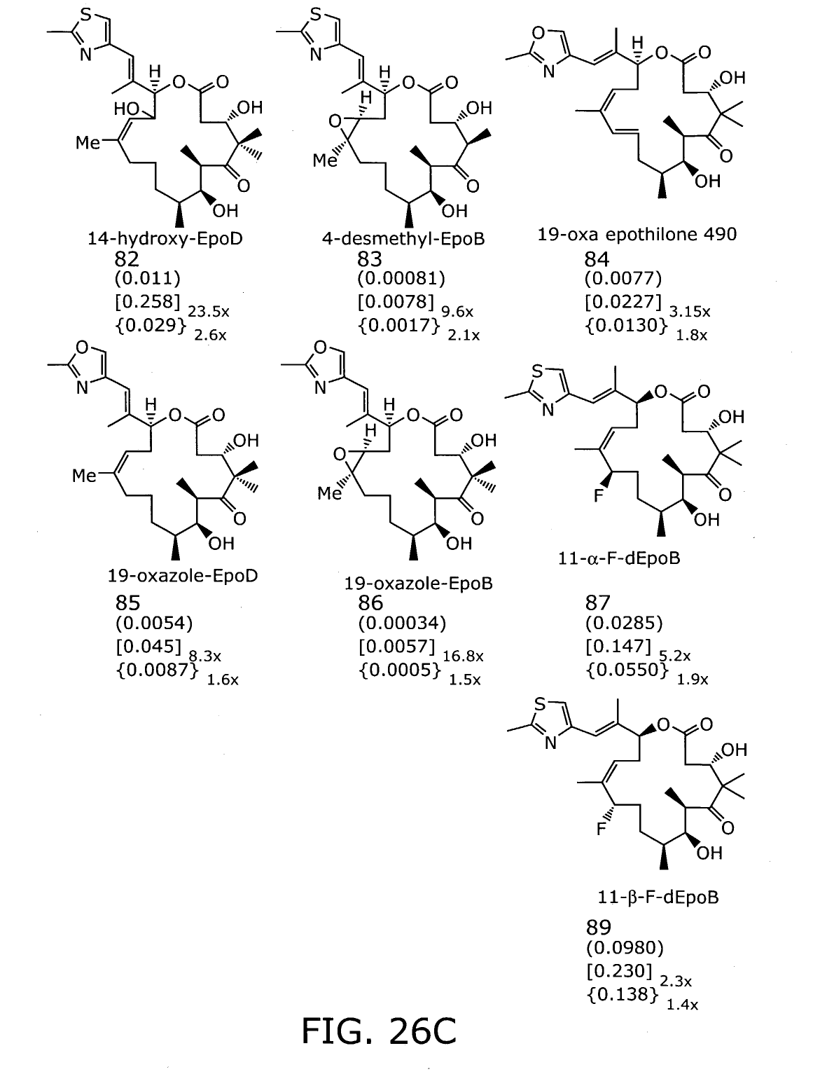

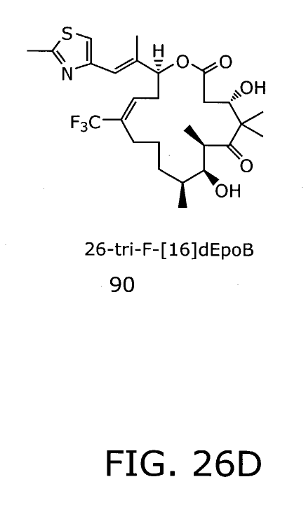

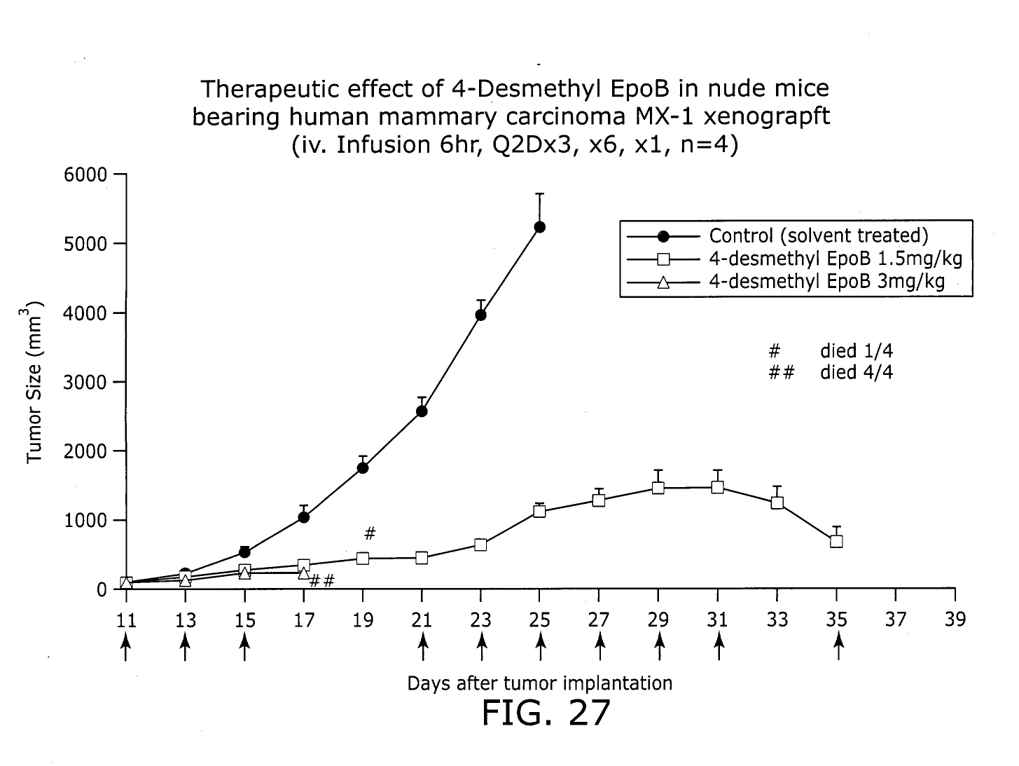

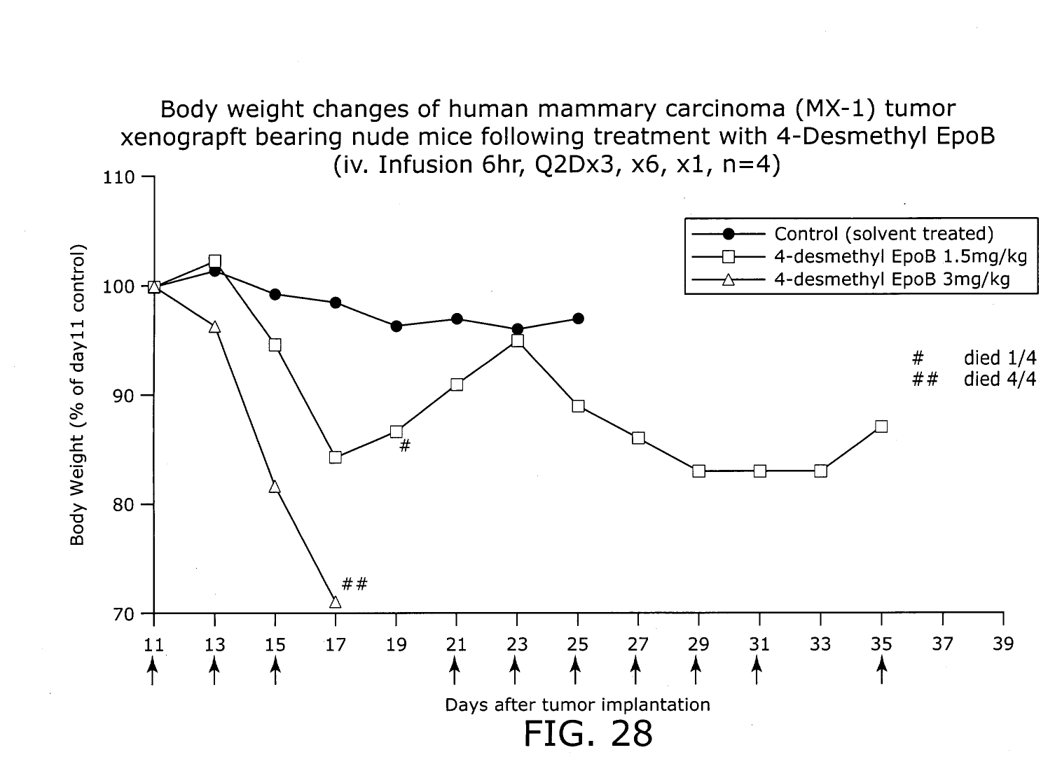

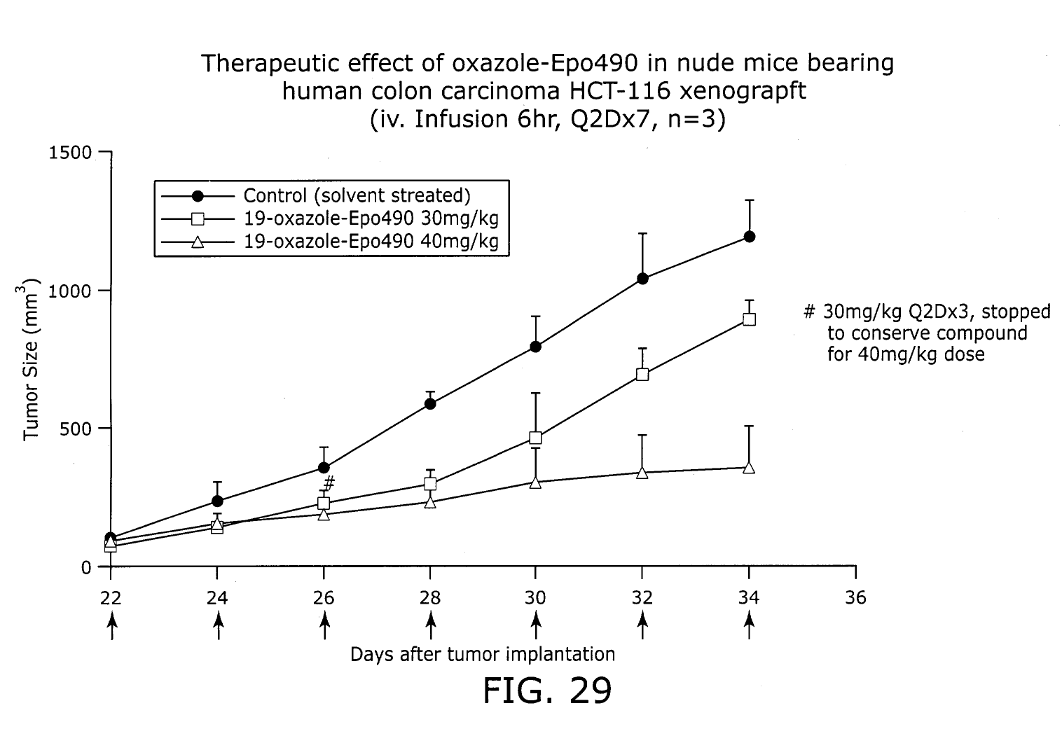

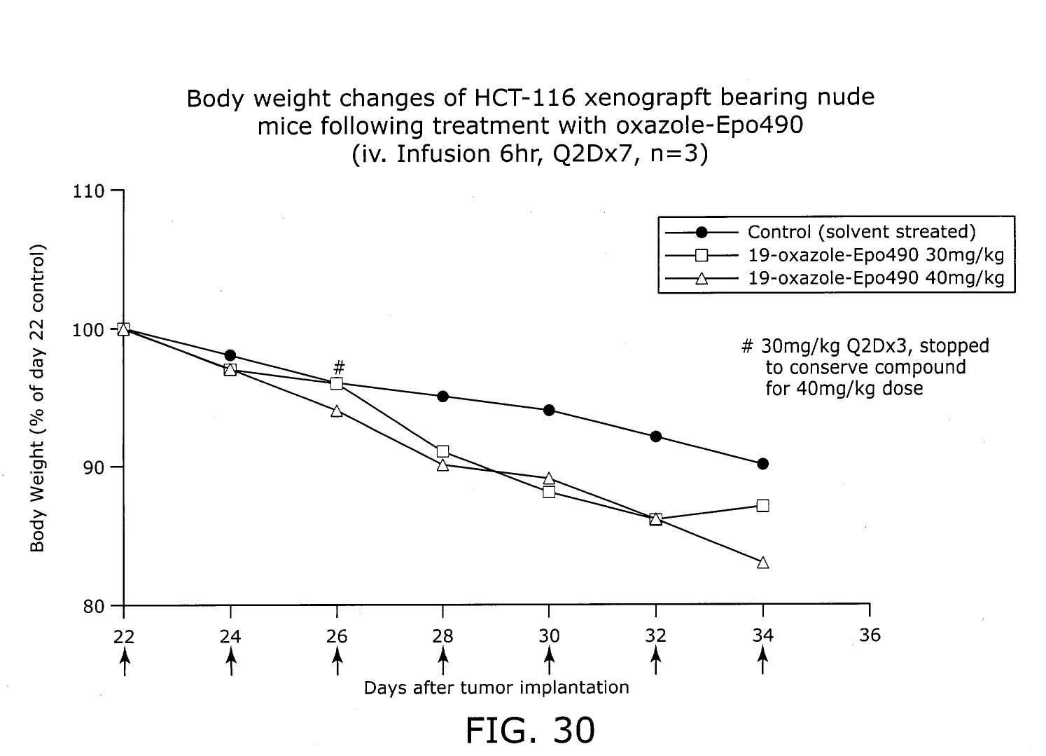

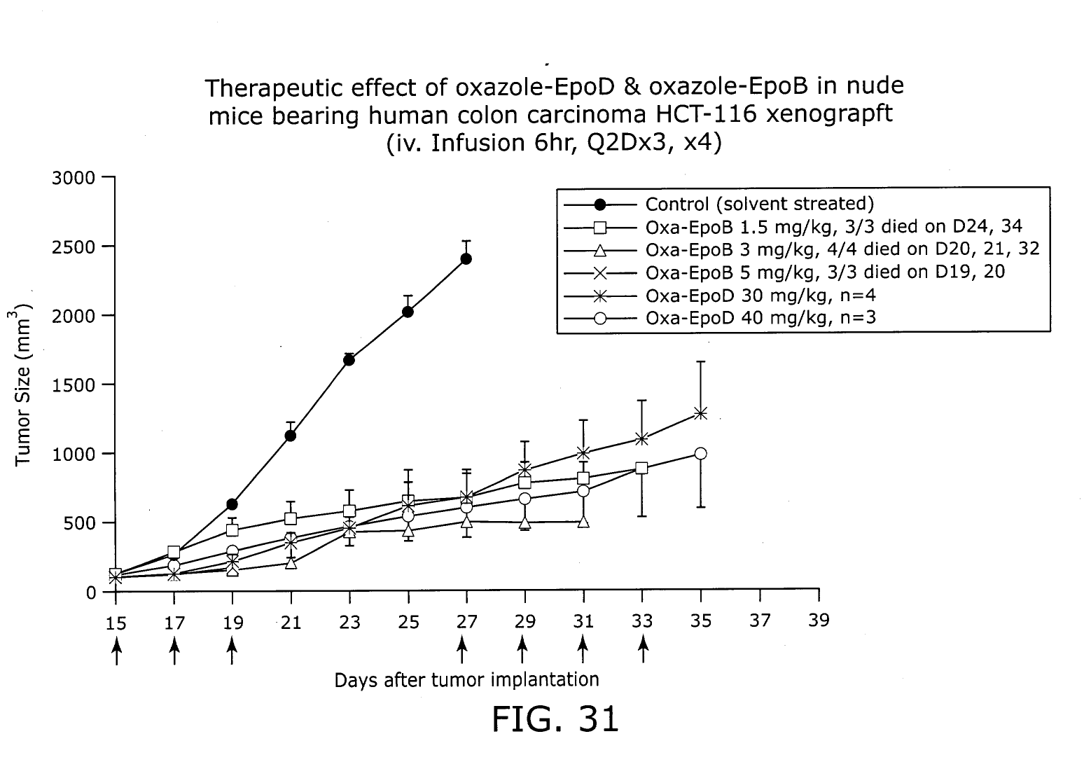

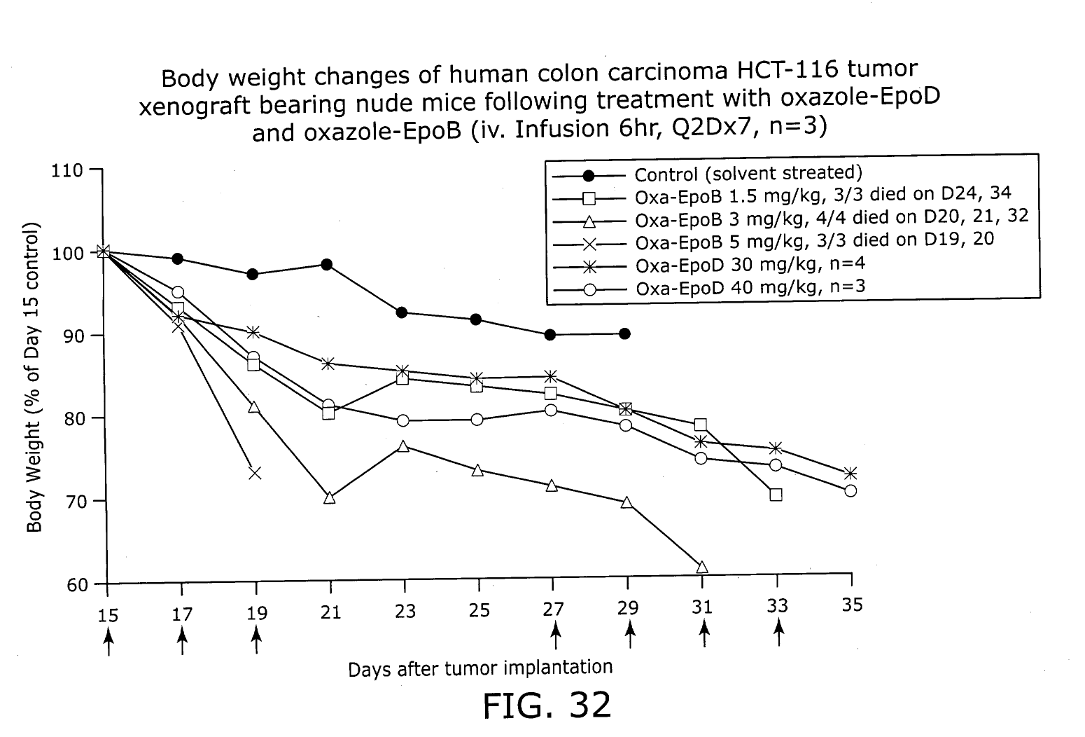

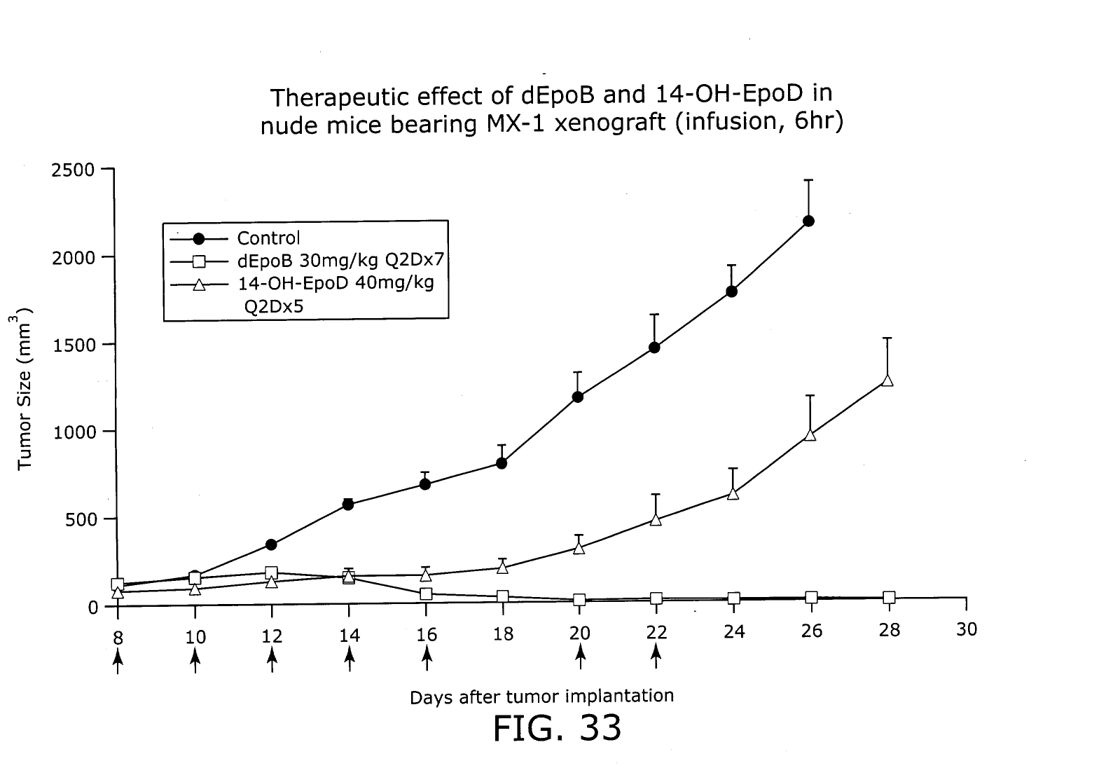

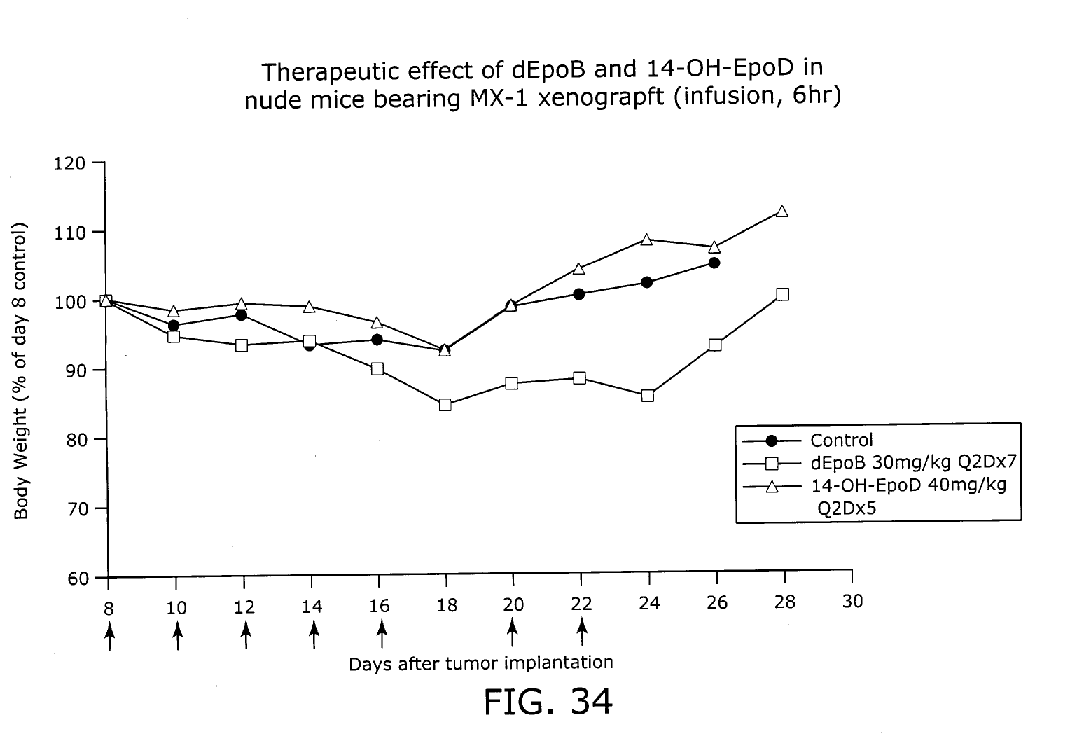

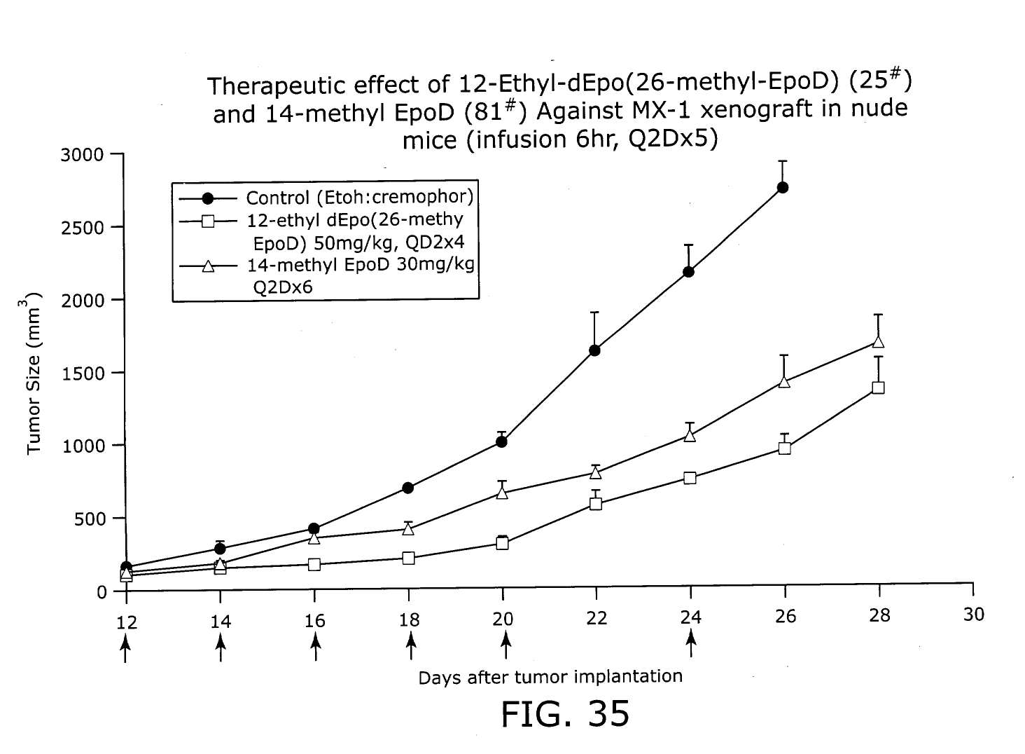

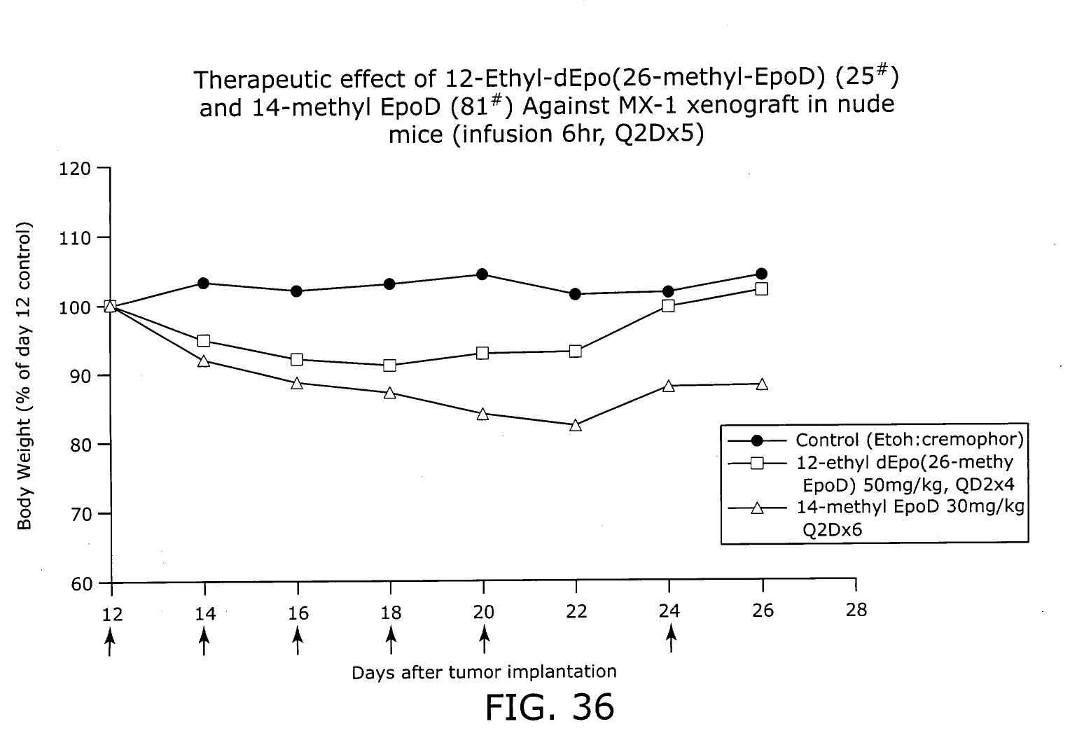

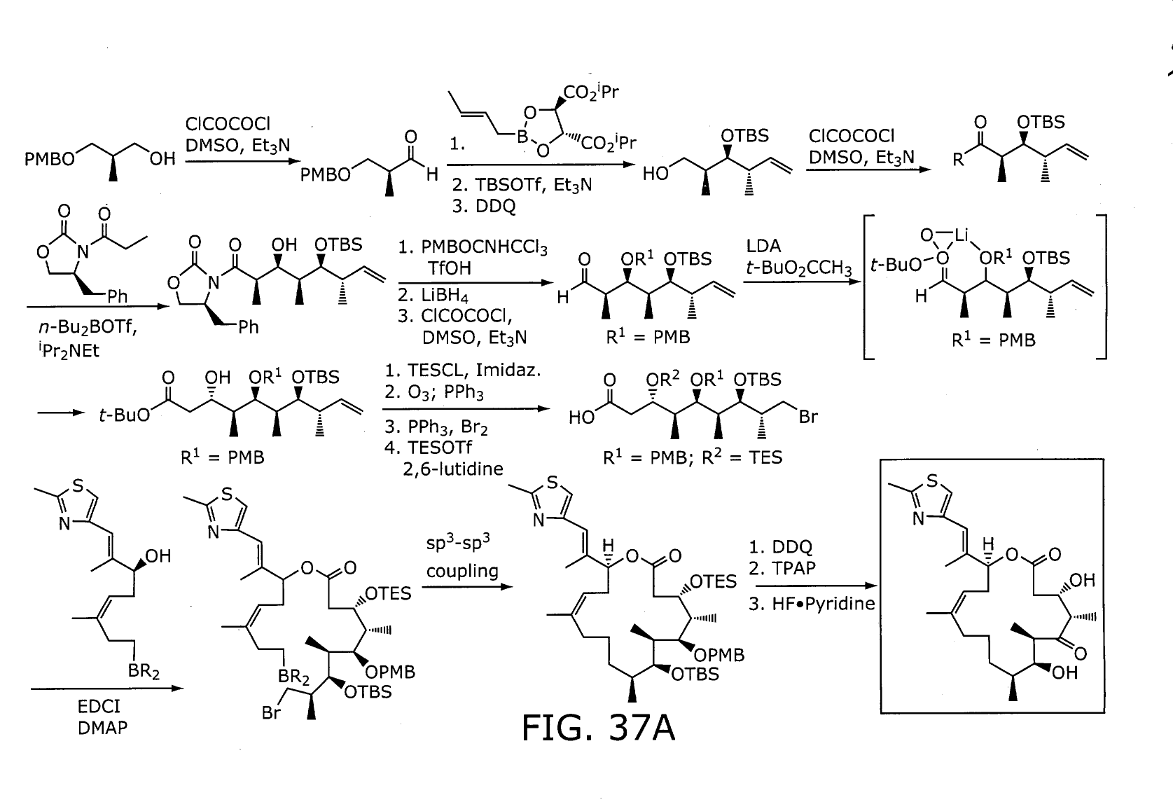

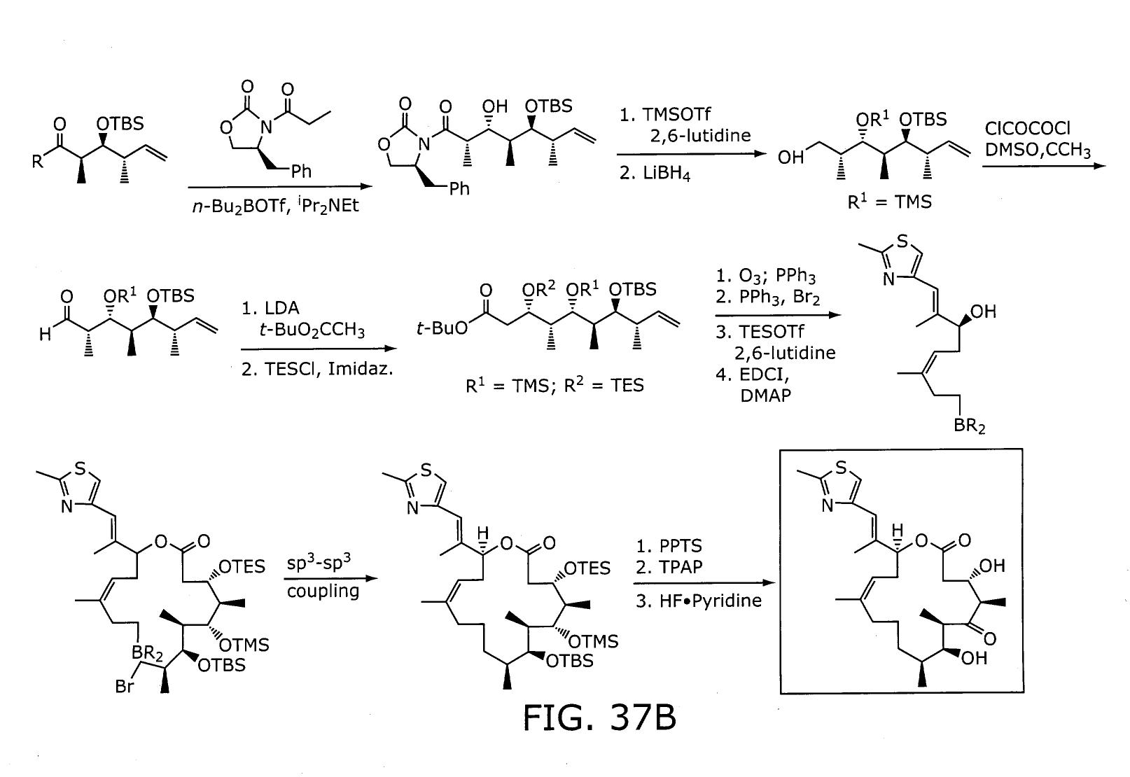

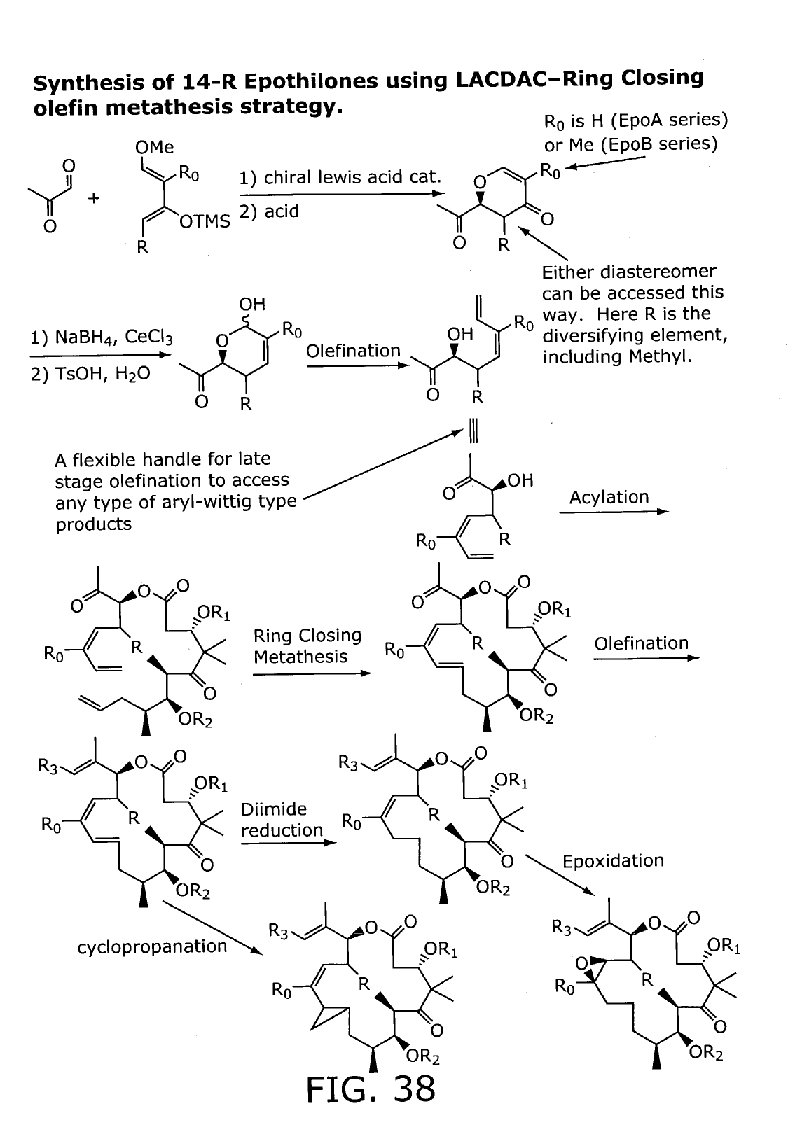

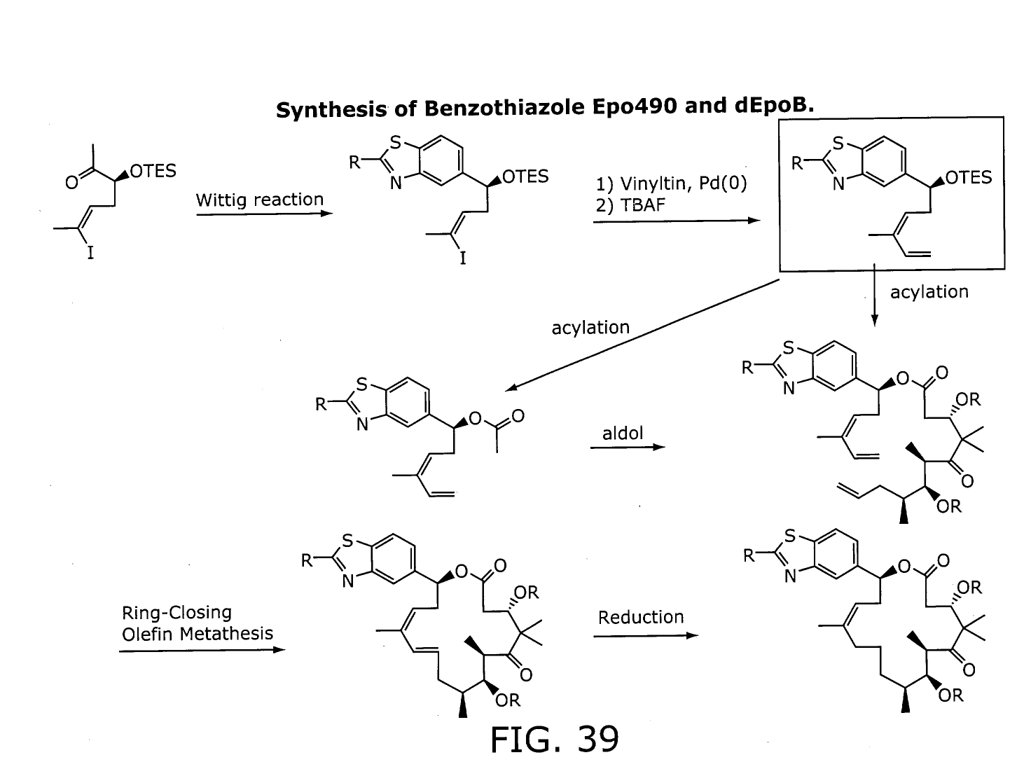

The lactam version of Epo490 was prepared via the ring closing metathesis route shown in FIG. 23.

### Example 11

Synthesis of Epo490

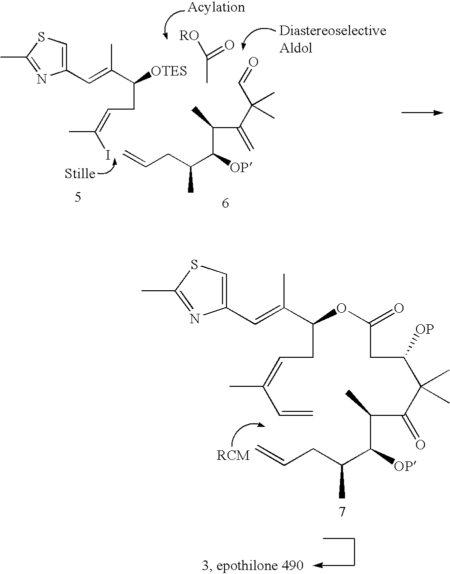

The synthetic plan for Epo 490 envisaged a construction of a “seco” acyclic triene 7 positioned for diene-ene RCM for macrolide formation. We drew upon previously disclosed and highly accessible building blocks to pursue a new synthesis of the epothilone synthesis problem. These are vinyl iodide 5, (Chappell, M. D.; Stachel, S. J.; Lee, C. B.; Danishefsky, S. J. Org. Lett. 2000, 2, 1633; incorporated herein by reference) and aldehyde 6. (Lee, C. B.; Wu, Z.; Zhang, F.; Chappell, M. D.; Stachel, S. J.; Chou, T. C.; Guan, Y.; Danishefksy, S. J. J. Am. Chem. Soc. 2001, 123, 5249; Wu, Z.; Zhang, F.; Danishefsky, S. J. Angew. Chem., Int. Ed. 2000, 39, 4505; each of which is incorporated herein by reference).

The “seco” compound 7 could be accessed from a reassembly of advanced synthetic intermediates. The C11-C15 domain can be acylated with an appropriate C1 acid moiety to construct the C1-C 15 ester linkage. The stereoselective formation of the C3 alcohol (in its native S-configuration) developed into a major challenge in our earlier efforts, especially in the epothilone F series (Lee, C. B.; Chou, T.-C.; Zhang, X. G.; Wang, Z. G.; Kuduk, S. D.; Chappell, M. D.; Stachel, S. J.; Danishefsky, S. J. J. Org. Chem 2000, 65, 6525; incorporated herein by reference). Extensive investigations revealed that the best yields were obtained from a chiral titanium-mediated tert-butyl acetate aldol reaction with aldehyde 6, affording the correct C3 alcohol, after construction of the C6, C7, and C8 stereocenters (Wu, Z.; Zhang, F.; Danishefsky, S. J. Angew. Chem., Int. Ed. 2000, 39, 4505; incorporated herein by reference) For the synthesis of our cyclization precursor, acylation with acetic anhydride to generate the C15 acetate (vide infra), followed by a diastereoselective aldol reaction with aldehyde 6 would generate the target compound, with concomitant formation of the C3 stereocenter. With these design elements in mind, we embarked first upon the total synthesis of epothilone 490.

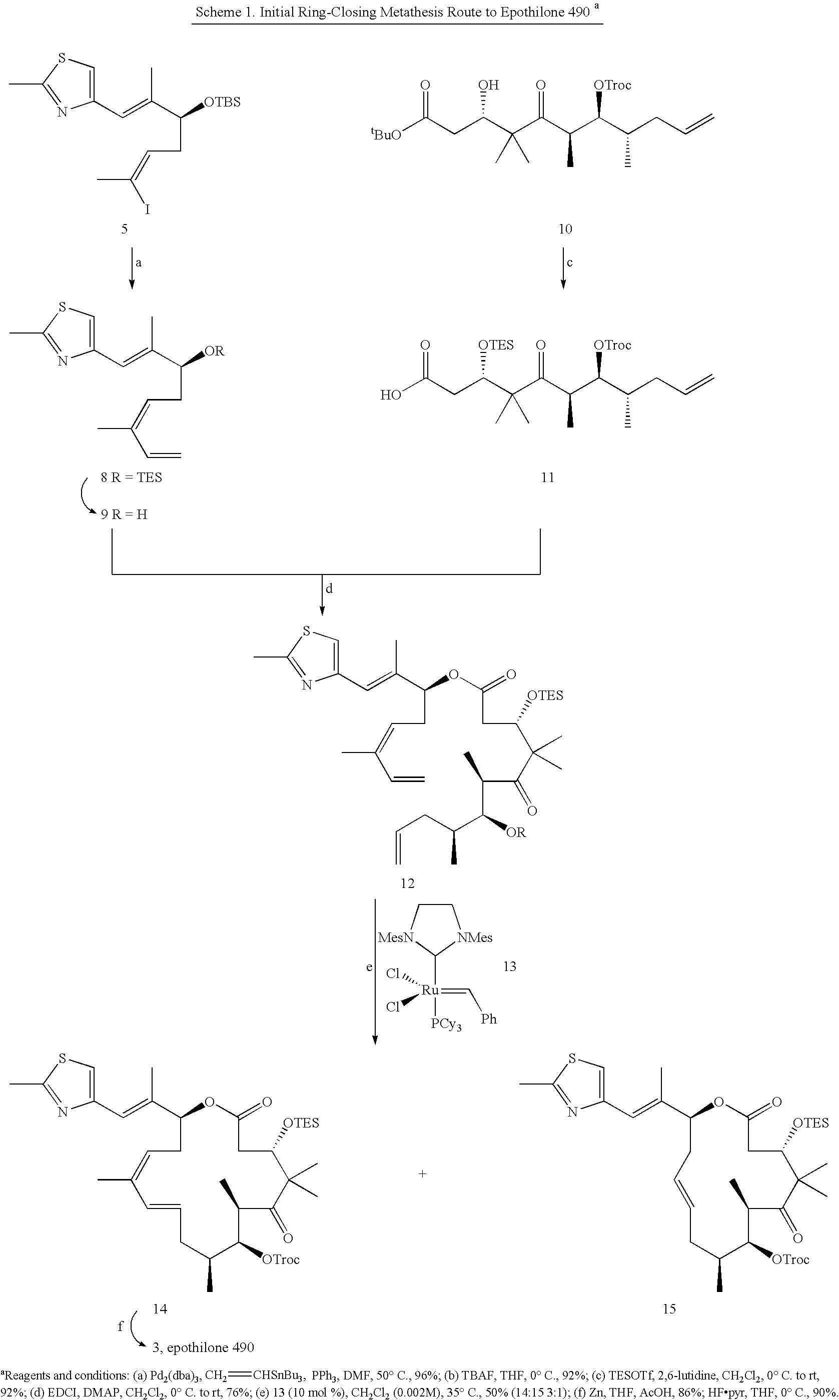

A convergent solution was used to accomplish the C1-C2 interpolation and the creation of the diene functionality. Stille coupling (Farina, V.; Krishnamurthy, V.; Scott, W. J. Org. React. 1997, 50, 1; incorporated herein by reference) of 5 with vinyl n-tributyltin afforded 8 (Scheme 1). Cleavage of the silyl-protecting group afforded 9. Our initial approach commenced with EDCI/DMAP-mediated esterification of the resulting allylic alcohol 9 with the C1 acid fragment 11, obtained by deprotection of known tert-butyl ester 12 (Lee, C. B.; Wu, Z.; Zhang, F.; Chappell, M. D.; Stachel, S. J.; Chou, T. C.; Guan, Y.; Danishefksy, S. J. J. Am. Chem. Soc. 2001, 123, 5249; incorporated herein by reference). This reaction yielded the cyclization precursor, triene 12. Exposure of 12 to the RCM reaction with the second-generation ruthenium metathesis catalyst 13 (Initial report: Scholl, M.; Trnka, T. M.; Morgan, J. P.; Grubbs, R. H. Tetrahedron Lett. 1999, 40, 2247; incorporated herein by reference) in methylene chloride gave a mixture of two compounds in a 3:1 ratio, with a total yield of 50% . No reaction was observed with the first-generation bis(cyclohexyl)ruthenium Grubbs catalyst, while treatment with the Schrock molybdenum catalyst led to decomposition of the starting material. The major component of the product mixture was identified as the desired trans-substituted diene product 14, along with the 14-membered macrolide 15 as a minor product, seemingly arising from a metathesis reaction involving the internal 12,13-olefin. Deprotection of the Troc and silyl groups led to fully synthetic epothilone 490 (3), identical in all respects to an authentic sample. The formation of the E-10,11-double bond was highly stereoselective and helped to confirm the stereochemistry of epothilone 490 to be as shown.

Following a similar series of reactions, the 21-hydroxyl variant of the new compound, the 10,11-dehydro version of desoxyepothilone F, compound 4 was synthesized. Starting with the known Troc-protected 21-hydroxy vinyl iodide 16 (Lee, C. B.; Chou, T.-C.; Zhang, X. G.; Wang, Z. G.; Kuduk, S. D.; Chappell, M. D.; Stachel, S. J.; Danishefsky, S. J. J. Org. Chem 2000, 65, 6525; incorporated herein by reference), Stille coupling gave diene 17 (Scheme 2). Deprotection of the silyl group followed by esterification and RCM afforded 20. Deprotection of the Troc and triethylsilyl groups afforded 21-hydroxy diene 4.

Only the desired E-olefin in the reaction mixture was observed. Examination of the sequence of steps that led to the construction of the cyclization precursor suggested a different order of conjoining the fragments in fewer total steps. Since the C3 (S)-stereocenter is constructed by a chiral titanium-mediated acetate aldol reaction (Wu, Z.; Zhang, F.; Danishefsky, S. J. Angew. Chem., Int. Ed. 2000, 39, 4505; incorporated herein by reference), we decided to attempt this reaction at a late stage, with the entire O-alkyl fragment serving as part of the chiral nucleophile as its C15 acetate. In this context, the allylic alcohol 9 was acylated to obtain the desired acetate 21 (Scheme 3). Following the protocol of Duthaler, (Duthaler, R. O.; Herold, P.; Lottenbach, W.; Oretle, K.; Reidiker, M. Angew. Chem., Int. Ed. Engl. 1989, 28, 495; incorporated herein by reference), the lithium enolate of 21 was treated with the chiral titanium reagent to generate the chiral titanium enolate. Addition of aldehyde 6 afforded the desired aldol product, 22, as a single diastereomer. The identity of the product was verified by treatment with TESCI to generate the C3 TES ether, which was identical to 12, as determined by ¹H and ¹³C NMR and optical rotation. Furthermore, 22 was converted to epothilone 490, verifying the (S)-stereochemistry at C3.

Mindful of the fact that the newer ruthenium metathesis catalysts are tolerant of a wide variety of functional groups, an RCM reaction on 22 without protection of the C3 alcohol moiety was attempted. Treatment of 22 with catalyst 13 afforded the desired product in 41% yield, with none of the 14-membered macrolide being observed. Deprotection of the C7 Troc-protecting group in the usual way afforded epothilone 490.

The change in ratios of the 16- and 14-membered macrolide rings upon deprotection of the C3 alcohol suggested a surprising substrate effect on the macrocyclization step. A series of RCM reactions in which we varied the protection status of the C3 and the C7 alcohols in all of the possible combinations was performed (Table 1 below). The results were dependent on the presence of the protecting groups. The 14-membered macrolide was observed only when the substrate was fully protected. More importantly, the yield of the reaction almost doubled upon use of a substrate where C7 is free. In fact, RCM of the fully deprotected substrate afforded the product epothilone 490 in 64% yield, with no observed Z-isomer of the C10-C11 olefin. Interestingly, when we carried out this same series of reactions in refluxing toluene, this substrate effect was diminished, with 55-58% yields observed across the various substrates. Toluene is a preferred solvent for scale-up processes; indeed, compound 22, derived from the acetate aldol as shown in Scheme 3, was successfully subjected to metathesis conditions at 1 mmol scale in toluene at 110° C. as a proof of principle experiment.

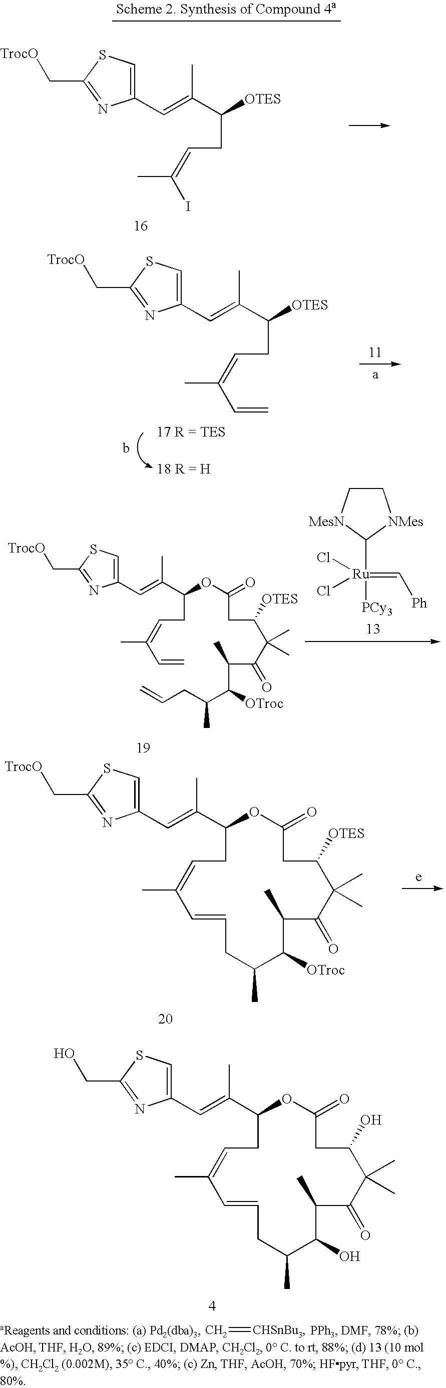

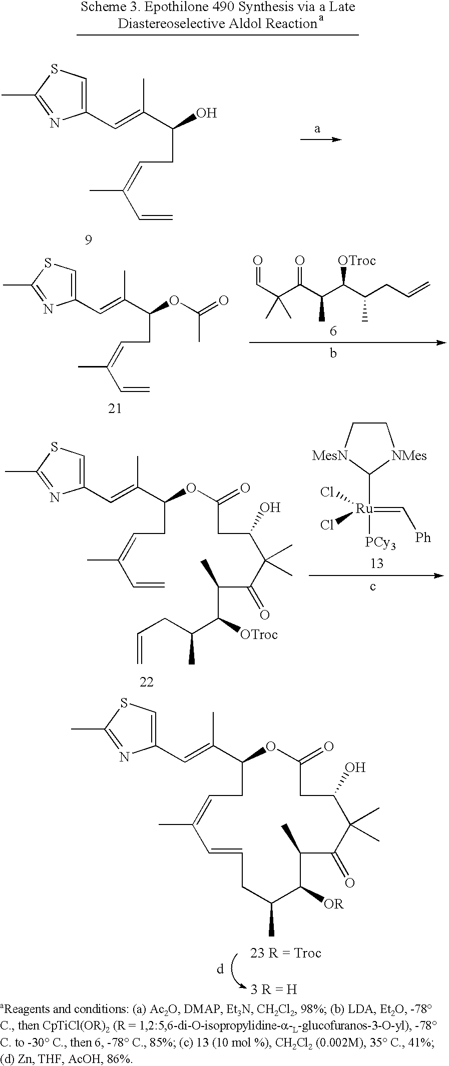

TABLE 1
Effect of Alcohol Protection and Different Solvents on RCM Yield^{a}

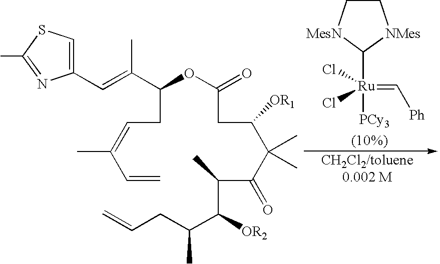

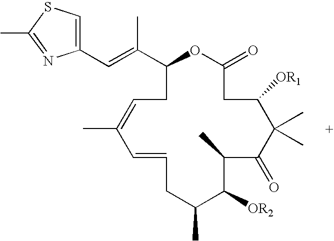

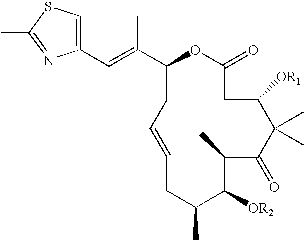

| | | | |
|---|---|---|---|
| 12 | R₁ = TES₁ R₂ = Troc | 35%/58%^{b} | 15%/6%^{b} |
| 22 | R₁ = H₁ R₂ = Troc | 41%/57% | 0%/0% |
| 24 | R₁ = TES R₂ = H | 57%/n.d.^{c} | 0%/n.d.^{c} |
| 25 | R₁ = H₁ R₂ = H | 64%/55% | 0%/0% |

^{a}Reactions in CH₂Cl₂ were run for 5.5 h at 35° C., reactions in toluene for 25 min at 110° C.
^{b}Done with 20 mol % catalyst at 0.0005 M dilution.
^{c}Not determined.

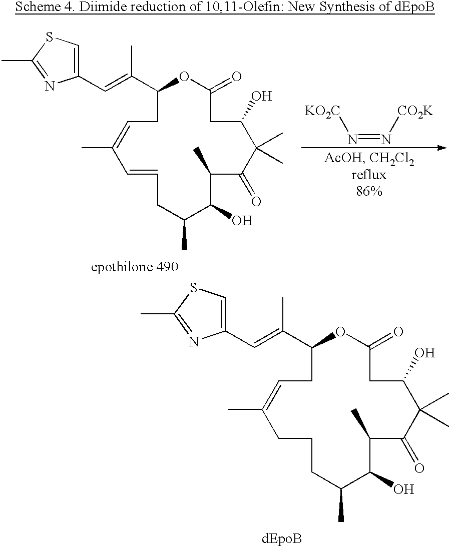

Selective Diimide Reduction of 10,11-Olefin: A New Route to dEpoB. The successful application of RCM to the synthesis of the diene epothilones of the 490 series led us to examine whether we could access dEpoB by this newly described end game. Attainment of this goal would involve a selective hydrogenation of the disubstituted C10-C11 E-olefin, in the presence of the trisubstituted C12-C13 Z-olefin and the “benzylic” trisubstituted C16-C17 olefin. Diimide-based reductions are known to be extremely sensitive to steric effects in distinguishing differentially substituted olefins (Corey, E. J.; Mock, W. L.; Pasto, D. J. Tetrahedron Lett. 1961, 347; Pasto, D. J.; Taylor, R. T. Org. React. 1991, 40, 91; each of which is incorporated herein by reference). Therefore, we turned our attention to diimide as a reducing agent to convert epothilone 490 to dEpoB. This goal was successfully accomplished by treatment of fully synthetic 3 with in situ generated diimide (86% yield, Scheme 4).

By focusing on a new section of the carbon skeleton for generation of an olefin, we have been able to successfully access the epothilone framework using an RCM-reduction protocol. During this process, we utilized the syntheses of advanced intermediates 5 and 6, and fashioned the epothilone scaffold by a novel sequence of highly efficient reactions.

Selective Functionalization of the 10,11-Olefin. The successful reduction reaction also indicated that selective functionalization of the newly generated C10-C11 olefin was feasible to enable a SAR profile of that sector of epothilones. Therefore, we report on the synthesis and preliminary evaluation of some novel epothilones available via epothilone 490. We subjected dienes in this series to dihydroxylation, epoxidation, and cyclopropanation conditions. Treatment of 3 with catalytic osmium tetroxide in the presence of NMO resulted in the formation of a 10:1 mixture, where the major product was identified as 26 (Scheme 5). The minor product arises from the dihydroxylation of the 12,13-olefin.

The stereochemistry at C10 and C11 of 26 was determined by X-ray crystallography, as depicted in FIG. 5a. Inspection of a Macromodel-derived minimized (MM2) structure of epothilone 490 shows that the “external” face of the 10-11 olefin is more available to reagents. This model suggests a rationalization of the product stereochemistry we observe in the dihydroxylation reaction.

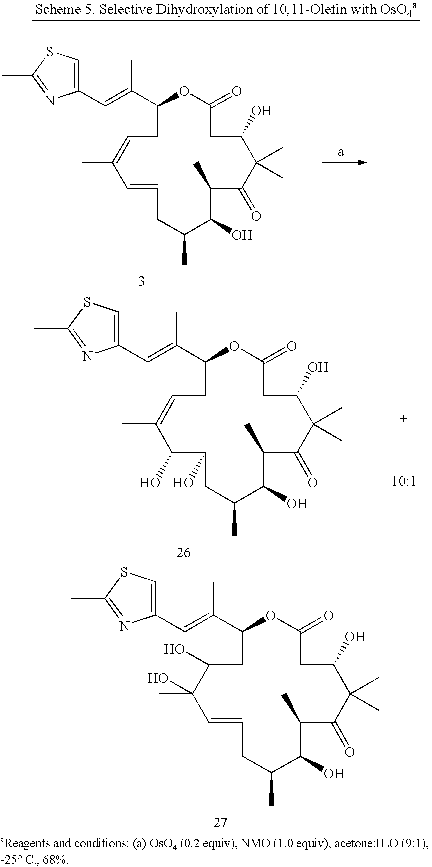

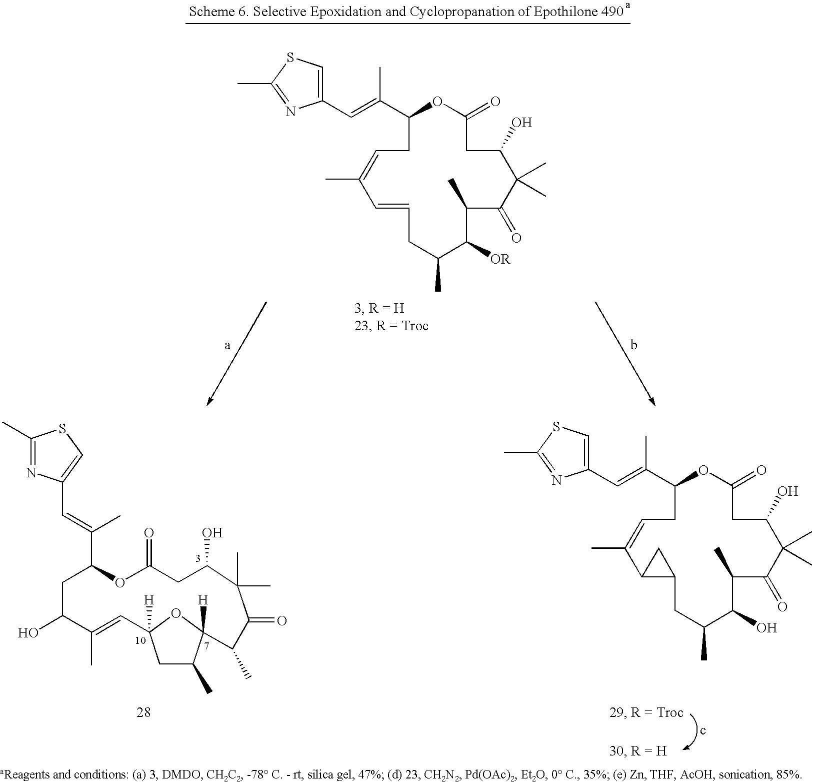

Interestingly, exposure of 3 to the action of 2,2′-dimethyldioxirane, with the intent of generating an epoxide, gave rise to tetrahydrofuran-containing macrocycle 28 upon silica gel purification. The stereochemistry of macrocycle 28 was assigned on the basis of the analysis of 2D COSY and NOESY spectra, assuming that all the existing stereochemistry remained untouched under the mild reaction conditions. Compound 28 arises from epoxidation of the 12,13-olefin and S_{N}2′-type participation of the C-7 hydroxyl group (Scheme 6). Finally, treatment of 23 with diazomethane in the presence of Pd(OAc)₂, (Denmark, S. E.; Scavenger, R. A.; Faucher, A.-M.; Edwards, J. P. J. Org. Chem. 1997, 62, 3375 and references therein; each of which is incorporated herein by reference) followed by deprotection, afforded the vinyl cyclopropane 30.

The new analogues obtained from epothilone 490 exhibited a range of in vitro cytotoxities (Chou, T. C.; O'Connor, O. A.; Tong, W. P.; Guan, Y.; Zhang, Z.-G.; Stachel, S. J.; Lee, C.; Danishefsky, S. Proc. Natl. Acad. Sci. U.S.A. 2001, 98, 8113; incorporated herein by reference) and microtubule stabilizing ability (Gaskin, F.; Cantor, C. R.; Shelanski, M. L. J. Mot. Biol. 1974, 89, 737; incorporated herein by reference) as shown in Table 2. Indeed, the microtubule stabilizing ability closely parallels the observed cytotoxicity data.

Epothilone 490 exhibited impressive cell growth inhibition across a range of drug-resistant tumors. Surprisingly, epothilone 490 did not demonstrate statistically inhibitory effect on the growth of the implanted tumors, as compared to control mice (See Example 13). This result was surprising in view of the favorable results of the in vitro studies.

TABLE 2
In Vitro Cytotoxicities (IC₅₀) with Tumor Cell Lines^{a}
and Microtubule Binding

| | CCRF- | CCRF- | CCRF- | CCRF- | |
|---|---|---|---|---|---|
| | CEM | CEM/_{VBL100} | CEM/_{VMI} | CEM/_{TAXOL} | % tubulin |
| cmpd | (μM) | (μM) | (μM) | (μM) | binding^{b} |
| 1 (dEpoB) | 0.011 | 0.015 | 0.016 | 0.007 | 100 |
| 3 | 0.025 | 0.091 | 0.035 | 0.032 | 89 |
| 4 | 0.030 | 0.202 | 0.061 | 0.051 | 77 |
| 26 | 1.001 | 99.0 | 2.35 | 16.76 | 31 |
| 28 | 0.761 | 8.76 | n.d.^{c} | 4.24 | inactive |
| 30 | 0.077 | 0.114 | n.d.^{c} | n.d.^{c} | 84 |
| Taxol | 0.0021 | 0.827 | 0.003 | 0.081 | n.d.^{c} |
| vinblastine | 0.0008 | 0.122 | 0.0014 | 0.018 | n.d.^{c} |

^{a}XTT assay following 72-h inhibition. CCRF-CEM is a human T-cell acute lymphoblastic leukemia cell line. The CCRF-CEM/_{VBL100}, CCRF-CEM/_{VMI}, and CCRF-CEM/_{TAXOL} cell lines all overexpress P-glycoprotein and display a multidrug resistance phenotype to MDR-associated oncolytics. (Chou, T. C.; O'Connor, O. A.; Tong, W. P.; Guan, Y.; Zhang, Z. -G.; Stachel, S. J.; Lee, C.; Danishefsky, S. J. Proc. Natl. Acad. Sci. U.S.A. 2001, 98, 8113; incorporated herein
by reference). ^{b}Formation of microtubules in the presence of the compounds. Microtubules formed in the presence of dEpoB is defined as 100%. (See Su et al. Angew. Chem. Int. Ed. Engl. 36: 757, 1997, incorporated herein by reference, for experimental details.).
^{c}Not determined.

However, the apparently disappointing murine in vivo results should be viewed in the context of reports that dEpoB itself evidenced a degree of bioinstability in murine plasma; yet had much longer plasma half-lives in higher organisms, including humans (Chou, T. C.; O'Connor, O. A.; Tong, W. P.; Guan, Y.; Zhang, Z.-G.; Stachel, S. J.; Lee, C.; Danishefsky, S. J. Proc. Natl. Acad. Sci. U.S.A. 2001, 98, 8113; incorporated herein by reference). The observed discrepancy in efficacy between mice and other mammals, including humans, has been ascribed to higher esterase levels in rodents. Indeed, on exposure ot 1 and 3 to murine plasma, a faster degradation of epothilone 490 compounds as compared to dEpoB was observed (FIG. 21). However, no measurable degradation of 3 was observed after more than 3 hours of exposure in human plasma.

In view of such data, those having skill in the pharacological arts will therefore understand that the observed discrepancy between the excellent in vitro activity of epothilone 490 and its degree of activity in the murine assay is likely to be merely an artifact of murine biochemistry.

### Experimentals

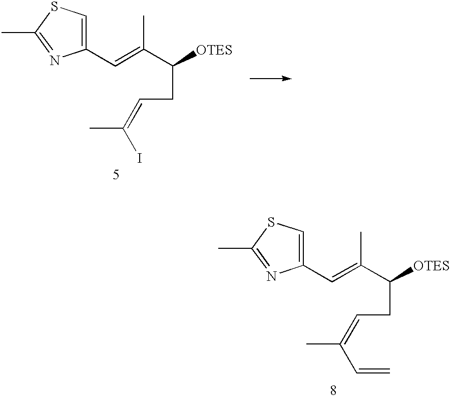

Compound 8. To a stirred solution of vinyl iodide 5 (1.30 g, 2.8 mmol) in DMF (25 mL) were added vinyltributyltin (2.45 mL, 8.4 mmol, 3.0 equiv) and triphenylphosphine (295 mg, 1.1 mmol, 0.4 equiv), followed by Pd₂(dba)₃ (512 mg, 0.5 mmol, 0.2 equiv). The reaction mixture was heated at 50° C. for 45 min, cooled to room temperature, diluted with EtOAc (75 mL) and washed with water (2×50 mL), brine (50 mL), dried over MgSO₄ and concentrated in vacuo. Purification using silica gel chromatography employing 4% EtOAc/hexane as the eluent afforded diene 8 (970 mg, 96% yield) as a clear oil: [α]_{D} +8.0° (c 1.48, CHCl₃); IR (neat) 2953, 2910 2875, 1652, 1595, 1506, 1457, 1438, 1418, 1377, 1238, 1182, 1074, 1005 cm⁻¹; ¹H NMR (400 MHz, CDCl₃) δ6.90 (s, 1H), 6.74 (dd, J=17.4, 10.9 Hz, 1H), 6.46 (s, 1H), 5.38 (t, J=7.4 Hz, 1H), 5.17 (d, J=16.7 Hz, 1H), 5.07-5.05 (m, 1H), 4.12 (t, J=6.5 Hz, 1H), 2.69 (s, 3H), 2.46-2.40 (m, 2H), 1.99 (d, J=1.1 Hz, 3H), 1.78 (s, 3H), 0.90 (t, J=7.9 Hz, 9H), 0.56 (q, J=7.9 Hz, 6H); ¹³C NMR (100 MHz, CDCl₃) δ164.3, 153.1, 142.2, 133.8, 133.6, 127.1, 118.8, 115.0, 113.5, 78.4, 34.7, 19.8, 19.2, 13.8, 6.8, 4.7; HRMS (FAB) calcd. for C₂₀H₃₄NOSSi (M+H⁺) 364.2130, found 364.2134.

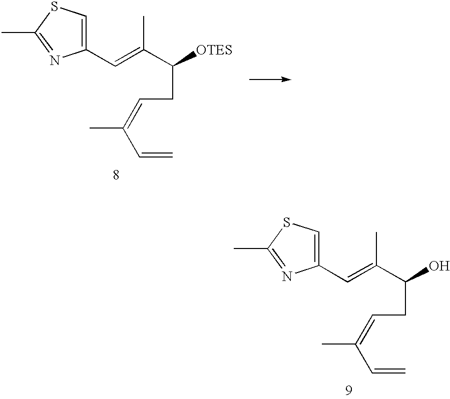

Compound 9. Diene 8 (2.7 g, 7.4 mmol) was dissolved in THF (50 mL) and cooled to 0° C. The reaction mixture was treated with TBAF (11.1 mL of a 1.0 M solution in THF, 11.1 mmol, 1.5 equiv), stirred at 0° C. for 30 min, diluted with a saturated solution of NaHCO₃ (100 mL) and extracted with Et₂O (3×150 mL). The combined organic layers were dried over MgSO₄ and concentrated in vacuo. Purification using silica gel chromatography employing 35% EtOAc/hexane+1% Et₃N as the eluent afforded alcohol 9 (1.69 g, 92% o yield) as a clear oil: [α]_{D} −36.0° (c 0.76, CHCl₃); IR (neat) 3353, 3088, 2923, 1725, 1650, 1594, 1508, 1440, 1378, 1307, 1270, 1186,901 cm⁻¹; ¹H NMR (400 MHz, CDCl₃) δ6.96 (s, 1H), 6.79 (dd, J=17.6, 10.7 Hz, 1 H), 6.58 (s, 1H), 5.44 (t, J=7.4 Hz, 1H), 5.26 (d, J=17.2 Hz, 1H), 5.15 (d, J=10.8 Hz, 1H), 4.22 (t, J=6.3 Hz, 1H), 2.72 (s, 3H), 2.60-2.50 (m, 2H), 2.06 (s, 3H) 1.92 (bs, 1H), 1.86 (s, 3H); ¹³C NMR (100 MHz, CDCl₃) δ164.5, 152.5, 141.7, 134.4, 133.3, 126.2, 118.9, 115.1, 114.0, 76.9, 33.2, 19.7, 18.8, 14.1; HRMS (FAB) calcd. for C₁₄H₂₀NOS (M+H⁺) 250.1265, found 250.1275.

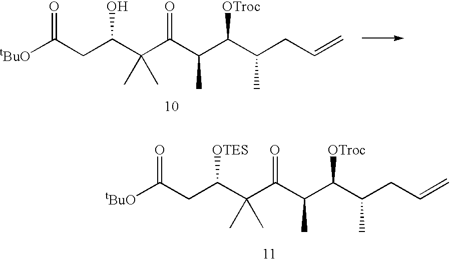

Compound 11. tert-Butyl ester 10 (900 mg, 1.7 mmol) was dissolved in methylene chloride (15 mL) and treated with 2,6-lutidine (1.9 mL, 17 mmol, 10.0 equiv). After cooling the reaction mixture to ) 0° C., TESOTf (2.3 mL, 9.8 mmol, 6.0 equiv) was added in a dropwise fashion. The reaction mixture was allowed to warm to rt while stirring over 15 h, diluted with EtOAc (50 mL), and washed with 1N HCl (3×35 mL). The organic layer was dried over MgSO₄ and concentrated in vacuo. Purification using silica gel chromatography employing 50% EtOAc/hexane as the eluent afforded acid 11 (920 mg, 92% yield) as a clear oil: [α]_{D} −28.5° (c 1.4, CDCl₃); IR (neat) 2955, 2884, 1755, 1708, 1384, 1249, 1091, 991, 926, 738 cm⁻¹; ¹H NMR (400 MHz, CDCl₃) 8 5.75-5.65 (m, 1H), 5.04-4.99 (m, 2H), 4.84 (d, J=11.9 Hz, 1H) 4.78 (dd, J=7.6, 3.9 Hz, 1H), 4.68 (d, J=11.9 Hz, 1H), 4.23 (dd, J=7.5, 2.9 Hz, 1H), 3.48-3.44 (m, 1H), 2.61 (dd, J=16.8, 2.9 Hz, 1H), 2.30-2.24 (m, 1H), 2.23 (dd, J=16.8, 7.5 Hz, 1H), 1.94-1.83 (m, 2H), 1.34 (s, 3H), 1.08-1.06 (m, 6H), 0.96-0.91 (m, 12H), 0.65-0.59 (m, 6H); ¹³C NMR (100 MHz, CDCl₃) δ215.2, 178.1, 154.1, 135.6, 117.2, 94.6, 81.8, 75.0, 53.4, 42.3, 39.5, 36.4, 34.4, 22.5, 20.3, 15.7, 10.9, 6.9, 5.0; HRMS (FAB) calcd. for C₂₄H₄₁Cl₃NaO₇Si (M+Na⁺) 597.1587, found 597.1587.

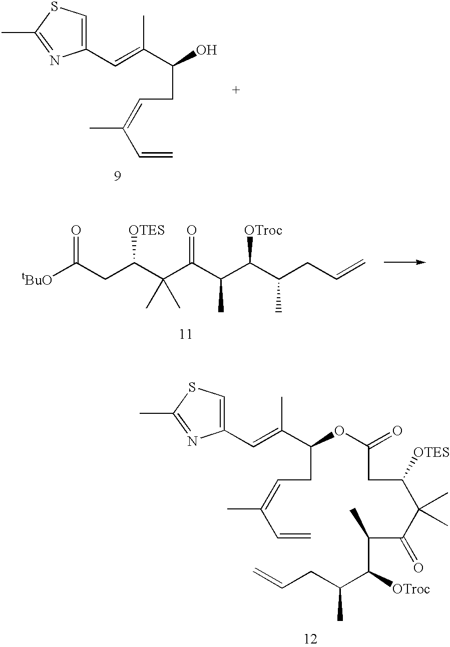

Compound 12. To a stirred solution of alcohol 9 (37 mg, 0.14 mmol, 1.5 equiv) in methylene chloride (0.6 mL) at 0° C. were added EDCI (30 mg, 0.15 mmol, 1.6 equiv) and DMAP (19 mg, 0.15 mmol, 1.6 equiv). A solution of acid 11 (55 mg, 0.09 mmol) in methylene chloride (0.6 mL) was added to the reaction mixture in a dropwise fashion, which was warmed to room temperature. The reaction was concentrated after 2 h, and purified using silica gel chromatography employing 8% EtOAc/hexane as the eluent to afford ester 12 (58 mg, 76% yield) as a clear oil: [α]_{D} −57.2° (c 0.5, CHCl₃); IR (neat) 2956, 1757, 1733, 1700, 1453, 1381, 1251, 1181, 1096, 1068 cm⁻¹; ¹H NMR (400 MHz, CDCl₃) δ6.94 (s, 1H), 6.74 (dd, J=17.2, 10.8 Hz, 1H), 6.50 (s, 1H), 5.74-5.64 (m, 1H), 5.31 (t, J=7.2 Hz, 1H), 5.27-5.20 (m, 2H), 5.12 (d, J=10.8 Hz, 1H), 5.02-4.96 (m, 2H), 4.82 (d, J=12.0 Hz, 1H), 4.72 (dd, J=7.6, 3.1 Hz, 1H), 4.66 (d, J=12.0 Hz, 1H), 4.21 (dd, J=6.8, 2.9 Hz, 1H), 3.51-3.45 (m, 1H), 2.70 (s, 3H), 2.69-2.49 (m, 3H), 2.26-2.16 (m, 2H), 2.06 (s, 3H), 1.95-1.80 (m, 2H), 1.75 (s, 3H), 1.36 (s, 3H), 1.06 (d, J=6.7 Hz, 3H), 1.01 (s, 3H), 0.97-0.93 (m, 12H), 0.63 (q, J=8.0 Hz, 6H); ¹³C NMR (100 MHz, CDCl₃) δ215.1, 171.1, 164.5, 153.9, 152.4, 136.7, 135.5, 134.7, 133.3, 124.7, 121.1, 117.0, 116.4, 114.4, 94.6, 81.6, 79.2, 76.6, 75.1, 53.3, 42.1, 39.6, 36.5, 34.2, 30.9, 22.1, 20.8, 19.7, 19.1, 15.4, 14.4, 10.4, 6.9, 4.9; LRMS (ESI) calcd. for C₃₈H₅₈Cl₃NO₇SSi 805.2, found 828.4 (M+Na⁺).

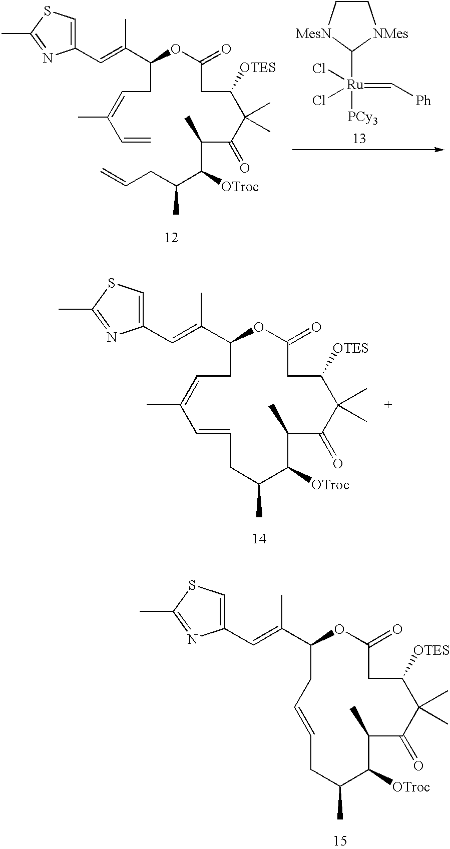

Compounds 14 and 15. A solution of the diene ester 12 (67 mg, 0.08 mmol) and tricyclohexylphosphine[1,3-bis(2,4,6-trimethylphenyl)-4,5-dihydroimidazole-2-ylidene-[benzylidine]ruthenium(IV) dichloride (Grubb's catalyst) (13) (7 mg, 0.008 mmol, 0.1 equiv) in 40 mL of methylene chloride was stirred at 35° C. for 5.5 hr. The solution was cooled to room temperature and passed through a plug of silica gel using 50% hexane/EtOAc. The combined filtrate was concentrated in vacuo and purified using silica gel chromatography employing 17% EtOAc/hexane as the eluent, which yielded a 3:1 mixture (31 mg, 50% yield) of the desired product 15 and the 14-membered ring product 15. The compounds were characterized after deprotection of the C7 Troc groups, after which they were separable by silica gel chromatography (vide infra).

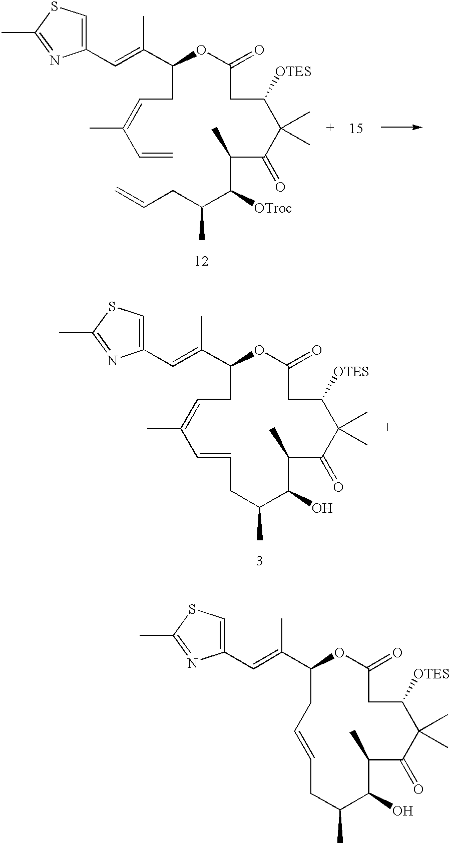

Epothilone 490. To a stirred solution of a 3:1 mixture of RCM products 14 and 15 (22 mg) in 1:1 THF/HOAc (1.2 mL) was added a spatula tip of nanosize Zn° (˜2 mg). The reaction mixture was sonicated for 10 min and then filtered through celite, washing the celite cake with EtOAc. The combined filtrate was washed with saturated NaHCO₃ (10 mL), brine (10 mL), and dried over MgSO₄. Removal of the solvent in vacuo followed by purification of the residue on silica gel chromatography using 25% EtOAc/hexane as the eluent yielded the C7 alcohol from 15 (3.4 mg, 21% yield) and the C7 alcohol from 14 (9.9 mg, 56% yield):

C7 Alcohol from 15: [α]_{D} −11° (c 0.25, CHCl₃); ¹H NMR (500 MHz, CDCl₃) 6.90 (s, 1H), 6.51 (s, 1H), 6.49 (d, J=14.8 Hz, 1H), 5.68 (ddd, J=14.8, 8.8, 5.4 Hz, 1H), 5.26 (d, J=10.7 Hz, 1H), 5.22 (dd, J=10.7, 6.0 Hz, 1H), 4.30 (dd, J=6.3, 6.30 Hz, 1H), 3.58 (d, J=5.7 Hz, 1H), 3.45 (bs, 1H), 3.10 (qd, J=6.6, 1.6 Hz, 1H ), 2.89 (ddd, J=14.5, 10.7, 10.7 Hz, 1H), 2.64 (s, 3H), 2.55-2.49 (m, 1H), 2.46-2.41 (m, 2H), 2.15-2.08 (m, 1H), 2.09 (s, 3H), 2.04-1.97 (m, 2H), 1.70 (s, 3H), 1.08 (s, 3H), 1.04 (d, J=6.6 Hz, 6H), 0.91 (s, 3H), 0.82 (t, J=7.9 Hz, 9H), 0.50 (q, J=7.9 Hz, 6H); ¹³C NMR (125 MHz, CDCl₃) 220.0, 169.9, 164.8, 152.4, 137.7, 135.6, 129.6, 129.0, 123.2, 120.8, 116.6, 79.4, 73.8, 71.4, 54.9, 40.6, 40.1, 36.6, 35.8, 32.3, 29.7, 21.1, 20.3, 20.1, 19.2, 16.7, 14.9, 12.0, 6.9, 5.5; LRMS (ESI) calcd. for C₃₀H₄₉NO₅SSi 563.3, found 586.4 (M+Na⁺).

C7 Alcohol from 14: [α]_{D} −87° (c 0.095, CHCl₃); IR (neat) 3509, 2957, 1734, 1684, 1456, 1106, 743 cm⁻¹; ¹H NMR (500 MHz, CDCl₃) 6.85 (s, 1H), 6.48 (s, 1H), 5.50 (ddd, J=15.3, 7.9, 7.5 Hz, 1H), 5.36 (ddd, J=15.3, 7.5, 6.9 Hz, 1H), 5.22 (dd, J=7.9, 4.1 Hz, 1H), 4.46 (dd, J=8.4, 2.7 Hz, 1H), 3.40 (d, J=8.3 Hz, 1H), 3.16 (q, J=6.9 Hz, 1H), 2.94 (bs, 1H), 2.64 (s, 3H), 2.64-2.62 (m, 1H), 2.47 (ddd, J=12.9, 7.9, 4.4 Hz, 1H), 2.43 (dd, J=16.1, 8.4 Hz, 1H), 2.23 (dd, J=16.1, 2.7 Hz, 1H), 2.07 (s, 3H), 1.91-1.85 (m, 1H), 1.83-1.77 (m, 1H), 1.15 (s, 3H), 0.98 (d, J=6.9 Hz, 3H), 0.93 (s, 3H), 0.90 (d, J=7.3 Hz, 3H), 0.81 (t, J=7.9 Hz, 9H), 0.590-047 (m, 6H); ¹³C NMR (125 MHz, CDCl₃) 220.3, 169.9, 164.6, 152.5, 136.9, 130.5, 127.5, 120.4, 116.2, 78.6, 71.0, 70.2, 55.9, 41.7, 40.5, 36.5, 34.9, 34.5, 22.1, 19.3, 16.33, 16.27, 15.2, 9.9, 5.3; LRMS (ESI) calcd. for C₃₃H₅₃NO₅SSi 603.3, found 626.3 (M+Na⁺).

HF.Py (0.02 ml-) was added to a solution of the C7 alcohol from 14 (4.6 mg, 0.0076 mmol) in THF (0.2 mL). The resulting solution was stirred at room temperature for 3 h, and then carefully poured into saturated NaHCO₃ solution, which was extracted with EtOAc (3×5 mL).

The combined organic layers were washed with brine (5 mL), dried over MgSO₄ and concentrated in vacuo. The residue was purified using silica gel chromatography employing 40% EtOAc/hexane as the eluent, which furnished epothilone 490 (3.5 mg, 90% yield): [α]_{D} −50.0° (c 0.085, CHCl₃); ¹H NMR (500 MHz, CDCl₃) δ6.91 (s, 1H), 6.55 (s, 1H), 6.46 (d, J=15.5 Hz, 1 H), 5.70 (ddd, J=15.5, 8.5, 5.4 Hz, 1H), 5.24-5.18 (m, 2H), 4.17 (d, J=10.7, 2.8 Hz, 1H), 3.65 (d, J=6.6 Hz, 1H), 3.20 .(q, J=7.3 Hz, 1H), 3.09 (s, 1H), 2.73 (ddd, J=14.8, 10.7, 10.4 Hz, 1 H), 2.66 (s, 3H), 2.47 (ddd, J=14.8, 5.0, 4.7 Hz, 1 H), 2.37 (dd, J=15.7, 10.7 Hz, 1H), 2.28 (dd, J=15.7, 2.8 Hz, 1H), 2.24 (dd, J=14.3, 6.2 Hz, 1H), 2.26-2.22 (m, 1H), 2.03 (s, 3H), 2.30-2.19 (m, 1H), 1.94-1.89 (m, 1H), 1.73 (s, 3H), 1.25 (s, 3H), 1.05 (d, J=6.9 Hz, 3H), 1.01 (d, J=7.3 Hz, 3H), 0.97 (s, 3H); ¹³C NMR (100 MHz, CDCl₁₃) δ220.4, 170.3, 165.0, 152.0, 138.3, 135.6, 129.4, 129.2, 123.2, 119.1, 116.0, 78.2, 71.7, 71.6, 53.5, 41.1, 39.5, 36.9, 36.0, 32.1, 22.2, 21.1, 19.0, 18.9, 16.8, 15.8, 11.5; HRMS (FAB) calcd. for C₂₇H₃₉NNaO₅S (M+Na⁺) 512.2446, found 512.2445.

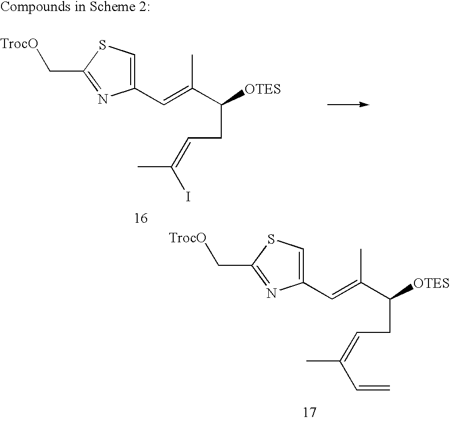

Compound 17. To a stirred solution of vinyl iodide 16 (1.50 g, 2.3 mmol) in DMF (25 mL) were added vinyltributyltin (2.02 mL, 6.8 mmol, 3.0 equiv) and triphenylphosphine (120 mg, 0.4 mmol, 0.2 equiv), followed by Pd₂(dba)₃ (210 mg, 0.2 mmol, 0.1 equiv). The reaction mixture was heated at 50° C. for 2 h, cooled to room temperature, diluted with EtOAc (75 mL) and washed with water (2×50 mL), brine (50 mL), dried over MgSO₄ and concentrated in vacuo. Purification using silica gel chromatography employing 4% EtOAc/hexane as the eluent afforded diene 17 (870 mg, 78% yield) as a clear oil: [α]_{D} +33.7° (c 1.0, CHCl₃); IR (neat) 2954, 2875, 1765, 1456, 1381, 1238, 1070 cm⁻¹; ¹H NMR (400 MHz, CDCl₃) 6 7.15 (s, 1H), 6.77 (dd, J=17.1. 10.8 Hz, 1H), 6.52 (s, 1H), 5.53 (s, 2H), 5.41 (t, J=7.4 Hz, 1H), 5.21 (d 5.10 (d, J=10.8 Hz, 1H), 4.84 (s, 2H), 4.16 (t, J=6.3 Hz, 1H), 2.47 (m, 2H), 2.04 (s, 3H), 1.82 (s, 3H), 0.94 (t, J=7.9 Hz, 9H), 0.59 (q, J=7.9 Hz, 6H); ¹³C NMR (100 MHz, CDCl₃)δ; 161.1, 153.9. 153.6, 143.4, 133.7, 126.9. 118.0, 117.2, 113.6, 94.0, 78.2, 77.2, 77.1, 66.6, 34.7, 19.8, 14.0, 6.8, 4.7; HRMS (FAB) calcd. for C23H34C13NO₄SSi (M+H⁺) 554.1121, found 554.1132.

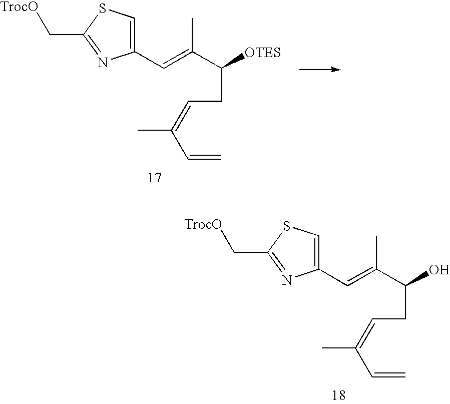

Compound 18. The silyl ether (748 mg, 1.3 mmol) was dissolved in 15 mL of a 3:1:1 solution of Ac QH:THF:H₂0, and stirred at rt for 30 min. The reaction mixture was diluted with EtOAc (25 mL) and washed with a saturated solution of NaHCO₃ (3×20 mL), brine (15 mL), dried over Na₂SO4 and concentrated in vacuo. Purification using silica gel chromatography employing 50% EtOAc/hexane+1% Et₃N as the eluent afforded alcohol 18 (525 mg, 89% yield) as a clear oil: [α]_{D} −17.6° (c 0.9, CHCl₃); IR (neat) 3387, 2956, 1762, 1503, 1438, 1383, 1307 cm⁻¹; ¹H NMR (400 MHz, CDCl₃) δ7.18 (s, 1H), 6.80 (dd, J=17.2, 10.8 Hz, 1H), 6.61 (s, 1H), 5.52 (s, 2H), 5.44 (t, J=7.3 Hz, 1H), 5.28 (d, J=17.3 Hz, 1H), 5.16 (d, J=10.8 Hz, 1H), 4.84 (s, 2H), 4.23 (t, J=6.3 Hz, 1H), 2.55 (m, 2H), 2.09 (s, 3H), 1.87 (s, 3H); ¹³C NMR (100 MHz, CDCl₃) δ161.4, 153.6, 142.5, 135.5, 133.2, 125.7, 118.3, 117.7, 114.6, 94.0, 77.2, 77.1, 66.5, 33.5, 30.9, 19.9, 14.5; LRMS (ESI) calcd. for C₁₇H₂₀Cl₃NO₄S 439.0, found 462.0 (M+Na⁺).

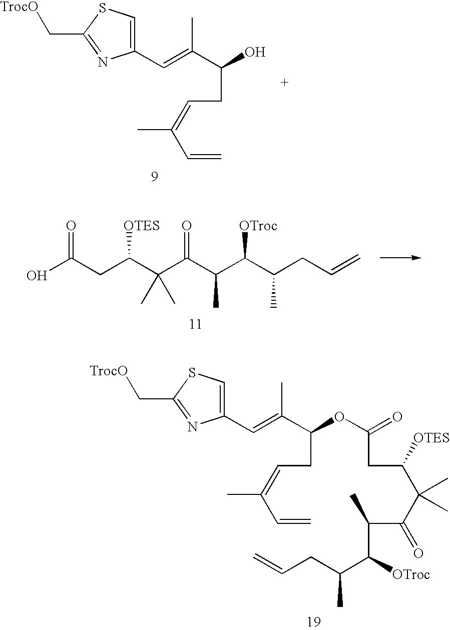

Compound 19. To a stirred solution of alcohol 18 (505 mg, 1.1 mmol) in methylene chloride (8 mL) at 0° C. were added EDCI (352 mg, 1.8 mmol, 1.6 equiv) and DMAP (225 mg, 1.8 mmol, 1.6 equiv). A solution of acid 11 (920 mg, 1.6 mmol, 1.4 equiv) in methylene chloride (4 mL) was added to the reaction mixture in a dropwise fashion, which was warmed to room temperature. The reaction was concentrated after 4 h, and purified using silica gel chromatography employing 15% EtOAc/hexane as the eluent to afford ester 19 (1.0 g, 88% yield) as a clear oil: [α]_{D} +9.0° (c 0.5, CHCl₃); IR (neat) 3445, 2957, 1759, 1733, 1699, 1382, 1249, 1096 cm⁻¹; ¹H NMR (400 MHz, CHCl₃) δ7.17 (s, 1H), 6.74 (dd, J=17.3, 10.8 Hz, 1H) 6.52 (s, 1H), 5.73-5.65 (m, 1H), 5.50 (s, 2H), 5.31 (t, J=6.9 Hz, 1H), 5.27-5.22 (m, 2H), 5.14 (d, J=10.8 Hz, 1 H), 5.05-4.97 (m, 2H), 4.83 (d, J=11.7 Hz, 1H), 4.83 (s, 2H), 4.73 (m, 1H), 4.67 (d, J=12.1 Hz, 1H), 4.21 (dd, J=6.9, 3.0 Hz, 1H), 3.50-3.46 (m, 1H), 2.70-2.54 (m, 3H), 2.23-2.17 (m, 2H), 2.06 (s, 3H), 1.87 (m, 2H), 1.81 (s, 3H), 1.31 (s, 3H), 1.02 (d, J=8.3 Hz, 3H), 1.00 (s, 3H), 0.98-0.94 (m, 12H), 0.64 (q, J=7.8 Hz, 6H); ¹³C NMR (100 MHz, CDCl₃) δ215.2, 171.2, 161.4, 154.0, 153.5, 153.2, 138.0, 135.6, 134.9, 133.2, 124.5, 120.4, 118.4, 117.1, 114.5, 94.6, 81.6, 79.1, 75.1, 66.5, 53.3, 42.2, 39.6, 36.5, 34.3, 31.5, 30.9, 22.6, 22.2, 20.8, 19.8, 15.5, 14.5, 14.1, 10.5, 6.5, 4.9; HRMS (FAB) calcd. for C₄₁,H₅₉Cl₆NNaO₁₀SSi (M+Na⁺) 1018.1657, found 1018.1675.

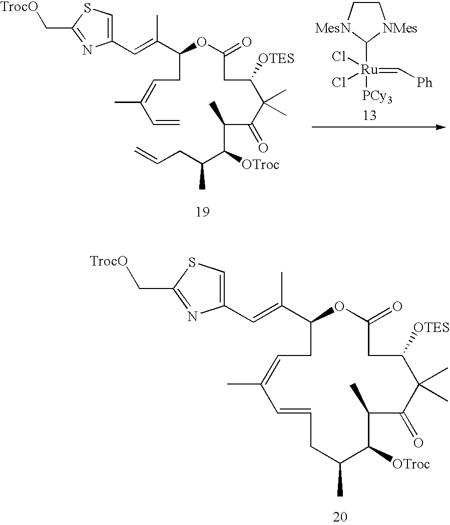

Compound 20. A solution of 19 (90 mg, 0.09 mmol) and tricyclohexylphosphine[1,3-bis(2,4,6-trimethylphenyl)-4,5-dihydroimidazole-2-ylidene-[benzylidine]ruthenium(IV) dichloride (Grubb's catalyst) (13) (10 mg, 0.009 mmol, 0.1 equiv) in 40 mL of methylene chloride was stirred at 35° C. for 5.5 hr. The solution was cooled to room temperature and passed through a plug of silica gel using 50% hexane/EtOAc. The combined filtrate was concentrated in vacuo and purified using silica gel chromatography employing 20% EtOAc/hexane as the eluent, affording diene 20 (35 mg, 40% yield): [α]_{D} −16.6° (c 0.75, CHCl₃); IR (neat) 2955, 1760, 1699, 1440, 1383, 1294, 1161, 1112, 1021 cm⁻¹; ¹H NMR (400 MHz, CDCl₃) δ7.19 (s, 1H), 6.74 (d, J=15.6 Hz, 1H), 6.58 (s, 1H), 5.66 (ddd, J=14.4, 10.6, 2.8 Hz, 1H), 5.51 (s, 2H), 5.42 (t, J=8.3 Hz, 1H), 5.22 (d, J=8.8 Hz, 1H), 5.11 (d, J=10.1 Hz, 1H), 4.84-4.77 (m, 4H), 4.04 (d, J=8.4 Hz, 1H), 3.30 (m, 1H), 2.91-2.80 (m, 2H), 2.56 (dd, J=16.6, 9.5 Hz, 1H), 2.41 (t, J=11.5 Hz, 1H), 2.20-2.12 (m, 5H), 1.91-1.83 (m, 1H), 1.78 (s, 3H), 1.19 (s, 3H), 1.14-1.12 (m, 9H), 0.87 (t, J=8.0 Hz, 9H), 0.64 (q, J=8.0 Hz, 6H); ¹³C NMR (100 MHz, CDCl₃) 62 21.20, 170.1, 165.5, 154.4, 135.6, 153.2, 139.1, 136.3, 129.6, 127.6, 123.3, 119.4, 118.4, 94.7, 94.0, 84.6, 79.5, 77.2, 75.3, 66.5, 53.6, 44.9, 39.3, 35.9, 35.1, 31.8, 29.6, 24.1, 22.5, 20.5, 18.8, 15.5, 14.7, 6.9, 5.1; HRMS (FAB) calcd. for C₃₉H₅₅Cl₆NNaO₁₀SSi (M+Na⁺) 990.1344, found 990.1380.

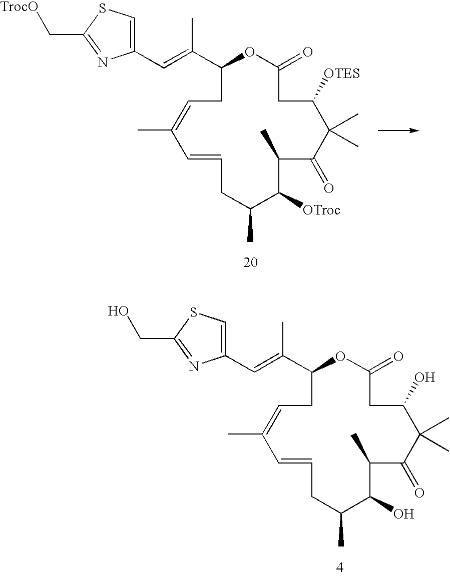

Compound 4. A stirred solution of 20 (48 mg, 0.05 mmol) in 1:1 THF/HOAc (2 mL) was treated with Zn° (15 mg, freshly activated by washing with dil. HCl and drying under vacuum). The reaction mixture was sonicated for 20 min and then filtered through celite, washing the celite cake with EtOAc. The combined filtrate was washed with saturated NaHCO₃, (10 mL), brine (10 mL), and dried over MgSO₄. Removal of the solvent in vacuo followed by purification of the residue on silica gel chromatography using 25% EtOAc/hexane as the eluent yielded the C7 alcohol from 20 (21 mg, 70% ): [α]_{D} −67.2° (c 1.15, CHCl₃); IR (neat) 3409, 2955, 1734, 1684, 1456, 1380, 1240 cm⁻¹; ¹H NMR (400 MHz, CDCl₃) δ7.12 (s, 1H), 6.60 (s, 1H), 6.56 (d, J=16.0 Hz, 1H), 5.76 (m, 1H), 5.34 (d, J=10.0 Hz, 1H), 5.33-5.27 (m, 1H), 4.37 (t, J=6.1 Hz, 1H), 3.65 (d, J=5.3 Hz, 1H), 3.54 (bs, 1H), 3.16 (m, 1H), 3.01-2.92 (m, 1H), 2.61-2.56 (m, 1H), 2.55-2.48 (m, 1H), 2.21-2.14 (m, 5H), 2.07-2.05 (m, 2H), 1.77 (s, 3H), 1.15 (s, 3H). 1.12 (s, 3H), 1.11 (d, J=6.7 Hz, 6H), 0.89 (t, J=7.9 Hz, 9H), 0.56 (q, J=7.9 Hz, 6H) ¹³C NMR (100 MHz, CDCl₃) δ219.8, 169.9, 169.6, 152.5, 138.2, 135.6, 129.6, 129.0, 123.0, 120.5, 117.1, 79.3, 73.7, 71.4, 62.2, 54.9, 40.6, 40.0, 36.5, 35.7, 32.2, 21.0, 20.3, 20.0, 16.6, 14.9, 11.9, 6.9, 5.3; HRMS (FAB) calcd. for C₃₃H₅₃NNaO₆SSi (M+Na⁺) 642.3260, found 642.3258.

HF.Py (0.1 mL) was added to a solution of the C7 alcohol from 20 (6 mg, 0.008 mmol) in THF (0.5 mL). The resulting solution was stirred at room temperature for 3 h, and then carefully poured into saturated NaHCO₃ solution, which was extracted with EtOAc (3×5 mL). The combined organic layers were washed with brine (5 mL), dried over MgSO₄ and concentrated in vacuo. The residue was purified using silica gel chromatography employing 60% EtOAc/hexane as the eluent, which furnished 4 (4 mg, 80% yield): [α]_{D} −73.1° (c 0.45, CHCl₃); IR (neat) 3045, 2918, 1718, 1678, 1448, 1384, 1255, 1149 cm⁻¹; ¹H NMR (500 MHz, CDCl₃) δ7.05 (s, 1H), 6.55 (s, 1H), 6.50 (d, J=15.6 Hz, 1H), 5.78 (ddd, J=14.7, 8.6, 5.6 Hz, 1H), 5.31-5.26 (m, 2H), 4.96 (s, 2H), 4.25 (dd, J=10.0, 2.8 Hz, 1H), 3.70 (d, J=5.4 Hz, 1H), 3.26 (m, 1H), 3.15 (bs, 1H), 2.82-2.75 (m, 1H), 2.56-2.53 (m, 1H), 2.44 (dd, J=15.3, 10.4 Hz, 1H), 2.37-2.30 (m, 2H), 2.12-2.06 (m, 4H), 2.03-1.98 (m, 1H), 1.80 (s, 3H), 1.32 (s, 3H), 1.12 (d, J=6.8 Hz, 3H), 1.07 (d, J=7.1 Hz, 3H), 1.04 (s, 3H); ¹³C NMR (125 MHz, CDCl₃) δ220.1, 170.3, 169.9, 149.4, 139.0, 135.7, 129.4, 129.2, 122.9, 118.5, 116.6, 77.9, 71.7, 71.6, 61.5, 53.4, 41.1, 39.4, 36.8, 36.0, 32.0, 21.4, 21.0, 18.7, 15.3, 11.3; LRMS (ESI) calcd. for C₂₇H₃₉NO₆S 505.2, found 528.2 (M+Na⁺).

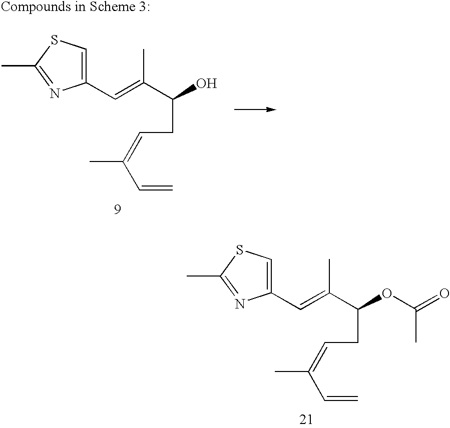

Compound 21. A solution of alcohol 9 (268 mg, 1.1 mmol) in methylene chloride; (10 mL) was treated with DMAP (200 mg, 1.6 mmol, 1.5 equiv), triethylamine (0.75 mL, %0.3 mmol, %0.0 equiv) and acetic anhydride (0.3 mL, 3.22 mmol, 3.0 equiv) and stirred at rt for 45 min. The reaction mixture was diluted with Et₂O (25 mL), washed with a saturated solution of NaHCO₃(2×15 mL), brine (15 mL), dried over MgSO₄ and concentrated in vacuo. Purification using silica gel chromatography employing 20% EtOAc/hexane as the eluent afforded acetate 21 (312 mg, 98% yield) as a clear oil: [α]_{D} −23.8° (c 1.12, CHCl₃); IR (neat) 1734, 1652, 1558, 1368, 1236, 1018 cm⁻¹; ¹H NMR (400 MHz, CDCl₃) δ6.96 (s, 1H), 6.76 (dd, J=17.3, 10.8 Hz, 1H), 6.54 (s, 1H), 5.35-5.28 (m, 2H), 5.24 (d, J=17.2 Hz, 1H), 5.13 (d, J=10.8 Hz, 1H), 2.71 (s, 3H), 2.68-2.63 (m, 1H), 2.58-2.51 (m, 1H), 2.08 (s, 3H), 2.06 (s, 3H), 1.81 (s, 3H); ¹³C NMR (100 MHz, CDCl₃) δ170.1, 164.5, 152.4, 137.1, 134.8, 133.3, 124.7, 120.6, 116.2, 114.3, 78.4, 31.0, 21.1, 19.8, 19.1, 14.8; HRMS (FAB) calcd. for C₁₆H₂₂NO₂S (M+H⁺) 292.1371, found 292.1378.

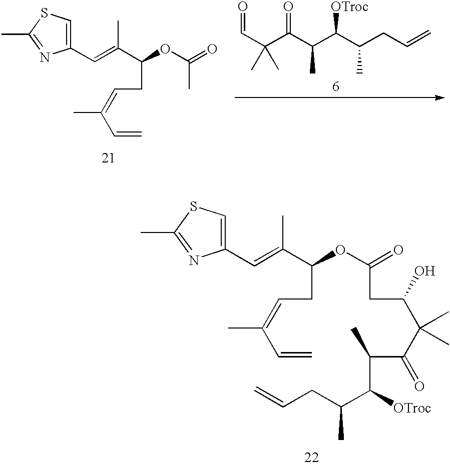

Compound 22. A solution of acetate 21 (310 mg, 1.0 mmol, 1.6 equiv) in Et₂O (1.5 mL) was cooled to −78° C. and added to a freshly prepared solution of LDA (1.1 mL of a 1.0 M solution in Et₂O, 1.1 mmol, 1.7 equiv) in Et₂O (2 mL) at −78° C. The reaction mixture was stirred for 70 min, followed by the addition of CpTiCl(OR)₂ (12.5 mL of a 0.1 M solution in Et₂O, 1.2 mmol, 1.9 equiv) in a dropwise fashion. The reaction was maintained at −78° C. for 15 min, warmed to −30° C. for 1 h, and again cooled back to −78° C. for 15 min. A solution of aldehyde 6 (233 mg, 0.6 mmol) in Et₂O (1 mL) was added to the reaction mixture in a dropwise fashion over 15 min. The reaction mixture was maintained at −78° C. for 75 min, quenched with 5 mL of a solution of H₂O: THF (1:9), warmed to rt stirred for 2 h. The suspension was filtered through celite, diluted with Et₂O (10 mL), and washed with brine (15 mL). The aqueous layer was extracted with Et₂O (2×15 mL). The combined organic layers were dried over over MgSO₄ and concentrated in vacuo. Purification using silica gel chromatography employing 12% EtOAc/hexane as the eluent afforded aldol adduct 22 (340 mg, 85% yield) as a yellow oil: [α]_{D} +1.7° (c 1.4, CHCl₃); IR (neat) 1757, 1733, 1699, 1558, 1456, 1381, 1240, 1178 cm⁻¹; ¹H NMR (500 MHz, CDCl₃) δ6.98 (s, 1H), 6.75 (dd, J=17.2, 10.8 Hz, 1H), 6.54 (s, 1H), 5.74-5.65 (m, 1H), 5.35-5.30 (m, 2H), 5.25 (d, J=17.3 Hz, 1H), 5.14 (d, J=10.9 Hz, 1H), 5.10-5.04 (m, 2H), 4.87 (t, J=6.2 Hz, 1H), 4.84 (d, J=12.0 Hz, 1H), 4.73 (d, J=11.9 Hz, 1H), 4.18 (d, J=10.5 Hz, 1H), 3.49-3.42 (m, 1H), 3.21 (d, J=3.6 Hz, 1H), 2.72 (s, 3H), 2.69-2.65 (m, 1H), 2.59-2.53 (m, 1H), 2.49 (dd, J=16.3, 1.9 Hz, 1H), 2.34 (dd, J=16.4, 5.8 Hz, 1H), 2.33-2.26 (m, 1H), 2.10 (s, 3H), 1.95-1.89 (m, 2H), 1.81 (s, 3H), 1.22 (s, 3H), 1.18 (s, 3H), 1.12 (d, J=6.8 Hz, 3H), 0.96 (d, J=6.4 Hz, 3H); ¹³C NMR (125 MHz, CDCl₃) δ215.7, 172.1, 164.7, 154.2, 152.3, 136.7, 135.8, 135.1, 133.3, 124.5, 121.1, 117.0, 116.5, 114.6, 94.6, 82.4, 79.2, 72.9, 51.9, 41.4, 36.5, 36.0, 34.3, 31.0, 22.0, 19.8, 19.2, 19.0, 16.0, 14.7, 11.9; HRMS (FAB) calcd. for C₃₂H₄₄Cl₃NNaO₇S (M+Na⁺) 714.1801, found 714.1799.

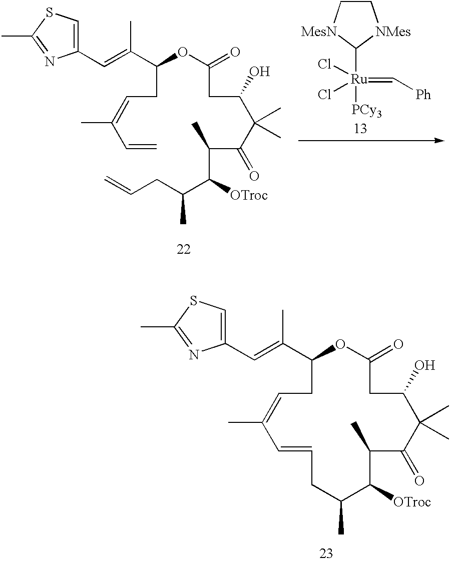

Compound 23. A solution of 22 (21 mg, 0.03 mmol) and tricyclohexylphosphine[1,3-bis(2,4,6-trimethylphenyl)-4,5-dihydroimidazole-2-ylidene-[benzylidine]ruthenium(IV) dichloride (Grubb's catalyst) (13) (2 mg, 0.003 mmol, 0.1 equiv) in 15 mL of methylene chloride was stirred at 35° C. for 5.5 hr. The solution was cooled to room temperature and passed through a plug of silica gel using 60% hexane/EtOAc. The combined filtrate was concentrated in vacuo and purified using silica gel chromatography employing 30% EtOAc/hexane as the eluent, affording diene 23 (8 mg, 41% ): [α]_{D} −46.0° (c 0.1, CHCl₃); IR (neat) 3420, 1757, 1717, 1699 1558, 1452, 1394, 1240 cm⁻¹; ¹H NMR (500 MHz, CDCl₃) δ6.91 (s, 1H), 6.49 (d, J=15.0 Hz, 1H), 6.49 (s, 1H), 5.67 (ddd, J=15.0, 9.5, 4.3 Hz, 1H), 5.29 (dd, J=10.0, 10.0, 1H), 5.24 (d, J=8.8 Hz, 1H), 5.06 (dd, J=7.5, 2.9 Hz, 1H), 4.82 (d, J=12.0 Hz, 1H), 4.72 (d, J=12.0 Hz, 1H), 3.97 (dd, J=9.8, 2.0 Hz, 1H), 3.46 (dq, J=6.9, 6.9 Hz, 1H), 3.07 (bs, 1H), 2.76 (ddd, J=14.0, 10.0, 8.8 Hz, 1H), 2.64 (s, 3H), 2.46 (dd, J=15.8, 2.0 Hz, 1H), 2.38 (dd, J=15.8, 9.8 Hz, 1H), 2.31-2.20 (m, 2H), 2.14-2.08 (m, 1H), 2.06 (s, 3H), 1.76-1.73 (m, 1H), 1.73 (s, 3H), 1.22 (s, 3H), 1.11 (d, J=6.9 Hz, 3H), 1.05 (d, J=6.9 Hz, 3H), 1.00 (s, 3H); ¹³C NMR (125 MHz, CDCl₃) δ216.1, 170.2, 164.9, 154.3, 135.5, 129.1, 128.2, 124.9, 123.3, 120.0, 116.4, 112.1, 94.7, 83.3, 79.2, 72.3, 52.3, 43.4, 38.8, 36.0, 34.6, 31.7, 21.8, 20.8, 20.3, 19.1, 18.2, 15.3, 14.7, 12.9; LRMS (ESI) calcd. for C₃₀H₄₀Cl₃NO₇S 663.1, found 686.1 (M+Na⁺), 664.1 (M+H⁺).

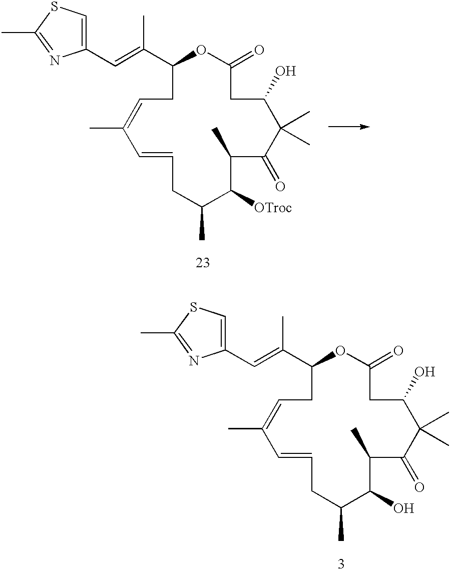

Epothilone 490. A solution of carbonate 23 (8 mg, 0.01 mmol) in 0.5 mL of THF:AcOH (1:1) was treated with Zn (1 mg, nanosize). The reaction mixture was sonicated for 10 min and then filtered through celite, washing the celite cake with EtOAc. The combined filtrate was washed with saturated NaHCO₃ (2 mL), brine (2 mL), and dried over MgSO4. Removal of the solvent in vacuo followed by purification of the residue on silica gel chromatography using 35% EtOAc/hexane as the eluent yielded the epothilone 490 (3) (5 mg, 86% yield).

Representative RCM Reactions from Table 1:

Methylene Chloride as Solvent.

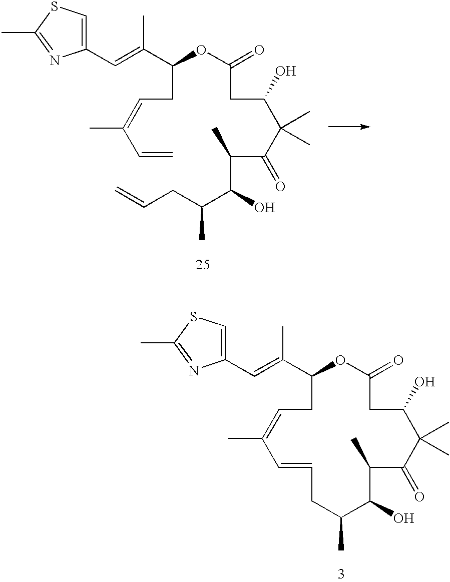

A solution of 25 (56 mg, 0.1 mmol) and tricyclohexylphosphine[1,3-bis(2,4,6-trimethylphenyl)-4,5-dihydroimidazole-2-ylidene-[benzylidine]ruthenium(IV) dichloride 13 (10.0 mg, 0.01 mmol, 0.1 equiv) in 50 mL of CH₂Cl₂, was stirred at 35° C. for 5 hr. The solution was cooled to room temperature and treated with 5 mL dimethyl sulfoxide and stirred at rt for 12 h to remove ruthenium impurities. The reaction mixture was passed through a plug of silica gel using 50% hexane/EtOAc. The combined filtrate was concentrated in vacuo and purified using silica gel chromatography employing 35% EtOAc/hexane as the eluent, affording epothilone 490 (33 mg, 64% ) as a white solid.

1 mmol Scale RCM in Toluene as Solvent.

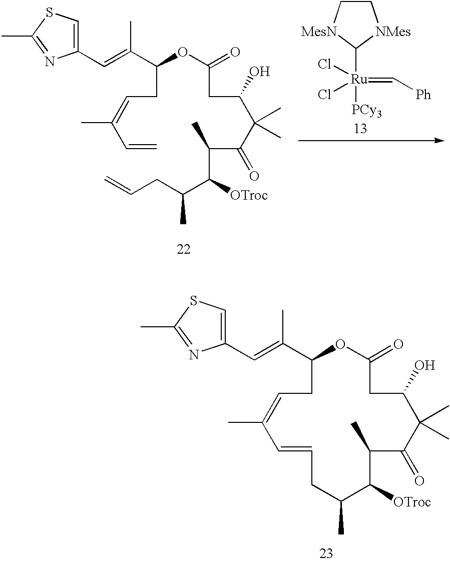

A solution of 22 (700 mg, 1 mmol) in toluene (500 mL) was heated to 110° C. and treated with tricyclohexylphosphine[1,3-bis(2,4,6-trimethylphenyl)-4,5-dihydroimidazole-2-ylidene-[benzylidine]ruthenium(IV) dichloride 13 (85 mg, 0.1 mmol, 0.1 equiv). The reaction was stirred for 25 min, cooled to rt, filtered through a plug of silica gel using 50% hexane/EtOAc as eluant. The combined filtrate was concentrated in vacuo and purified using silica gel chromatography employing 35% EtOAc/hexane as the eluent, affording 23 (370 mg, 57% ) as a white solid.

Scheme 4:

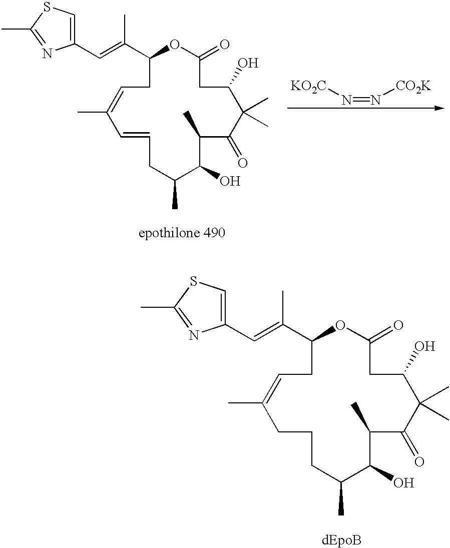

dEpoB. A solution of 0.5 M AcOH (0.12 mL) in CH₂Cl₂ was added dropwise to a mixture of diene 3 (14.4 mg, 0.029 mmol), potassium diazodicarboxylate (68 mg, 0.24 mmol, 12 equiv) and CH₂Cl₂ (5 mL) at reflux. The resulting mixture was heated at reflux. The reaction was monitored by HPLC (reverse phase, Dynamax 60A C18 column, 4.6×300 mm, water/acetonitrile 1:1) until the starting material was consumed (24 h). The reaction was cooled to rt and filtered on a plug of silica gel, which was rinsed with EtOAc. The combined filtrate was concentrated in vacuo and the residue was purified by silica gel chromatography employing 50% EtOAc/hexane as the elaunt, to give 12.3 mg (86% ) of 1 as a white solid. The spectral data of 1 was identical to those reported of dEpoB.

### Example 12

In vitro Studies

A typical experiment invovles culturing cells (e.g., CCRF-CEM) at an initial density of 2-5×10⁴ cells per ml. They are maintained in a 5% CO₂-humidified atmosphere at 37° C. in RPMI medium 1640 (GIBCO/BRL) containing penicillin (100 units/ml), streptomycin (100 μg/ml) (GIBCO/BRL), and 5% heat-inactivated fetal bovine serum. For cells that were grown in suspension (such as CCRF-CEM and its sublines), cytotoxicitiy is measured by using the 2,-3-bis(2-methoxy-4-nitro-5-sulfophenyl)-5 carboxanilide)-2H terazodium hydroxide (XTT)-microculture tetrazonium method in duplicate in 96-well microtiter plates. For both methods, the absorbance of each well is measured with a microplate reader (EL-340, Bio-Tek, Burlington, Vt.). Each run entails six or seven concentrations of the tested drugs. Dose-effect relationship data are analyzed with the median-effect plot.

The CCRF-CEM human T cells, acute lymphoblastic leukemic cells, its teniposide-resistant subline (CCRF-CEM/VM₁) and vinblastine-resistant subline (CCRF-CEM/VBL₁₀₀) are obtained from W. T. Beck (University of Illinois, Chicago, Ill.).

In a typical experiment, as outlined generally above, certain of the inventive compounds (e.g., Epo 490, 26-fluoro-dEpoB; 10, 11-di-OH-dEpoB; 10,11-di-dehydro-dEpoF; and 10, 11-ketal-dEpoB) demonstrated activity in CCRF-CEM cell lines and CCRF-CEM cell lines resistant to Taxol. Certain of these compounds exhibit IC₅₀s in the range of 0.0015 to about 0.120 for CCRF-CEM cell lines. Certain other compounds exhibit IC₅₀s in the range of 0.0015 to about 10.5. Certain of these compounds also exhibit IC₅₀s in the range of 0.011 to about 0.80 for CCRF-CEM/Taxol resistant cell lines and certain other compounds exhibit IC₅₀s in the range of about 0.011 to about 13.0 μM. In certain embodiments, 26F-EpoD exhibits activities in the range of 0.0015 μM for CCRF-CEM cell lines and in the range of 0.011 μM for CCRF-CEM/Taxol resistant cell lines.

Additional studies have been performed to test the ability of a 17-membered ring analogue, Homo-epo-490 (Homo-ddEpoB) to inhibit the growth of tumor cell lines. Specifically, for CCRF-CEM tumor cell lines, Homo-Epo-490 exhibits activity in the range of 0.051 μM. For CCRF-CEM/VBL₁₀₀ resistant cell lines, Homo-Epo-490 exhibits activity in the range of 0.137 μM. For CCRF-CEM/VM₁ resistant cell lines, Homo-Epo-490 exhibits activity in the range of 0.055 μM. For CCRF-CEM/Taxol resistant cell lines, Homo-Epo-490 exhibits activity-in the range of 0.049 μM.

### Example 13

In vivo Studies

Athymic nude mice bearing the nu/nu gene are typically used for tumor xenografts. Outbred, Swiss-background mice were obtained from Charles River Laboratories. Male mice 8 weeks or older weighing 22 g and up were used for most experiments. The drug was administered via the tail vein for 6 hr.—i.v. infusion. Each individual mouse was confined in a perforated Falcon polypropylene tube restrainer for drug administration. Tumor volume was assessed by measuring length×width×height (or width) using a caliper. The programmable Harvard PHD2000 syringe pump (Harvard Apparatus) with multi-track was used for i.v. infusion. All animal studies were conducted in accordance with the guidelines of the National Institutes of Health “Guide for the Care and Use of Animals” and the protocol approved by the Memorial Sloan-Kettering Cancer Center's Institutional Animal Care and Use Committee. In keeping with the policy of this committee for the humane treatment of tumor-bearing animals, mice were euthanized when tumors reached ≧10% of their total body weight.

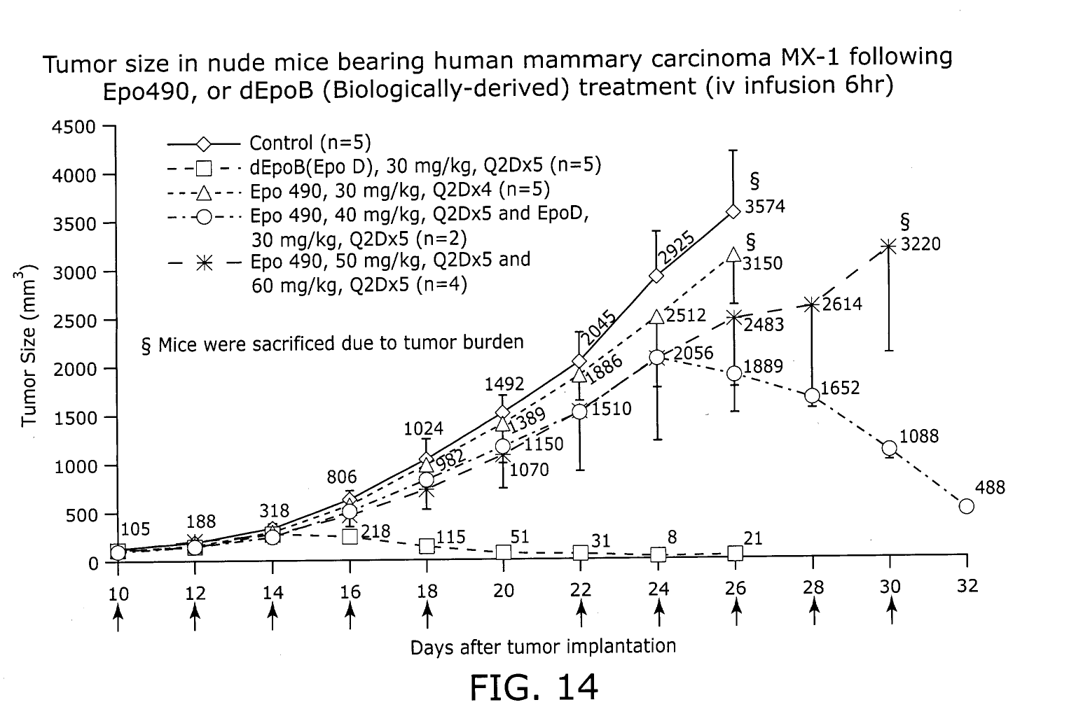

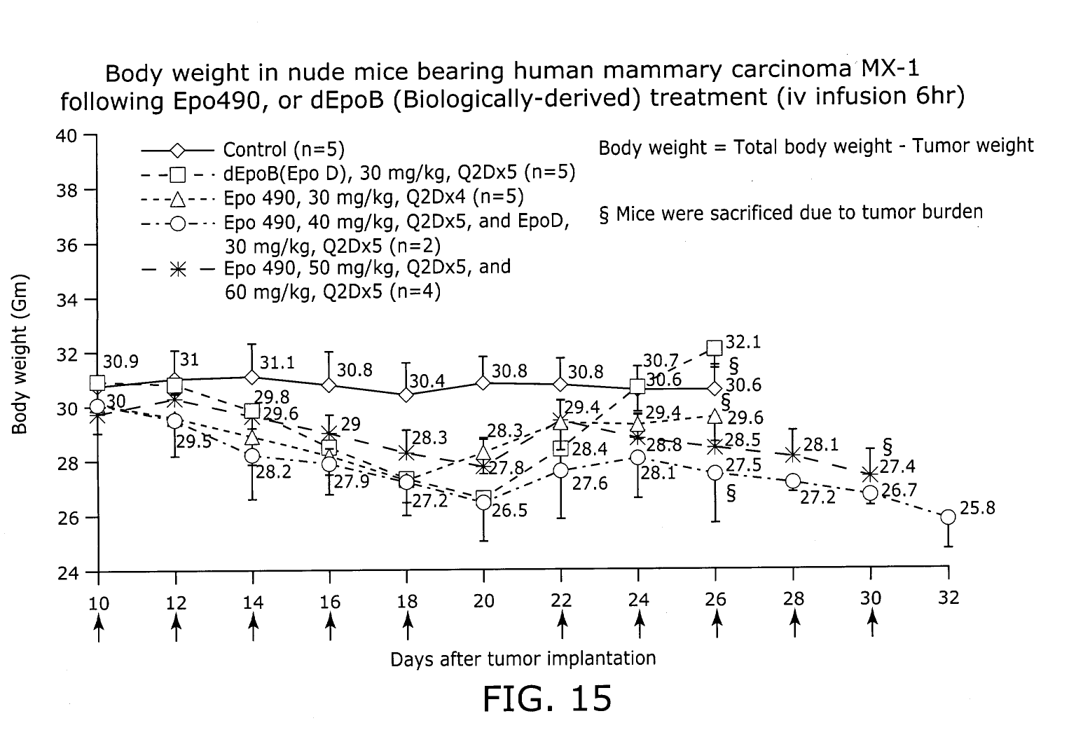

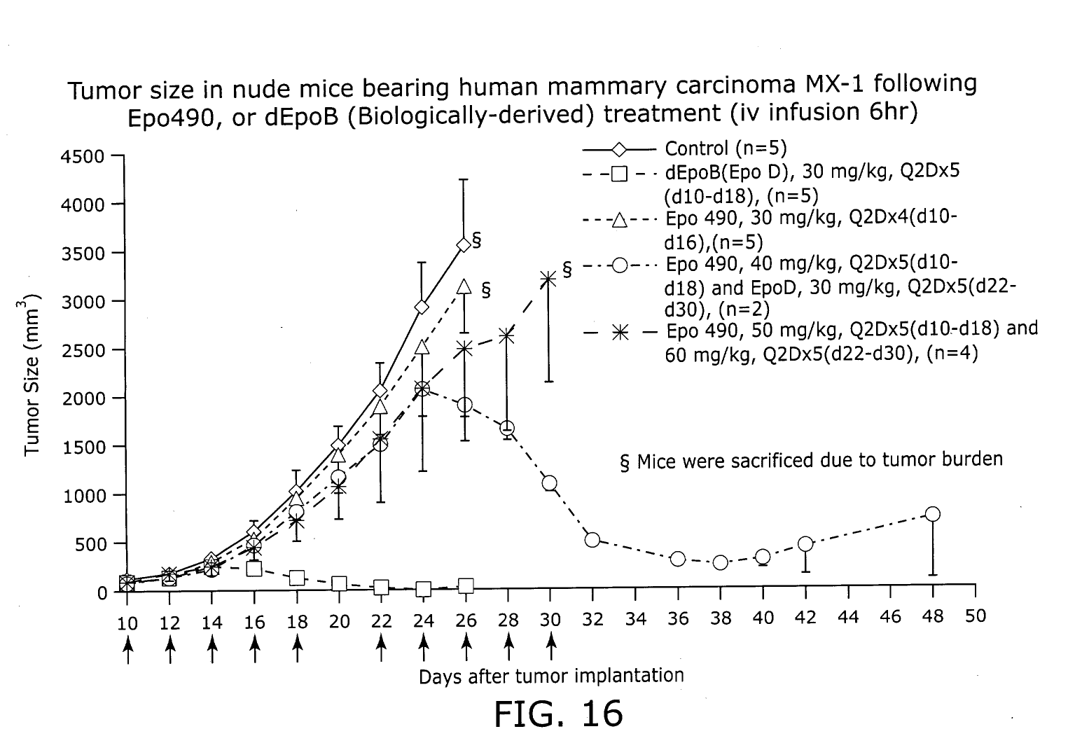

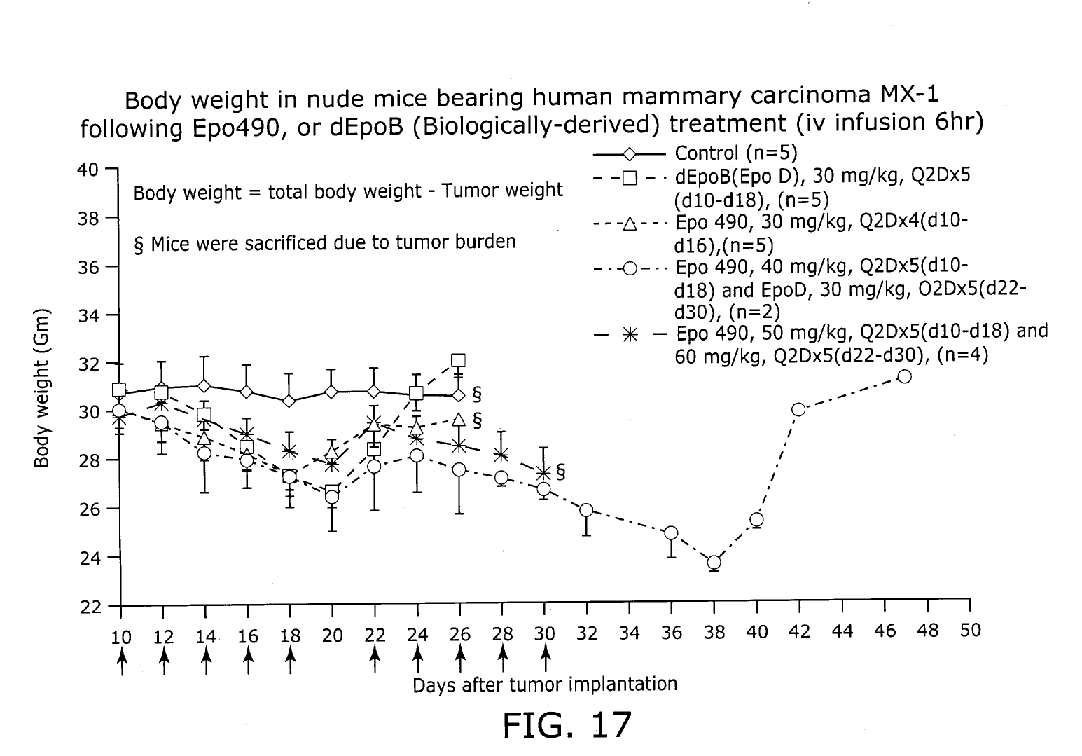

As depicted in FIGS. 14, 15, 16, and 17, Epo490 was tested in nude mice bearing human mammary carcinoma MX-1 following treatment with Epo490 or dEpoB (i.v. infusions for 6 hours). In general, Epo490 was formulated as follows: Epo-490 was dissolved in ethanol and Cremophor was added (1:1) at a concentration of 20 mg/ml. 90 ml of this solution was diluted with 2 ml of saline (total volume 2.09 μl, and concentration: 1.8 mg/2.90 ml=0.861 mg/ml). The diluted solution was used for i.v. infusion within one hour. Tumor size and body weight were then measured using dosages of 30 mg/kg, 40 mg/kg or 50 mg/kg over 32 and 50 days.

1. A compound of formula (I): 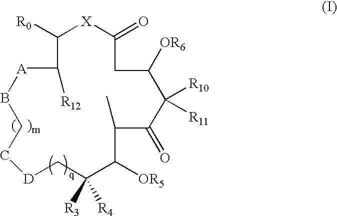
wherein R₀ is a substituted or unsubstituted aryl, heteroaryl, arylalkyl, arylalkenyl, arylalkynyl, or heteroarylalkyl, heteroarylalkenyl, heteroarylalkynyl moiety;
R₃ and R₄ are each independently hydrogen; or substituted or unsubstituted, linear or branched, cyclic or acyclic aliphatic, heteroaliphatic, aryl, heteroaryl, arylalkyl, arylalkenyl, arylalkynyl, or heteroarylalkyl, heteroarylalkenyl, heteroarylalkynyl moiety, optionally substituted by one or more of hydroxy, protected hydroxy, alkoxy, carboxy, carboxaldehyde, linear or branched alkyl or cyclic acetal, fluorine, amino, protected amino, amino substituted with one or two alkyl or aryl moieties, N-hydroximino, or N-alkoxyimino;
R₅ and R₆ are each independently hydrogen or a protecting group;
R₁₀ and R₁₁ are each independently hydrogen; or substituted or unsubstituted, linear or branched, cyclic or acyclic aliphatic, heteroaliphatic, aryl, heteroaryl, arylalkyl, arylalkenyl, arylalkynyl, or heteroarylalkyl, heteroarylalkenyl, heteroarylalkynyl moiety, optionally substituted by one or more of hydroxy, protected hydroxy, alkoxy, carboxy, carboxaldehyde, linear or branched alkyl or cyclic acetal, fluorine, amino, protected amino, amino substituted with one or two alkyl or aryl moieties, N-hydroximino, or N-alkoxyimino;
R₁₂ is hydrogen; halogen, hydroxy, alkoxy, amino, dialkylamino, alkylamino, fluoro, or substituted or unsubstituted, linear or branched, cyclic or acyclic aliphatic, heteroaliphatic, aryl, heteroaryl, arylalkyl, arylalkenyl, arylalkynyl, or heteroarylalkyl, heteroarylalkenyl, heteroarylalkynyl moiety, optionally substituted by one or more of hydroxy, protected hydroxy, alkoxy, carboxy, carboxaldehyde, linear or branched alkyl or cyclic acetal, fluorine, amino, protected amino, amino substituted with one or two alkyl or aryl moieties, N-hydroximino, or N-alkoxyimino;
X is O, S, C(R₇)₂, or NR₇, wherein each occurrence of R₇ is independently hydrogen or lower alkyl;
m is an integer from 0 to 3 and q is an integer from 1 to 3 with the limitation that the sum of m+q is an integer from 1 to 4;
A—B represents CR_{A}═CR_{B}—; C(R_{A})₂—C(R_{B})₂—; or C(R_{A})₂—CR_{B}═;
C—D represents —CR_{C}═CR_{D}—; —C(R_{C})₂—C(R_{D})₂—; ═CR_{C}—C(R_{D})₂—; or —C≡C—;
when m is 0, B—C represents ═CR_{B}—CR_{C}═; —C(R_{B})₂—CR_{C}═; ═CR_{B}—C(R_{C})₂—; ═CR_{B}—C≡; or —C(R_{B})₂—C(R_{C})₂—;
wherein each occurrence of R_{A} independently hydrogen; halogen; —OR_{A′}; —SR_{A′}; —N(R_{A′})₂; —C(O)OR_{A′}; —C(O)R_{A′}; —CONHR_{A′}; —O(C═O)R_{A′}; —O(C═O)OR_{A′}; —NR_{A′}(C═O)R_{A′}; N₃; N₂R_{A′}; cyclic acetal; or cyclic or acyclic, linear or branched aliphatic, heteroaliphatic, aryl, or heteroaryl, optionally substituted with one or more of hydrogen; halogen; —OR_{A′}; —SR_{A′}; —N(R_{A′})₂; —C(O)OR_{A′}; —C(O)R_{A′}; —CONHR_{A′}; —O(C═O)R_{A′}; —O(C═O)OR_{A′}; —NR_{A′}(C═O)R_{A′}; N₃; N₂R_{A′}; cyclic acetal; or cyclic or acyclic, linear or branched substituted or unsubstituted aliphatic, heteroaliphatic, aryl, or heteroaryl moiety; or is an epothilone, desoxyepothilone, or analogues thereof; or is a polymer; carbohydrate; photoaffinity label; or radiolabel;
R_{B} is, independently for each occurrence, hydrogen; halogen; —OR_{B′}; —SR_{B′}; —N(R_{B′})₂; —C(O)OR_{B′}; —C(O)R_{B′}; —CONHR_{B′}; —O(C═O)R_{B′}; —O(C═O)OR_{B′}; —NR_{B′}(C═O)R_{B′}; N₃; N₂R_{B′}; cyclic acetal; or cyclic or acyclic, linear or branched aliphatic, heteroaliphatic, aryl, or heteroaryl, optionally substituted with one or more of hydrogen; halogen; —OR_{B′}; —SR_{B′}; —N(R_{B′})₂; —C(O)OR_{B′}; —C(O)R_{B′}; —CONHR_{B′}; —O(C═O)R_{B′}; —O(C═O)OR_{B′}; —NR_{B′}(C═O)R_{B′}; N₃; N₂R_{B′}; cyclic acetal; or cyclic or acyclic, linear or branched substituted or unsubstituted aliphatic, heteroaliphatic, aryl, or heteroaryl moiety; or is an epothilone, desoxyepothilone, or analogues thereof; or is a polymer; carbohydrate; photoaffinity label; or radiolabel;
R_{C} is, independently for each occurrence, hydrogen; halogen; —OR_{C′}; —SR_{C′}; —N(R_{C′})₂; —C(O)OR_{C′}; —C(O)R_{C′}; —CONHR_{C′}; —O(C═O)R_{C′}; —O(C═O)OR_{C′}; —NR_{C′}(C═O)R_{C′}; N₃; N₂R_{C′}; cyclic acetal; or cyclic or acyclic, linear or branched aliphatic, heteroaliphatic, aryl, or heteroaryl, optionally substituted with one or more of hydrogen; halogen; —OR_{C′}; —SR_{C′}; —N(R_{C′})₂; —C(O)OR_{C′}; —C(O)R_{C′}; —CONHR_{C′}; —O(C═O)R_{C′}; —O(C═O)OR_{C′}; —NR_{C′}(C═O)R_{C′}; N₃; N₂R_{C′}; cyclic acetal; or cyclic or acyclic, linear or branched substituted or unsubstituted aliphatic, heteroaliphatic, aryl, or heteroaryl moiety; or is an epothilone, desoxyepothilone, or analogues thereof; or is a polymer; carbohydrate; photoaffinity label; or radiolabel;
R_{D} is, independently for each occurrence, hydrogen; halogen; —OR_{D′}; —SR_{D′}; —N(R_{D′})₂; —C(O)OR_{D′}; —C(O)R_{D′}; —CONHR_{D′}; —O(C═O)R_{D′}; —O(C═O)OR_{D′}; —NR_{D′}(C═O)R_{D′}; N₃; N₂R_{D′}; cyclic acetal; or cyclic or acyclic, linear or branched aliphatic, heteroaliphatic, aryl, or heteroaryl, optionally substituted with one or more of hydrogen; halogen; —OR_{D′}; —SR_{D′}; —N(R_{D′})₂; —C(O)OR_{D′}; —C(O)R_{D′}; —CONHR_{D′}; —O(C═O)R_{D} ; —O(C═O)OR_{D′}; —NR_{D′}(C═O)R_{D′}; N₃; N₂R_{D′}; cyclic acetal; or cyclic or acyclic, linear or branched substituted or unsubstituted aliphatic, heteroaliphatic, aryl, or heteroaryl moiety; or is an epothilone, desoxyepothilone, or analogues thereof; or is a polymer; carbohydrate; photoaffinity label; or radiolabel; or
wherein any two of R_{A}, R_{B}, R_{C} or R_{D} taken together may form a cyclic moiety and may be linked through an oxygen, sulfur, carbon or nitrogen atom, or any two adjacent groups R_{A}, R_{B}, R_{C}, or R_{D}, taken together, may form a 3-6-membered substituted or unsubstituted aliphatic, heteroaliphatic, aryl or heteroaryl ring;
wherein each occurrence of R_{A′}, R_{B′}, R_{C′} and R_{D′} is independently hydrogen; a protecting group; a linear or branched, substituted or unsubstituted, cyclic or acyclic, aliphatic, heteroaliphatic, aryl, heteroaryl, arylalkyl, arylalkenyl, arylalkynyl, or heteroarylalkyl, heteroarylalkenyl, heteroarylalkynyl moiety; or is an epothilone, desoxyepothilone, or analogues thereof; or a polymer; carbohydrate; photoaffinity label; or radiolabel; and
pharmaceutically acceptable derivatives thereof.

2. The compound of claim 1 wherein the compound has the formula: 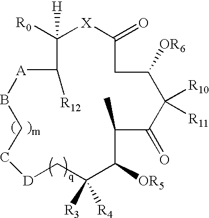
wherein R₀ is a substituted or unsubstituted aryl, heteroaryl, arylalkyl, arylalkenyl, arylalkynyl, or heteroarylalkyl, heteroarylalkenyl, heteroarylalkynyl moiety;
R₃ and R₄ are each independently hydrogen; or substituted or unsubstituted, linear or branched, cyclic or acyclic aliphatic, heteroaliphatic, aryl, heteroaryl, arylalkyl, arylalkenyl, arylalkynyl, or heteroarylalkyl, heteroarylalkenyl, heteroarylalkynyl moiety, optionally substituted by one or more of hydroxy, protected hydroxy, alkoxy, carboxy, carboxaldehyde, linear or branched alkyl or cyclic acetal, fluorine, amino, protected amino, amino substituted with one or two alkyl or aryl moieties, N-hydroximino, or N-alkoxyimino;
R₅ and R₆ are each independently hydrogen or a protecting group;
R₁₀ and R₁₁ are each independently hydrogen; or substituted or unsubstituted, linear or branched, cyclic or acyclic aliphatic, heteroaliphatic, aryl, heteroaryl, arylalkyl, arylalkenyl, arylalkynyl, or heteroarylalkyl, heteroarylalkenyl, heteroarylalkynyl moiety, optionally substituted by one or more of hydroxy, protected hydroxy, alkoxy, carboxy, carboxaldehyde, linear or branched alkyl or cyclic acetal, fluorine, amino, protected amino, amino substituted with one or two alkyl or aryl moieties, N-hydroximino, or N-alkoxyimino;
R₁₂ is hydrogen; halogen, hydroxy, alkoxy, amino, dialkylamino, alkylamino, fluoro, or substituted or unsubstituted, linear or branched, cyclic or acyclic aliphatic, heteroaliphatic, aryl, heteroaryl, arylalkyl, arylalkenyl, arylalkynyl, or heteroarylalkyl, heteroarylalkenyl, heteroarylalkynyl moiety, optionally substituted by one or more of hydroxy, protected hydroxy, alkoxy, carboxy, carboxaldehyde, linear or branched alkyl or cyclic acetal, fluorine, amino, protected amino, amino substituted with one or two alkyl or aryl moieties, N-hydroximino, or N-alkoxyimino;
X is O, S, C(R₇)₂, or NR₇, wherein each occurrence of R₇ is independently hydrogen or lower alkyl;
m is an integer from 0 to 3 and q is an integer from 1 to 3 with the limitation that the sum of m+q is an integer from 1 to 4;
A—B represents CR_{A}═CR_{B}—; C(R_{A})₂—C(R_{B})₂—; or C(R_{A})₂—CR_{B}═;
C—D represents —CR_{C}═CR_{D}—; —C(R_{C})₂—C(R_{D})₂—; ═CR_{C}—C(R_{D})₂—; or —C≡C—;
when m is 0, B—C represents ═CR_{B}—CR_{C}═; —C(R_{B})₂—CR_{C}═; ═CR_{B}—C(R_{C})₂—; ═CR_{B}—C≡; or —C(R_{B})₂—C(R_{C})₂—;
wherein each occurrence of R_{A} independently hydrogen; halogen; —OR_{A′}; —SR_{A′}; —N(R_{A′})₂; —C(O)OR_{A′}; —C(O)R_{A′}; —CONHR_{A′}; —O(C═O)R_{A′}; —O(C═O)OR_{A′}; —NR_{A′}(C═O)R_{A′}; N₃; N₂R_{A′}; cyclic acetal; or cyclic or acyclic, linear or branched aliphatic, heteroaliphatic, aryl, or heteroaryl, optionally substituted with one or more of hydrogen; halogen; —OR_{A′}; —SR_{A′}; —N(R_{A′})₂; —C(O)OR_{A′}; —C(O)R_{A′}; —CONHR_{A′}; —O(C═O)R_{A′}; —O(C═O)OR_{A′}; —NR_{A′}(C═O)R_{A′}; N₃; N₂R_{A′}; cyclic acetal; or cyclic or acyclic, linear or branched substituted or unsubstituted aliphatic, heteroaliphatic, aryl, or heteroaryl moiety; or is an epothilone, desoxyepothilone, or analogues thereof; or is a polymer; carbohydrate; photoaffinity label; or radiolabel;
R_{B} is, independently for each occurrence, hydrogen; halogen; —OR_{B′}; —SR_{B′}; —N(R_{B′})₂; —C(O)OR_{B′}; —C(O)R_{B′}; —CONHR_{B′}; —O(C═O)R_{B′}; —O(C═O)OR_{B′}; —NR_{B′}(C═O)R_{B′}; N₃; N₂R_{B′}; cyclic acetal; or cyclic or acyclic, linear or branched aliphatic, heteroaliphatic, aryl, or heteroaryl, optionally substituted with one or more of hydrogen; halogen; —OR_{B′}; —SR_{B′}; —N(R_{B′})₂; —C(O)OR_{B′}; —C(O)R_{B′}; —CONHR_{B′}; —O(C═O)R_{B′}; —O(C═O)OR_{B′}; —NR_{B′}(C═O)R_{B′}; N₃; N₂R_{B′}; cyclic acetal; or cyclic or acyclic, linear or branched substituted or unsubstituted aliphatic, heteroaliphatic, aryl, or heteroaryl moiety; or is an epothilone, desoxyepothilone, or analogues thereof; or is a polymer; carbohydrate; photoaffinity label; or radiolabel;
R_{C} is, independently for each occurrence, hydrogen; halogen; —OR_{C′}; —SR_{C′}; —N(R_{C′})₂; —C(O)OR_{C′}; —C(O)R_{C′}; —CONHR_{C′}; —O(C═O)R_{′}; —O(C═O)OR_{C′}; —NR_{C′}(C═O)R_{C′}; N₃; N₂R_{C′}; cyclic acetal; or cyclic or acyclic, linear or branched aliphatic, heteroaliphatic, aryl, or heteroaryl, optionally substituted with one or more of hydrogen; halogen; —OR_{C′}; —SR_{C′}; —N(R_{C′})₂; —C(O)OR_{C′}; —C(O)R_{C′}; —CONHR_{C′}; —O(C═O)R_{C′}; —O(C═O)OR_{C′}; —NR_{C′}(C═O)R_{C′}; N₃; N₂R_{C′}; cyclic acetal; or cyclic or acyclic, linear or branched substituted or unsubstituted aliphatic, heteroaliphatic, aryl, or heteroaryl moiety; or is an epothilone, desoxyepothilone, or analogues thereof; or is a polymer; carbohydrate; photoaffinity label; or radiolabel;
R_{D} is, independently for each occurrence, hydrogen; halogen; —OR_{D′}; —SR_{D′}; —N(R_{D′})₂; —C(O)OR_{D′}; —C(O)R_{D′}; —CONHR_{D′}; —O(C═O)R_{D′}; —O(C═O)OR_{D′}; —NR_{D′}(C═O)R_{D′}; N₃; N₂R_{D′}; cyclic acetal; or cyclic or acyclic, linear or branched aliphatic, heteroaliphatic, aryl, or heteroaryl, optionally substituted with one or more of hydrogen; halogen; —OR_{D′}; —SR_{D′}; —N(R_{D′})₂; —C(O)OR_{D′}; —C(O)R_{D′}; —CONHR_{D′}; —O(C═O)R_{D′}; —O(C═O)OR_{D′}; —NR_{D′}(C═O)R_{D′}; N₃; N₂R_{D′}; cyclic acetal; or cyclic or acyclic, linear or branched substituted or unsubstituted aliphatic, heteroaliphatic, aryl, or heteroaryl moiety; or is an epothilone, desoxyepothilone, or analogues thereof; or is a polymer; carbohydrate; photoaffinity label; or radiolabel; or
wherein any two of R_{A}, R_{B}, R_{C} or R_{D} taken together may form a cyclic moiety and may be linked through an oxygen, sulfur, carbon or nitrogen atom, or any two adjacent groups R_{A}, R_{B}, R_{C}, or R_{D}, taken together, may form a 3-6-membered substituted or unsubstituted aliphatic, heteroaliphatic, aryl or heteroaryl ring;
wherein each occurrence of R_{A′}, R_{B′}, R_{C′} and R_{D′} is independently hydrogen; a protecting group; a linear or branched, substituted or unsubstituted, cyclic or acyclic, aliphatic, heteroaliphatic, aryl, heteroaryl, arylalkyl, arylalkenyl, arylalkynyl, or heteroarylalkyl, heteroarylalkenyl, heteroarylalkynyl moiety; or is an epothilone, desoxyepothilone, or analogues thereof; or a polymer; carbohydrate; photoaffinity label; or radiolabel; and
pharmaceutically acceptable derivatives thereof.

3. The compound of claim 1 wherein the compound has the formula (I″): 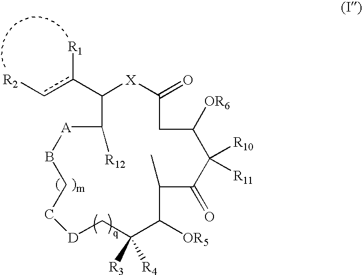
wherein R₁ is hydrogen, lower alkyl, or in conjunction with R₂ may form a cyclic, heterocyclic, aryl, or heteroaryl moitey;
R₂ is a substituted or unsubstituted aryl, heteroaryl, arylalkyl, arylalkenyl, arylalkynyl, or heteroarylalkyl, heteroarylalkenyl, heteroarylalkynyl moiety, which may in conjunction with R₁ form a cyclic, heterocyclic, aryl, or heteroaryl moiety;
the dashed line represents a bond or the absence of a bond;
R₃ and R₄ are each independently hydrogen; or substituted or unsubstituted, linear or branched, cyclic or acyclic aliphatic, heteroaliphatic, aryl, heteroaryl, arylalkyl, arylalkenyl, arylalkynyl, or heteroarylalkyl, heteroarylalkenyl, heteroarylalkynyl moiety, optionally substituted by one or more of hydroxy, protected hydroxy, alkoxy, carboxy, carboxaldehyde, linear or branched alkyl or cyclic acetal, fluorine, amino, protected amino, amino substituted with one or two alkyl or aryl moieties, N-hydroximino, or N-alkoxyimino;
R₅ and R₆ are each independently hydrogen or a protecting group;
R₁₀ and R₁₁ are each independently hydrogen; or substituted or unsubstituted, linear or branched, cyclic or acyclic aliphatic, heteroaliphatic, aryl, heteroaryl, arylalkyl, arylalkenyl, arylalkynyl, or heteroarylalkyl, heteroarylalkenyl, heteroarylalkynyl moiety, optionally substituted by one or more of hydroxy, protected hydroxy, alkoxy, carboxy, carboxaldehyde, linear or branched alkyl or cyclic acetal, fluorine, amino, protected amino, amino substituted with one or two alkyl or aryl moieties, N-hydroximino, or N-alkoxyimino;
R₁₂ is hydrogen; halogen, hydroxy, alkoxy, amino, dialkylamino, alkylamino, fluoro, or substituted or unsubstituted, linear or branched, cyclic or acyclic aliphatic, heteroaliphatic, aryl, heteroaryl, arylalkyl, arylalkenyl, arylalkynyl, or heteroarylalkyl, heteroarylalkenyl, heteroarylalkynyl moiety, optionally substituted by one or more of hydroxy, protected hydroxy, alkoxy, carboxy, carboxaldehyde, linear or branched alkyl or cyclic acetal, fluorine, amino, protected amino, amino substituted with one or two alkyl or aryl moieties, N-hydroximino, or N-alkoxyimino;
X is O, S, C(R₇)₂, or NR₇, wherein each occurrence of R₇ is independently hydrogen or lower alkyl;
m is an integer from 0 to 3 and q is an integer from 1 to 3 with the limitation that the sum of m+q is an integer from 1 to 4;
A—B represents CR_{A}═CR_{B}—; C(R_{A})₂—C(R_{B})₂—; or C(R_{A})₂—CR_{B}═;
C—D represents —CR_{C}═CR_{D}—; —C(R_{C})₂—C(R_{D})₂—; ═CR_{C}—C(R_{D})₂—; or —C≡C—;
when m is 0, B—C represents ═CR_{B}—CR_{C}═; —C(R_{B})₂—CR_{C}═; ═CR_{B}—C(R_{C})₂—; ═CR_{B}—C≡; or —C(R_{B})₂—C(R_{C})₂—;
wherein each occurrence of R_{A} independently hydrogen; halogen; —OR_{A′}; —SR_{A′}; —N(R_{A′})₂; —C(O)OR_{A′}; —C(O)R_{A′}; —CONHR_{A′}; —O(C═O)R_{A′}; —O(C═O)OR_{A′}; —NR_{A′}(C═O)R_{A′}; N₃; N₂R_{A′}; cyclic acetal; or cyclic or acyclic, linear or branched aliphatic, heteroaliphatic, aryl, or heteroaryl, optionally substituted with one or more of hydrogen; halogen; —OR_{A′}; —SR_{A′}; —N(R_{A′})₂; —C(O)OR_{A′}; —C(O)R_{A′}; —CONHR_{A′}; O(C═O)R_{A′}; —O(C═O)OR_{A′}; —NR_{A′}(C═O)R_{A′}; N₃; N₂R_{A′}; cyclic acetal; or cyclic or acyclic, linear or branched substituted or unsubstituted aliphatic, heteroaliphatic, aryl, or heteroaryl moiety; or is an epothilone, desoxyepothilone, or analogues thereof; or is a polymer; carbohydrate; photoaffinity label; or radiolabel;
R_{B} is, independently for each occurrence, hydrogen; halogen; —OR_{B′}; —SR_{B′}; —N(R_{B′})₂; —C(O)OR_{B′}; —C(O)R_{B′}; —CONHR_{B′}; —O(C═O)R_{B′}; —O(C═O)OR_{B′}; —NR_{B′}(C═O)R_{B′}; N₃; N₂R_{B′}; cyclic acetal; or cyclic or acyclic, linear or branched aliphatic, heteroaliphatic, aryl, or heteroaryl, optionally substituted with one or more of hydrogen; halogen; —OR_{B′}; —SR_{B′}; —N(R_{B′})₂; —C(O)OR_{B′}; —C(O)R_{B′}; —CONHR_{B′}; —O(C═O)R_{B′}; —O(C═O)OR_{B′}; —NR_{B′}(C═O)R_{B′}; N₃; N₂R_{B′}; cyclic acetal; or cyclic or acyclic, linear or branched substituted or unsubstituted aliphatic, heteroaliphatic, aryl, or heteroaryl moiety; or is anepothilone, desoxyepothilone, or analogues thereof; or is a polymer; carbohydrate; photoaffinity label; or radiolabel;
R_{C} is, independently for each occurrence, hydrogen; halogen; —OR_{C′}; —SR_{C′}; —N(R_{C′})₂; —C(O)OR_{C′}; —C(O)R_{C′}; —CONHR_{C′}; —O(C═O)R_{C′}; —O(C═O)OR_{C′}; —NR_{C′}(C═O)R_{C′}; N₃; N₂R_{′}; cyclic acetal; or cyclic or acyclic, linear or branched aliphatic, heteroaliphatic, aryl, or heteroaryl, optionally substituted with one or more of hydrogen; halogen; —OR_{C′}; —SR_{C′}; —N(R_{C′})₂; —C(O)OR_{C′}; —C(O)R_{C′}; —CONHR_{C′}; —O(C═O)R_{C′}; —O(C═O)OR_{C′}; —NR_{C′}(C═O)R_{C′}; N₃; N₂R_{C′}; cyclic acetal; or cyclic or acyclic, linear or branched substituted or unsubstituted aliphatic, heteroaliphatic, aryl, or heteroaryl moiety; or is an epothilone, desoxyepothilone, or analogues thereof; or is a polymer; carbohydrate; photoaffinity label; or radiolabel;
R_{D} is, independently for each occurrence, hydrogen; halogen; —OR_{D′}; —SR_{D′}; —N(R_{D′})₂; —C(O)OR_{D′}; —C(O)R_{D′}; —CONHR_{D′}; —O(C═O)R_{D′}; —O(C═O)OR_{D′}; —NR_{D′}(C═O)R_{D′}; N₃; N₂R_{D′}; cyclic acetal; or cyclic or acyclic, linear or branched aliphatic, heteroaliphatic, aryl, or heteroaryl, optionally substituted with one or more of hydrogen; halogen; —OR_{D′}; —SR_{D′}; —N(R_{D′})₂; —C(O)OR_{D′}; —C(O)R_{D′}; —CONHR_{D′}; —O(C═O)R_{D′}; —O(C═O)OR_{D′}; —NR_{D′}(C═O)R_{D′}; N₃; N₂R_{D′}; cyclic acetal; or cyclic or acyclic, linear or branched substituted or unsubstituted aliphatic, heteroaliphatic, aryl, or heteroaryl moiety; or is an epothilone, desoxyepothilone, or analogues thereof; or is a polymer; carbohydrate; photoaffinity label; or radiolabel; or
wherein any two of R_{A}, R_{B}, R_{C} or R_{D} taken together may form a cyclic moiety and may be linked through an oxygen, sulfur, carbon or nitrogen atom, or any two adjacent groups R_{A}, R_{B}, R_{C}, or R_{D}, taken together, may form a 3-6-membered substituted or unsubstituted aliphatic, heteroaliphatic, aryl or heteroaryl ring;
wherein each occurrence of R_{A′}, R_{B′}, R_{C′} and R_{D′} is independently hydrogen; a protecting group; a linear or branched, substituted or unsubstituted, cyclic or acyclic, aliphatic, heteroaliphatic, aryl, heteroaryl, arylalkyl, arylalkenyl, arylalkynyl, or heteroarylalkenyl, heteroarylalkynyl moiety; or is an epothilone, desoxyepothilone, or analogues thereof; or a polymer; carbohydrate; photoaffinity label; or radiolabel; and
pharmaceutically acceptable derivatives thereof.

4. The compound of claim 1 wherein the compound has the formula: 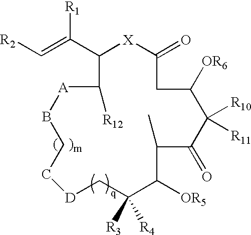
wherein R₁ is hydrogen, or lower alkyl moitey;
R₂ is a substituted or unsubstituted aryl, heteroaryl, arylalkyl, arylalkenyl, arylalkynyl, or heteroarylalkyl, heteroarylalkenyl, heteroarylalkynyl moiety;
R₃ and R₄ are each independently hydrogen; or substituted or unsubstituted, linear or branched, cyclic or acyclic aliphatic, heteroaliphatic, aryl, heteroaryl, arylalkyl, arylalkenyl, arylalkynyl, or heteroarylalkyl, heteroarylalkenyl, heteroarylalkynyl moiety, optionally substituted by one or more of hydroxy, protected hydroxy, alkoxy, carboxy, carboxaldehyde, linear or branched alkyl or cyclic acetal, fluorine, amino, protected amino, amino substituted with one or two alkyl or aryl moieties, N-hydroximino, or N-alkoxyimino;
R₅ and R₆ are each independently hydrogen or a protecting group;
R₁₀ and R₁₁ are each independently hydrogen; or substituted or unsubstituted, linear or branched, cyclic or acyclic aliphatic, heteroaliphatic, aryl, heteroaryl, arylalkyl, arylalkenyl, arylalkynyl, or heteroarylalkyl, heteroarylalkenyl, heteroarylalkynyl moiety, optionally substituted by one or more of hydroxy, protected hydroxy, alkoxy, carboxy, carboxaldehyde, linear or branched alkyl or cyclic acetal, fluorine, amino, protected amino, amino substituted with one or two alkyl or aryl moieties, N-hydroximino, or N-alkoxyimino;
R₁₂ is hydrogen; halogen, hydroxy, alkoxy, amino, dialkylamino, alkylamino, fluoro, or substituted or unsubstituted, linear or branched, cyclic or acyclic aliphatic, heteroaliphatic, aryl, heteroaryl, arylalkyl, arylalkenyl, arylalkynyl, or heteroarylalkyl, heteroarylalkenyl, heteroarylalkynyl moiety, optionally substituted by one or more of hydroxy, protected hydroxy, alkoxy, carboxy, carboxaldehyde, linear or branched alkyl or cyclic acetal, fluorine, amino, protected amino, amino substituted with one or two alkyl or aryl moieties, N-hydroximino, or N-alkoxyimino;
X is O, S, C(R₇)₂, or NR₇, wherein each occurrence of R₇ is independently hydrogen or lower alkyl;
m is an integer from 0 to 3 and q is an integer from 1 to 3 with the limitation that the sum of m+q is an integer from 1 to 4;
A—B represents CR_{A}═CR_{B}—; C(R_{A})₂—C(R_{B})₂—; or C(R_{A})₂—CR_{B}═;
C—D represents —CR_{C}═CR_{D}—; —C(R_{C})₂—C(R_{D})₂—; ═CR_{C}—C(R_{D})₂—; or —C≡C—;
when m is 0, B—C represents ═CR_{B}—CR_{C}═; —C(R_{B})₂—CR_{C}═; ═CR_{B}—C(R_{C})₂—; ═CR_{B}—C≡; or —C(R_{B})₂—C(R_{C})₂—;
wherein each occurrence of R_{A} independently hydrogen; halogen; —OR_{A′}; —SR_{A′}; —N(R_{A′})₂; —C(O)OR_{A′}; —C(O)R_{A′}; —CONHR_{A′}; —O(C═O)R_{A′}; —O(C═O)OR_{A′}; —NR_{A′}(C═O)R_{A′}; N₃; N₂R_{A′}; cyclic acetal; or cyclic or acyclic, linear or branched aliphatic, heteroaliphatic, aryl, or heteroaryl, optionally substituted with one or more of hydrogen; halogen; —OR_{A′}; —SR_{A′}; —N(R_{A′})₂; —C(O)OR_{A′}; —C(O)R_{A′}; —CONHR_{A′}; —O(C═O)R_{A′}; —O(C═O)OR_{A′}; —NR_{A′}(C═O)R_{A′}; N₃; N₂R_{A′}; cyclic acetal; or cyclic or acyclic, linear or branched substituted or unsubstituted aliphatic, heteroaliphatic, aryl, or heteroaryl moiety; or is an epothilone, desoxyepothilone, or analogues thereof; or is a polymer; carbohydrate; photoaffinity label; or radiolabel;
R_{B} is, independently for each occurrence, hydrogen; halogen; —OR_{B′}; —SR_{B′}; —N(R_{B′})₂; —C(O)OR_{B′}; —C(O)R_{B′}; —CONHR_{B′}; —O(C═O)R_{B′}; —O(C═O)OR_{B′}; —NR_{B′}(C═O)R_{B′}; N₃; N₂R_{B′}; cyclic acetal; or cyclic or acyclic, linear or branched aliphatic, heteroaliphatic, aryl, or heteroaryl, optionally substituted with one or more of hydrogen; halogen; —OR_{B′}; —SR_{B′}; —N(R_{B′})₂; —C(O)OR_{B′}; —C(O)R_{B′}; —CONHR_{B′}; —O(C═O)R_{B′}; —O(C═O)OR_{B′}; —NR_{B′}(C═O)R_{B′}; N₃; N₂R_{B′}; cyclic acetal; or cyclic or acyclic, linear or branched substituted or unsubstituted aliphatic, heteroaliphatic, aryl, or heteroaryl moiety; or is an epothilone, desoxyepothilone, or analogues thereof; or is a polymer; carbohydrate; photoaffinity label; or radiolabel;
R_{C} is, independently for each occurrence, hydrogen; halogen; —OR_{C′}; —SR_{C′}; —N(R_{C′})₂; —C(O)OR_{C′}; —C(O)R_{C′}; —CONHR_{C′}; —O(C═O)R_{C′}; —O(C═O)OR_{C′}; —NR_{C′}(C═O)R_{C′}; N₃; N₂R_{C′}; cyclic acetal; or cyclic or acyclic, linear or branched aliphatic, heteroaliphatic, aryl, or heteroaryl, optionally substituted with one or more of hydrogen; halogen; —OR_{C′}; —SR_{C′}; —N(R_{C′})₂; —C(O)OR_{C′}; —C(O)R_{C′}; —CONHR_{C′}; —O(C═O)R_{C′}; —O(C═O)OR_{C′}; —NR_{C′}(C═O)R_{C′}; N₃; N₂R_{C′}; cyclic acetal; or cyclic or acyclic, linear or branched substituted or unsubstituted aliphatic, heteroaliphatic, aryl, or heteroaryl moiety; or is an epothilone, desoxyepothilone, or analogues thereof; or is a polymer; carbohydrate; photoaffinity label; or radiolabel;
R_{D} is, independently for each occurrence, hydrogen; halogen; —OR_{D′}; —SR_{D′}; —N(R_{D′})₂; —C(O)OR_{D′}; —C(O)R_{D′}; —CONHR_{D′}; —O(C═O)R_{D′}; —O(C═O)OR_{D′}; —NR_{D′}(C═O)R_{D′}; N₃; N₂R_{D′}; cyclic acetal; or cyclic or acyclic, linear or branched aliphatic, heteroaliphatic, aryl, or heteroaryl, optionally substituted with one or more of hydrogen; halogen; —OR_{D′}; —SR_{D′}; —N(R_{D′})₂; —C(O)OR_{D′}; —C(O)R_{D′}; —CONHR_{D′}; —O(C═O)R_{D′}; —O(C═O)OR_{D′}; —NR_{D′}(C═O)R_{D′}; N₃; N₂R_{D′}; cyclic acetal; or cyclic or acyclic, linear or branched substituted or unsubstituted aliphatic, heteroaliphatic, aryl, or heteroaryl moiety; or is an epothilone, desoxyepothilone, or analogues thereof; or is a polymer; carbohydrate; photoaffinity label; or radiolabel; or
wherein any two of R_{A}, R_{B}, R_{C} or R_{D} taken together may form a cyclic moiety and may be linked through an oxygen, sulfur, carbon or nitrogen atom, or any two adjacent groups R_{A}, R_{B}, R_{C}, or R_{D}, taken together, may form a 3-6-membered substituted or unsubstituted aliphatic, heteroaliphatic, aryl or heteroaryl ring;
wherein each occurrence of R_{A′}, R_{B′}, R_{C′} and R_{D′} is independently hydrogen; a protecting group; a linear or branched, substituted or unsubstituted, cyclic or acyclic, aliphatic, heteroaliphatic, aryl, heteroaryl, arylalkyl, arylalkenyl, arylalkynyl, or heteroarylalkyl, heteroarylalkenyl, heteroarylalkynyl moiety; or is an epothilone, desoxyepothilone, or analogues thereof; or a polymer; carbohydrate; photoaffinity label; or radiolabel; and
pharmaceutically acceptable derivatives thereof.

5. The compound of claim 4 including at least one feature selected from the group consisting of:
1) A—B and C—D are both double bonds;
2) C—D is —C(R_{C})₂—C(R_{D})₂—, wherein at least one R_{C} is not hydrogen;
3) R₁₀ is methyl, and R₁₁ is hydrogen; and
4) R_{B} is —CH₂F, —CHF₂, or —CF₃.

6. The compound of claim 4, wherein R₂ is one of: 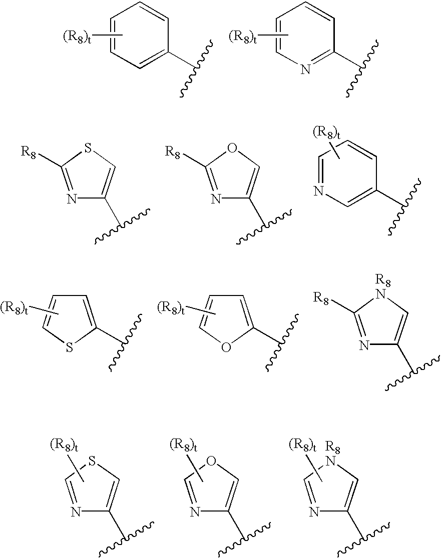
wherein each occurrence of R₈ is independently hydrogen, halogen, —OR₉, —SR₉, —N(R₉)₂, —(CV₂)ₙOR₉, —(CV₂)ₙN(R₉)₂, —(CV₂)ₙSR₉, —(C═O)R₉, —O(C═O)R₉, —(C═O)OR₉; —O(C═O)OR₉; —NH(C═O)R₉, —NH(C═O)OR₉, —(C═O)NHR₉, or a cyclic or acyclic, linear or branched aliphatic, heteroaliphatic, aryl, heteroaryl, arylalkyl, arylalkenyl, arylalkynyl, or heteroarylalkyl, heteroarylalkenyl, heteroarylalkynyl moiety optionally substituted with one or more occurrences of halogen, —OR₉, —SR₉, —N(R₉)₂, —(CV₂)ₙOR₉, —(CV₂)ₙN(R₉)₂, —(CV₂)ₙSR₉, —(C═O)R₉, —O(C═O)R₉, —(C═O)OR₉, —O(C═O)OR₉; —NH(C═O)R₉, —NH(C═O)OR₉, —(C═O)NHR₉, or a cyclic or acyclic, linear or branched, substituted or unsubstituted aliphatic, heteroaliphatic, aryl, heteroaryl, arylalkyl, arylalkenyl, arylalkynyl, or heteroarylalkyl, heteroarylalkenyl, heteroarylalkynyl moiety,
wherein each occurrence of R₉ is independently hydrogen; a protecting group; a cyclic or acyclic, linear or branched, substituted or unsubstituted aliphatic, heteroaliphatic, aryl, or heteroaryl moiety; or is an epothilone, desoxyepothilone or analogues thereof; a polymer; carbohydrate; photoaffinity label; or radiolabel;
wherein each occurrence of V is independently hydrogen, halogen, hydroxyl, thio, amino, alkylamino, or protected hydroxyl, thio or amino; each occurrence of t is independently 0, 1 or 2; and each occurrence of n is independently 0-10.

7. The compound of claim 4, wherein R₂ is one of: 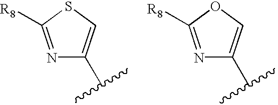
wherein each occurrence of R8 is independently hydrogen, halogen, —OR₉, —SR₉, —N(R₉)₂, —(CV₂)ₙOR₉, —(CV₂)ₙN(R₉)₂, —(CV₂)ₙSR₉, —(C═O)R₉, —O(C═O)R₉, —(C═O)OR₉, —O(C═O)R₉; —NH(C═O)R₉, —NH(C═O)OR₉, —(C═O)NHR₉, or a cyclic or acyclic, linear or branched aliphatic, heteroaliphatic, aryl, heteroaryl, arylalkyl, arylalkenyl, arylalkynyl, or heteroarylalkyl, heteroarylalkenyl, heteroarylalkynyl moiety optionally substituted with one or more occurrences of halogen, —OR₉, —SR₉, —N(R₉)₂, —(CV₂)ₙOR₉, —(CV₂)ₙN(R₉)₂, —(CV₂)ₙSR₉, —(C═O)R₉, —O(C═O)R₉, —(C═O)OR₉, —O(C═O)OR₉; —NH(C═O)R₉, —NH(C═O)OR₉, —(C═O)NHR₉, or a cyclic or acyclic, linear or branched, substituted or unsubstituted aliphatic, heteroaliphatic, aryl, heteroaryl, arylalkyl, arylalkenyl, arylalkynyl, or heteroarylalkyl, heteroarylalkenyl, heteroarylalkynyl moiety,
wherein each occurrence of R₉ is independently hydrogen; a protecting group; a cyclic or acyclic, linear or branched, substituted or unsubstituted aliphatic, heteroaliphatic, aryl, or heteroaryl moiety; or is an epothilone, desoxyepothilone or analogues thereof; a polymer; carbohydrate; photoaffinity label; or radiolabel;
wherein each occurrence of V is independently hydrogen, halogen, hydroxyl, thio, amino, alkylamino, or protected hydroxyl, thio or amino.

8. The compound of claim 4, wherein R₈ is selected from the group consisting of —CH₃, —CH₂OH, and —CH₂NH₂.

9. The compound of claim 3, wherein the sum of m is 0 and q is 1.

10. The compound of claim 3, wherein X is O.

11. The compound of claim 3, wherein X is NH.

12. The compound of claim 3, wherein m is 0 and q is 1 and the compound has the structure:

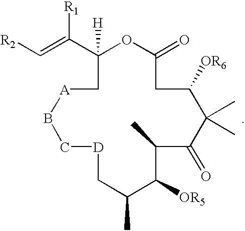

13. The compound of claim 3, wherein m is 0 and q is 1 and the compound has the formula:

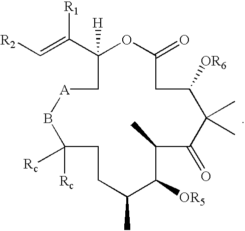

14. The compound of claim 13, wherein R_{C} is, independently for each occurrence, hydrogen, halogen, hydroxyl, alkoxy, amino, or alkylamino.

15. The compound of claim 3, wherein R_{B} is —CF₃, —CF₂H, or —CFH₂.

16. The compound of claim 3, wherein the compound has the formula:

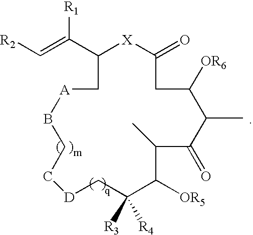

17. The compound of claim 3, wherein each of R₁₀ and R₁₁ are methyl.

18. The compound of claim 3, wherein the the formula: 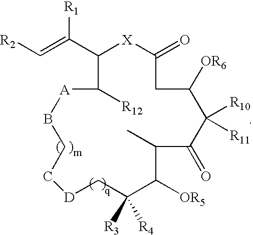
wherein R₀ is a substituted or unsubstituted aryl, heteroaryl, arylalkyl, arylalkenyl, arylalkynyl, or heteroarylalkyl, heteroarylalkenyl, heteroarylalkynyl moiety;
R₃ and R₄ are each independently hydrogen; or substituted or unsubstituted, linear or branched, cyclic or acyclic aliphatic, heteroaliphatic, aryl, heteroaryl, arylalkyl, arylalkenyl, arylalkynyl, or heteroarylalkyl, heteroarylalkenyl, heteroarylalkynyl moiety, optionally substituted by one or more of hydroxy, protected hydroxy, alkoxy, carboxy, carboxaldehyde, linear or branched alkyl or cyclic acetal, fluorine, amino, protected amino, amino substituted with one or two alkyl or aryl moieties, N-hydroximino, or N-alkoxyimino;
R₅ and R₆ are each independently hydrogen or a protecting group;
R₁₀ and R₁₁ are each independently hydrogen; or substituted or unsubstituted, linear or branched, cyclic or acyclic aliphatic, heteroaliphatic, aryl, heteroaryl, arylalkyl, arylalkenyl, arylalkynyl, or heteroarylalkyl, heteroarylalkenyl, heteroarylalkynyl moiety, optionally substituted by one or more of hydroxy, protected hydroxy, alkoxy, carboxy, carboxaldehyde, linear or branched alkyl or cyclic acetal, fluorine, amino, protected amino, amino substituted with one or two alkyl or aryl moieties, N-hydroximino, or N-alkoxyimino;
R₁₂ is hydrogen; halogen, hydroxy, alkoxy, amino, dialkylamino, alkylamino, fluoro, or substituted or unsubstituted, linear or branched, cyclic or acyclic aliphatic, heteroaliphatic, aryl, heteroaryl, arylalkyl, arylalkenyl, arylalkynyl, or heteroarylalkyl, heteroarylalkenyl, heteroarylalkynyl moiety, optionally substituted by one or more of hydroxy, protected hydroxy, alkoxy, carboxy, carboxaldehyde, linear or branched alkyl or cyclic acetal, fluorine, amino, protected amino, amino substituted with one or two alkyl or aryl moieties, N-hydroximino, or N-alkoxyimino;
X is O, S, C(R₇)₂, or NR₇, wherein each occurrence of R₇ is independently hydrogen or lower alkyl;
m is an integer from 0 to 3 and q is an integer from 1 to 3 with the limitation that the sum of m+q is an integer from 1 to 4;
A—B represents CR_{A}═CR_{B}—; C(R_{A})₂—C(R_{B})₂—; or C(R_{A})₂—CR_{B}═;
C—D represents —CR_{C}═CR_{D}—; —C(R_{C})₂—C(R_{D})₂—; ═CR_{C}—C(R_{D})₂—; or —C≡C—;
when m is 0, B—C represents ═CR_{B}—CR_{C}═; —C(R_{B})₂—CR_{C}═; ═CR_{B}—C(R_{C})₂—; ═CR_{B}—C≡; or —C(R_{B})₂—C(R_{C})₂—;
wherein each occurrence of R_{A} independently hydrogen; halogen; —OR_{A′}; —SR_{A′}; —N(R_{A′})₂; —C(O)OR_{A′}; —C(O)R_{A′}; —CONHR_{A′}; —O(C═O)R_{A′}; —O(C═O)OR_{A′}; —NR_{A′}(C═O)R_{A′}; N₃; N₂R_{A′}; cyclic acetal; or cyclic or acyclic, linear or branched aliphatic, heteroaliphatic, aryl, or heteroaryl, optionally substituted with one or more of hydrogen; halogen; —OR_{A′}; —SR_{A′}; —N(R_{A′})₂; —C(O)OR_{A′}; —C(O)R_{A′}; —CONHR_{A′}; —O(C═O)R_{A′}; —O(C═O)OR_{A′}; —NR_{A′}(C═O)R_{A′}; N₃; N₂R_{A′}; cyclic acetal; or cyclic or acyclic, linear or branched substituted or unsubstituted aliphatic, heteroaliphatic, aryl, or heteroaryl moiety; or is an epothilone, desoxyepothilone, or analogues thereof, or is a polymer; carbohydrate; photoaffinity label; or radiolabel;
R_{B} is, independently for each occurrence, hydrogen; halogen; —OR_{B′}; —SR_{B′}; —N(R_{B′})₂; —C(O)OR_{B′}; —C(O)R_{B′}; —CONHR_{B′}; —O(C═O)R_{B′}; —O(C═O)OR_{B′}; —NR_{B′}(C═O)R_{B′}; N₃; N₂R_{B′}; cyclic acetal; or cyclic or acyclic, linear or branched aliphatic, heteroaliphatic, aryl, or heteroaryl, optionally substituted with one or more of hydrogen; halogen; —OR_{B′}; —SR_{B′}; —N(R_{B′})₂; —C(O)OR_{B′}; —C(O)R_{B′}; —CONHR_{B′}; —O(C═O)R_{B′}; —O(C═O)OR_{B′}; —NR_{B′}(C═O)R_{B′}; N₃; N₂R_{B′}; cyclic acetal; or cyclic or acyclic, linear or branched substituted or unsubstituted aliphatic, heteroaliphatic, aryl, or heteroaryl moiety; or is an epothilone, desoxyepothilone, or analogues thereof; or is a polymer; carbohydrate; photoaffinity label; or radiolabel;
R_{C} is, independently for each occurrence, hydrogen; halogen; —OR_{C′}; —SR_{C′}; —N(R_{C′})₂; —C(O)OR_{C′}; —C(O)R_{C′}; —CONHR_{C′}; —O(C═O)R_{C′}; —O(C═O)OR_{C′}; —NR_{C}(C═O)R_{C′}; N₃; N₂R_{C′}; cyclic acetal; or cyclic or acyclic, linear or branched aliphatic, heteroaliphatic, aryl, or heteroaryl, optionally substituted with one or more of hydrogen; halogen; —OR_{C′}; —SR_{C′}; —N(R_{C′})₂; —C(O)OR_{C′}; —C(O)R_{C′}; —CONHR_{C′}; —O(C═O)R_{C′}; —O(C═O)OR_{C′}; —NR_{C′}(C═O)R_{C′}; N₃; N₂R_{C′}; cyclic acetal; or cyclic or acyclic, linear or branched substituted or unsubstituted aliphatic, heteroaliphatic, aryl, or heteroaryl moiety; or is an epothilone, desoxyepothilone, or analogues thereof; or is a polymer; carbohydrate; photoaffinity label; or radiolabel;
R_{D} is, independently for each occurrence, hydrogen; halogen; —OR_{D′}; —SR_{D′}; —N(R_{D′})₂; —C(O)OR_{D′}; —C(O)R_{D′}; —CONHR_{D′}; —O(C═O)R_{D′}; —O(C═O)OR_{D′}; —NR_{D′}(C═O)R_{D′}; N₃; N₂R_{D′}; cyclic acetal; or cyclic or acyclic, linear or branched aliphatic, heteroaliphatic, aryl, or heteroaryl, optionally substituted with one or more of hydrogen; halogen; —OR_{D′}; —SR_{D′}; —N(R_{D′})₂; —C(O)OR_{D′}; —C(O)R_{D′}; —CONHR_{D′}; —O(C═O)R_{D′}; —O(C═O)OR_{D′}; —NR_{D′}(C═O)R_{D′}; N₃; N₂R_{D′}; cyclic acetal; or cyclic or acyclic, linear or branched substituted or unsubstituted aliphatic, heteroaliphatic, aryl, or heteroaryl moiety; or is an epothilone, desoxyepothilone, or analogues thereof; or is a polymer; carbohydrate; photoaffinity label; or radiolabel; or
wherein any two of R_{A}, R_{B}, R_{C} or R_{D} taken together may form a cyclic moiety and may be linked through an oxygen, sulfur, carbon or nitrogen atom, or any two adjacent groups R_{A}, R_{B}, R_{C}, or R_{D}, taken together, may form a 3-6-membered substituted or unsubstituted aliphatic, heteroaliphatic, aryl or heteroaryl ring;
wherein each occurrence of R_{A′}, R_{B′}, R_{C′} and R_{D′} is independently hydrogen; a protecting group; a linear or branched, substituted or unsubstituted, cyclic or acyclic, aliphatic, heteroaliphatic, aryl, heteroaryl, arylalkyl, arylalkenyl, arylalkynyl, or heteroarylalkyl, heteroarylalkenyl, heteroarylalkynyl moiety; or is an epothilone, desoxyepothilone, or analogues thereof; or a polymer; carbohydrate; photoaffinity label; or radiolabel; and
pharmaceutically acceptable derivatives thereof.

19. A pharmaceutical composition for the treatment of cancer comprising a compound of claim 1 and a pharmaceutically acceptable excipient.

20. A method for the synthesis of a compound having the structure below as described in classes and subclasses herein: 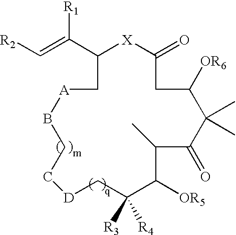
which method comprises:
(1) reacting each of the intermediates (A), (B), (C), (D), and (E) or reacting the intermediates (B), (C), (D), and (E): 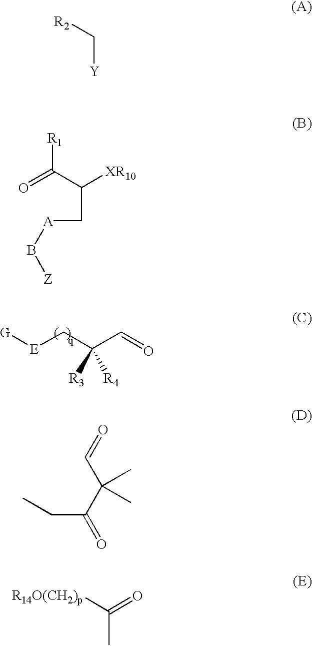
wherein A—B, R₁, R₂, R₃, R₄ and R_{B} are as defined generally herein and in classes and subclasses described herein, and wherein XR₁₀ is NR₇R₁₀, OR₁₀, SR₁₀ or C(R₇)₂R₁₀, wherein R₁₀ is hydrogen, a protecting group, or —(C═O)CH₃; Y is halogen, or a phosphorus ylide; Z is halogen or —(CH₂)ₘ—CR₁₆═C(R₁₇)₂, wherein R₁₆ is hydrogen or a heteroatom substituent (e.g., O, N, S or halogen) and each occurrence of R₁₇ is independently hydrogen or a heteroatom substituent (e.g., O, N, S or halogen); R₁₄ is hydrogen or a protecting group; G—E together represent HC≡C, or CR₁₅R_{C}═CR_{D}, wherein R_{C} and R_{D} are as defined herein, R₁₅ is hydrogen, disubstituted borane, trisubstituted tin, or trisubstituted silane; m is 0-3; q is 1-3, wherein the sum of m and q is 1, 2, 3, 4 or 5; and p is 0-2, in any order and under suitable conditions to generate an intermediate having any one of the structures (F), (G), (H), (I) or (J): 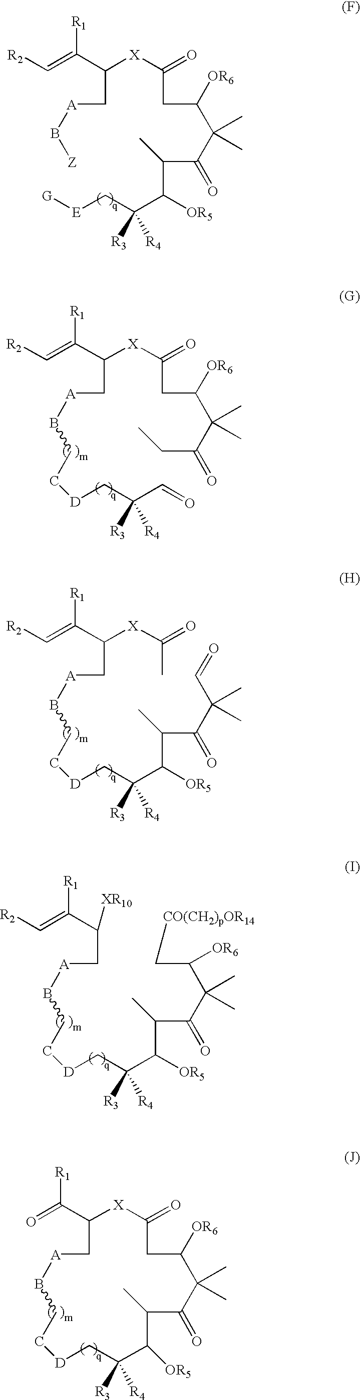
(2) reacting any one of the intermediates (F), (G), (H), or (I), in the presence of a macrocyclization reagent, or reacting the intermediate (J) with (A) under suitable conditions, and optionally further reacting with one or more additional reagents to generate the compound (I′″).
